(19) United States
(12) Patent Application Publication
(10) Pub. No.: US 2021/0371537 A1
(43) Pub. Date: Dec. 2, 2021

(54) ANTI-TNFR2 ANTIBODIES AND USES THEREOF

(71) Applicant: Merrimack Pharmaceuticals, Inc., Cambridge, MA (US)

(72) Inventors: Eric M. TAM, Cooperstown, NY (US); Marco MUDA, New Haven, CT (US); Klaus Andreas RAUE, Cambridge, MA (US); Vinodh B. KURELLA, Malden, MA (US); Daryl Clark DRUMMOND, Lincoln, MA (US); Ross Bane FULTON, Southborough, MA (US); Fabien DEPIS, Brookline, MA (US); Anne-Sophie DUGAST, Boston, MA (US); Jian TANG, Newton, MA (US); Sandeep KUMAR, Lexington, MA (US)

(21) Appl. No.: 17/276,686

(22) PCT Filed: Sep. 18, 2019
(86) PCT No.: PCT/US2019/051756

§ 371 Date: Mar. 16, 2021

Related U.S. Application Data
(60) Provisional application No. 62/812,859, filed on Mar. 1, 2019.
(60) Provisional application No. 62/760,777, filed on Nov. 13, 2018.
(60) Provisional application No. 62/732,846, filed on Sep. 18, 2018.

(51) Int. Cl. C07K 16/28; A61K 47/68; A61P 35/00; A61K 39/395; A61P 37/06

(52) U.S. Cl. CPC C07K 16/2878; A61K 47/6849; A61P 35/00; A61K 2039/505; A61P 37/06; C07K 16/2818; C07K 16/2827; A61K 39/3955

(57) ABSTRACT

Anti-TNFR2 antibodies which bind to particular human TNFR2 epitopes, therapeutic compositions comprising the anti-TNFR2 antibodies, and methods of using such antibodies and compositions in the treatment of cancer are disclosed.

### RELATED APPLICATIONS

This application claims priority to, and the benefit of, U.S. Provisional Application No. 62/732,846, filed Sep. 18, 2018; U.S. Provisional Application No. 62/760,777, filed Nov. 13, 2018; and U.S. Provisional Application No. 62/812,859, filed Mar. 1, 2019. The contents of the aforementioned applications are hereby incorporated by reference.

### BACKGROUND

Recent studies have shown that enhancing the body's own ability to fight disease through the regulation of immune responses is an attractive alternative and/or complement to traditional therapeutic platforms. For example, studies have shown that enhancing the activity to T-lymphocytes to target and treat various diseases (e.g., cancer or infectious disease) is therapeutically beneficial. Inhibiting the ability of T-regulatory cells (Tregs) to suppress the activity of T-lymphocytes is one potential mechanism to increase immune responses against disease.

Tumor Necrosis Factor Receptor 2 (TNFR2), also known as TNFRSF1B and CD120b, is a co-stimulatory member of the tumor necrosis factor receptor superfamily (TNFRSF), which includes proteins such as GITR, OX40, CD27, CD40, and 4-1BB (CD137). TNFR2 is a cell-surface receptor that is expressed on T cells and has been shown to enhance the activation of effector T (Teff) cells and decrease Treg-mediated suppression. Through the regulation of TRAF2/3 and NF-kB signaling, TNFR2 can mediate the transcription of genes that promote cell survival and proliferation. TNFR2 can be expressed on cancer cells, tumor-infiltrating Tregs, and effector T cells. Given the ongoing need for improved strategies for targeting diseases such as cancer, benefits from enhanced immune responses, in particular, T cell responses, novel agents and methods that modulate Treg activity are highly desirable.

### SUMMARY

Provided herein are isolated antibodies, such as recombinant monoclonal antibodies (e.g., human antibodies), that specifically bind to particular epitopes on TNFR2 (e.g., human TNFR2) and have therapeutically desirable properties. Accordingly, the antibodies described herein can be used to, e.g., inhibit tumor growth, treat cancer, treat autoimmune diseases, treat graft-versus-host disease, and promote graft survival and/or reduce graft rejection.

In one embodiment, provided herein are antibodies (e.g., isolated monoclonal antibodies) that bind all or a portion of amino acid residues 23-54 of human TNFR2 (SEQ ID NO: 1), and do not bind one or more amino acid residues within 55-77 (e.g., do not bind one or more amino acids within 60-77, 65-77, 70-77, 75-77, 55-75, 55-70, 55-65, or 55-60) of human TNFR2 (SEQ ID NO: 1). In some embodiments, the antibodies do not bind one or more amino acid residues within 78-118, 120-143, and/or 161-200 of human TNFR2 (SEQ ID NO: 1).

In another embodiment, provided herein are isolated antibodies that exhibit reduced binding (e.g., at least 50% reduced binding, at least 60% reduced binding, at least 70% reduced binding, at least 80% reduced binding, or at least 90% reduced binding) to a mutant human TNFR2 comprising a substitution (e.g., a non-conservative amino acid substitution, e.g., an alanine substitution) at amino acid residue 48 or amino acid residue 68 of human TNFR2 (SEQ ID NO: 1). In some embodiments, the antibodies do not exhibit reduced binding (e.g., not more than 20% reduced binding or not more than 10% reduced binding) to a mutant human TNFR2 comprising a substitution (e.g., a non-conservative amino acid substitution, e.g., an alanine substitution) at one or more amino acid residues selected from the group consisting of residues 37, 39, 42, 49, 51, 56, 65, 66, 69, 86, 89, and 91. In other embodiments, the antibodies do not bind amino acid residues 97-118, 120-143 and/or 161-200 of human TNFR2 (SEQ ID NO: 1). In other embodiments, reduced binding of the antibodies to the mutant TNFR2 is assessed by yeast surface display.

In another embodiment, provided herein are isolated antibodies, which bind to TNFR2 chimera 3 (SEQ ID NO: 11 or 12), and do not bind TNFR2 chimera 0 (SEQ ID NO: 5 or 6). In some embodiments, the antibodies do not bind TNFR2 chimera 0 with a K_{D} of less than 1×10⁻⁵ M, less than 1×10⁻⁶ M, less than 1×10⁻⁷ M, or less than 1×10⁻⁸ M).

In another embodiment, provided herein are isolated antibodies that bind all or a portion of amino acid residues 55-96 of human TNFR2 (SEQ ID NO: 1), and do not bind one or more amino acid residues within 23-54 (e.g., do not bind one or more amino acids within 23-44, 23-36, 23-30, 23-25, 25-44, 30-44, 35-44, or 40-44) of human TNFR2 (SEQ ID NO: 1). In some embodiments, the antibodies do not bind amino acid residues 97-118, 120-143 and/or 161-200 of human TNFR2 (SEQ ID NO: 1).

In another embodiment, provided herein are isolated antibodies that exhibit reduced binding (e.g., at least 50% reduced binding, at least 60% reduced binding, at least 70% reduced binding, at least 80% reduced binding, or at least 90% reduced binding) to a mutant human TNFR2 comprising a substitution (e.g., a non-conservative amino acid substitution, e.g., an alanine substitution) at one or more amino acid residues selected from the group consisting of (i) residues 37, 44, 51, 52, 55, 58, 59, 61, 62, 72, 74, 76, 78, and 87 of human TNFR2 (SEQ ID NO: 1), or (ii) residues 55 and 72 of human TNFR2 (SEQ ID NO: 1), as compared to wild-type human TNFR2 (SEQ ID NO: 1). In some embodiments, the antibodies do not exhibit reduced binding (e.g., not more than 20% reduced binding or not more than 10% reduced binding) to a mutant human TNFR2 comprising a substitution (e.g., a non-conservative amino acid substitution, e.g., an alanine substitution) at one or more amino acid residues selected from the group consisting of residues 39, 41, 80, 112, and 113. In other embodiments, the antibodies do not bind amino acid residues 97-118, 120-143 and/or 161-200 of human TNFR2 (SEQ ID NO: 1). In other embodiments, binding of the antibodies to the mutant human TNFR2 is reduced by at least about 50%, as assessed by yeast surface display. In other embodiments, reduced binding of the antibodies to the mutant TNFR2 is assessed by yeast surface display.

In another embodiment, provided herein are isolated antibodies that bind TNFR2 chimera 7 (SEQ ID NO: 19 or 20) (e.g., bind TNFR2 chimera 7 with a K_{D} of less than 1×10⁻⁷ M), and do not bind TNFR2 chimera 4 (SEQ ID NO: 13 or 14) (e.g., do not bind TNFR2 chimera 4 with a K_{D} of less than 1×10⁻⁷ M).

In another embodiment, provided herein are isolated antibodies that bind all or a portion of amino acid residues 78-118 of human TNFR2 (SEQ ID NO: 1), and do not bind one or more amino acid residues within 23-77 of human TNFR2 (SEQ ID NO: 1).

In another embodiment, provided herein are isolated antibodies that bind TNFR2 chimera 1 (SEQ ID NO: 7 or 8) and do not bind TNFR2 chimera 2 (SEQ ID NO: 9 or 10) (e.g., do not bind TNFR2 chimera 2 (SEQ ID NO: 9 or 10) with a K_{D} of less than 1×10⁻⁷ M).

In another embodiment, provided herein are isolated antibodies that bind all or a portion of amino acid residues 120-257 of human TNFR2 (SEQ ID NO: 1), and do not significantly inhibit binding of TNF-alpha to human TNFR2.

In another embodiment, provided herein are isolated antibodies which bind to human TNFR2 (e.g., such as those described herein), wherein the antibodies have been modified to enhance effector function relative to the same antibodies in unmodified form, for example, by introducing amino acid substitutions that enhance effector function. In some embodiments, the antibodies exhibit increased anti-tumor activity relative to the same antibodies in unmodified form. In other embodiments, the antibodies exhibit one of the following: (a) bind all or a portion of amino acid residues 23-54 of human TNFR2 (SEQ ID NO: 1), and do not bind one or more amino acid residues within 55-77 of human TNFR2 (SEQ ID NO: 1); (b) bind all or a portion of amino acid residues 55-96 of human TNFR2 (SEQ ID NO: 1), and inhibit binding of TNF-alpha to human TNFR2 by at least about 50%, (c) bind all or a portion of amino acid residues 78-118 of human TNFR2 (SEQ ID NO: 1), and do not bind one or more amino acid residues within 23-77 or 119-200 of human TNFR2 (SEQ ID NO: 1), or (d) bind all or a portion of amino acid residues 120-257 of human TNFR2 (SEQ ID NO: 1), do not bind one or more amino acid residues within 78-118 of human TNFR2 (SEQ ID NO: 1), and do not inhibit the binding of TNF-alpha to human TNFR2.

In some embodiments, the antibodies described herein agonize TNFR2 activity. In other embodiments, the antibodies described herein inhibit tumor growth independent of their ability to agonize TNFR2 signaling and/or independent of their ability to inhibit TNF-alpha binding to TNFR2.

In another embodiment, provided herein are isolated antibodies which bind to human TNFR2 and comprise heavy and light chain CDRs of the heavy and light chain variable regions comprising the amino acid sequences set forth in (a) SEQ ID NOs: 71 and 72, respectively, (b) SEQ ID NOs: 74 and 86, respectively, (c) SEQ ID NOs: 170 and 171, respectively, (d) SEQ ID NOs: 148 and 149, respectively, or (e) SEQ ID NOs: 126 and 127, respectively.

In another embodiment, provided herein are isolated antibodies which bind to human TNFR2 and comprise (a) heavy chain CDR1, CDR2, and CDR3 sequences comprising SEQ ID NOs: 47, 48, and 49, respectively, and light chain CDR1, CDR2, and CDR3 sequences comprising SEQ ID NOs: 50, 51, and 52, respectively, (b) heavy chain CDR1, CDR2, and CDR3 sequences comprising SEQ ID NOs: 53, 54, and 55, respectively, and light chain CDR1, CDR2, and CDR3 sequences comprising SEQ ID NOs: 56, 57, and 58, respectively, (c) heavy chain CDR1, CDR2, and CDR3 sequences comprising SEQ ID NOs: 59, 60, and 61, respectively, and light chain CDR1, CDR2, and CDR3 sequences comprising SEQ ID NOs: 62, 63, and 64, respectively, (d) heavy chain CDR1, CDR2, and CDR3 sequences comprising SEQ ID NOs: 65, 66, and 67, respectively, and light chain CDR1, CDR2, and CDR3 sequences comprising SEQ ID NOs: 68, 69, and 70, respectively, (e) heavy chain CDR1, CDR2, and CDR3 sequences comprising SEQ ID NOs: 152, 153, and 154, respectively, and light chain CDR1, CDR2, and CDR3 sequences comprising SEQ ID NOs: 155, 156, and 157, respectively, (f) heavy chain CDR1, CDR2, and CDR3 sequences comprising SEQ ID NOs: 158, 159, and 160, respectively, and light chain CDR1, CDR2, and CDR3 sequences comprising SEQ ID NOs: 161, 162, and 163, respectively, (g) heavy chain CDR1, CDR2, and CDR3 sequences comprising SEQ ID NOs: 164, 165, and 166, respectively, and light chain CDR1, CDR2, and CDR3 sequences comprising SEQ ID NOs: 167, 168, and 169, respectively, (h) heavy chain CDR1, CDR2, and CDR3 sequences comprising SEQ ID NOs: 130, 131, and 132, respectively, and light chain CDR1, CDR2, and CDR3 sequences comprising SEQ ID NOs: 133, 134, and 135, respectively, or (i) heavy chain CDR1, CDR2, and CDR3 sequences comprising SEQ ID NOs: 108, 109, and 110, respectively, and light chain CDR1, CDR2, and CDR3 sequences comprising SEQ ID NOs: 111, 112, and 113, respectively. In some embodiments, the antibodies are human, humanized, or chimeric antibodies.

In another embodiment, provided herein are isolated antibodies which bind to human TNFR2 and comprise a heavy chain variable region and a light chain variable region, wherein the heavy chain variable region comprises an amino acid sequence selected from the group consisting of SEQ ID NOs: 71, 73, 74, 75, 76, 77, 78, 79, 80, 81, 82, 83, 84, 126, 148, and 170 or an amino acid sequence which is at least 80%, 85%, 90%, 95%, 96%, 97%, 98%, or 99% identical to an amino acid sequence selected from the group consisting of SEQ ID NOs: 71, 73, 74, 75, 76, 77, 78, 79, 80, 81, 82, 83, 84, 126, 148, and 170, and/or the light chain variable region comprises an amino acid sequence selected from the group consisting of SEQ ID NOs: 72, 85, 86, 87, 88, 89, 90, 91, 92, 93, 94, 95, 96, 97, 98, 99, 100, 127, 149, and 171, or an amino acid sequence which is at least 80%, 85%, 90%, 95%, 96%, 97%, 98%, or 99% identical to an amino acid sequence selected from the group consisting of SEQ ID NOs: 72, 85, 86, 87, 88, 89, 90, 91, 92, 93, 94, 95, 96, 97, 98, 99, 100, 127, 149, and 171.

In another embodiment, provided herein are isolated antibodies which bind to human TNFR2 and comprise a heavy chain variable region comprising the amino acid sequence of SEQ ID NO: 71, and a light chain variable region comprising the amino acid sequence of SEQ ID NO: 72. In some embodiments, the antibodies comprise heavy and light chain variable region sequences which are at least 80% (e.g., at least 85%, 90%, 95%, 96%, 97%, 98%, or 99%) identical to the amino acid sequences set forth in SEQ ID NOs: 71 and 72, respectively.

In another embodiment, provided herein are isolated antibodies which bind to human TNFR2 and comprise a heavy chain variable region comprising the amino acid sequence of SEQ ID NO: 74, and a light chain variable region comprising the amino acid sequence of SEQ ID NO: 86. In some embodiments, the antibodies comprise heavy and light chain variable region sequences which are at least 80% (e.g., at least 85%, 90%, 95%, 96%, 97%, 98%, or 99%) identical to the amino acid sequences set forth in SEQ ID NOs: 74 and 86, respectively.

In another embodiment, provided herein are isolated antibodies which bind to human TNFR2 and comprise a heavy chain variable region comprising the amino acid sequence of SEQ ID NO: 170, and a light chain variable region comprising the amino acid sequence of SEQ ID NO: 171. In some embodiments, the antibodies comprise heavy and light chain variable region sequences which are at least 80% (e.g., at least 85%, 90%, 95%, 96%, 97%, 98%, or 99%) identical to the amino acid sequences set forth in SEQ ID NOs: 170 and 171, respectively.

In another embodiment, provided herein are isolated antibodies which bind to human TNFR2 and comprise a heavy chain variable region comprising the amino acid sequence of SEQ ID NO: 148, and a light chain variable region comprising the amino acid sequence of SEQ ID NO: 149. In some embodiments, the antibodies comprise heavy and light chain variable region sequences which are at least 80% (e.g., at least 85%, 90%, 95%, 96%, 97%, 98%, or 99%) identical to the amino acid sequences set forth in SEQ ID NOs: 148 and 149, respectively.

In another embodiment, provided herein are isolated antibodies which bind to human TNFR2 and comprise a heavy chain variable region comprising the amino acid sequence of SEQ ID NO: 126, and a light chain variable region comprising the amino acid sequence of SEQ ID NO: 127. In some embodiments, the antibodies comprise heavy and light chain variable region sequences which are at least 80% (e.g., at least 85%, 90%, 95%, 96%, 97%, 98%, or 99%) identical to the amino acid sequences set forth in SEQ ID NOs: 126 and 127, respectively.

In another embodiment, provided herein are isolated antibodies which bind to human TNFR2 and comprise heavy and light chain sequences which are at least 80% (e.g., at least 85%, 90%, 95%, 96%, 97%, 98%, or 99%) or 100% identical to the amino acid sequences set forth in SEQ ID NOs: 101 and 102, respectively.

In another embodiment, provided herein are isolated antibodies which bind to human TNFR2 and comprise heavy and light chain sequences which are at least 80% (e.g., at least 85%, 90%, 95%, 96%, 97%, 98%, or 99%) or 100% identical to the amino acid sequences set forth in SEQ ID NOs: 150 and 151, respectively.

In another embodiment, provided herein are isolated antibodies which bind to human TNFR2 and comprise heavy and light chain sequences which are at least 80% (e.g., at least 85%, 90%, 95%, 96%, 97%, 98%, or 99%) or 100% identical to the amino acid sequences set forth in SEQ ID NOs: 128 and 129, respectively.

In another embodiment, provided herein are isolated antibodies which bind to human TNFR2 and comprise heavy and light chain sequences which are at least 80% (e.g., at least 85%, 90%, 95%, 96%, 97%, 98%, or 99%) or 100% identical to the amino acid sequences set forth in SEQ ID NOs: 106 and 107, respectively.

In some embodiments, the antibodies described herein are agonistic antibodies. For example, the antibodies activate NF-κB signaling, promote T cell proliferation (e.g., CD4+ and CD8+ T cells), and/or co-stimulate T cells. In other embodiments, the antibodies decrease the abundance of regulatory T cells (e.g., in the T cell compartment). In other embodiments, the antibodies induce a long-term anti-cancer effect, for example, by inducing the development of anti-cancer memory T cells.

In some embodiments, the antibodies described herein are IgG1, IgG2, IgG3, or IgG4, or variants thereof. In other embodiments, the antibodies comprise a variant Fc region. In other embodiments, the variant Fc region increases binding to Fcγ receptors (e.g., FcγRIIb receptor) relative to binding observed with the corresponding non-variant Fc region. In other embodiments, the variant Fc region increases antibody clustering relative to the corresponding non-variant Fc region. In other embodiments, the antibody co-stimulates T cells (e.g., CD8+ T cells). In other embodiments, the variant Fc region is a variant IgG1 Fc region. In other embodiments, the variant IgG1 Fc region comprises a substitution or substitutions selected from the group consisting of: (a) S267E, (b) S267E/L328F, (c) G237D/P238D/P271G/A330R, (d) E233D/P238D/H268D/P271G/A330R, (e) G237D/P238D/H268D/P271G/A330R, and (f) E233D/G237D/P238D/H268D/P271G/A330R.

In some embodiments, the antibodies described herein bind a discontinuous epitope on TNFR2. In other embodiments, the antibodies described herein are monoclonal antibodies. In other embodiments, the antibodies described herein are human, humanized, or chimeric antibodies. In other embodiments, the antibodies described herein are multi-specific antibodies (e.g., bispecific antibodies) or immunoconjugates comprising the antigen-binding domains (e.g., variable regions or heavy and light chains) of the anti-TNFR2 antibodies described herein. In other embodiments, the antibodies are selected from the group consisting of a single-chain antibody, Fab, Fab′, F(ab′)2, Fd, Fv, or domain antibody.

In some embodiments, the antibodies described herein bind to one or more of the following positions on human TNFR2 (numbering according to SEQ NO: 104): Y24, Q26, Q29, M30, and K47.

In some embodiments, provided herein are antibodies which bind to the same epitope on human TNFR2 as the anti-TNFR2 antibodies described herein. In other embodiments, provided herein are antibodies which compete for binding to human TNFR2 with the anti-TNFR2 antibodies described herein.

In some embodiments, the antibodies described herein are antibodies produced by the hybridoma designated ABV3, ABV4, ABV7, ABV12, ABV13, ABV14, ABV15, ABV18, and/or ABV19. In some embodiments, the hybridoma antibodies have been humanized. In other embodiments, the antibodies comprise the VHCDR1-3 and VLCDR1-3 sequences of an antibody produced by the hybridoma designated ABV3, ABV4, ABV7, ABV12, ABV13, ABV14, ABV15, ABV18, or ABV19.

In another aspect, provided herein are nucleic acids encoding the heavy and/or light chain variable region(s) of the antibodies described herein. Also provided are expression vectors comprising the nucleic acids and cells (e.g., host cells) transformed with the expression vectors.

In another aspect, provided herein are compositions (e.g., pharmaceutical compositions), which comprise an antibody described herein, and a carrier (e.g., a pharmaceutically acceptable carrier). Also provided are kits comprising the antibodies described herein, and instructions for use.

In another aspect, provided herein are methods of increasing T cell proliferation, co-stimulating an effector T cell, and/or reducing or depleting the number of regulatory T cells in a subject comprising administering an effective amount of an antibody described herein to the subject to achieve increased T cell proliferation, effector T cell co-stimulation, and/or a reduction in or depletion of the number of regulatory T cells.

In another aspect, provided herein are methods of treating cancer comprising administering to a subject in need thereof a therapeutically effective amount of an anti-TNFR2 antibody described herein. In some embodiments, provided is the use of an anti-TNFR2 antibody described herein for the manufacture of a medicament for the treatment of a subject having cancer, or an anti-TNFR2 antibody described herein for use in the treatment of a subject having cancer.

In another aspect, provided herein are methods of treating cancer comprising administering to a subject in need thereof a therapeutically effective amount of an anti-TNFR2 antibody described herein, wherein the antibody has effector function and does not significantly inhibit binding of TNF-alpha to TNFR2. In some embodiments, provided is the use of an anti-TNFR2 antibody for the manufacture of a medicament for the treatment of cancer, or an anti-TNFR2 antibody for use in the treatment of a subject having cancer, wherein the antibody has effector function and does not significantly inhibit binding of TNF-alpha to TNFR2.

In another aspect, provided herein are methods of treating cancer comprising administering to a subject in need thereof a therapeutically effective amount of an anti-TNFR2 antibody described herein, wherein the antibody has effector function and agonizes TNFR2 receptor signaling. In some embodiments, provided is the use of an anti-TNFR2 antibody for the manufacture of a medicament for the treatment of a subject having cancer, or an anti-TNFR2 antibody for use in the treatment of a subject having cancer, wherein the antibody has effector function and agonizes TNFR2 receptor signaling.

In another aspect, provided herein are methods of treating cancer comprising administering to a subject in need thereof a therapeutically effective amount of an anti-TNFR2 antibody described herein, wherein the antibody has effector function. In some embodiments, provided is the use of an anti-TNFR2 antibody for the manufacture of a medicament for the treatment of a subject having cancer, or an anti-TNFR2 antibody for use in the treatment of a subject having cancer, wherein the antibody has effector function.

In some embodiments, the cancer to be treated is non-small cell lung cancer, breast cancer, ovarian cancer, or colorectal cancer.

In some embodiments, one or more additional therapeutic agents (e.g., immunomodulatory drug, cytotoxic drug, targeted therapeutic, cancer vaccine) are administered in the methods of treating cancer described above. In other embodiments, the method, use, or antibody described herein induces a long-term anti-cancer effect. In other embodiments, the method, use, or antibody described herein induces the development of anti-cancer memory T cells.

In another aspect, provided herein are methods of treating autoimmune diseases or disorders comprising administering to a subject in need thereof a therapeutically effective amount of an anti-TNFR2 antibody described herein. In some embodiments, provided is the use of an anti-TNFR2 antibody described herein for the manufacture of a medicament for the treatment of a subject having an autoimmune disease or disorder, or an anti-TNFR2 antibody described herein for use in the treatment of a subject having an autoimmune disease or disorder.

In some embodiments, the autoimmune disease or disorder to be treated is graft-versus-host disease, rheumatoid arthritis, Crohn's disease, multiple sclerosis, colitis, psoriasis, autoimmune uveitis, pemphigus, epidermolysis bullosa, or type 1 diabetes. In other embodiments, one or more additional therapeutic agents are administered in the methods of treating autoimmune diseases or disorders.

In another aspect, provided herein are methods of promoting graft survival or reducing graft rejection in a subject who has received or will receive a cell, tissue, or organ transplant comprising administering to the subject an effective amount (e.g., a therapeutically effective amount) of an anti-TNFR2 antibody described herein to promote graft survival or reduce graft rejection. In some embodiments, provided is the use of an anti-TNFR2 antibody described herein for the manufacture of a medicament for promoting graft survival or reducing graft rejection in a subject who has received or will receive a cell, tissue, or organ transplant, or an anti-TNFR2 antibody described herein for use in promoting graft survival or reducing graft rejection in a subject who has received or will receive a cell, tissue, or organ transplant.

In some embodiments, the graft is an allograft (e.g., a cell, tissue, or organ allograft). In other embodiments, the graft rejection is in a recipient who has received or will receive a cell, tissue, or organ allograft. In other embodiments, one or more additional therapeutic agents are administered in the methods of promoting graft survival or reducing graft rejection.

In another aspect, provided herein are methods of treating, preventing, or reducing graft-versus-host disease in a subject who has or will receive a cell, tissue, or organ transplant comprising administering to the subject an effective amount (e.g., a therapeutically effective amount) of an anti-TNFR2 antibody described herein. In some embodiments, provided is the use of an anti-TNFR2 antibody described herein for the manufacture of a medicament for treating, preventing, or reducing graft-versus-host disease in a subject who has or will receive a cell, tissue, or organ transplant, or an anti-TNFR2 antibody described herein for use in treating, preventing, or reducing graft-versus-host disease in a subject who has or will receive a cell, tissue, or organ transplant. In other embodiments, one or more additional therapeutic agents are administered in the methods of treating, preventing, or reducing graft-versus-host disease.

Also provided herein are methods of detecting TNFR2 (e.g., human TNFR2) comprising contacting a sample (e.g., a biological sample) with an anti-TNFR2 antibody described herein under conditions that allow for formation of a complex between the antibody and TNFR2 protein and detecting the formation of a complex. In some embodiments, provided is the use of an anti-TNFR2 antibody described herein for detecting TNFR2 (e.g., human TNFR2) in a sample (e.g., a biological sample), comprising contacting the sample with the anti-TNFR2 antibody under conditions that allow for formation of a complex between the antibody and TNFR2 proteins, and detecting the formation of the complex.

### BRIEF DESCRIPTION OF THE DRAWINGS

FIG. 1 is an alignment of human and mouse TNFR2 amino acid sequences.

FIG. 2A is a graph showing on-cell binding of the indicated anti-mouse TNFR2 antibodies on CHO cells overexpressing TNFR2. FIG. 2B is a graph showing binding of the indicated anti-mouse TNFR2 antibodies on wild-type CHO cells.

FIG. 3 is a graph showing the binding affinities of the indicated antibodies (monovalent K_{D}) for his-tagged mouse TNFR2 using the ForteBio assay.

FIG. 4 is a graph showing the ability of the indicated anti-mouse TNFR2 antibodies to block the binding of TNF to TNFR2, as assessed by ELISA.

FIG. 5A is a schematic summarizing the structure of chimeric receptors of mouse and human TNFR2 used for epitope mapping. FIG. 5B is a schematic showing binding of the indicated anti-mouse TNFR2 antibodies to each chimera (dark shading: binding; no shading: no binding).

FIG. 6 is a homology model of mouse TNFR2 (space-filling model) bound to mouse TNF (ribbon model). Amino acid positions at which M3 binding was significantly disrupted by mutations are mapped (−, black; +, dark grey).

FIG. 7A is a graph showing the effects of the indicated anti-mouse TNFR2 antibodies on tumor growth in the CT26 mouse model. FIG. 7B shows a histogram representation of tumor size at day 18 post-randomization. FIG. 7C is a survival curve showing the survival of animals as determined by time to reach a human end-point based on tumor size. FIG. 7D is a survival curve showing survival of previously cured mice re-challenged with CT26 tumor cells.

FIG. 8A is a graph showing the effects of 1 mg or 0.3 mg M36, with or without mutations that affect effector function, on tumor growth in the CT26 mouse model. FIG. 8B shows a histogram representation of tumor size at day 18 post-randomization. FIG. 8C is a graph showing the effects of 0.3 mg M3, with or without mutations that affect effector function, on tumor growth in the CT26 mouse model. FIG. 8D shows a histogram representation of tumor size at day 18 post-randomization. CT26 cells (5×10E5) were inoculated subcutaneously in 6-week-old female Balb/c mice (7 mice/group).

FIGS. 8E-8J are graphs showing the effects of 3×0.3 mg Y9, with or without mutations that affect effector function, on tumor growth in a CT26 (FIGS. 8E-8G) or Wehi164 (FIGS. 8H-8J) mouse model).

FIG. 9A is a graph showing the effects of the indicated anti-mouse TNFR2 antibodies on tumor growth in the CT26 mouse model. FIG. 9B shows a histogram representation of tumor size at day 18 post-randomization.

FIGS. 10A-10I are graphs showing the effects of 1 mg (FIGS. 10A-10F) or 0.3 mg (FIGS. 10G-10I) of the indicated antibodies on tumor growth in the EMT6 mouse model.

FIGS. 11A and 11B are graphs showing the anti-tumor response of antibody Y9 and an anti-PD-1 antibody on anti-PD-1 resistant (MBT-2) and anti-PD-1 sensitive (SaI/N) tumor models.

FIG. 12 shows a series of graphs on the anti-tumor activity of antibody Y9 alone, anti-PD-1 antibody alone, and the combination of Y9 and the anti-PD-1 antibody in various syngeneic models (WEHI164, SaI/N, MBT2, CT26, and EMT6).

FIG. 13 is a graph showing the effects of antibody Y9 and an anti-CTLA4 antibody on body weight of healthy mice.

FIG. 14 is a graph showing the effects of antibody Y9 and an anti-CTLA4 antibody on spleen weight of healthy mice.

FIGS. 15A and 15B are graphs showing the effects of antibody Y9 and an anti-CTLA4 antibody on levels of alanine aminotransferase (ALT; FIG. 15A) and aspartate aminotransferase (AST; FIG. 15B) in healthy mice.

FIGS. 16A-16D show the effects of antibody Y9 and an anti-CTLA4 antibody on immune cell phenotypes of peripheral blood lymphocytes and dendritic cells isolated from skin-draining lymph nodes. FIG. 16A is a graph showing the effects of the indicated treatments on the proliferation of CD4+ T cells. FIG. 16B is a graph showing the effects of the indicated treatments on the proliferation of CD8+ T cells. FIG. 16C shows a series of dot plots describing the gating strategy for flow cytometry. FIG. 16D is a graph showing the effects of the indicated treatments on expression of CD86 (B7.2), a co-stimulatory molecule important in dendritic cell activation of T cells.

FIG. 17 shows a series of graphs on the anti-tumor activity of antibody Y9 in wild-type mice, FcGR2BKO mice, and Fc common gamma KO mice in the CT26 syngeneic mouse tumor model.

FIG. 18 shows a series of graphs on the anti-tumor activity of antibody Y9 having different antibody isotypes and variant Fc regions in the CT26 syngeneic mouse tumor model.

FIG. 19 shows a series of graphs showing the effects of antibody Y9 on various aspects of CD8+ T cells, including proliferation, percent CD25+ cells, percent GrnB+ cells, and percent PD-1+ cells.

FIG. 20 is a homology model of mouse TNFR2 (space-filling model) bound to mouse TNF (ribbon model). Amino acid positions at which Y9 binding was significantly disrupted by mutations are mapped (−, black).

FIGS. 21A-21D are a series of graphs demonstrating the antitumor response of a single dose of PBS anti-TNFR2 antibody (1 mg, 0.3 mg, and 0.1 mg) in a syngeneic tumor model with colorectal CT26 cancer cells.

FIGS. 22A-22D are a series of graphs demonstrating the antitumor response of a single dose of PBS or anti-TNFR2 antibody (1 mg, 0.3 mg, and 0.1 mg) in a syngeneic tumor model with EMT6 breast cancer cells.

FIGS. 23A-23D are a series of graphs demonstrating the antitumor response of a single dose of PBS or anti-TNFR2 antibody (1 mg, 0.3 mg, and 0.1 mg) in a syngeneic tumor model with Wehi64 fibrosarcoma cells.

FIGS. 24A-24D are a series of graphs demonstrating the antitumor response of a single dose of PBS or anti-TNFR2 antibody (1 mg, 0.3 mg, and 0.1 mg) in a syngeneic tumor model with A20 B cell lymphoma cells.

FIG. 25 is a graph demonstrating sustained antitumor response of a single dose of anti-TNFR2 antibody (1 mg, 0.3 mg, and 0.1 mg) in a syngeneic tumor model with Wehi64 fibrosarcoma cells vs. untreated age-matched controls.

FIGS. 26A and 26B are graphs showing the effects of antibody Y9 and Y9 DANA on CTLA4 expression in CD4+ conventional T cells, Tregs, and CD8+ T cells in tumors and tumor draining lymph node of a EMT-6 syngeneic model.

FIG. 27A-27C are graphs showing the effects of antibody Y9 and Y9 DANA on GITR (FIG. 27A), GARP (FIG. 27B), and PD-1 (FIG. 27C) expression in CD4+ conventional T cells, Tregs, and CD8+ T cells in tumors of a EMT-6 syngeneic model.

FIG. 28A-28C are graphs showing the effects of antibody Y9 and Y9 DANA on TNFR2 expression in CD4+ conventional T cells (FIG. 28A), Tregs (FIG. 28B), and CD8+ T cells (FIG. 28C) in tumors of CT26, MC38, and WEHI-164 syngeneic models.

FIG. 29 is a graph depicting binding of hybridoma antibodies (ABV3, ABV4, ABV7, ABV12, ABV13, ABV14, ABV15, ABV18 and ABV19) to chimera 0 (hatched), chimera 3 (white), mouse TNFR2 (checkered) and human TNFR2 (black) as measured by ELISA.

FIGS. 30A-30D show alignments of humanized ABV2 antibody heavy and light chain variable region sequences.

FIG. 31 is a graph showing the dose-dependent effects of antibody ABV2 chimera (ABV2c) on NF-kB reporter activity.

FIG. 32A is a graph showing the effects of ABV2c on the percentage of Tregs in CD4+ cells in cultures of ovarian cancer ascites. FIG. 32B shows the gating strategy for the flow cytometry analysis in FIG. 32A.

FIG. 33A is a graph showing the effects of ABV2c on ADCC activity of human cells using NK cells isolated from healthy donors cultured together with carboxyfluorescein succinimidyl ester (CFSE)-labeled JJN3 (plasma cell myeloma) target cells. As target cells die, per-cell fluorescence of CFSE decreases and this can be detected by flow cytometry. FIG. 33B shows the gating strategy for the flow cytometry analysis in FIG. 33A.

FIGS. 34A and 34B show in vitro expansion, induction of activation markers, and cytokines on CD4+ T cells by chimeric anti-human TNFR2 antibody, ABV2c. Naïve CD45RA+CD8+ or CD4+ T cells were stimulated for 4 days with 5 ug/mL plate bound CD3, 1 ug/mL soluble CD28, and various concentrations of plate bound isotype control, anti-TNFR2 (ABV2c), anti-4-1BB (Urelumab), or anti-GITR (TRX518) mAb. FIGS. 34A and 34B show data from 3 individuals and are normalized to samples stimulated in the absence of any anti-TNFRSF antibody. Asterisks show statistical significance between isotype and ABV2c.

FIG. 35 shows the effect of chimeric anti-human TNFR2 antibody, ABV2c, on survival in a xenogeneic GvHD model.

FIG. 36A is a graph showing the effects of various mutations in the CRD1 region of human TNFR2 on the binding of chimeric anti-human TNFR2 antibody, ABV2c, as assessed by flow cytometry. Binding to human TNFR2 was assessed by flow cytometry. Binding curves were fitted using four-parameter dose response. FIG. 36B is a structural model in which mutations that resulted in no antibody binding to human TNFR2 for ABV2c (Y24, Q26, Q29, M30, and K47; numbering based on human TNFR2 without leader sequence) are highlighted in black (human TNFR2 is in white, and TNF is in gray).

FIG. 37A is a graph showing the effects of various mutations in the CRD1 region of mouse TNFR2 on the binding of mouse anti-TNFR2 antibody Y9, as assessed by flow cytometry. Binding to mouse TNFR2 was assessed by flow cytometry. Binding curves were fitted using four-parameter dose response. FIGS. 37B and 37C are graphs showing the effects of additional mutations in the CRD1 region of mouse TNFR2 (Y25T, K28E, and M31A) on the binding of mouse anti-TNFR2 antibody Y9, as assessed by flow cytometry. Binding curves were fitted using four-parameter dose response. FIG. 37D is a homology model in which R27 and N47 are highlighted in black (human TNFR2 is in white, and TNF is in gray). FIG. 37E is a homology model in which all five mutations that resulted in a loss in Y9 antibody binding to mouse TNFR2 (Y25, R27, K28, M31, and N47) are highlighted in black (human TNFR2 is in white, and TNF is in gray).

FIG. 38 is a graph showing anti-tumor activity of chimeric anti-human TNFR2 antibody ABV2c in a patient-derived xenograph model in humanized mice. Shown are tumor growth kinetics with mean and standard error of mean (N=9 animals per arm). Statistical significance was assessed at the end of study at day 72 using ANOVA and Tukey's honestly significant difference procedure for multiple comparison correction.

FIGS. 39A-39C are graphs showing the effects of antibodies ABV2c, ABV2.13, and ABV2.7 on CD4+ T cell proliferation (FIG. 39A), CD4+ T cell expansion, as reflected by the total number of live cells (FIG. 39B), and the percent of CD4+ T cells which are PD-1 positive (FIG. 39C), as assessed by flow cytometry. Data are shown from a single donor and representative of 2 individual donors.

FIGS. 40A and 40B are graphs showing the effects of antibodies ABV2c, ABV2.13, and ABV2.7 on the percent of CD4+ T cells positive for intracellular IFN-γ (FIG. 40A) and intracellular IL-2 (FIG. 40B), as assessed by flow cytometry. Data are from a single donor and are representative of 2 individual donors.

FIGS. 41A-41G are graphs showing the effects of antibodies ABV2c, ABV2.13, and ABV2.7 on the amount of Th-1 associated cytokines (FIG. 41A: IL-2, FIG. 41B: IFN-γ, FIG. 41C: TNF, FIG. 41D: GM-CSF) and Th2-associated cytokines (FIG. 41E: IL-4, FIG. 41F: IL-5, FIG. 41G: IL-13) produced by stimulated CD4+ T cells, as assessed with the Luminex platform. Data are from a single donor and are representative of 2 individual donors.

FIG. 42 is a graph showing the effects of antibodies ABV2c, ABV2.13, ABV2.15, and ABV2.7 on NF-kB reporter activity in a human TNFR2 reporter cell line.

FIGS. 43A-43E are graphs showing the effects of antibodies ABV2.1, ABV2.15, and prior art comparator antibodies A-C on CD4+ T cell proliferation (FIGS. 43A and 43B), CD4+ T cell expansion (FIG. 43C), percent PD-1-positive CD4+ T cells (FIG. 43D), and NF-kB activity (FIG. 43E).

### DETAILED DESCRIPTION

### I. Overview

Provided herein are isolated antibodies, particularly recombinant, monoclonal antibodies, e.g., human monoclonal antibodies, which specifically bind to particular epitopes on TNFR2 (e.g., human TNFR2). Also provided herein are methods of making the antibodies, immunoconjugates and multispecific molecules and pharmaceutical compositions comprising the antibodies, as well as methods of inhibiting tumor growth, treating cancer, treating autoimmune diseases, treating graft-versus-host diseases, and promoting graft survival and/or reducing graft rejection using the antibodies.

### II. Definitions

In order that the present description may be more readily understood, certain terms are first defined. Additional definitions are set forth throughout the detailed description.

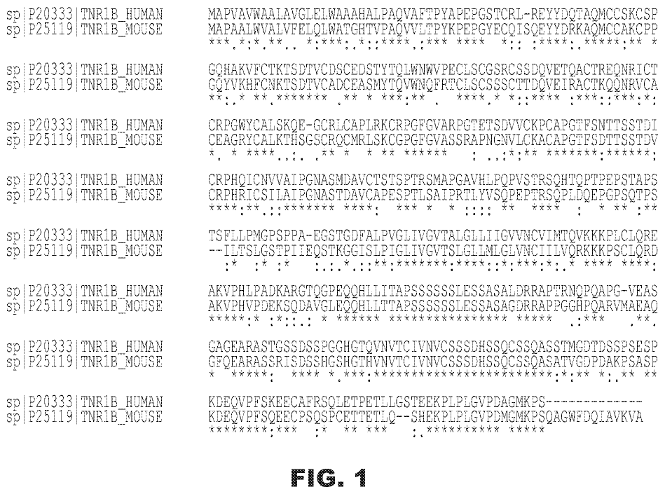

The terms “tumor necrosis factor receptor 2,” “TNFR2,” “CD120b,” “p75,” “p75TNFR,” “p80 TNF-alpha receptor,” “TBPII,” “TNFBR,” “TNFR1B,” “TNF-R75,” and “TNFR80,” are used interchangeably herein, are inclusive of all family members, mutants, alleles, fragments, and species, and refer to a protein having the amino acid sequences (human and mouse) set forth below. The extracellular domain of TNFR2 includes four cysteine-rich domains (CRD1-CRD4), the sequences of which are summarized in Table 1. The numbering of CRD regions in Table 1 is based on human and mouse TNFR2 with the leader sequence (i.e., SEQ ID NOs: 1 and 3). An alignment of mouse and human TNFR2 amino acid sequences is provided in FIG. 1.

Human TNFR2 (NP_001057) (leader sequence is
underlined):
(SEQ ID NO: 1)
MAPVAVWAALAVGLELWAAAHALPAQVAFTPYAPEPGSTCRLREYYDQT
AQMCCSKCSPGQHAKVFCTKTSDTVCDSCEDSTYTQLWNWVPECLSCGS
RCSSDQVETQACTREQNRICTCRPGWYCALSKQEGCRLCAPLRKCRPGF
GVARPGTETSDVVCKPCAPGTFSNTTSSTDICRPHQICNVVAIPGNASM
DAVCTSTSPTRSMAPGAVHLPQPVSTRSQHTQPTPEPSTAPSTSFLLPM
GPSPPAEGSTGDFALPVGLIVGVTALGLLIIGVVNCVIMTQVKKKPLCL
QREAKVPHLPADKARGTQGPEQQHLLITAPSSSSSSLESSASALDRRAP
TRNQPQAPGVEASGAGEARASTGSSDSSPGGHGTQVNVTCIVNVCSSSD
HSSQCSSQASSTMGDTDSSPSESPKDEQVPFSKEECAFRSQLETPETLL
GSTEEKPLPLGVPDAGMKPS
Mouse TNFR2 (NP_035740) (leader sequence is
underlined):
(SEQ ID NO: 3)
MAPAALWVALVFELQLWATGHTVPAQVVLTPYKPEPGYECQISQEYYDR
KAQMCCAKCPPGQYVKHFCNKTSDTVCADCEASMYTQVWNQFRTCLSCS
SSCTTDQVEIRACTKQQNRVCACEAGRYCALKTHSGSCRQCMRLSKCGP
GFGVASSRAPNGNVLCKACAPGTFSDTTSSTDVCRPHRICSILAIPGNA
STDAVCAPESPTLSAIPRTLYVSQPEPTRSQPLDQEPGPSQTPSILTSL
GSTPIIEQSTKGGISLPIGLIVGVTSLGLLMLGLVNCIILVQRKKKPSC
LQRDAKVPHVPDEKSQDAVGLEQQHLLTTAPSSSSSSLESSASAGDRRA
PPGGHPQARVMAEAQGFQEARASSRISDSSHGSHGTHVNVTCIVNVCSS
SDHSSQCSSQASATVGDPDAKPSASPKDEQVPFSQEECPSQSPCETTET
LQSHEKPLPLGVPDMGMKPSQAGWFDQIAVKVA

TABLE 1

| Cysteine-rich | Mouse amino acid | Human amino acid |
|---|---|---|
| domain (CRD) | residues^{A} | residues⁰ |
| CRD1 | 39-77 | 39-76 |
| CRD1 A1 | 40-55 | 40-53 |
| CRD1 B2 | 56-76 | 54-75 |
| CRD2 | 78-120 | 77-118 |
| CRD2 A1 | 79-94 | 78-93 |
| CRD2 B2 | 97-119 | 96-118 |
| CRD3 | 120-164 | 119-162 |
| CRD3 A2 | 121-139 | 120-137 |
| CRD3 B1 | 145-163 | 143-161 |
| CRD4 | 165-203 | 163-201 |
| CRD4 A1 | 166-180 | 164-179 |
| CRD4 B1 | 187-202 | 185-200 |

^{A}Mouse TNFR2 (UniProt ID: P25119)
^{B}Human TNFR2 (UniProt ID: P20333)

TNFR2, together with TNFR1, mediate the activity of TNFα. TNFR1 is a 55 kD membrane-bound protein, whereas TNFR2 is a 75 kD membrane-bound protein. TNFR2 can regulate the binding of TNFα to TNFR1, and thus may regulate the levels of TNFα necessary to stimulate the action of NF-kB. TNFR2 can also be cleaved by metalloproteases (or be subjected to alternative splicing), generating soluble receptors that maintain affinity for TNFα.

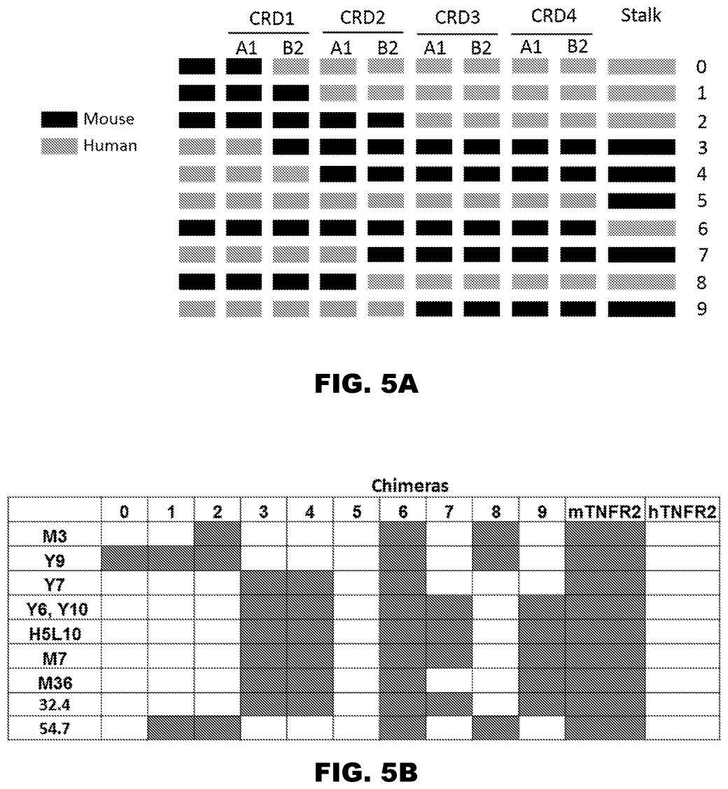

“TNFR2 chimera,” as used herein, refer to a human TNFR2 protein having certain regions within the extracellular domain replaced with corresponding mouse TNFR2 sequences. A schematic of exemplary TNFR2 chimeras is provided in FIG. 5A, with details regarding swapped domains provided in Table 2.

The term “antibody” or “immunoglobulin,” as used interchangeably herein, includes whole antibodies and any antigen binding fragment (antigen-binding portion) or single chain cognates thereof. An “antibody” comprises at least one heavy (H) chain and one light (L) chain. In naturally occurring IgGs, for example, these heavy and light chains are inter-connected by disulfide bonds and there are two paired heavy and light chains, these two also inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR) or Joining (J) regions (JH or JL in heavy and light chains respectively). Each V_{H} and V_{L} is composed of three CDRs, three FRs and a J domain, arranged from amino-terminus to carboxy-terminus in the following order: FR1, CDR1, FR2, CDR2, FR3, CDR3, J. The variable regions of the heavy and light chains bind with an antigen. The constant regions of the antibodies may mediate the binding of the immunoglobulin to host tissues or factors, including various cells of the immune system (e.g., effector cells) or humoral factors such as the first component (Clq) of the classical complement system. It has been shown that fragments of a full-length antibody can perform the antigen-binding function of an antibody. Examples of binding fragments denoted as an antigen-binding portion or fragment of an antibody include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb including VH and VL domains; (vi) a dAb fragment (Ward et al. (1989) Nature 341, 544-546), which consists of a V_{H} domain; (vii) a dAb which consists of a VH or a VL domain; and (viii) an isolated complementarity determining region (CDR) or (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions are paired to form monovalent molecules (such a single chain cognate of an immunoglobulin fragment is known as a single chain Fv (scFv). Such single chain antibodies are also intended to be encompassed within the term “antibody”. Antibody fragments are obtained using conventional techniques known to those with skill in the art, and the fragments are screened for utility in the same general manner as are intact antibodies. Antigen-binding portions can be produced by recombinant DNA techniques, or by enzymatic or chemical cleavage of intact immunoglobulins. Unless otherwise specified, the numbering of amino acid positions in the antibodies described herein (e.g., amino acid residues in the Fc region) and identification of regions of interest, e.g., CDRs, use the Kabat system (Kabat, E. A., et al. (1991) Sequences of Proteins of Immunological Interest, Fifth Edition, U.S. Department of Health and Human Services, NIH Publication No. 91-3242).

The term “monoclonal antibody” as used herein refers to an antibody obtained from a population of substantially homogeneous antibodies, i.e., the individual antibodies comprising the population are identical except for possible naturally occurring mutations that may be present in minor amounts. Antigen binding fragments (including scFvs) of such immunoglobulins are also encompassed by the term “monoclonal antibody” as used herein. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. Monoclonal antibodies can be prepared using any art recognized technique and those described herein such as, for example, a hybridoma method, a transgenic animal, recombinant DNA methods (see, e.g., U.S. Pat. No. 4,816,567), or using phage antibody libraries using the techniques described in, for example, U.S. Pat. No. 7,388,088 and U.S. patent application Ser. No. 09/856,907 (PCT Int. Pub. No. WO 00/31246). Monoclonal antibodies include chimeric antibodies, human antibodies, and humanized antibodies and may occur naturally or be produced recombinantly.

As used herein, “isotype” refers to the antibody class (e.g., IgG1, IgG2, IgG3, IgG4, IgM, IgA1, IgA2, IgD, and IgE antibody) that is encoded by the heavy chain constant region genes.

The term “recombinant antibody,” refers to antibodies that are prepared, expressed, created or isolated by recombinant means, such as (a) antibodies isolated from an animal (e.g., a mouse) that is transgenic or transchromosomal for immunoglobulin genes (e.g., human immunoglobulin genes) or a hybridoma prepared therefrom, (b) antibodies isolated from a host cell transformed to express the antibody, e.g., from a transfectoma, (c) antibodies isolated from a recombinant, combinatorial antibody library (e.g., containing human antibody sequences) using phage display, and (d) antibodies prepared, expressed, created or isolated by any other means that involve splicing of immunoglobulin gene sequences (e.g., human immunoglobulin genes) to other DNA sequences. Such recombinant antibodies may have variable and constant regions derived from human germline immunoglobulin sequences. In certain embodiments, however, such recombinant human antibodies can be subjected to in vitro mutagenesis and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “chimeric immunoglobulin” or “chimeric antibody” refers to an immunoglobulin or antibody whose variable regions derive from a first species and whose constant regions derive from a second species. Chimeric immunoglobulins or antibodies can be constructed, for example by genetic engineering, from immunoglobulin gene segments belonging to different species.

The term “humanized antibody” refers to an antibody that includes at least one humanized antibody chain (i.e., at least one humanized light or heavy chain). The term “humanized antibody chain” (i.e., a “humanized immunoglobulin light chain”) refers to an antibody chain (i.e., a light or heavy chain, respectively) having a variable region that includes a variable framework region substantially from a human antibody and complementarity determining regions (CDRs) (e.g., at least one CDR, two CDRs, or three CDRs) substantially from a non-human antibody. In some embodiments, the humanized antibody chain further includes constant regions (e.g., one constant region or portion thereof, in the case of a light chain, and preferably three constant regions in the case of a heavy chain).

The term “human antibody,” as used herein, is intended to include antibodies having variable regions in which both the framework and CDR regions are derived from human germline immunoglobulin sequences as described, for example, by Kabat et al. (See Kabat, et al. (1991) Sequences of proteins of Immunological Interest, Fifth Edition, U.S. Department of Health and Human Services, NIH Publication No. 91-3242). Furthermore, if the antibody contains a constant region, the constant region also is derived from human germline immunoglobulin sequences. The human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The human antibody can have at least one or more amino acids replaced with an amino acid residue, e.g., an activity enhancing amino acid residue that is not encoded by the human germline immunoglobulin sequence. Typically, the human antibody can have up to twenty positions replaced with amino acid residues that are not part of the human germline immunoglobulin sequence. In a particular embodiment, these replacements are within the CDR regions as described in detail below.

A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

“Isolated,” as used herein, is intended to refer to an antibody that is substantially free of other antibodies having different antigenic specificities. In addition, an isolated antibody is typically substantially free of other cellular material and/or chemicals.

An “effector function” refers to the interaction of an antibody Fc region with an Fc receptor or ligand, or a biochemical event that results therefrom. Exemplary “effector functions” include Clq binding, complement dependent cytotoxicity (CDC), Fc receptor binding, FcγR-mediated effector functions such as ADCC and antibody dependent cell-mediated phagocytosis (ADCP), and downregulation of a cell surface receptor (e.g., the B cell receptor; BCR). Such effector functions generally require the Fc region to be combined with a binding domain (e.g., an antibody variable domain).

An “Fc region,” “Fc domain,” or “Fc” refers to the C-terminal region of the heavy chain of an antibody. Thus, an Fc region comprises the constant region of an antibody excluding the first constant region immunoglobulin domain (e.g., CH1 or CL).

An “antigen” is an entity (e.g., a proteinaceous entity or peptide) to which an antibody binds, e.g., TNFR2.

The terms “specific binding,” “specifically binds,” “selective binding,” and “selectively binds,” mean that an antibody exhibits appreciable affinity for a particular antigen or epitope and, generally, does not exhibit significant cross-reactivity with other antigens and epitopes. “Appreciable” or preferred binding includes binding with a K_{D} of 10⁻⁷, 10⁻⁸, 10⁻⁹, or 10⁻¹⁰ M or better. The K_{D} of an antibody antigen interaction (the affinity constant) indicates the concentration of antibody at which 50% of antibody and antigen molecules are bound together. Thus, at a suitable fixed antigen concentration, 50% of a higher (i.e., stronger) affinity antibody will bind antigen molecules at a lower antibody concentration than would be required to achieve the same percent binding with a lower affinity antibody. Thus a lower K_{D} value indicates a higher (stronger) affinity. As used herein, “better” affinities are stronger affinities, and are of lower numeric value than their comparators, with a K_{D} of 10⁻⁷ M being of lower numeric value and therefore representing a better affinity than a K_{D} of 10⁻⁶ M. Affinities better (i.e., with a lower K_{D} value and therefore stronger) than 10⁻⁷ M, preferably better than 10⁻⁸ M, are generally preferred. Values intermediate to those set forth herein are also contemplated, and a preferred binding affinity can be indicated as a range of affinities, for example preferred binding affinities for anti-TNFR2 antibodies disclosed herein are, 10⁻⁷ to 10⁻¹² M, more preferably 10⁻⁸ to 10⁻¹² M. An antibody that “does not exhibit significant cross-reactivity” or “does not bind with a physiologically-relevant affinity” is one that will not appreciably bind to an off-target antigen (e.g., a non-TNFR2 protein) or epitope. For example, in one embodiment, an antibody that specifically binds to TNFR2 will exhibit at least a two, and preferably three, or four or more orders of magnitude better binding affinity (i.e., binding exhibiting a two, three, or four or more orders of magnitude lower K_{D} value) for TNFR2 than, e.g., a protein other than TNFR2. Specific or selective binding can be determined according to any art-recognized means for determining such binding, including, for example, according to Scatchard analysis, Biacore analysis, bio-layer interferometry, and/or competitive (competition) binding assays as described herein.

The term “K_{D},” as used herein, is intended to refer to the dissociation equilibrium constant of a particular antibody-antigen interaction or the affinity of an antibody for an antigen, which is obtained from the ratio of k_{d} to kₐ (i.e., k_{d}/kₐ) and is expressed as a molar concentration (M). K_{D} values for antibodies can be determined using methods well established in the art. In some embodiments, an antibody binds an antigen with an affinity (K_{D}) of approximately less than 10⁻⁷ M, such as approximately less than 10⁻⁸ M, 10⁻⁹ M or 10⁻¹⁰ M or even lower when determined by bio-layer interferometery with a Pall ForteBio Octet RED96 Bio-Layer Interferometry system or surface plasmon resonance (SPR) technology in a BIACORE 3000 instrument using recombinant TNFR2 as the analyte and the antibody as the ligand, and binds to the predetermined antigen with an affinity that is at least two-fold greater than its affinity for binding to a non-specific antigen (e.g., BSA, casein) other than the predetermined antigen or a closely-related antigen. Other methods for determining K_{D} include equilibrium binding to live cells expressing TNFR2 via flow cytometry (FACS) or in solution using KinExA® technology. K_{D} values as used herein refer to monovalent K_{D}.

The term “k_{assoc}” or “kₐ”, as used herein, is intended to refer to the association rate of a particular antibody-antigen interaction, whereas the term “k_{dis}” or “k_{d},” as used herein, is intended to refer to the dissociation rate of a particular antibody-antigen interaction.

The term “epitope” or “antigenic determinant” refers to a site on an antigen to which an immunoglobulin or antibody specifically binds. Epitopes can be formed both from contiguous amino acids (usually a linear epitope) or noncontiguous amino acids juxtaposed by tertiary folding of a protein (usually a conformational epitope). Epitopes formed from contiguous amino acids are typically, but not always, retained on exposure to denaturing solvents, whereas epitopes formed by tertiary folding are typically lost on treatment with denaturing solvents. Methods for determining what epitopes are bound by a given antibody (i.e., epitope mapping) are well known in the art and include, for example, immunoblotting and immunoprecipitation assays, wherein overlapping or contiguous peptides are tested for reactivity with a given antibody. Methods of determining spatial conformation of epitopes include techniques in the art, for example, x-ray crystallography, 2-dimensional nuclear magnetic resonance and HDX-MS (see, e.g., Epitope Mapping Protocols in Methods in Molecular Biology, Vol. 66, G. E. Morris, Ed. (1996)). The term “epitope mapping” refers to the process of identification of the molecular determinants for antibody-antigen recognition.

The term “binds to the same epitope” with reference to two or more antibodies means that the antibodies bind to the same segment of amino acid residues, as determined by a given method. Techniques for determining whether antibodies bind to the “same epitope on TNFR2” with the antibodies described herein include, for example, epitope mapping methods, such as, x-ray analyses of crystals of antigen:antibody complexes which provides atomic resolution of the epitope and hydrogen/deuterium exchange mass spectrometry (HDX-MS). Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competition assays can be conducted as described, for example, in Ed Harlow and David Lane, Cold Spring Harb Protoc; 2006; doi:10.1101/pdb.prot4277 or in Chapter 11 of “Using Antibodies” by Ed Harlow and David Lane, Cold Spring Harbor Laboratory Press, Cold Spring Harbor, N.Y., USA 1999. Competing antibodies bind to the same epitope, an overlapping epitope or to adjacent epitopes (e.g., as evidenced by steric hindrance). Other competitive binding assays include: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using I-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)).

The term “nucleic acid molecule,” as used herein, is intended to include DNA molecules and RNA molecules. A nucleic acid molecule may be single-stranded or double-stranded, but preferably is double-stranded DNA.

The term “isolated nucleic acid molecule,” as used herein in reference to nucleic acids encoding antibodies or antibody fragments (e.g., V_{H}, V_{L}, CDR3), is intended to refer to a nucleic acid molecule in which the nucleotide sequences are essentially free of other genomic nucleotide sequences, e.g., those encoding antibodies that bind antigens other than TNFR2, which other sequences may naturally flank the nucleic acid in human genomic DNA.

The term “modifying,” or “modification,” as used herein, refers to changing one or more amino acids in an antibody or antigen-binding portion thereof, or on a recombinant TNFR2 protein (e.g., for epitope mapping). The change can be produced by adding, substituting or deleting an amino acid at one or more positions. The change can be produced using known techniques, such as PCR mutagenesis. For example, in some embodiments, an antibody or an antigen-binding portion thereof identified using the methods provided herein can be modified, to thereby modify the binding affinity of the antibody or antigen-binding portion thereof to TNFR2.

“Conservative amino acid substitutions” in the sequences of the antibodies refer to nucleotide and amino acid sequence modifications which do not abrogate the binding of the antibody encoded by the nucleotide sequence or containing the amino acid sequence, to the antigen (e.g., TNFR2). Conservative amino acid substitutions include the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution. Thus, a predicted nonessential amino acid residue in an anti-TNFR2 antibody is preferably replaced with another amino acid residue from the same class. Methods of identifying nucleotide and amino acid conservative substitutions which do not eliminate antigen binding are well-known in the art.

The term “non-conservative amino acid substitution” refers to the substitution of an amino acid in one class with an amino acid from another class; for example, substitution of an Ala, a class II residue, with a class III residue such as Asp, Asn, Glu, or Gln.

Alternatively, in another embodiment, mutations (conservative or non-conservative) can be introduced randomly along all or part of an anti-TNFR2 antibody coding sequence, such as by saturation mutagenesis, and the resulting modified anti-TNFR2 antibodies can be screened for binding activity.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. The terms, “plasmid” and “vector” may be used interchangeably. However, other forms of expression vectors, such as viral vectors (e.g., replication defective retroviruses, adenoviruses and adeno-associated viruses), which serve equivalent functions are also contemplated.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

Also provided are “conservative sequence modifications” of the sequences set forth herein, i.e., amino acid sequence modifications which do not abrogate the binding of the antibody encoded by the nucleotide sequence or containing the amino acid sequence, to the antigen. Such conservative sequence modifications include conservative nucleotide and amino acid substitutions, as well as, nucleotide and amino acid additions and deletions. For example, modifications can be introduced into a sequence in Table 10 by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an anti-TNFR2 antibody is preferably replaced with another amino acid residue from the same side chain family. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)). Alternatively, in another embodiment, mutations can be introduced randomly along all or part of an anti-TNFR2 antibody coding sequence, such as by saturation mutagenesis, and the resulting modified anti-TNFR2 antibodies can be screened for binding activity.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% of the amino acids, usually at least about 90% to 95%, and more preferably at least about 98% to 99.5% of the amino acids.

The percent identity between two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or two amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the XBLAST program, score=50, wordlength=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See www.ncbi.nlm.nih.gov.

The term “inhibition” as used herein, refers to any statistically significant decrease in biological activity, including partial and full blocking of the activity. For example, “inhibition” can refer to a statistically significant decrease of about 10%, 20%, 30%, 40%, 50%, 60%, 70%, 80%, 90%, 95%, 96%, 97%, 98%, 99%, or 100% in biological activity.

The phrase “inhibit TNFR2 ligand binding to TNFR2,” as used herein, refers to the ability of an antibody to statistically significantly decrease the binding of an TNFR2 ligand (e.g., TNFα) to TNFR2, relative to the TNFR2 ligand binding in the absence of the antibody (control). In other words, in the presence of the antibody, the amount of the TNFR2 ligand that binds to TNFR2 relative to a control (no antibody), is statistically significantly decreased. The amount of an TNFR2 ligand which binds to TNFR2 may be decreased in the presence of an anti-TNFR2 antibody disclosed herein by at least about 10%, or at least about 20%, or at least about 30%, or at least about 40%, or at least about 50%, or at least about 60%, or at least about 70%, or at least about 80%, or at least about 90%, or about 100% relative to the amount in the absence of the antibody (control). A decrease in TNFR2 ligand binding can be measured using art-recognized techniques that measure the level of binding of labeled TNFR2 ligand (e.g., radiolabelled TNFα) to cells expressing TNFR2 in the presence or absence (control) of the antibody.

As used herein, the term “inhibits growth” of a tumor includes any measurable decrease in the growth of a tumor, e.g., the inhibition of growth of a tumor by at least about 10%, for example, at least about 20%, at least about 30%, at least about 40%, at least about 50%, at least about 60%, at least about 70%, at least about 80%, at least about 90%, at least about 99%, or about 100%.

The terms “treat,” “treating,” and “treatment,” as used herein, refer to therapeutic or preventative measures described herein. The methods of “treatment” employ administration to a subject with a tumor or cancer or a subject who is predisposed to having such a disease or disorder, an anti-TNFR2 antibody (e.g., anti-human TNFR2 antibody) described herein, in order to prevent, cure, delay, reduce the severity of, or ameliorate one or more symptoms of the disease or disorder or recurring disease or disorder, or in order to prolong the survival of a subject beyond that expected in the absence of such treatment.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, small-cell lung cancer, non-small cell lung cancer, gastric cancer, pancreatic cancer, glial cell tumors such as glioblastoma and neurofibromatosis, cervical cancer, ovarian cancer, liver cancer, bladder cancer, hepatoma, breast cancer, colon cancer, melanoma, colorectal cancer, endometrial carcinoma, salivary gland carcinoma, kidney cancer, renal cancer, prostate cancer, vulval cancer, thyroid cancer, hepatic carcinoma and various types of head and neck cancer.

The phrase “long-term anti-cancer effect” as used herein, refers to the ability of an antibody to induce suppression of cancer growth for a sustained period of time (e.g., at least 6 or more months) after initial treatment with the antibody. The sustained anti-cancer effect may be assessed, e.g., by measuring tumor growth or by periodically testing blood samples of a subject in remission for the presence of memory T cells against the original cancer (e.g., testing for reactivity to original biopsy samples).

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the disorder being treated and the general state of the patient's own immune system.

The term “therapeutic agent” in intended to encompass any and all compounds that have an ability to decrease or inhibit the severity of the symptoms of a disease or disorder, or increase the frequency and/or duration of symptom-free or symptom-reduced periods in a disease or disorder, or inhibit or prevent impairment or disability due to a disease or disorder affliction, or inhibit or delay progression of a disease or disorder, or inhibit or delay onset of a disease or disorder, or inhibit or prevent infection in an infectious disease or disorder. Non-limiting examples of therapeutic agents include small organic molecules, monoclonal antibodies, bispecific antibodies, recombinantly engineered biologics, RNAi compounds, and commercial antibodies.

As used herein, “administering” refers to the physical introduction of a composition comprising a therapeutic agent to a subject, using any of the various methods and delivery systems known to those skilled in the art. Exemplary routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

The term “patient” includes human and other mammalian subjects that receive either prophylactic or therapeutic treatment.

The term “subject” includes any mammal. For example, the methods and compositions herein disclosed can be used to treat a subject having cancer. In a particular embodiment, the subject is a human.

The term “sample” refers to tissue, body fluid, or a cell (or a fraction of any of the foregoing) taken from a patient or a subject. Normally, the tissue or cell will be removed from the patient, but in vivo diagnosis is also contemplated. In the case of a solid tumor, a tissue sample can be taken from a surgically removed tumor and prepared for testing by conventional techniques. In the case of lymphomas and leukemias, lymphocytes, leukemic cells, or lymph tissues can be obtained (e.g., leukemic cells from blood) and appropriately prepared. Other samples, including urine, tears, serum, plasma, cerebrospinal fluid, feces, sputum, cell extracts etc. can also be useful for particular cancers.

As used herein, the term “about” means plus or minus 10% of a specified value.

As used herein, the term “and/or” includes any and all combinations of one or more of the associated listed items. For example, the phrase “A, B, and/or C” is intended to encompass A; B; C; A and B; A and C; B and C; and A, B, and C.

As used in the description of the invention and the appended claims, the singular forms “a,” “an” and “the” are intended to include the plural forms as well, unless the context clearly indicates otherwise.

Various aspects of the disclosure are described in further detail in the following subsections.

### III. Anti-TNFR2 Antibodies

The anti-TNFR2 antibodies (e.g., anti-human TNFR2 antibodies), and antigen-binding fragments thereof, disclosed herein, can be characterized by particular functional features or properties. For example, the antibodies bind to the extracellular domain of human TNFR2. The anti-TNFR2 antibodies may also induce a long-term anti-cancer effect or the development of anti-cancer memory T cells.

In some embodiments, the antibodies bind to a portion or all of one or more cysteine-rich domain(s) (CRD) of human TNFR2. Amino acid residues corresponding to human and mouse TNFR2 CRDs are summarized in Table 1.

Accordingly, in one aspect, provided herein are anti-TNFR2 antibodies that bind to all or a portion of amino acid residues 23-54 of human TNFR2 (SEQ ID NO: 1), and do not bind one or more amino acid residues within 55-77 of human TNFR2 (SEQ ID NO: 1). In one embodiment, the anti-TNFR2 antibodies bind to all or a portion of amino acid residues 23-54 of human TNFR2 (SEQ ID NO: 1), and do not bind to one or more amino acids within 55-77, 60-77, 65-77, 70-77, 75-77, 55-75, 55-70, 55-65, or 55-60 of human TNFR2 (SEQ ID NO: 1). In another embodiment, the anti-TNFR2 antibodies bind to all or a portion of amino acid residues 23-54 of human TNFR2 (SEQ ID NO: 1), and do not bind to one or more amino acid residues within 55-77, 78-118, 120-143, or 161-200 of human TNFR2 (SEQ ID NO: 1). In another embodiment, the anti-TNFR2 antibodies bind to all or a portion of amino acid residues 23-54 of human TNFR2 (SEQ ID NO: 1), and do not bind to one or more amino acid residues within 55-77, 78-118, 120-143, and 161-200 of human TNFR2 (SEQ ID NO: 1). In another embodiment, the anti-TNFR2 antibodies bind to all or a portion of amino acid residues 23-54 of human TNFR2 (SEQ ID NO: 1), and do not bind to amino acid residues within 55-77, 78-118, 120-143, and 161-200 of human TNFR2 (SEQ ID NO: 1). In some embodiments of this aspect, the anti-TNFR2 antibodies significantly inhibit the binding of TNF-alpha to human TNFR2 (e.g., inhibit the binding of TNF-alpha to human TNFR2 by greater than 50% (e.g., greater than 60%, greater than 70%, greater than 80%, greater than 90%, or greater than 95%), as assessed by ELISA (e.g., as described in Example 3)).

In another aspect, provided herein are anti-TNFR2 antibodies that bind to all or a portion of amino acid residues 55-96 of human TNFR2 (SEQ ID NO: 1), and do not significantly inhibit binding of TNF-alpha to human TNFR2 (e.g., inhibit binding of TNF-alpha to human TNFR2 by less than 50% as assessed by, e.g., ELISA (for example, as described in Example 3)). In one embodiment, the anti-TNFR2 antibodies bind to all or a portion of amino acid residues 55-96 of human TNFR2 (SEQ ID NO: 1), and do not bind to one or more amino acids within 23-54 of human TNFR2 (SEQ ID NO: 1). In one embodiment, the anti-TNFR2 antibodies bind to all or a portion of amino acid residues 55-96 of human TNFR2 (SEQ ID NO: 1), and do not bind to one or more amino acids within 23-54, 23-44, 23-36, 23-30, 23-25, 25-54, 30-54, 35-54, or 40-54 of human TNFR2 (SEQ ID NO: 1). In another embodiment, the anti-TNFR2 antibodies bind to all or a portion of amino acid residues 55-96 of human TNFR2 (SEQ ID NO: 1), and do not bind to one or more amino acid residues within 23-54, 97-118, 120-143, or 161-200 of human TNFR2 (SEQ ID NO: 1). In another embodiment, the anti-TNFR2 antibodies bind to all or a portion of amino acid residues 55-96 of human TNFR2 (SEQ ID NO: 1), and do not bind to one or more amino acid residues within 23-54, 97-118, 120-143, and 161-200 of human TNFR2 (SEQ ID NO: 1). In another embodiment, the anti-TNFR2 antibodies bind to all or a portion of amino acid residues 55-96 of human TNFR2 (SEQ ID NO: 1), and do not bind to amino acid residues within 23-54, 97-118, 120-143, and 161-200 of human TNFR2 (SEQ ID NO: 1). In another embodiment, the anti-TNFR2 antibodies bind to all or a portion of amino acid residues 55-96 of human TNFR2 (SEQ ID NO: 1), and exhibit reduced binding to a mutant human TNFR2 comprising a substitution (e.g., a non-conservative substitution) at one or more amino acid residues selected from the group consisting of 37, 44, 51, 52, 55, 58, 59, 61, 62, 72, 74, 76, 78, and 87 of human TNFR2 (SEQ ID NO: 1) as compared to wild-type human TNFR2. In some embodiments of this aspect, the anti-TNFR2 antibodies do not significantly inhibit the binding of TNF-alpha to human TNFR2 (e.g., inhibit the binding of TNF-alpha to human TNFR2 by less than 50%, as assessed by ELISA (e.g., as described in Example 3)). In some embodiments of this aspect, the anti-TNFR2 antibodies exhibit TNFR2 agonist activity (e.g., induce IKBa degradation in Treg cells (e.g., as described in Example 10) with at least 50% of the effectiveness of TNF-alpha).

In another aspect, provided herein are anti-TNFR2 antibodies that exhibit reduced binding (e.g., at least 50% reduced binding, at least 80% reduced binding, or at least 90% reduced binding) to a mutant human TNFR2 comprising one or more amino acid substitutions selected from the group consisting of: G37D, E44A, Q51A, M52A, S55A, S58A, P59A, Q61A, H62A, D72A, V74A, D76A, S78A, and W87A of human TNFR2 (SEQ ID NO: 1) as compared to wild-type human TNFR2. In some embodiments, the anti-TNFR2 antibodies do not exhibit reduced binding (e.g., do not exhibit greater than 50% reduced binding, greater than 40% reduced binding, greater than 30% reduced binding, greater than 25% reduced binding, greater than 20% reduced binding, greater than 15% reduced binding, greater than 10% reduced binding, or greater than 5% reduced binding) to a mutant human TNFR2 comprising one or more amino acid substitutions selected from the group consisting of: T39A, R41A, L42A, R43A, K64A, V65A, K69A, T70A, S71A, E79A, D80A, R112A, and/or E113A. In some embodiments of this aspect, the anti-TNFR2 antibodies do not significantly inhibit the binding of TNF-alpha to human TNFR2 (e.g., inhibit the binding of TNF-alpha to human TNFR2 by less than 50%, as assessed by ELISA (e.g., as described in Example 3)). In some embodiments of this aspect, the anti-TNFR2 antibodies exhibit TNFR2 agonist activity (e.g., induce IKBa degradation in Treg cells (e.g., as described in Example 10) with at least 50% of the effectiveness of TNF-alpha).

In another aspect, provided herein are anti-TNFR2 antibodies that bind to human TNFR2 (SEQ ID NO: 1) at one or more amino acid residues selected from the group consisting of G37, E44, Q51, M52, S55, S58, P59, Q61, H62, D72, V74, D76, S78, and W87. In some embodiments, the anti-TNFR2 antibodies bind to human TNFR2 at amino acid residues S55 and D72. In some embodiments, the anti-TNFR2 antibodies do not bind to human TNFR2 at amino acid residues T39, R41, D80, R112, and/or E113. In some embodiments, the anti-TNFR2 antibodies do not bind to human TNFR2 at amino acid residues T39, R41, L42, R43, K64, V65, K69, T70, S71, E79, D80, R112, and/or E113. In some embodiments of this aspect, the anti-TNFR2 antibodies do not significantly inhibit the binding of TNF-alpha to human TNFR2 (e.g., inhibit the binding of TNF-alpha to human TNFR2 by less than 50%, as assessed by ELISA (e.g., as described in Example 3)). In some embodiments of this aspect, the anti-TNFR2 antibodies exhibit TNFR2 agonist activity (e.g., induce IKBa degradation in Treg cells (e.g., as described in Example 10) with at least 50% of the effectiveness of TNF-alpha).

In another aspect, provided herein are isolated antibodies that exhibit reduced binding (e.g., at least 50% reduced binding, at least 60% reduced binding, at least 70% reduced binding, at least 80% reduced binding, or at least 90% reduced binding) to a mutant human TNFR2 comprising a substitution (e.g., a non-conservative amino acid substitution, e.g., an alanine substitution) at amino acid residue 48 and/or amino acid residue 68 of human TNFR2 (SEQ ID NO: 1). In one embodiment, provided herein are isolated antibodies that bind to amino acid residue 48 and/or amino acid residue 68 of human TNFR2 (SEQ ID NO: 1). In some embodiments, the antibodies do not exhibit reduced binding (e.g., not more than 20% reduced binding or not more than 10% reduced binding) to a mutant human TNFR2 comprising a substitution (e.g., a non-conservative amino acid substitution, e.g., an alanine substitution) at one or more amino acid residues selected from the group consisting of residues 37, 39, 42, 49, 51, 56, 65, 66, 69, 86, 89, and 91. In some embodiments, the antibodies do not bind to one or more amino acid residues selected from the group consisting of residues 37, 39, 42, 49, 51, 56, 65, 66, 69, 86, 89, and 91. In some embodiments, the antibodies do not bind amino acid residues 97-118, 120-143 and/or 161-200 of human TNFR2 (SEQ ID NO: 1). In some embodiments, reduced binding of the antibodies to the mutant TNFR2 is assessed by yeast surface display.

In another aspect, provided herein are anti-TNFR2 antibodies that bind to all or a portion of amino acid residues 78-118, and do not bind to one or more amino acids within 23-77 or 119-200 of human TNFR2 (SEQ ID NO: 1). In one embodiment, the anti-TNFR2 antibodies bind to all or a portion of amino acid residues 78-118, and do not bind to one or more amino acids within 23-77, 23-75, 23-70, 23-65, 23-60, 23-55, 23-50, 23-45, 23-35, 23-30, 23-25 or 119-120 of human TNFR2 (SEQ ID NO: 1). In another embodiment, the anti-TNFR2 antibodies bind to all or a portion of amino acid residues 78-118, and do not bind to one or more amino acids within 23-77, 119-120, 120-143, or 161-200 of human TNFR2 (SEQ ID NO: X). In another embodiment, the anti-TNFR2 antibodies bind to all or a portion of amino acid residues 78-118, and do not bind to one or more amino acids within 23-77, 119-120, 120-143, and 161-200 of human TNFR2 (SEQ ID NO: 1). In another embodiment, the anti-TNFR2 antibodies bind to all or a portion of amino acid residues 78-118, and do not bind to amino acids within 23-77, 119-120, 120-143, and 161-200 of human TNFR2 (SEQ ID NO: 1). In some embodiments of this aspect, the anti-TNFR2 antibodies significantly inhibit the binding of TNF-alpha to human TNFR2 (e.g., inhibit the binding of TNF-alpha to human TNFR2 by greater than 50% (e.g., greater than 60%, greater than 70%, greater than 80%, greater than 90%, or greater than 95%), as assessed by ELISA (e.g., as described in Example 3).

In another aspect, provided herein are anti-TNFR2 antibodies that (1) bind all or a portion of amino acid residues 120-257 of human TNFR2 (SEQ ID NO: 1), and (2) do not significantly inhibit the binding of TNF-alpha to human TNFR2 (SEQ ID NO: 1). In one embodiment, the anti-TNFR2 antibodies (1) bind to all or a portion of amino acid residues 120-257, (2) do not bind one or more amino acid residues within 78-118 of human TNFR2 (SEQ ID NO: 1) (e.g., do not bind to one or more amino acids within 78-118, 78-115, 78-110, 78-105, 78-100, 78-95, 78-90, 78-85, 78-80, or 23-77 of human TNFR2 (SEQ ID NO: 1)), and (3) do not significantly inhibit the binding of TNF-alpha to human TNFR2 (SEQ ID NO: 1). In another embodiment, the anti-TNFR2 antibodies (1) bind to all or a portion of amino acid residues 120-257, (2) do not bind to one or more amino acids within 23-77 or 78-118 of human TNFR2 (SEQ ID NO: 1), and (3) do not significantly inhibit the binding of TNF-alpha to human TNFR2 (SEQ ID NO: 1). In another embodiment, the anti-TNFR2 antibodies (1) bind to all or a portion of amino acid residues 120-257, (2) do not bind to one or more amino acids within 23-77 and 78-118 of human TNFR2 (SEQ ID NO: 1), and (3) do not significantly inhibit the binding of TNF-alpha to human TNFR2 (SEQ ID NO: 1). In another embodiment, the anti-TNFR2 antibodies (2) bind to all or a portion of amino acid residues 120-257, (2) do not bind to amino acids within 23-77 and 78-118 of human TNFR2 (SEQ ID NO: 1), and (3) do not significantly inhibit the binding of TNF-alpha to human TNFR2 (SEQ ID NO: 1). In some embodiments of this aspect, the anti-TNFR2 antibodies do not significantly inhibit the binding of TNF-alpha to human TNFR2 (e.g., inhibit the binding of TNF-alpha to human TNFR2 by less than 50%, as assessed by ELISA (e.g., as described in Example 3)).

In another aspect, provided herein are anti-TNFR2 antibodies that bind all or a portion of amino acid residues 120-257 of human TNFR2 (SEQ ID NO: 1), and do not bind one or more amino acid residues within 78-118 of human TNFR2 (SEQ ID NO: 1). In one embodiment, the anti-TNFR2 antibodies bind all or a portion of amino acid residues 120-257 of human TNFR2 (SEQ ID NO: 1), and do not bind to one or more amino acids within 78-118, 78-115, 78-110, 78-105, 78-100, 78-95, 78-90, 78-85, 78-80, or 23-77 of human TNFR2 (SEQ ID NO: 1). In another embodiment, the anti-TNFR2 antibodies bind all or a portion of amino acid residues 120-257 of human TNFR2 (SEQ ID NO: 1), and do not bind to one or more amino acids within 23-77 or 78-118 of human TNFR2 (SEQ ID NO: 1). In another embodiment, the anti-TNFR2 antibodies bind all or a portion of amino acid residues 120-257 of human TNFR2 (SEQ ID NO: 1), and do not bind to one or more amino acids within 23-77 and 78-118 of human TNFR2 (SEQ ID NO: 1). In another embodiment, the anti-TNFR2 antibodies bind all or a portion of amino acid residues 120-257 of human TNFR2 (SEQ ID NO: 1), and do not bind to amino acids within 23-77 and 78-118 of human TNFR2 (SEQ ID NO: 1).

In another aspect, provided herein are anti-TNFR2 antibodies that bind to all or a portion of amino acid residues 78-118 of human TNFR2 (SEQ ID NO: 1), and do not bind to one or more amino acids within 23-77. In one embodiment, the anti-TNFR2 antibodies bind to all or a portion of amino acid residues 78-118 of human TNFR2 (SEQ ID NO: 1), and do not bind to one or more amino acids within 23-77, 23-75, 23-70, 23-65, 23-60, 23-55, 23-50, 23-45, 23-40, 23-35, 23-30, or 23-25 of human TNFR2 (SEQ ID NO: 1). In another embodiment, the anti-TNFR2 antibodies bind to all or a portion of amino acid residues 78-118 of human TNFR2 (SEQ ID NO: 1), and do not bind to one or more amino acids within 161-200 of human TNFR2 (SEQ ID NO: 1). In another embodiment, the anti-TNFR2 antibodies (1) bind to all or a portion of amino acid residues 78-118 of human TNFR2 (SEQ ID NO: 1), (2) bind to all or a portion of amino acids residues 120-143, and (3) do not bind to one or more amino acids within 161-200 of human TNFR2 (SEQ ID NO: 1). In another embodiment, the anti-TNFR2 antibodies bind to all or a portion of amino acid residues 78-118 of human TNFR2 (SEQ ID NO: 1), and do not bind to one or more amino acids within 120-143 or 161-200 of human TNFR2 (SEQ ID NO: 1). In another embodiment, the anti-TNFR2 antibodies bind to all or a portion of amino acid residues 78-118 of human TNFR2 (SEQ ID NO: 1), and do not bind to one or more amino acids within 120-143 and 161-200 of human TNFR2 (SEQ ID NO: 1). In another embodiment, the anti-TNFR2 antibodies bind to all or a portion of amino acid residues 78-118 of human TNFR2 (SEQ ID NO: 1), and do not bind to amino acids within 120-143 and 161-200 of human TNFR2 (SEQ ID NO: 1).

The anti-TNFR2 antibodies (e.g., anti-human TNFR2 antibodies) described herein may also be characterized by their binding to one of more chimeric receptors comprising a human TNFR2 extracellular domain, wherein certain regions in the extracellular domain are replaced with portions of the corresponding mouse TNFR2 regions (i.e., a “TNFR2 chimera”). Table 2 summarizes exemplary TNFR2 chimeras, which can optionally be fused to an antibody Fc region (for example, when used in binding assays; sequences of chimera-Fc fusions are provided in Table 5). A schematic of the TNFR2 chimeras is provided in FIG. 5A.

TABLE 2

| | |
|---|---|
| TNFR2 chimera 0 | Residues 23-54 of human TNFR2 (SEQ ID NO: 1) are replaced with |
| (SEQ ID NO: 5) | residues 23-55 of mouse TNFR2 (SEQ ID NO: 3). |
| TNFR2 chimera 1 | Residues 23-77 of human TNFR2 (SEQ ID NO: 1) are replaced with |
| (SEQ ID NO: 7) | residues 23-78 of mouse TNFR2 (SEQ ID NO: 3). |
| TNFR2 chimera 2 | Residues 23-118 of human TNFR2 (SEQ ID NO: 1) are replaced with |
| (SEQ ID NO: 9) | residues 23-119 of mouse TNFR2 (SEQ ID NO: 3). |
| TNFR2 chimera 3 | Residues 55-257 of human TNFR2 (SEQ ID NO: 1) are replaced with |
| (SEQ ID NO: 11) | residues 56-258 of mouse TNFR2 (SEQ ID NO: 3). |
| TNFR2 chimera 4 | Residues 76-257 of human TNFR2 (SEQ ID NO: 1) are replaced with |
| (SEQ ID NO: 13) | residues 77-258 of mouse TNFR2 (SEQ ID NO: 3). |
| TNFR2 chimera 5 | Residues 201-257 of human TNFR2 (SEQ ID NO: 1) are replaced with |
| (SEQ ID NO: 15) | residues 203-258 of mouse TNFR2 (SEQ ID NO: 3). |
| TNFR2 chimera 6 | Residues 23-200 of human TNFR2 (SEQ ID NO: 1) are replaced with |
| (SEQ ID NO: 17) | residues 23-202 of mouse TNFR2 (SEQ ID NO: 3). |
| TNFR2 chimera 7 | Residues 97-257 of human TNFR2 (SEQ ID NO: 1) are replaced with |
| (SEQ ID NO: 19) | residues 98-258 of mouse TNFR2 (SEQ ID NO: 3). |
| TNFR2 chimera 8 | Residues 23-96 of human TNFR2 (SEQ ID NO: 1) are replaced with |
| (SEQ ID NO: 21) | residues 23-97 of mouse TNFR2 (SEQ ID NO: 3). |
| TNFR2 chimera 9 | Residues 119-257 of human TNFR2 (SEQ ID NO: 1) are replaced with |
| (SEQ ID NO: 23) | residues 120-258 of mouse TNFR2 (SEQ ID NO: 3). |

Accordingly, in one aspect, provided herein are anti-human TNFR2 antibodies that bind to TNFR2 chimera 3 (SEQ ID NO: 11 or 12) (e.g., with a K_{D} less than 1×10⁻⁵ M, less than 1×10⁻⁶ M, less than 1×10⁻⁷ M, less than 1×10⁻⁸ M, or less than 1×10⁻⁹ M), and do not bind TNFR2 chimera 0 (SEQ ID NO: 5 or 6) (e.g., do not bind with a K_{D} less than 1×10⁻⁵ M, less than 1×10⁻⁶ M, less than 1×10⁻⁷ M, or less than 1×10⁻⁸ M). In one embodiment, the anti-TNFR2 antibodies bind to TNFR2 chimera 3 (SEQ ID NO: 11 or 12) with at least ten-fold (e.g., at least 20-fold, at least 50-fold, at least 100-fold, at least 200-fold, at least 500-fold, at least 1000-fold, ate last 2000-fold, at least 5000-fold, or at least 10,000-fold) better affinity than the anti-TNFR2 antibodies bind to TNFR2 chimera 0 (SEQ ID NO: 5 or 6).

Exemplary mouse anti-human TNFR2 antibodies that bind chimera 3, and do not bind chimera 0, include antibodies produced by hybridomas ABV3, ABV4, ABV7, ABV12, ABV13, ABV14, ABV15, ABV18, and ABV19.

In one embodiment, the anti-TNFR2 antibody is the antibody produced by hybridoma ABV3. In another embodiment, the anti-TNFR2 antibody comprises the VHCDR1-3 and VLCDR1-3 sequences of the antibody produced by hybridoma ABV3. In another embodiment, the anti-TNFR2 antibody is a humanized or chimeric form of the antibody produced by hybridoma ABV3.

In one embodiment, the anti-TNFR2 antibody is the antibody produced by hybridoma ABV4. In another embodiment, the anti-TNFR2 antibody comprises the VHCDR1-3 and VLCDR1-3 sequences of the antibody produced by hybridoma ABV4. In another embodiment, the anti-TNFR2 antibody is a humanized or chimeric form of the antibody produced by hybridoma ABV4.

In one embodiment, the anti-TNFR2 antibody is the antibody produced by hybridoma ABV7. In another embodiment, the anti-TNFR2 antibody comprises the VHCDR1-3 and VLCDR1-3 sequences of the antibody produced by hybridoma ABV7. In another embodiment, the anti-TNFR2 antibody is a humanized or chimeric form of the antibody produced by hybridoma ABV7.

In one embodiment, the anti-TNFR2 antibody is the antibody produced by hybridoma ABV12. In another embodiment, the anti-TNFR2 antibody comprises the VHCDR1-3 and VLCDR1-3 sequences of the antibody produced by hybridoma ABV12. In another embodiment, the anti-TNFR2 antibody is a humanized or chimeric form of the antibody produced by hybridoma ABV12.

In one embodiment, the anti-TNFR2 antibody is the antibody produced by hybridoma ABV13. In another embodiment, the anti-TNFR2 antibody comprises the VHCDR1-3 and VLCDR1-3 sequences of the antibody produced by hybridoma ABV13. In another embodiment, the anti-TNFR2 antibody is a humanized or chimeric form of the antibody produced by hybridoma ABV13.

In one embodiment, the anti-TNFR2 antibody is the antibody produced by hybridoma ABV14. In another embodiment, the anti-TNFR2 antibody comprises the VHCDR1-3 and VLCDR1-3 sequences of the antibody produced by hybridoma ABV14. In another embodiment, the anti-TNFR2 antibody is a humanized or chimeric form of the antibody produced by hybridoma ABV14.

In one embodiment, the anti-TNFR2 antibody is the antibody produced by hybridoma ABV15. In another embodiment, the anti-TNFR2 antibody comprises the VHCDR1-3 and VLCDR1-3 sequences of the antibody produced by hybridoma ABV15. In another embodiment, the anti-TNFR2 antibody is a humanized or chimeric form of the antibody produced by hybridoma ABV15.

In one embodiment, the anti-TNFR2 antibody is the antibody produced by hybridoma ABV18. In another embodiment, the anti-TNFR2 antibody comprises the VHCDR1-3 and VLCDR1-3 sequences of the antibody produced by hybridoma ABV18. In another embodiment, the anti-TNFR2 antibody is a humanized or chimeric form of the antibody produced by hybridoma ABV18.

In one embodiment, the anti-TNFR2 antibody is the antibody produced by hybridoma ABV19. In another embodiment, the anti-TNFR2 antibody comprises the VHCDR1-3 and VLCDR1-3 sequences of the antibody produced by hybridoma ABV19. In another embodiment, the anti-TNFR2 antibody is a humanized or chimeric form of the antibody produced by hybridoma ABV19.

In one embodiment, the anti-TNFR2 antibodies described herein bind to TNFR2 chimeras 3, 4, 5, 7, and/or 9 (SEQ ID NOs: 11, 13, 15, and 23 (or SEQ ID NOs: 12, 14, 15, and 24), respectively) (e.g., with a K_{D} less than 1×10⁻⁵ M, less than 1×10⁻⁶ M, less than 1×10⁻⁷ M, less than 1×10⁻⁸ M, or less than 1×10⁻⁹ M), but do not bind to TNFR2 chimeras 0, 1, 2, 6, and/or 8 (SEQ ID NOs: 5, 7, 9, 17, and 21 (or SEQ ID NOs: 6, 8, 10, 18, and 22), respectively) (e.g., do not bind with a K_{D} less than 1×10⁻⁵ M, less than 1×10⁻⁶ M, less than 1×10⁻⁷ M, or less than 1×10⁻⁸ M). In another embodiment, the anti-TNFR2 antibodies described herein bind to TNFR2 chimeras 3, 4, 5, 7, and 9 (SEQ ID NOs: 11, 13, 15, 19, and 23 (or SEQ ID NOs: 12, 14, 16, 20, and 24), respectively) with a K_{D} less than 1×10⁻⁷ M. In another embodiment, the anti-TNFR2 antibodies described herein bind to TNFR2 chimeras 3, 4, 5, 7, and 9 (SEQ ID NOs: 11, 13, 15, 19, and 23 (or SEQ ID NOs: 12, 14, 16, 20, and 24), respectively) with a K_{D} less than 1×10⁻⁷ M, but do not bind to TNFR2 chimeras 0, 1, 2, 6, and 8 (SEQ ID NOs: 5, 7, 9, 17, and 21 (or SEQ ID NOs: 6, 8, 10, 18, and 22), respectively) with a K_{D} less than 1×10⁻⁷ M. In some embodiments of this aspect, the anti-TNFR2 antibodies significantly inhibit the binding of TNF-alpha to human TNFR2 (e.g., inhibit the binding of TNF-alpha to human TNFR2 by greater than 50% (e.g., greater than 60%, greater than 70%, greater than 80%, greater than 90%, or greater than 95%), as assessed by ELISA (e.g., as described in Example 3)).

In another aspect, provided herein are anti-human TNFR2 antibodies that bind to TNFR2 chimera 7 (SEQ ID NO: 19 or 20) (e.g., with a K_{D} less than 1×10⁻⁵ M, less than 1×10⁻⁶ M, less than 1×10⁻⁷ M, less than 1×10⁻⁸ M, or less than 1×10⁻⁹ M), but do not bind to TNFR2 chimera 4 (SEQ ID NO: 13 or 14) (e.g., do not bind with a K_{D} less than 1×10⁻⁵ M, less than 1×10⁻⁶ M, less than 1×10⁻⁷ M, or less than 1×10⁻⁸ M). In one embodiment, the anti-TNFR2 antibodies bind to TNFR2 chimera 7 (SEQ ID NO: 19 or 20) with at least ten-fold (e.g., at least 20-fold, at least 50-fold, at least 100-fold, at least 200-fold, at least 500-fold, at least 1000-fold, at least 2000-fold, at least 5000-fold, or at least 10,000-fold) better affinity than the anti-TNFR2 antibodies bind to TNFR2 chimera 4 (SEQ ID NO: 13 or 14). In one embodiment, the anti-TNFR2 antibodies do not significantly inhibit the binding of TNF-alpha to human TNFR2 (SEQ ID NO: 1) (e.g., inhibit the binding of TNF-alpha to human TNFR2 (SEQ ID NO: 1) by less than about 50%, as assessed by, e.g., ELISA (for example, as described in Example 3)). In another embodiment, the anti-TNFR2 antibodies described herein bind to TNFR2 chimeras 5, 7, and/or 9 (SEQ ID NOs: 15, 19, and 23 (or SEQ ID NOs: 16, 20, and 24), respectively) (e.g., with a K_{D} less than 1×10⁻⁵ M, less than 1×10⁻⁶ M, less than 1×10⁻⁷ M, less than 1×10⁻⁸ M, or less than 1×10⁻⁹ M), but do not bind to TNFR2 chimeras 0, 1, 2, 3, 4, 6, and/or 8 (SEQ ID NOs: 5, 7, 9, 11, 13, 17, and 21 (or SEQ ID NOs: 6, 8, 10, 12, 14, 18, and 22), respectively) (e.g., does not bind with a K_{D} less than 1×10⁻⁵ M, less than 1×10⁻⁶ M, less than 1×10⁻⁷ M, or less than 1×10⁻⁸ M). In another embodiment, the anti-TNFR2 antibodies described herein bind to TNFR2 chimeras 5, 7, and 9 (SEQ ID NOs: 15, 19, and 23 (or SEQ ID NOs: 16, 20, and 24), respectively) with a K_{D} less than 1×10⁻⁷ M. In another embodiment, the anti-TNFR2 antibodies described herein bind to TNFR2 chimeras 5, 7, and 9 (SEQ ID NOs: 15, 19, and 23 (or SEQ ID NOs: 16, 20, and 24), respectively) (e.g., with a K_{D} less than 1×10⁻⁵ M, less than 1×10⁻⁶ M, less than 1×10⁻⁷ M, less than 1×10⁻⁸ M, or less than 1×10⁻⁹ M), but do not bind to TNFR2 chimeras 0, 1, 2, 3, 4, 6, and 8 (SEQ ID NOs: 5, 7, 9, 11, 13, 17, and 21 (or SEQ ID NOs: 6, 8, 10, 12, 14, 18, and 22), respectively) with a K_{D} less than 1×10⁻⁷ M. In some embodiments of this aspect, the anti-TNFR2 antibodies do not significantly inhibit the binding of TNF-alpha to human TNFR2 (e.g., inhibit the binding of TNF-alpha to human TNFR2 by less than 50%, as assessed by ELISA (e.g., as described in Example 3)). In some embodiments of this aspect, the anti-TNFR2 antibodies exhibit TNFR2 agonist activity (e.g., induce IKBa degradation in Treg cells (e.g., as described in Example 10) with at least 50% of the effectiveness of TNF-alpha).

In another aspect, provided herein are anti-human TNFR2 antibodies that bind to TNFR2 chimera 1 (SEQ ID NO: 7 or 8) (e.g., with a K_{D} less than 1×10⁻⁵ M, less than 1×10⁻⁶ M, less than 1×10⁻⁷ M, less than 1×10⁻⁸ M, or less than 1×10⁻⁹ M), but do not bind to TNFR2 chimera 2 (SEQ ID NO: 9 or 10) (e.g., do not bind with a K_{D} less than 1×10⁻⁵ M, less than 1×10⁻⁶ M, less than 1×10⁻⁷ M, or less than 1×10⁻⁸ M). In one embodiment, the anti-TNFR2 antibodies bind to TNFR2 chimera 1 (SEQ ID NO: 7 or 8) with at least ten-fold (e.g., at least 20-fold, at least 50-fold, at least 100-fold, at least 200-fold, at least 500-fold, at least 1000-fold, ate last 2000-fold, at least 5000-fold, or at least 10,000-fold) better affinity than the anti-TNFR2 antibodies bind to TNFR2 chimera 2 (SEQ ID NO: 9 or 10). In one embodiment, the anti-TNFR2 antibodies described herein bind to TNFR2 chimeras 0, 1, and/or 5 (SEQ ID NOs: 5, 7, and 15 (or SEQ ID NOs: 6, 8, and 16), respectively) (e.g., with a K_{D} less than 1×10⁻⁵ M, less than 1×10⁻⁶ M, less than 1×10⁻⁷ M, less than 1×10⁻⁸ M, or less than 1×10⁻⁹ M), but do not bind to TNFR2 chimeras 2, 3, 4, 6, 7, 8, and/or 9 (SEQ ID NOs: 9, 11, 13, 17, 19, 21, and 23 (or SEQ ID NOs: 10, 12, 14, 18, 20, 22, and 24), respectively) (e.g., do not bind with a K_{D} less than 1×10⁻⁵ M, less than 1×10⁻⁶ M, less than 1×10⁻⁷ M, or less than 1×10⁻⁸ M). In another embodiment, the anti-TNFR2 antibodies described herein bind to TNFR2 chimeras 0, 1, and 5 (SEQ ID NOs: 5, 7, and 15 (or SEQ ID NOs: 6, 8, and 16), respectively) with a K_{D} less than 1×10⁻⁷. In another embodiments, the anti-TNFR2 antibodies described herein bind to TNFR2 chimeras 0, 1, and 5 (SEQ ID NOs: 5, 7, and 15 (or SEQ ID NOs: 6, 8, and 16), respectively) (e.g., with a K_{D} less than 1×10⁻⁵ M, less than 1×10⁻⁶ M, less than 1×10⁻⁷ M, less than 1×10⁻⁸ M, or less than 1×10⁻⁹ M), but do not bind to TNFR2 chimeras 2, 3, 4, 6, 7, 8, and 9 (SEQ ID NOs: 9, 11, 13, 17, 19, 21, and 23 (or SEQ ID NOs: 10, 12, 14, 18, 20, 22, and 24), respectively) with a K_{D} less than 1×10⁻⁷ M. In some embodiments of this aspect, the anti-TNFR2 antibodies significantly inhibit the binding of TNF-alpha to human TNFR2 (e.g., inhibit the binding of TNF-alpha to human TNFR2 by greater than 50% (e.g., greater than 60%, greater than 70%, greater than 80%, greater than 90%, or greater than 95%), as assessed by ELISA (e.g., as described in Example 3)).

In another aspect, provided herein are anti-human TNFR2 antibodies that bind to TNFR2 chimeras 0, 1, 2, 5, and/or 8 (SEQ ID NOs: 5, 7, 9, 15, and 21 (or SEQ ID NOs: 6, 8, 10, 16, and 22), respectively) (e.g., with a K_{D} less than 1×10⁻⁵ M, less than 1×10⁻⁶ M, less than 1×10⁻⁷ M, less than 1×10⁻⁸ M, or less than 1×10⁻⁹ M), but do not bind to TNFR2 chimeras 3, 4, 6, 7, and/or 9 (SEQ ID NOs: 11, 13, 17, 19, and 23 (or SEQ ID NOs: 12, 14, 18, 20, and 24), respectively) (e.g., do not bind with a K_{D} less than 1×10⁻⁵ M, less than 1×10⁻⁶ M, less than 1×10⁻⁷ M, or less than 1×10⁻⁸ M). In one embodiment, the anti-TNFR2 antibodies do not inhibit the binding of TNF-alpha to human TNFR2 (SEQ ID NO: 1). In another embodiment, the anti-TNFR2 antibodies described herein bind to TNFR2 chimeras 0, 1, 2, 5, and 8 (SEQ ID NOs: 5, 7, 9, 15, and 21 (or SEQ ID NOs: 6, 8, 10, 16, and 22), respectively) with a K_{D} less than 1×10⁻⁷ M. In some embodiments, the anti-TNFR2 antibodies described herein bind to TNFR2 chimeras 0, 1, 2, 5, and 8 (SEQ ID NOs: 5, 7, 9, 15, and 21 (or SEQ ID NOs: 6, 8, 10, 16, and 22), respectively) (e.g., with a K_{D} less than 1×10⁻⁵ M, less than 1×10⁻⁶ M, less than 1×10⁻⁷ M, less than 1×10⁻⁸ M, or less than 1×10⁻⁹ M), but do not bind to TNFR2 chimeras 3, 4, 6, 7, and 9 (SEQ ID NOs: 11, 13, 17, 19, and 23 (or SEQ ID NOs: 12, 14, 18, 20, and 24), respectively) with a K_{D} less than 1×10⁻⁷. In some embodiments, the antibodies do not bind to TNFR2 chimera 8 with a K_{D} less than 1×10⁻⁵ M, less than 1×10⁻⁶ M, less than 1×10⁻⁷ M, or less than 1×10⁻⁸ M. In some embodiments of this aspect, the anti-TNFR2 antibodies do not significantly inhibit the binding of TNF-alpha to human TNFR2 (e.g., inhibit the binding of TNF-alpha to human TNFR2 by less than 50%, as assessed by ELISA (e.g., as described in Example 3)).

In another aspect, provided herein are anti-human TNFR2 antibodies that bind to TNFR2 chimeras 0, 1, 2, 5, and/or 8 (SEQ ID NOs: 5, 7, 9, 15, and 21 (or SEQ ID NOs: 6, 8, 10, 16, and 22), respectively) (e.g., with a K_{D} less than 1×10⁻⁵ M, less than 1×10⁻⁶ M, less than 1×10⁻⁷ M, less than 1×10⁻⁸ M, or less than 1×10⁻⁹ M), but do not bind to TNFR2 chimeras 3, 4, 6, 7, and/or 9 (SEQ ID NOs: 11, 13, 17, 19, and 23 (or SEQ ID NOs: 12, 14, 18, 20, and 24), respectively) (e.g., do not bind with a K_{D} less than 1×10⁻⁵ M, less than 1×10⁻⁶ M, less than 1×10⁻⁷ M, or less than 1×10⁻⁸ M). In one embodiment, the anti-TNFR2 antibodies described herein bind to TNFR2 chimeras 0, 1, 2, 5, and/or 8 (SEQ ID NOs: 5, 7, 9, 15, and 21 (or SEQ ID NOs: 6, 8, 10, 16, and 22), respectively) with a K_{D} less than 1×10⁻⁷ M. In some embodiments, the anti-TNFR2 antibodies described herein bind to TNFR2 chimeras 0, 1, 2, 5, and 8 (SEQ ID NOs: 5, 7, 9, 15, and 21 (or SEQ ID NOs: 6, 8, 10, 16, and 22), respectively), but do not bind to TNFR2 chimeras 3, 4, 6, 7, and 9 (SEQ ID NOs: 11, 13, 17, 19, and 23 (or SEQ ID NOs: 12, 14, 18, 20, and 24), respectively) with a K_{D} less than 1×10⁻⁷ M.

Anti-TNFR2 antibodies disclosed herein can also be characterized by particular functional and structural features (e.g., CDRs, variable regions, heavy and light chains).

Accordingly, in one embodiment, the antibody binds to human TNFR2 and comprises heavy and light chain CDR1, CDR2, and CDR3 sequences of the heavy and light chain variable region pair comprising the amino acid sequences set forth in (a) SEQ ID NOs: 71 and 72, respectively, (b) SEQ ID NOs: 74 and 86, respectively, (c) SEQ ID NOs: 170 and 171, respectively, (d) SEQ ID NOs: 148 and 149, respectively, or (e) SEQ ID NOs: 126 and 127, respectively. In some embodiments, the CDR sequences are defined using Kabat numbering. In other embodiments, the CDR sequences are defined using Chothia numbering. In other embodiments, the CDR sequences are defined using IMGT numbering.

In some embodiments, provided herein are anti-TNFR2 antibodies comprising heavy chain CDR1, CDR2, and CDR3 sequences comprising SEQ ID NOs: 47, 48, and 49, respectively, and light chain CDR1, CDR2, and CDR3 sequences comprising SEQ ID NOs: 50, 51, and 52, respectively.

In some embodiments, provided herein are anti-TNFR2 antibodies comprising heavy chain CDR1, CDR2, and CDR3 sequences comprising SEQ ID NOs: 53, 54, and 55, respectively, and light chain CDR1, CDR2, and CDR3 sequences comprising SEQ ID NOs: 56, 57, and 58, respectively.

In some embodiments, provided herein are anti-TNFR2 antibodies comprising heavy chain CDR1, CDR2, and CDR3 sequences comprising SEQ ID NOs: 59, 60, and 61, respectively, and light chain CDR1, CDR2, and CDR3 sequences comprising SEQ ID NOs: 62, 63, and 64, respectively.

In some embodiments, provided herein are anti-TNFR2 antibodies comprising heavy chain CDR1, CDR2, and CDR3 sequences comprising SEQ ID NOs: 65, 66, and 67, respectively, and light chain CDR1, CDR2, and CDR3 sequences comprising SEQ ID NOs: 68, 69, and 70, respectively.

In some embodiments, provided herein are anti-TNFR2 antibodies comprising heavy chain CDR1, CDR2, and CDR3 sequences comprising SEQ ID NOs: 152, 153, and 154, respectively, and light chain CDR1, CDR2, and CDR3 sequences comprising SEQ ID NOs: 155, 156, and 157, respectively.

In some embodiments, provided herein are anti-TNFR2 antibodies comprising heavy chain CDR1, CDR2, and CDR3 sequences comprising SEQ ID NOs: 158, 159, and 160, respectively, and light chain CDR1, CDR2, and CDR3 sequences comprising SEQ ID NOs: 161, 162, and 163, respectively.

In some embodiments, provided herein are anti-TNFR2 antibodies comprising heavy chain CDR1, CDR2, and CDR3 sequences comprising SEQ ID NOs: 164, 165, and 166, respectively, and light chain CDR1, CDR2, and CDR3 sequences comprising SEQ ID NOs: 167, 168, and 169, respectively.

In some embodiments, provided herein are anti-TNFR2 antibodies comprising heavy chain CDR1, CDR2, and CDR3 sequences comprising SEQ ID NOs: 130, 131, and 132, respectively, and light chain CDR1, CDR2, and CDR3 sequences comprising SEQ ID NOs: 133, 134, and 135, respectively.

In some embodiments, provided herein are anti-TNFR2 antibodies comprising heavy chain CDR1, CDR2, and CDR3 sequences comprising SEQ ID NOs: 136, 137, and 138, respectively, and light chain CDR1, CDR2, and CDR3 sequences comprising SEQ ID NOs: 139, 140, and 141, respectively.

In some embodiments, provided herein are anti-TNFR2 antibodies comprising heavy chain CDR1, CDR2, and CDR3 sequences comprising SEQ ID NOs: 142, 143, and 144, respectively, and light chain CDR1, CDR2, and CDR3 sequences comprising SEQ ID NOs: 145, 146, and 147, respectively.

In some embodiments, provided herein are anti-TNFR2 antibodies comprising heavy chain CDR1, CDR2, and CDR3 sequences comprising SEQ ID NOs: 108, 109, and 110, respectively, and light chain CDR1, CDR2, and CDR3 sequences comprising SEQ ID NOs: 111, 112, and 113, respectively.

In some embodiments, provided herein are anti-TNFR2 antibodies comprising heavy chain CDR1, CDR2, and CDR3 sequences comprising SEQ ID NOs: 114, 115, and 116, respectively, and light chain CDR1, CDR2, and CDR3 sequences comprising SEQ ID NOs: 117, 118, and 119, respectively.

In some embodiments, provided herein are anti-TNFR2 antibodies comprising heavy chain CDR1, CDR2, and CDR3 sequences comprising SEQ ID NOs: 120, 121, and 122, respectively, and light chain CDR1, CDR2, and CDR3 sequences comprising SEQ ID NOs: 123, 124, and 125, respectively.

In some embodiments, the anti-TNFR2 antibody comprises the heavy chain CDR sequences above, and a constant region, e.g., a human IgG constant region (e.g., IgG1, IgG2, IgG3, or IgG4, or variants thereof). In other embodiments, a heavy chain variable region comprising the heavy chain CDR sequences described above may be linked to a constant domain to form a heavy chain (e.g., a full length heavy chain). Similarly, a light chain variable region comprising the light chain CDR sequences described above may be linked to a constant region to form a light chain (e.g., a full length light chain). A full length heavy chain (with the exception of the C-terminal lysine (K) or with the exception of the C-terminal glycine and lysine (GK), which may be absent or removed) and full length light chain combine to form a full length antibody.

In some embodiments, the anti-TNFR2 antibody comprises a heavy chain variable region and a light chain variable region, wherein the heavy chain variable region comprises an amino acid sequence selected from the group consisting of SEQ ID NOs: 71, 73, 74, 75, 76, 77, 78, 79, 80, 81, 82, 83, 84, 126, 148, and 170. In other embodiments, the anti-TNFR2 antibody comprises a heavy chain variable region and a light chain variable region, wherein the light chain variable region comprises an amino acid sequence selected from the group consisting of SEQ ID NOs: 72, 85, 86, 87, 88, 89, 90, 91, 92, 93, 94, 95, 96, 97, 98, 99, 100, 127, 149, and 171. In other embodiments, the anti-TNFR2 antibody comprises a heavy chain variable region and a light chain variable region, wherein the heavy chain variable region comprises an amino acid sequence selected from the group consisting of SEQ ID NOs: 71, 73, 74, 75, 76, 77, 78, 79, 80, 81, 82, 83, 84, 126, 148, and 170, and the light chain variable region comprises an amino acid sequence selected from the group consisting of SEQ ID NOs: 72, 85, 86, 87, 88, 89, 90, 91, 92, 93, 94, 95, 96, 97, 98, 99, 100, 127, 149, and 171.

In some embodiments, the anti-TNFR2 antibody comprises a heavy chain variable region and a light chain variable region, wherein the heavy chain variable region and/or light chain variable region sequence is at least 80%, 85%, 90%, 95%, 96%, 97%, 98%, or 99% identical to the heavy chain and/or light chain variable region sequences described above (e.g., SEQ ID NOs: 71-100, 126, 127, 148, 149, 170, and 171). In other embodiments, the heavy chain and/or light chain variable region sequences (e.g., SEQ ID NOs: 71-100, 126, 127, 148, 149, 170, and 171) have 1, 2, 3, 4, 5, 1-2, 1-3, 1-4, or 1-5 amino acid substitutions (e.g., conservative amino acid substitutions).

In some embodiments, the anti-TNFR2 antibody comprises the heavy chain variable region sequences of any of SEQ ID NOs: 71, 73, 74, 75, 76, 77, 78, 79, 80, 81, 82, 83, 84, 126, 148, and 170 and a constant region, e.g., a human IgG constant region (e.g., IgG1, IgG2, IgG3, or IgG4, or variants thereof). In other embodiments, the heavy chain variable region sequences of any of SEQ ID NOs: 71, 73, 74, 75, 76, 77, 78, 79, 80, 81, 82, 83, 84, 126, 148, and 170 may be linked to a constant domain to form a heavy chain (e.g., a full length heavy chain). Similarly, the light chain variable region sequences of any of SEQ ID NOs: 72, 85, 86, 87, 88, 89, 90, 91, 92, 93, 94, 95, 96, 97, 98, 99, 100, 127, 149, and 171 may be linked to a constant region to form a light chain (e.g., a full length light chain). A full length heavy chain (with the exception of the C-terminal lysine (K) or with the exception of the C-terminal glycine and lysine (GK), which may be absent or removed) and full length light chain combine to form a full length antibody.

In some embodiments, the anti-TNFR2 antibody comprises heavy and light chain variable region sequences which are at least 80%, at least 85%, at least 90%, at least 91%, at least 92%, at least 93%, at least 94%, at least 95%, at least 96%, at least 97%, at least 98%, at least 99%, or are 100% identical to the amino acid sequences set forth in SEQ ID NOs: 71 and/or 72, respectively. In other embodiments, the anti-TNFR2 antibody comprises a heavy chain variable region comprising the amino acid sequence of SEQ ID NO: 71, and a light chain variable region comprising the amino acid sequence of SEQ ID NO: 72.

In some embodiments, the anti-TNFR2 antibody comprises heavy and light chain variable region sequences which are at least 80%, at least 85%, at least 90%, at least 91%, at least 92%, at least 93%, at least 94%, at least 95%, at least 96%, at least 97%, at least 98%, at least 99%, or are 100% identical to the amino acid sequences set forth in SEQ ID NOs: 74 and/or 86, respectively. In other embodiments, the anti-TNFR2 antibody comprises a heavy chain variable region comprising the amino acid sequence of SEQ ID NO: 74, and a light chain variable region comprising the amino acid sequence of SEQ ID NO: 86.

In some embodiments, the anti-TNFR2 antibody comprises heavy and light chain variable region sequences which are at least 80%, at least 85%, at least 90%, at least 91%, at least 92%, at least 93%, at least 94%, at least 95%, at least 96%, at least 97%, at least 98%, at least 99%, or are 100% identical to the amino acid sequences set forth in SEQ ID NOs: 170 and/or 171, respectively. In other embodiments, the anti-TNFR2 antibody comprises a heavy chain variable region comprising the amino acid sequence of SEQ ID NO: 170, and a light chain variable region comprising the amino acid sequence of SEQ ID NO: 171.

In some embodiments, the anti-TNFR2 antibody comprises heavy and light chain variable region sequences which are at least 80%, at least 85%, at least 90%, at least 91%, at least 92%, at least 93%, at least 94%, at least 95%, at least 96%, at least 97%, at least 98%, at least 99%, or are 100% identical to the amino acid sequences set forth in SEQ ID NOs: 148 and/or 149, respectively. In other embodiments, the anti-TNFR2 antibody comprises a heavy chain variable region comprising the amino acid sequence of SEQ ID NO: 148, and a light chain variable region comprising the amino acid sequence of SEQ ID NO: 149.

In some embodiments, the anti-TNFR2 antibody comprises heavy and light chain variable region sequences which are at least 80%, at least 85%, at least 90%, at least 91%, at least 92%, at least 93%, at least 94%, at least 95%, at least 96%, at least 97%, at least 98%, at least 99%, or are 100% identical to the amino acid sequences set forth in SEQ ID NOs: 126 and/or 127, respectively. In other embodiments, the anti-TNFR2 antibody comprises a heavy chain variable region comprising the amino acid sequence of SEQ ID NO: 126, and a light chain variable region comprising the amino acid sequence of SEQ ID NO: 127.

In some embodiments, the heavy chain and/or light chain variable region sequences above have 1, 2, 3, 4, 5, 1-2, 1-3, 1-4, or 1-5 amino acid substitutions (e.g., conservative amino acid substitutions).

In some embodiments, antibodies comprising the heavy and light chain CDR sequences or heavy and light chain variable region sequences described herein are human, humanized, or chimeric antibodies (e.g., recombinant human, humanized, or chimeric antibodies).

In some embodiments, the anti-human TNFR2 antibody comprises the heavy chain variable region sequences above, and a constant region, e.g., a human IgG constant region (e.g., IgG1, IgG2, IgG3, or IgG4, or variants thereof) to form a heavy chain (e.g., a full length heavy chain). Similarly, a light chain variable region comprising the light chain variable region sequences described above may be linked to a constant region to form a light chain (e.g., a full length light chain). A full length heavy chain (with the exception of the C-terminal lysine (K) or with the exception of the C-terminal glycine and lysine (GK), which may be absent or removed) and full length light chain combine to form a full length antibody.

In some embodiments, provided herein are anti-TNFR2 antibodies comprising heavy and light chain sequences which are at least 80%, at least 85%, at least 90%, at least 91%, at least 92%, at least 93%, at least 94%, at least 95%, at least 96%, at least 97%, at least 98%, at least 99%, or are 100% identical to the amino acid sequences set forth in SEQ ID NOs: 101 and/or 102, respectively.

In some embodiments, provided herein are anti-TNFR2 antibodies comprising heavy and light chain sequences which are at least 80%, at least 85%, at least 90%, at least 91%, at least 92%, at least 93%, at least 94%, at least 95%, at least 96%, at least 97%, at least 98%, at least 99%, or are 100% identical to the amino acid sequences set forth in SEQ ID NOs: 150 and/or 151, respectively.

In some embodiments, provided herein are anti-TNFR2 antibodies comprising heavy and light chain sequences which are at least 80%, at least 85%, at least 90%, at least 91%, at least 92%, at least 93%, at least 94%, at least 95%, at least 96%, at least 97%, at least 98%, at least 99%, or are 100% identical to the amino acid sequences set forth in SEQ ID NOs: 128 and/or 129, respectively.

In some embodiments, provided herein are anti-TNFR2 antibodies comprising heavy and light chain sequences which are at least 80%, at least 85%, at least 90%, at least 91%, at least 92%, at least 93%, at least 94%, at least 95%, at least 96%, at least 97%, at least 98%, at least 99%, or are 100% identical to the amino acid sequences set forth in SEQ ID NOs: 106 and/or 107, respectively.

In some embodiments, the heavy chain and/or light chain sequences above have 1, 2, 3, 4, 5, 1-2, 1-3, 1-4, or 1-5 amino acid substitutions (e.g., conservative amino acid substitutions).

In some embodiments, the anti-TNFR2 antibodies bind to the extracellular domain of TNFR2 (e.g., human TNFR2), or a particular human TNFR2 epitope (such as those discussed above), for example, with a K_{D} of 10⁻⁷ M or less, 10⁻⁸ M or less, 10⁻⁹ M or less, 10⁻¹⁰ M or less, 10⁻¹¹ M or less, 10⁻¹² M or less, 10⁻¹² M to 10⁻⁷ M, 10⁻¹¹ M to 10⁻⁷ M, 10⁻¹⁰ M to 10⁻⁷ M, or 10⁻⁹ M to 10⁻⁷ M, as assessed by, e.g., bio-layer interferometery.

In some embodiments, the anti-TNFR2 antibodies bind to a discontinuous epitope on human TNFR2.

In some embodiments, the anti-TNFR2 antibodies do not inhibit the binding of TNFR2 ligand (e.g., TNFα) to TNFR2. In some embodiments, the anti-TNFR2 antibodies partially inhibit the binding of TNFR2 ligand (e.g., TNFα) to TNFR2. In some embodiments, the anti-TNFR2 antibodies inhibit the binding of TNFR2 ligand (e.g., TNFα) to TNFR2. In some embodiments, the anti-TNFR2 antibodies inhibit the binding of TNFR2 ligand (e.g., TNFα) to TNFR2 by at least 10%, for example, by at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99%, relative to a control antibody (e.g., an antibody which does not bind to TNFR2).

In other embodiments, the anti-TNFR2 antibodies described herein activate TNFR2 signaling pathways in cells (i.e., agonist antibodies).

In some embodiments, the anti-TNFR2 antibodies increase NF-kB activity, e.g., as assessed by NF-kB reporter cell lines (e.g., NF-kB reporter cell lines engineered to express human TNFR2). In other embodiments, the anti-TNFR2 antibodies increase NF-kB activity by, e.g., at least 2-fold, at least 3-fold, at least 4-fold, at least 5-fold, at least 10-fold, at least 15-fold, or at least 20-fold relative to a control (e.g., an isotype control antibody or the NF-kB reporter cell line which does not express human TNFR2).

In some embodiments, the anti-TNFR2 antibodies decrease the percentage of regulatory T cells (Tregs) within the CD4+ T cell compartment relative to a control (e.g., no antibody control or isotype antibody control). In some embodiments, the anti-TNFR2 antibodies decrease the percentage of Treg cells within the CD4+ T cell compartment by about 10%, about 20%, about 30%, about 40%, about 50%, about 60%, about 70%, or about 80% relative to a control (e.g., no antibody control or isotype antibody control).

In some embodiments, the anti-TNFR2 antibodies induce ADCC in the presence of NK cells.

In some embodiments, the anti-TNFR2 antibodies enhance T cell activation. In some embodiments, the anti-TNFR2 antibodies described herein enhance the activation of CD4+ and CD8+ T cells, e.g., as reflected in the increased expression of activation markers (e.g., CD25, PD1), as assessed by, e.g., flow cytometry.

In some embodiments, the anti-TNFR2 antibodies increase T cell proliferation. In some embodiments, the anti-TNFR2 antibodies described herein increase the proliferation of CD4+ T cells and CD8+ T cells.

In some embodiments, the anti-TNFR2 antibodies reduce (protect against) graft rejection, e.g., as assessed in a graft-versus-host disease (GvHD) model. Reduced graft rejection can be assessed, e.g., by comparison with a control (e.g., improved survival relative to treatment with a control antibody or vehicle or an unrelated antibody).

In some embodiments, the anti-TNFR2 antibodies inhibit tumor growth, for example, by 10% or more, 20% or more, 30% or more, 40% or more, 50% or more, 60% or more, 70% or more, 80% or more, 90% or more, or 95% or more, relative to a control therapy.

In some embodiments, the anti-TNFR2 antibodies inhibit tumor growth independent of the ability to agonize TNFR2 signaling.

In some embodiments, the anti-TNFR2 antibodies inhibit tumor growth independent of the ability to inhibit TNF-alpha binding to TNFR2.

In some embodiments, the anti-TNFR2 antibodies induce a long-term anti-cancer effect (e.g., inhibit and/or suppress tumor growth for a sustained period of time after treatment with the anti-TNFR2 antibodies). In a particular embodiment, the anti-TNFR2 antibodies induce the development of anti-cancer memory T cells, as compared to control (e.g., subjects not treated with anti-TNFR2 antibodies).

Also provided herein are methods of inducing a long-term anti-cancer effect comprising administering the anti-TNFR2 antibodies described herein to a subject with cancer.

In one embodiment, a long-term anti-cancer effect can be measured in mouse models of human cancer (e.g., transgenic models, humanized models, and/or chimeric, allograft, and xenograft models). Tumor recurrence (or suppression) can be monitored, e.g., for at least 6 months, in mice which exhibited tumor regression after initial treatment with anti-TNFR2 antibodies. In other embodiments, tumor recurrence (or suppression) can be monitored for at least 1 or more years or at least 2 or more years.

In another embodiment, to determine whether cytotoxic T lymphocytes (CTLs) have develop into memory T cells, various doses of the same tumor cells can be reinoculated into the tumor-regressed mice at different time points after the tumor regression, and then monitor tumor grow in the recipient mouse. Wildtype mice can be inoculated with the same tumor as controls. To determine the frequency of tumor specific memory T cells in tumor regressed mice, in vitro cytotoxicity assay can be performed using particular cancer cell antigens as targets.

In some embodiments, the anti-TNFR2 antibodies are monoclonal antibodies, e.g., monoclonal human antibodies.

In some embodiments, the anti-TNFR2 antibodies are human, humanized, or chimeric antibodies.

An antibody that exhibits one or more of the functional properties described above (e.g., biochemical, immunochemical, cellular, physiological or other biological activities, or the like) as determined according to methodologies known to the art and described herein, will be understood to relate to a statistically significant difference in the particular activity relative to that seen in the absence of the antibody (e.g., or when a control antibody of irrelevant specificity is present). Preferably, the anti-TNFR2 antibody-induced increases in a measured parameter effects a statistically significant increase by at least 10% of the measured parameter, more preferably by at least 20%, 30%, 40%, 50%, 60%, 70%, 80%, 90%, 95%, or 100% (i.e., 2-fold), 3 fold, 5 fold or 10 fold. Conversely, anti-TNFR2 antibody-induced decreases in a measured parameter (e.g., tumor volume, TNFα binding to TNFR2) effects a statistically significant decrease by at least 10%, 20%, 30%, 40%, 50%, 60%, 70%, 80%, 90%, 95%, 97%, 98%, 99%, or 100%.

In some embodiments, a VH domain of the anti-TNFR2 antibodies is linked to a constant domain to form a heavy chain, e.g., a full-length heavy chain. In other embodiments, the VH domain is linked to the constant domain of a human IgG, e.g., IgG1, IgG2, IgG3, or IgG4, or variants thereof (e.g., variants comprising Fc regions with enhanced effector function). Similarly, a VL domain of the anti-TNFR2 antibodies described herein described herein is linked to a constant domain to form a light chain, e.g., a full-length light chain.

Antibodies disclosed herein include all known forms of antibodies and other protein scaffolds with antibody-like properties. For example, the antibody can be a human antibody, a humanized antibody, a bispecific antibody, an immunoconjugate, a chimeric antibody, or a protein scaffold with antibody-like properties, such as fibronectin or ankyrin repeats. The antibody also can be a Fab, Fab′2, scFv, AFFIBODY, avimer, nanobody, or a domain antibody. The antibody also can have any isotype, including any of the following isotypes: IgG1, IgG2, IgG3, IgG4, IgM, IgA1, IgA2, IgAsec, IgD, and IgE. Full-length antibodies can be prepared from V_{H} and V_{L} sequences using standard recombinant DNA techniques and nucleic acid encoding the desired constant region sequences to be operatively linked to the variable region sequences.

In some embodiments, the anti-TNFR2 antibodies bind to one or more of the following positions (e.g., one, two, three, four, or all five positions) on human TNFR2 (numbering according to SEQ ID NO: 104): Y24, Q26, Q29, M30, and K47. In other embodiments, the anti-TNFR2 antibodies bind to an epitope on human TNFR2 which consists of one or more of the following positions (e.g., one, two, three, four, or all five positions) on human TNFR2 (numbering according to SEQ ID NO: 104): Y24, Q26, Q29, M30, and K47. In other embodiments, the anti-TNFR2 antibodies bind to an epitope on human TNFR2 that spans, is in between, and/or overlaps with amino acid positions 24-47 of human TNFR2 (numbering according to SEQ ID NO: 104).

In some embodiments, the anti-TNFR2 antibodies bind to the same epitope on TNFR2 as the anti-TNFR2 antibodies described herein. In other embodiments, the antibodies compete for binding to TNFR2 with the anti-TNFR2 antibodies described herein.

In some embodiments, the anti-TNFR2 antibodies are modified to enhance effector function relative to the same antibody in unmodified form. In other embodiments, the anti-TNFR2 antibodies exhibit increased anti-tumor activity relative to the same antibody in unmodified form.

Accordingly, the variable regions of the anti-TNFR antibodies may be linked to a non-naturally occurring Fc region, e.g., an Fc with enhanced binding to one or more activating Fc receptors (FcγI, FcγIIa or FcγIIIa). In general, the variable regions described herein may be linked to an Fc comprising one or more modification (e.g., an amino acid substitution, deletion, and/or insertion), typically to enhance one or more functional properties of the antibody, such as serum half-life, complement fixation, Fc receptor binding, antibody-dependent cell-mediated cytotoxicity (ADCC), and/or antibody-dependent cellular phagocytosis (ADCP), relative to a parent Fc sequence (e.g., the unmodified Fc polypeptide). Furthermore, an antibody may be chemically modified (e.g., one or more chemical moieties can be attached to the antibody) or be modified to alter its glycosylation, to alter one or more functional properties of the antibody. Each of these embodiments is described in further detail below. The numbering of residues in the Fc region is that of the EU index of Kabat.

Fcγ receptor engagement of therapeutic antibodies can be important for their anti-tumor activity (Clynes et al., Nat Med 2000; 6:443-6). Both mice and humans have activating Fcγ receptors (e.g., mFcγRI, mFcγRIII, or mFcγRIV in mice and hFcγRI, hFcγRIIa, hFcγRIIc, mFcγRIIIa, or mFcγRIIIb in humans) and inhibitory Fcγ receptors (mFcγRIIb in mice and hFcγRIIb in humans) (Nimmerjahn et al., Nat Rev Immunol 2008; 8:34-47). Fcγ receptor engagement can indicate: 1) contribution of effector functions of the antibody such as antibody-dependent cellular cytotoxicity (ADCC), Opsonization or antibody-dependent cellular phagocytosis (ADCP) via activating Fcγ receptors (Dahan et al., Cancer Cell 2015; 28:285-95); or 2) enhanced agonism via clustering of the antibody on Fcγ receptor-expressing cell types (Nimmerjahn et al., Trends in Immunology 2015; 36:325-36. Accordingly, in some embodiments, provided herein are anti-TNFR2 antibodies that mediate the agonistic activity and co-stimulation of T cells. For enhanced agonism, the inhibitory Fcγ receptor FcγRIIb has been described as most important to facilitate agonism (see, e.g., Dahan et al., Cancer Cell 2016; 29:820-31).

The various antibody IgG isotypes have different preferences for binding certain Fcγ receptors (Bruhns et al., Blood 2012; 119:5640-9). In humans, IgG1 antibodies are the preferred isotype for mediating effector functions such as ADCC or ADCP because of their high affinity for activating Fcγ receptors. Various mutations for antibody Fc have been described that alter the binding profile to the various Fcγ receptors, and hence can modulate the activity of an antibody. The N297A mutation (NA), D265A/N297A mutations (DANA), or the D265A/N297G mutations (DANG) reduce or ablate bind to all Fcγ receptors (Lo et al., J Biol Chem 2017; 292:3900-8) and hence reduce capacity for effector functions or enhanced agonism. L234A/L235A mutations (LALA) reduce or ablate bind to all Fcγ receptors (Arduin et al., Mol Immunol 2015; 63:456-63). Similarly, mutations with enhanced binding to FcγRIIb and hence increased agonistic activity have been described (see, e.g., Dahan et al., Cancer Cell 2016; 29:820-31), such as the S267E mutation (SE), the S267E and L328F mutations (SELF), the G237D/P238D/P271G/A330R mutations (V9), the E233D/P238D/H268D/P271G/A330R mutations (V10), the G237D/P238D/H268D/P271G/A330R mutations (V11), or the E233D/G237D/P238D/H268D/P271G/A330R mutations (V12) (Mimoto et al., Protein Eng Des Sel 2013; 26:589-98).

Accordingly, the anti-TNFR2 antibodies may comprise a variant Fc region (e.g., a variant IgG1 Fc region). In some embodiments, the variant Fc region increases binding to Fcγ receptors relative to binding observed with the corresponding non-variant version of the Fc region (e.g., if the variant Fc region is a variant IgG1 Fc region, then the corresponding non-variant version is the wild-type IgG1 Fc region). In some embodiments, the variant Fc region (e.g., variant IgG1 Fc region) increases binding to the FcγRIIb receptor. In some embodiments, the variant Fc region increases antibody clustering relative to the corresponding wild-type Fc region. In some embodiments, the antibody comprises a variant Fc region and exhibits increased agonistic activity relative to an antibody with a corresponding non-variant version of the Fc region. In some embodiments, the antibody co-stimulates T cells. In some embodiments, the variant Fc region is a variant IgG1 Fc region. In some embodiments, the Fc region has a 267E mutation (SE), S267E/L328F mutations (SELF), G237D/P238D/P271G/A330R mutations, E233D/P238D/H268D/P271G/A330R mutations, G237D/P238D/H268D/P271G/A330R mutations, or E233D/G237D/P238D/H268D/P271G/A330R mutations. Other exemplary modifications to the Fc region for altering effector function are described below.

Modifications can be made in the Fc region to generate an Fc variant that (a) has increased antibody-dependent cell-mediated cytotoxicity (ADCC), (b) has increased antibody-dependent cellular phagocytosis (ADCP), (c) has increased complement mediated cytotoxicity (CDC), (d) has increased affinity for Clq and/or (e) has increased affinity for a Fc receptor relative to the parent Fc. Such Fc region variants will generally comprise at least one amino acid modification in the Fc region. Combining amino acid modifications is thought to be particularly desirable. For example, the variant Fc region may include two, three, four, five, etc. substitutions therein, e.g. of the specific Fc region positions identified herein.

In some embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320, and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in detail in U.S. Pat. Nos. 5,624,821 and 5,648,260, both by Winter et al.

In some embodiments, the Fc region may be modified to increase antibody dependent cellular cytotoxicity (ADCC) and/or to increase the affinity for an Fcγ receptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439. Exemplary substitutions include 236A, 239D, 239E, 268D, 267E, 268E, 268F, 324T, 332D, and 332E. Exemplary combinations of substitutions include 239D/332E, 236A/332E, 236A/239D/332E, 268F/324T, 267E/268F, 267E/324T, and 267E/268F/324T. Other modifications for enhancing FcγR and complement interactions include, but are not limited to, substitutions 298A, 333A, 334A, 326A, 2471, 339D, 339Q, 280H, 290S, 298D, 298V, 243L, 292P, 300L, 396L, 3051, and 396L. These and other modifications are reviewed in Strohl et al., Current Opinion in Biotechnology 2009; 20:685-691.

Fc modifications that increase binding to an Fcγ receptor include amino acid modifications at any one or more of amino acid positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 3338, 340, 360, 373, 376, 379, 382, 388, 389, 398, 414, 416, 419, 430, 434, 435, 437, 438, or 439 of the Fc region, wherein the numbering of the residues in the Fc region is that of the EU index as in Kabat (WO00/42072).

Fc variants that enhance affinity for an inhibitory receptor FcγRIIb may also be used. Such variants may provide an Fc fusion protein with immunomodulatory activities related to FcγRIIb⁺ cells, including for example B cells and monocytes. In one embodiment, the Fc variants provide selectively enhanced affinity to FcγRIIb relative to one or more activating receptors. Modifications for altering binding to FcγRIIb include one or more modifications at a position selected from the group consisting of 234, 235, 236, 237, 239, 266, 267, 268, 325, 326, 327, 328, and 332, according to the EU index. Exemplary substitutions for enhancing FcγRIIb affinity include, but are not limited to, 234D, 234E, 234F, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E. Other Fc variants for enhancing binding to FcγRIIb include 235Y/267E, 236D/267E, 239D/268D, 239D/267E, 267E/268D, 267E/268E, and 267E/328F.

The affinities and binding properties of an Fc region for its ligand may be determined by a variety of in vitro assay methods (biochemical or immunological based assays) known in the art including, but not limited to, equilibrium methods (e.g., enzyme-linked immunosorbent assay (ELISA), or radioimmunoassay (RIA)), or kinetics (e.g., BIACORE analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions.

In certain embodiments, the antibody is modified to increase its biological half-life. For example, this may be done by increasing the binding affinity of the Fc region for FcRn by mutating one or more of the following residues: 252, 254, 256, 433, 435, 436, as described in U.S. Pat. No. 6,277,375. Specific exemplary substitutions include one or more of the following: T252L, T254S, and/or T256F. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Other exemplary variants that increase binding to FcRn and/or improve pharmacokinetic properties include substitutions at positions 259, 308, 428, and 434, including for example 2591, 308F, 428L, 428M, 434S, 434H, 434F, 434Y, and 434M. Other variants that increase Fc binding to FcRn include: 250E, 250Q, 428L, 428F, 250Q/428L (Hinton et al., 2004, J. Biol. Chem. 279(8): 6213-6216, Hinton et al. 2006 Journal of Immunology 176:346-356), 256A, 272A, 286A, 305A, 307A, 307Q, 31 1A, 312A, 376A, 378Q, 380A, 382A, 434A (Shields et al, Journal of Biological Chemistry, 2001, 276(9):6591-6604), 252F, 252T, 252Y, 252W, 254T, 256S, 256R, 256Q, 256E, 256D, 256T, 309P, 311 S, 433R, 433S, 4331, 433P, 433Q, 434H, 434F, 434Y, 252Y/254T/256E, 433K/434F/436H, 308T/309P/311S (Dall Acqua et al. Journal of Immunology, 2002, 169:5171-5180, Dall'Acqua et al., 2006, Journal of Biological Chemistry 281:23514-23524). Other modifications for modulating FcRn binding are described in Yeung et al., 2010, J Immunol, 182:7663-7671.

The binding sites on human IgG1 for FcγR1, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding and ADCC activity: T256A/S298A, S298A/E333A, S298A/K224A, and S298A/E333A/K334A (Shields et al., supra). Other IgG1 variants with strongly enhanced binding to FcγRIIIa have been identified, including variants with S239D/I332E and S239D/I332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/I332E variant showed an enhanced capacity to deplete B cells in monkeys (Lazar et al., 2006). In addition, IgG1 mutants containing L235V, F243L, R292P, Y300L, and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants that may be used include: S298A/E333A/L334A, S239D/I332E, S239D/I332E/A330L, L235V/F243L/R292P/Y300L/P396L, and M428L/N434S.

In another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such an approach is described in further detail in U.S. Pat. Nos. 5,714,350 and 6,350,861 by Co et al. In one embodiment, glycosylation of the constant region on N297 may be prevented by mutating the N297 residue to another residue, e.g., N297A, and/or by mutating an adjacent amino acid, e.g., 298 to thereby reduce glycosylation on N297.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies to thereby produce an antibody with altered glycosylation. In some embodiments, mutations can be made to restore effector function in aglycosylated antibody, e.g., as described in U.S. Pat. No. 8,815,237. Exemplary mutations include E269D, D270E, N297D, N297H, S298A, S298G, S298T, T299A, T299G, T299H, K326E, K326I, A327E, A327Y, L328A, and L328G.

A variant Fc region may also comprise sequence alterations wherein amino acids involved in disulfide bond formation are removed or replaced with other amino acids. Such removal may avoid reaction with other cysteine-containing proteins present in the host cell used to produce the antibodies. Even when cysteine residues are removed, single chain Fc domains can still form a dimeric Fc domain that is held together non-covalently.

### IV. Antibodies which Bind to the Same Epitope as or Compete with Anti-TNFR2 Antibodies

Also provided are antibodies which bind to the same epitope on TNFR2 as the anti-TNFR2 antibodies described herein. In some embodiments, provided herein are antibodies which compete for binding to TNFR2 as the anti-TNFR2 antibodies described herein.

Cross-competing antibodies can be screened for based on their ability to cross-compete with the anti-TNFR2 antibodies described herein using standard binding assays (e.g., ELISA, Biacore).

Techniques for determining antibodies that bind to the “same epitope on TNFR2” with the antibodies described herein include x-ray analyses of crystals of antigen:antibody complexes, which provides atomic resolution of the epitope. Other methods monitor the binding of the antibody to antigen fragments or mutated variations of the antigen where loss of binding due to an amino acid modification within the antigen sequence indicates the epitope component. Methods may also rely on the ability of an antibody of interest to affinity isolate specific short peptides (either in native three-dimensional form or in denatured form) from combinatorial phage display peptide libraries or from a protease digest of the target protein. The peptides are then regarded as leads for the definition of the epitope corresponding to the antibody used to screen the peptide library. For epitope mapping, computational algorithms have also been developed that have been shown to map conformational discontinuous epitopes.

The epitope or region comprising the epitope can also be identified by screening for binding to a series of overlapping peptides spanning TNFR2. Alternatively, the method of Jespers et al. (1994) Biotechnology 12:899 may be used to guide the selection of antibodies having the same epitope and therefore similar properties to the anti-TNFR2 antibodies described herein. Using phage display, first, the heavy chain of the anti-TNFR2 antibody is paired with a repertoire of (e.g., human) light chains to select an TNFR2-binding antibody, and then the new light chain is paired with a repertoire of (e.g., human) heavy chains to select a (e.g., human) TNFR2-binding antibody having the same epitope or epitope region as an anti-TNFR2 antibody described herein. Alternatively, variants of an antibody described herein can be obtained by mutagenesis of cDNA sequences encoding the heavy and light chains of the antibody.

Alanine scanning mutagenesis, as described by Cunningham & Wells (1989) Science 244: 1081, or some other form of point mutagenesis of amino acid residues in TNFR2 may also be used to determine the functional epitope for an anti-TNFR2 antibody.

The epitope or epitope region (an “epitope region” is a region comprising the epitope or overlapping with the epitope) bound by a specific antibody may also be determined by assessing binding of the antibody to peptides comprising TNFR2 fragments. A series of overlapping peptides encompassing the TNFR2 sequence may be synthesized and screened for binding, e.g. in a direct ELISA, a competitive ELISA (where the peptide is assessed for its ability to prevent binding of an antibody to TNFR2 bound to a well of a microtiter plate), or on a chip. Such peptide screening methods may not be capable of detecting some discontinuous functional epitopes, i.e., functional epitopes that involve amino acid residues that are not contiguous along the primary sequence of the TNFR2 polypeptide chain.

An epitope may also be identified by MS-based protein footprinting, such as HDX-MS and Fast Photochemical Oxidation of Proteins (FPOP). HDX-MS may be conducted, e.g., as further described at Wei et al. (2014) Drug Discovery Today 19:95, the methods of which are specifically incorporated by reference herein. FPOP may be conducted as described, e.g., in Hambley & Gross (2005) J. American Soc. Mass Spectrometry 16:2057, the methods of which are specifically incorporated by reference herein.

The epitope bound by anti-TNFR2 antibodies may also be determined by structural methods, such as X-ray crystal structure determination (e.g., WO2005/044853), molecular modeling and nuclear magnetic resonance (NMR) spectroscopy, including NMR determination of the H-D exchange rates of labile amide hydrogens in TNFR2 when free and when bound in a complex with an antibody of interest (Zinn-Justin et al. (1992) Biochemistry 31:11335; Zinn-Justin et al. (1993) Biochemistry 32:6884).

In some embodiments, the anti-TNFR2 antibodies bind to one or more of the following positions (e.g., one two, three, four, or all five positions) on human TNFR2 (numbering according to SEQ ID NO: 104): Y24, Q26, Q29, M30, and K47. For example, in one embodiment, the anti-TNFR2 antibody binds to position Y24 on human TNFR2. In another embodiment, the anti-TNFR2 antibody binds to position Q26 on human TNFR2. In another embodiment, the anti-TNFR2 antibody binds to position Q29 on human TNFR2. In another embodiment, the anti-TNFR2 antibody binds to position M30 on human TNFR2. In another embodiment, the anti-TNFR2 antibody binds to position K47 on human TNFR2. In another embodiment, the anti-TNFR2 antibody binds to positions Y24 and Q26 on human TNFR2. In another embodiment, the anti-TNFR2 antibody binds to positions Y24 and Q29 on human TNFR2. In another embodiment, the anti-TNFR2 antibody binds to positions Y24 and M30 on human TNFR2. In another embodiment, the anti-TNFR2 antibody binds to positions Y24 and K47 on human TNFR2. In another embodiment, the anti-TNFR2 antibody binds to positions Q26 and Q29 on human TNFR2. In another embodiment, the anti-TNFR2 antibody binds to positions Q26 and M30 on human TNFR2. In another embodiment, the anti-TNFR2 antibody binds to positions Q26 and K47 on human TNFR2. In another embodiment, the anti-TNFR2 antibody binds to positions Q29 and M30 on human TNFR2. In another embodiment, the anti-TNFR2 antibody binds to positions Q29 and K47 on human TNFR2. In another embodiment, the anti-TNFR2 antibody binds to positions M30 and K47 on human TNFR2. In another embodiment, the anti-TNFR2 antibody binds to positions Y24, Q26, and Q29 on human TNFR2. In another embodiment, the anti-TNFR2 antibody binds to positions Y24, Q26, and M30 on human TNFR2. In another embodiment, the anti-TNFR2 antibody binds to positions Y24, Q26, and K47 on human TNFR2. In another embodiment, the anti-TNFR2 antibody binds to positions Y24, Q29, and M30 on human TNFR2. In another embodiment, the anti-TNFR2 antibody binds to positions Y24, Q29, and K47 on human TNFR2. In another embodiment, the anti-TNFR2 antibody binds to positions Y24, M30, and K47 on human TNFR2. In another embodiment, the anti-TNFR2 antibody binds to positions Q26, Q29, and M30 on human TNFR2. In another embodiment, the anti-TNFR2 antibody binds to positions Q26, Q29, and K47 on human TNFR2. In another embodiment, the anti-TNFR2 antibody binds to positions Q29, M30, and K47 on human TNFR2. In another embodiment, the anti-TNFR2 antibody binds to positions Y24, Q26, Q29, and M30 on human TNFR2. In another embodiment, the anti-TNFR2 antibody binds to positions Y24, Q26, Q29, and K47 on human TNFR2. In another embodiment, the anti-TNFR2 antibody binds to positions Q26, Q29, M30, and K47 on human TNFR2. In another embodiment, the anti-TNFR2 antibody binds to positions Y24, Q26, Q29, M30, and K47 on human TNFR2.

In some embodiments, the anti-TNFR2 antibodies bind to an epitope on human TNFR2 that consists of one or more of the following positions (e.g., one, two, three, four, or all five positions) on human TNFR2 (numbering according to SEQ ID NO: 104): Y24, Q26, Q29, M30, and K47. In other embodiments, the anti-TNFR2 antibodies bind to an epitope on human TNFR2 that spans, is in between, and/or overlaps with amino acid positions 24-47 of human TNFR2 (numbering according to SEQ ID NO: 104).

### V. Nucleic Acid Molecules

Also provided herein are nucleic acid molecules that encode the antibodies described herein. The nucleic acids may be present in whole cells, in a cell lysate, or in a partially purified or substantially pure form. A nucleic acid described herein can be, for example, DNA or RNA and may or may not contain intronic sequences. In certain embodiments, the nucleic acid is a cDNA molecule. The nucleic acids described herein can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

In some embodiments, provided herein are nucleic acid molecules that encode the VH and/or VL sequences, or heavy and/or light chain sequences, of any of the anti-TFNR2 antibodies described herein. Host cells comprising the nucleotide sequences (e.g., nucleic acid molecules) described herein are encompassed herein.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (hinge, CH1, CH2 and/or CH3). The sequences of human heavy chain constant region genes are known in the art (see e.g., Kabat, E. A., el al. (1991) Sequences of Proteins of Immunological Interest, Fifth Edition, U.S. Department of Health and Human Services, NIH Publication No. 91-3242) and DNA fragments encompassing these regions can be obtained by standard PCR amplification.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region.

Also provided herein are nucleic acid molecules with conservative substitutions that do not alter the resulting amino acid sequence upon translation of the nucleic acid molecule.

### VI. Methods for Screening and Producing Antibodies

The anti-TNFR2 antibodies (e.g., anti-human TNFR2 antibodies) provided herein typically are prepared by standard recombinant DNA techniques. Additionally, monoclonal antibodies can be produced using a variety of known techniques, such as the standard somatic cell hybridization technique, viral or oncogenic transformation of B lymphocytes, or yeast or phage display techniques using libraries of human antibody genes. In particular embodiments, the antibodies are fully human monoclonal antibodies.

In one embodiment, provided herein are methods for generating monoclonal anti-human TNFR2 antibodies. Monoclonal antibodies may be readily prepared using well-known techniques (see, e.g., Antibodies: A Laboratory Manual, Cold Spring Harbor Laboratory, 1988; incorporated herein by reference). Typically, this technique involves immunizing a suitable animal with a selected polypeptide (e.g., the extracellular domain of human TNFR2 or a polypeptide that includes a human TNFR2 epitope of interest) conjugated to a carrier protein (e.g., KLH, bovine serum albumin).

The immunizing composition is administered in a manner effective to stimulate antibody producing cells. Rodents such as mice and rats are preferred, however, the use of rabbit, sheep and frog cells is also possible. The use of rats may provide certain advantages (Goding, 1986, pp. 60-61; incorporated herein by reference), but mice are preferred, with the BALB/c mouse being most preferred as this is most routinely used and generally gives a higher percentage of stable fusions. Following immunization, B lymphocytes (B cells) are selected for use in the antibody generating protocol. These cells may be obtained from biopsied spleens, tonsils or lymph nodes, or from a peripheral blood sample. A panel of animals is typically immunized and the spleen of the animal with the highest antibody titer will be removed and the spleen lymphocytes obtained by homogenizing the spleen with a syringe. The anti-human TNFR2 antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Exemplary myeloma cells include, e.g., P3-X63/Ag8, X63-Ag8.653, NS1/1.Ag 4 1, Sp210-Ag14, FO, NSO/U, MPC-11, MPC11-X45-GTG 1.7 and S194/5XX0 Bul for mouse; R210.RCY3, Y3-Ag 1.2.3, IR983F, 4B210 or one of the above listed mouse cell lines for rats; and U-266, GM1500-GRG2, LICR-LON-HMy2 and UC729-6 are useful in connection with human cell fusions.

### Producing Hybridomas

Methods for generating hybrids of antibody-producing spleen or lymph node cells and myeloma cells usually comprise mixing somatic cells with myeloma cells in a 4:1 proportion, although the proportion may vary from about 20:1 to about 1:1, respectively, in the presence of an agent or agents (chemical or electrical) that promote the fusion of cell membranes. Fusion methods using Sendai virus or polyethylene glycol (PEG), such as 37% (v/v) PEG, are known in the art. The use of electrically induced fusion methods is also appropriate.

Viable, fused hybrids are differentiated from the parental, unfused cells by culturing in a selective medium which typically contains an agent that blocks the de novo synthesis of nucleotides in the tissue culture media. Exemplary agents are aminopterin, methotrexate, and azaserine. Where aminopterin or methotrexate is used, the media is supplemented with hypoxanthine and thymidine as a source of nucleotides (HAT medium). Where azaserine is used, the media is supplemented with hypoxanthine. When HAT medium is used, only cells capable of operating nucleotide salvage pathways are able to survive in HAT medium. The myeloma cells are defective in key enzymes of the salvage pathway, e.g., hypoxanthine phosphoribosyl transferase (HPRT), and thus cannot survive. The only cells that can survive in the selective media are those hybrids formed from myeloma and B cells. This culturing process provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired anti-human TNFR2 reactivity. Exemplary assays include radioimmunoassays, enzyme immunoassays, cytotoxicity assays, plaque assays, dot immunobinding assays, bio-layer interferometry, and the like.

Selected hybridomas are serially diluted and cloned into individual anti-human TNFR2 antibody-producing cell lines, which clones can then be propagated indefinitely to provide monoclonal antibodies. The cell lines may be used for monoclonal antibody production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide monoclonal antibodies in high concentration. The individual cell lines could also be cultured in vitro, where the monoclonal antibodies are naturally secreted into the culture medium from which they can be readily obtained in high concentrations. Monoclonal antibodies produced by either means will generally be further purified, e.g., using filtration, centrifugation and various chromatographic methods, such as HPLC or affinity chromatography, all of which purification techniques are well known to those of skill in the art. These purification techniques each involve fractionation to separate the desired antibody from other components of a mixture. Analytical methods particularly suited to the preparation of antibodies include, for example, protein A-Sepharose and/or protein G-Sepharose chromatography.

### High Throughput Screening of Anti-TNFR2 Antibodies

Also provided herein are methods for high throughput screening of libraries for molecules that bind to human TNFR2 epitopes (such as those described herein), e.g., phage display, bacterial display, yeast display, mammalian display, ribosome display, mRNA display, and cDNA display.

In one embodiment, provided herein are methods for screening anti-human TNFR2 antibodies using phagemid libraries. Exemplary phage display protocols can be found, e.g., in U.S. Pat. Nos. 7,846,892, 8,846,867, WO1997/002342, and WO2007/13291, herein incorporated by reference. Recombinant technology now allows the preparation of antibodies having the desired specificity from recombinant genes encoding a range of antibodies Certain recombinant techniques involve the isolation of the antibody genes by immunological screening of combinatorial immunoglobulin phage expression libraries prepared from RNA isolated from the spleen of an immunized animal (e.g., an animal immunized with the extracellular domain of human TNFR2 or a peptide that includes a human TNFR2 epitope of interest). For such methods, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 104 times as many antibodies can be produced and screened in a single round, and that new specificities are generated by H and L chain combination, which further increases the percentage of appropriate antibodies generated.

One method for the generation of a large repertoire of diverse antibody molecules in bacteria utilizes the bacteriophage lambda as the vector (Huse et al., 1989; incorporated herein by reference). Production of antibodies using the lambda vector involves the cloning of heavy and light chain populations of DNA sequences into separate starting vectors. The vectors are subsequently combined randomly to form a single vector that directs the co-expression of heavy and light chains to form antibody fragments. The heavy and light chain DNA sequences are obtained by amplification, preferably by PCR or a related amplification technique, of mRNA isolated from spleen cells (or hybridomas thereof) from an animal that has been immunized with a selected antigen (e.g., the extracellular domain of human TNFR2 or a peptide that includes a human TNFR2 epitope of interest). The heavy and light chain sequences are typically amplified using primers that incorporate restriction sites into the ends of the amplified DNA segment to facilitate cloning of the heavy and light chain segments into the starting vectors.

Another method for the generation and screening of large libraries of wholly or partially synthetic antibody combining sites, or paratopes, utilizes display vectors derived from filamentous phage such as M13, fl or fd. These filamentous phage display vectors, referred to as “phagemids”, yield large libraries of monoclonal antibodies having diverse and novel immunospecificities. The technology uses a filamentous phage coat protein membrane anchor domain as a means for linking gene-product and gene during the assembly stage of filamentous phage replication, and has been used for the cloning and expression of antibodies from combinatorial libraries. In a general sense, the method provides a system for the simultaneous cloning and screening of pre-selected ligand-binding specificities from antibody gene repertoires using a single vector system. Screening of isolated members of the library for a pre-selected ligand-binding capacity allows the correlation of the binding capacity of an expressed antibody molecule with a convenient means to isolate the gene that encodes the member from the library.

The diversity of a filamentous phage-based combinatorial antibody library can be increased by shuffling of the heavy and light chain genes, by altering one or more of the complementarity determining regions of the cloned heavy chain genes of the library, or by introducing random mutations into the library by error-prone polymerase chain reactions. Additional methods for screening phagemid libraries are described in U.S. Pat. Nos. 5,580,717; 5,427,908; 5,403,484; and 5,223,409, each incorporated herein by reference.

In another embodiment, provided herein are methods for screening anti-human TNFR2 antibodies using cell-based display techniques, such as yeast display (Boder et al., Nat Biotechnol 1997; 15:553) and bacterial display. Established procedures to generate and screen libraries of bacterial cells or yeast cells that express polypeptides, such as single-chain polypeptides, antibodies, or antibody fragments, containing randomized hypervariable regions can be found in, e.g., U.S. Pat. No. 7,749,501, US2013/0085072, de Bruin et al., Nat Biotechnol 1999; 17:397; the teachings of each which are incorporated herein by reference.

In another embodiment, provided herein are methods for screening anti-human TNFR2 antibodies using nucleotide display techniques, which use in vitro translation of randomized polynucleotide libraries encoding single-chain polypeptides, antibodies, or antigen-binding fragments that contain mutations within designated hypervariable regions (see, e.g., WO2006/072773, U.S. Pat. No. 7,074,557). Antibodies can also be generated using cDNA display, a technique analogous to mRNA display, with the exception that cDNA instead of mRNA is used. cDNA display techniques are described in, e.g., Ueno et al. Methods Mol. Biol. 2012; 805:113-135).

The in vitro display techniques described above can also be used to improve the affinity of the anti-TNFR2 antibodies described herein. For example, libraries of single-chain polypeptides, antibodies, and antigen-binding fragments thereof that have targeted mutations at specific sites within hypervariable regions of a particular anti-TNFR2 antibody can be used. Polynucleotides encoding these mutated antibodies or antigen-binding fragments thereof can then be used in ribosome display, mRNA display, cDNA display to screen for polypeptides that specifically bind to the human TNFR2 epitope of interest.

Combinatorial libraries of polypeptides can also be screened to identify anti-TNFR2 antibodies that bind to human TNFR2 epitopes of interest. Combinatorial polypeptide libraries, such as antibody or antibody fragment libraries, can be obtained, e.g., by expression of polynucleotides encoding randomized hypervariable regions of an antibody or antigen-binding fragment thereof in a eukaryotic or prokaryotic cell using art-recognized gene expression techniques. The resulting heterogeneous mixture of antibodies can be isolated from the cells using standard techniques and screened for the ability to bind to a peptide derived from TNFR2 immobilized to a surface. Non-binding antibodies are washed off using an appropriate buffer, and antibodies that remain bound can be detected using, an ELISA-based detection protocol. The sequence of an antibody fragment that specifically binds to the TNFR2 peptide can be determined by techniques known in the art, including, e.g., Edman degradation, tandem mass spectrometry, matrix-assisted laser-desorption time-of-flight mass spectrometry (MALDI-TOF MS), nuclear magnetic resonance (NMR), and 2D gel electrophoresis, among others (see, e.g., WO 2004/062553).

Producing Anti-TNFR2 Antibodies with Recombinant DNA Techniques, Host Cell Transfectomas, and Transgenic Animals

Also provided herein are methods of producing anti-human TNFR2 antibodies in a host cell transfectoma using, for example, a combination of recombinant DNA techniques and gene transfection methods known in the art (Morrison, S. (1985) Science 229:1202). For example, to express antibodies, or antibody fragments thereof, DNAs encoding partial or full-length light and heavy chains can be obtained by standard molecular biology techniques (e.g., PCR amplification or cDNA cloning using a hybridoma (such as those described above) that expresses the antibody of interest) and the DNAs can be inserted into expression vectors such that the genes are operatively linked to transcriptional and translational control sequences. In this context, the term “operatively linked” means that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or both genes are inserted into the same expression vector. The antibody genes are inserted into the expression vector(s) by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{L} segment is operatively linked to the C_{L} segment within the vector.

For expression of light and heavy chains, the expression vector(s) encoding the heavy and light chains is transfected into a host cell by standard techniques. Although it is possible to express the antibodies described herein in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Preferred mammalian host cells for expressing the recombinant antibodies described herein include Chinese Hamster Ovary (CHO cells) (including dhfr—CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

In yet another embodiment, human monoclonal antibodies directed against particular epitopes on human TNFR2 can be generated using transgenic or transchromosomic mice carrying parts of the human immune system rather than the mouse system (see e.g., U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al.).

In another embodiment, human antibodies can be raised against particular epitopes on human TNFR2 using a mouse that carries human immunoglobulin sequences on transgenes and transchomosomes, such as a mouse that carries a human heavy chain transgene and a human light chain transchromosome (see e.g., PCT Publication WO 02/43478 to Ishida et al.).

Still further, alternative transgenic animal systems expressing human immunoglobulin genes are available in the art and can be used to raise anti-human TNFR2 antibodies that recognize particular human TNFR2 epitopes. For example, an alternative transgenic system referred to as the Xenomouse (Abgenix, Inc.) can be used; such mice are described in, for example, U.S. Pat. Nos. 5,939,598; 6,075,181; 6,114,598; 6, 150,584 and 6,162,963 to Kucherlapati et al. Another suitable transgenic animal system is the HuMAb mouse (Medarex, Inc), which contains human immunoglobulin gene miniloci that encode unrearranged human heavy (μ and γ) and κ light chain immunoglobulin sequences, together with targeted mutations that inactivate the endogenous μ and κ chain loci (see e.g., Lonberg, et al. (1994) Nature 368(6474): 856-859). Yet another suitable transgenic animal system is the KM mouse, described in detail in PCT publication WO02/43478.

Alternative transchromosomic animal systems expressing human immunoglobulin genes are available in the art and can be used to raise anti-TNFR2 antibodies. For example, mice carrying both a human heavy chain transchromosome and a human light chain tranchromosome can be used. Furthermore, cows carrying human heavy and light chain transchromosomes have been described in the art and can be used to raise anti-TNFR2 antibodies.

In yet another embodiment, antibodies can be prepared using a transgenic plant and/or cultured plant cells (such as, for example, tobacco, maize and duckweed) that produce such antibodies. For example, transgenic tobacco leaves expressing antibodies can be used to produce such antibodies by, for example, using an inducible promoter. Also, transgenic maize can be used to express such antibodies and antigen binding portions thereof. Antibodies can also be produced in large amounts from transgenic plant seeds including antibody portions, such as single chain antibodies (scFv's), for example, using tobacco seeds and potato tubers.

In the above embodiments, the antigen used to immunize animals may be, for example, the extracellular domain of human TNFR2. When the extracellular domain of human TNFR2 is used as the antigen, the generated antibodies are further screened for the ability to selectively bind particular epitopes on human TNFR2, e.g., amino acids 23-54, 55-96, 78-96, and 120-257 of human TNFR2 (SEQ ID NO: 1). Screening can be performed, e.g., using assays (e.g., ELISA) to assess binding to peptides that include the human TNFR2 epitope of interest, or binding assays using the TNFR2 chimeras described herein. Anti-human TNFR2 antibodies that share the epitope or TNFR2 chimera binding characteristics of the anti-TNFR2 antibodies described herein are then selected.

In another embodiment, the antigen used to immunize animals or target used to screen libraries (e.g., phagemid libraries, yeast surface display libraries) is a peptide that includes a human TNFR2 epitope recognized by the anti-TNFR2 antibodies described herein. Exemplary epitopes of human TNFR2 that are recognized by the antibodies described herein include amino acids 23-54, 55-96, 78-96, and 120-257 of human TNFR2 (SEQ ID NO: 1). Peptides that include these sequences can be used to immunize animals or screen libraries using the techniques listed above. Anti-human TNFR2 antibodies generated using this method can be screened for binding to TNFR2 chimeras, e.g., using the method described in Example 5, or for selectively binding to the peptide used as the immunogen.

Producing Humanized and/or Chimeric TNFR2 Antibodies

Chimeric and/or humanized antibodies can be generated based on the sequence of a murine monoclonal antibody, such as those described herein. DNA encoding the heavy and light chain immunoglobulins can be obtained from the murine hybridoma of interest and engineered to contain non-murine (e.g., human) immunoglobulin sequences using standard molecular biology techniques.

For example, chimeric antibodies and antigen-binding fragments thereof comprise portions from two or more different species (e.g., mouse and human). To create a chimeric antibody, the murine variable regions can be linked to human constant regions using methods known in the art (see e.g., U.S. Pat. No. 4,816,567 to Cabilly et al.). In this manner, non-human antibodies can be modified to make them more suitable for human clinical application (e.g., methods for treating or preventing a cancer in a human subject).

Alternatively, humanized antibodies are antibodies from non-human species whose protein sequences have been modified to increase their similarity to antibody variants produced naturally in humans. The monoclonal antibodies of the present disclosure include “humanized” forms of the non-human (e.g., mouse) antibodies (e.g., humanized forms of the antibodies produced by hybridomas ABV3, ABV4, ABV7, ABV12, ABV13, ABV14, ABV15, ABV18, and/or ABV19). Humanized or CDR-grafted mAbs are particularly useful as therapeutic agents for humans because they are not cleared from the circulation as rapidly as mouse antibodies and do not typically provoke an adverse immune reaction.

Methods of preparing humanized antibodies are well known in the art. For example, humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)). Additionally, humanized TNFR2 antibodies described herein can be produced using a variety of techniques known in the art, including, but not limited to, CDR-grafting (see e.g., European Patent No. EP 239,400; International Publication No. WO 91/09967; and U.S. Pat. Nos. 4,816,567; 6,331,415, 5,225,539, 5,530,101, and 5,585,089, each of which is incorporated herein by reference), veneering or resurfacing (see, e.g., European Patent Nos. EP 592,106 and EP 519,596; Padlan, 1991, Molecular Immunology 28(4/5):489-498; Studnicka et al., 1994, Protein Engineering, 7(6):805-814; and Roguska et al., 1994, Proc. Natl. Acad. Sci., 91:969-973, each of which is incorporated herein by reference), chain shuffling (see, e.g., U.S. Pat. No. 5,565,332, which is incorporated herein by reference), and techniques disclosed in, e.g., U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al, Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein by reference. Often, framework (FW) residues in the FW regions will be substituted with the corresponding residue from the CDR donor antibody to alter, preferably improve, antigen binding. These FW substitutions are identified by methods well known in the art, e.g., by modeling of the interactions of the CDR and FW residues to identify FW residues important for antigen binding and sequence comparison to identify unusual FW residues at particular positions. (See, e.g., Queen et al., U.S. Pat. No. 5,585,089; and Riechmann et al, 1988, Nature, 332:323, which are incorporated herein by reference in their entireties.)

In some embodiments, humanized forms of non-human (e.g., mouse) antibodies are human antibodies (recipient antibody) in which hypervariable (CDR) region residues of the recipient antibody are replaced by hypervariable region residues from a non-human species (donor antibody) such as a mouse, rat, rabbit, or non-human primate having the desired specificity, affinity, and binding capacity. In some instances, framework region residues of the human immunoglobulin are also replaced by corresponding non-human residues (so called “back mutations”). In addition, phage display libraries can be used to vary amino acids at chosen positions within the antibody sequence. The properties of a humanized antibody are also affected by the choice of the human framework. Furthermore, humanized and/or chimeric antibodies can be modified to comprise residues that are not found in the recipient antibody or in the donor antibody in order to further improve antibody properties, such as, for example, affinity or effector function.

In such humanized chimeric antibodies, substantially less than an intact human variable domain has been substituted by the corresponding sequence from a nonhuman species. In practice, humanized antibodies are typically human antibodies in which some CDR residues and possibly some FW residues are substituted by residues from analogous sites in rodent antibodies. Humanization of anti-TNFR2 antibodies can also be achieved by veneering or resurfacing (EP 592,106; EP 519,596; Padlan, 1991, Molecular Immunology 28(4/5):489-498; Studnicka et al., Protein Engineering, 7(6):805-814 (1994); and Roguska et al., Proc. Natl. Acad. Sci., 91:969-973 (1994)) or chain shuffling (U.S. Pat. No. 5,565,332), the contents of which are incorporated herein by reference in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequences which are most closely related to that of the rodent are then screened for the presence of specific residues that may be critical for antigen binding, appropriate structural formation and/or stability of the intended humanized mAb (Sims et al., J. Immunol., 151:2296 (1993); Chothia et al., J. Mol. Biol., 196:901 (1987), the contents of which are incorporated herein by reference in their entirety). The resulting FW sequences matching the desired criteria are then be used as the human donor FW regions for the humanized antibody.

Another method uses a particular FW derived from the consensus sequence of all human antibodies of a particular subgroup of light or heavy chains. The same FW may be used for several different humanized anti-TNFR2 antibodies (Carter et al., Proc. Natl. Acad. Sci. USA, 89:4285 (1992); Presta et al., J. Immunol., 151:2623 (1993), the contents of which are incorporated herein by reference in their entirety).

Anti-TNFR2 antibodies can be humanized with retention of high affinity for human TNFR2 and other favorable biological properties. According to one aspect of the invention, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind TNFR2. In this way, FW residues can be selected and combined from the recipient and import sequences so that the desired antibody characteristic, for example affinity for TNFR2, is achieved. In general, the CDR residues are directly and most substantially involved in influencing antigen binding.

The binding specificity of monoclonal antibodies (or portions thereof) that bind TNFR2 prepared using any technique including those disclosed herein, can be determined by immunoprecipitation or by an in vitro binding assay, such as radioimmunoassay (RIA), enzyme-linked immunoabsorbent assay (ELISA), bio-layer interferometry (e.g., ForteBio assay), and/or Scatchard analysis.

In certain embodiments, an anti-TNFR2 antibody produced using any of the methods discussed above may be further altered or optimized to achieve a desired binding specificity and/or affinity using art recognized techniques, such as those described herein.

### VII. Multispecific Antibodies

Multispecific antibodies (e.g., bispecific antibodies) provided herein include at least a binding affinity for a particular epitope on TNFR2 (e.g., human TNFR2) as described herein, and at least one other binding specificity. In some embodiments, the non-TNFR2 binding specificity is a binding specificity for a cancer antigen. Multispecific antibodies can be prepared as full length antibodies or antibody fragments (e.g. F(ab′)2 antibodies).

Methods for making multispecific antibodies are well known in the art (see, e.g., WO 05117973 and WO 06091209). For example, production of full length multispecific antibodies can be based on the coexpression of two paired immunoglobulin heavy chain-light chains, where the two chains have different specificities. Various techniques for making and isolating multispecific antibody fragments directly from recombinant cell culture have also been described. For example, multispecific antibodies can be produced using leucine zippers. Another strategy for making multispecific antibody fragments by the use of single-chain Fv (sFv) dimers has also been reported.

In a particular embodiment, the multispecific antibody comprises a first antibody (or binding portion thereof) which binds to an epitope of interest on TNFR2 derivatized or linked to another functional molecule, e.g., another peptide or protein (e.g., another antibody or ligand for a receptor) to generate a multispecific molecule that binds to an epitope on TNFR2 and another target molecule. An antibody may be derivatized or linked to more than one other functional molecule to generate multispecific molecules that bind to more than two different binding sites and/or target molecules. To create a multispecific molecule, an antibody disclosed herein can be functionally linked (e.g., by chemical coupling, genetic fusion, noncovalent association or otherwise) to one or more other binding molecules, such as another antibody, antibody fragment, peptide or binding mimetic, such that a multispecific molecule results.

Accordingly, multispecific molecules comprising at least one first binding specificity for a particular epitope on TNFR2 (e.g., human TNFR2) and a second binding specificity for a second non-TNFR2 target epitope are contemplated. In a particular embodiment, the second target epitope is an Fc receptor, e.g., human FcγRI (CD64) or a human Fcα receptor (CD89). Therefore, multispecific molecules capable of binding both to FcγR, FcαR or FcεR expressing effector cells (e.g., monocytes, macrophages or polymorphonuclear cells (PMNs)), and to target cells expressing TNFR2 are also provided. These multispecific molecules target TNFR2-expressing cells to effector cells and trigger Fc receptor-mediated effector cell activities, such as phagocytosis of TNFR2-expressing cells, antibody dependent cell-mediated cytotoxicity (ADCC), cytokine release, or generation of superoxide anion.

In one embodiment, the multispecific molecules comprise as a binding specificity at least one antibody, or an antibody fragment thereof, including, e.g., an Fab, Fab′, F(ab′)₂, Fv, or a single chain Fv. The antibody may also be a light chain or heavy chain dimer, or any minimal fragment thereof such as a Fv or a single chain construct as described in Ladner et al. U.S. Pat. No. 4,946,778.

The multispecific molecules can be prepared by conjugating the constituent binding specificities, e.g., the anti-FcR and anti-TNFR2 binding specificities, using methods known in the art. For example, each binding specificity of the multispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC). Preferred conjugating agents are SATA and sulfo-SMCC, both available from Pierce Chemical Co. (Rockford, Ill.).

When the binding specificities are antibodies, they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the multispecific molecule is a mAb×mAb, mAb×Fab, Fab×F(ab′)₂ or ligand×Fab fusion protein. A multispecific molecule can be a single chain molecule comprising one single chain antibody and a binding determinant, or a single chain bispecific molecule comprising two binding determinants. Multispecific molecules may comprise at least two single chain molecules. Methods for preparing multispecific molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the multispecific molecules to their specific targets can be confirmed by, for example, enzyme-linked immunosorbent assay (ELISA), radioimmunoassay (RIA), FACS analysis, bioassay (e.g., growth inhibition), or western blot assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA). The radioactive isotope can be detected by such means as the use of a αγ-β counter or a scintillation counter or by autoradiography.

### VIII. Immunoconjugates

Immunoconjugates provided herein can be formed by conjugating the antibodies described herein (e.g., anti-human TNFR2 antibodies) to another therapeutic agent. Suitable agents include, for example, a cytotoxic agent (e.g., a chemotherapeutic agent), a toxin (e.g. an enzymatically active toxin of bacterial, fungal, plant or animal origin, or fragments thereof), and/or a radioactive isotope (i.e., a radioconjugate).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, neomycin, and the tricothecenes. Additional examples of cytotoxins or cytotoxic agents include, e.g., taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

A variety of radionuclides are available for the production of radioconjugated anti-TNFR2 antibodies. Examples include ²¹² Bi, ¹³¹I, ¹³¹ In, ⁹⁰Y and ¹⁸⁶ Re.

Immunoconjugates can also be used to modify a given biological response, and the drug moiety is not to be construed as limited to classical chemical therapeutic agents. For example, the drug moiety may be a protein or polypeptide possessing a desired biological activity (e.g., lymphokines, tumor necrosis factor, IFNγ, growth factors).

Immunoconjugates can be made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). Carbon-14-labeled 1-isothiocyanatobenzyl-3-methyldiethylene triaminepentaacetic acid (MX-DTPA) is an exemplary chelating agent for conjugation of radionucleotide to the antibody (see, e.g., WO94/11026).

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

### IX. Assays

Subsequent to producing antibodies (e.g., antibodies having the CDR sequences of the anti-TNFR2 antibodies disclosed herein), they can be screened or tested for various properties, such as those described herein (e.g., binding to TNFR2), using a variety of assays known in the art.

In one embodiment, the antibodies are screened or tested (e.g., by flow cytometry, ELISA, Biacore, or bio-layer interferometry) for binding to TNFR2 using, for example, purified TNFR2 (e.g., purified extracellular domain of human TNFR2 or a peptide that includes the epitope of interest in human TNFR2) and/or TNFR2-expressing cells. In some embodiments, the antibodies can be screened for their ability to bind particular epitopes on TNFR2 by using a panel of TNFR2 chimeras, as described in Example 5. Other methods monitor the binding of the antibody to antigen fragments or mutated variations of human TNFR2 where loss of binding due to a modification of an amino acid residue within the antigen sequence is often considered an indication of an epitope component.

In some embodiments, the antibodies are screened or tested for binding to TNFR2 by Western blotting. Briefly, cell extracts from cells expressing TNFR2 (e.g., the extracellular domain of TNFR2) can be prepared and subjected to sodium dodecyl sulfate polyacrylamide gel electrophoresis. After electrophoresis, the separated antigens will be transferred to nitrocellulose membranes, blocked with serum, and probed with the monoclonal antibodies to be tested. IgG binding can be detected using anti-IgG alkaline phosphatase and developed with BCIP/NBT substrate tablets (Sigma Chem. Co., St. Louis, Mo.).

In another embodiment, the antibodies are screened for the ability to bind to epitopes exposed upon binding to ligand, e.g., TNFα (i.e., do not inhibit the binding of TNFR2 ligands to TNFR). Such antibodies can be identified by, for example, contacting cells which express TNFR2 with a labeled TNFR2 ligand (e.g., radiolabeled or biotinylated TNFα) in the absence (control) or presence of the anti-TNFR2 antibody. If the antibody does not inhibit TNFα binding to TNFR2, then no statistically significantly decrease in the amount of label recovered, relative to the amount in the absence of the antibody, will be observed. Alternatively, if the antibody inhibits TNFα binding to TNFR2, then a statistically significantly decrease in the amount of label recovered, relative to the amount in the absence of the antibody, will be observed.

Methods for analyzing binding affinity, cross-reactivity, and binding kinetics of various anti-TNFR2 antibodies include standard assays known in the art, for example, Biacore™ surface plasmon resonance (SPR) analysis using a Biacore™ 2000 SPR instrument (Biacore AB, Uppsala, Sweden) or bio-layer interferometry (e.g., ForteBio assay), as described in the Examples.

In some embodiments, the anti-TNFR2 antibodies are screened or tested for the ability to inhibit the binding of TNF-alpha to TNFR2 using art-recognized methods, such as flow cytometry, surface plasmon resonance, and biolayer interferometry, e.g., as described in Example 3.

In some embodiments, the anti-TNFR2 antibodies are screened or tested for agonist activity. Agonist activity can be tested using reporter assays, e.g., NF-kB reporter assays. In some embodiments, the antibodies are contacted with reporter cell lines, and reporter activity is determined by flow cytometry, e.g., as described in Example 23. In some embodiments, the agonist activity of the anti-TNFR2 antibodies are determined by assessing the proliferation of and/or induction of activation marker expression in primary isolated T cells, for example, as described in Examples 15, 24, and 26.

The anti-TNFR2 antibodies described herein can also be screened or tested for their ability to induce ADCC. Briefly, effector cells (e.g., NK cells) are cultured together with target cells in the presence or absence of the antibody of interest (e.g., anti-TNFR2 antibody) and/or a control antibody (e.g., isotype control). Death of target cells are then assessed, e.g., based on the quantification of a detectable label (e.g., fluorescence if the target cells are fluorescently labeled) using, e.g., flow cytometry as described in Example 25.

Antibodies can also be tested for their ability to inhibit the proliferation or viability of cells (either in vivo or in vitro), such as tumor cells, using art recognized techniques, including the Cell Titer-Glo Assay or a tritium-labeled thymidine incorporation assay, or flow cytometry.

### X. Compositions

In another aspect, provided herein is a composition, e.g., a pharmaceutical composition, comprising an anti-TNFR2 antibody (e.g., an anti-human TNFR2 antibody) disclosed herein, formulated together with a pharmaceutically acceptable carrier. Pharmaceutical compositions are prepared using standard methods known in the art by mixing the active ingredient (e.g., anti-TNFR2 antibodies described herein) having the desired degree of purity with optional physiologically acceptable carriers, excipients or stabilizers (Remington's Pharmaceutical Sciences (20′ edition), ed. A. Gennaro, 2000, Lippincott, Williams & Wilkins, Philadelphia, Pa.). Preferred pharmaceutical compositions are sterile compositions, compositions suitable for injection, and sterile compositions suitable for injection by a desired route of administration, such as by intravenous injection.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, may be coated in a material to protect the compound from the action of acids and other natural conditions that may inactivate the compound.

Compositions can be administered alone or in combination therapy, i.e., combined with other agents. For example, the combination therapy can include a composition provided herein with at least one or more additional therapeutic agents, e.g., other compounds, drugs, and/or agents used for the treatment of cancer (e.g., an anti-cancer agent(s). Particular combinations of anti-TNFR2 antibodies may also be administered separately or sequentially, with or without additional therapeutic agents.

Compositions can be administered by a variety of methods known in the art. As will be appreciated by the skilled artisan, the route and/or mode of administration will vary depending upon the desired results. The antibodies can be prepared with carriers that will protect the antibodies against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art.

To administer compositions by certain routes of administration, it may be necessary to coat the constituents, e.g., antibodies, with, or co-administer the compositions with, a material to prevent its inactivation. For example, the compositions may be administered to a subject in an appropriate carrier, for example, liposomes, or a diluent. Acceptable diluents include saline and aqueous buffer solutions. Liposomes include water-in-oil-in-water CGF emulsions as well as conventional liposomes.

Acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional medium or agent is incompatible with the antibodies, use thereof in compositions provided herein is contemplated. Supplementary active constituents can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Including in the composition an agent that delays absorption, for example, monostearate salts and gelatin can bring about prolonged absorption of the injectable compositions.

Sterile injectable solutions can be prepared by incorporating the monoclonal antibodies in the required amount in an appropriate solvent with one or a combination of ingredients enumerated above, as required, followed by sterilization microfiltration. Generally, dispersions are prepared by incorporating the antibodies into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. For example, human antibodies may be administered once or twice weekly by subcutaneous injection or once or twice monthly by subcutaneous injection.

It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of antibodies calculated to produce the desired therapeutic effect in association with the required pharmaceutical carrier. The specification for the dosage unit forms provided herein are dictated by and directly dependent on (a) the unique characteristics of the antibodies and the particular therapeutic effect to be achieved, and (b) the limitations inherent in the art of compounding such antibodies for the treatment of sensitivity in individuals.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

For the therapeutic compositions, formulations include those suitable for oral, nasal, topical (including buccal and sublingual), rectal, and parenteral administration. Parenteral administration is the most common route of administration for therapeutic compositions comprising antibodies. The formulations may conveniently be presented in unit dosage form and may be prepared by any methods known in the art of pharmacy. The amount of antibodies that can be combined with a carrier material to produce a single dosage form will vary depending upon the subject being treated, and the particular mode of administration. This amount of antibodies will generally be an amount sufficient to produce a therapeutic effect. Generally, out of 100%, this amount will range from about 0.001% to about 90% of antibody by mass, preferably from about 0.005% to about 70%, most preferably from about 0.01% to about 30%.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Examples of suitable aqueous and nonaqueous carriers which may be employed in the pharmaceutical compositions provided herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Particular examples of adjuvants which are well-known in the art include, for example, inorganic adjuvants (such as aluminum salts, e.g., aluminum phosphate and aluminum hydroxide), organic adjuvants (e.g., squalene), oil-based adjuvants, virosomes (e.g., virosomes which contain a membrane-bound heagglutinin and neuraminidase derived from the influenza virus).

Prevention of presence of microorganisms may be ensured both by sterilization procedures and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of one or more agents that delay absorption such as aluminum monostearate or gelatin.

When compositions are administered as pharmaceuticals, to humans and animals, they can be given alone or as a pharmaceutical composition containing, for example, 0.001 to 90% (more preferably, 0.005 to 70%, such as 0.01 to 30%) of active ingredient in combination with a pharmaceutically acceptable carrier.

Regardless of the route of administration selected, compositions provided herein, may be used in a suitable hydrated form, and they may be formulated into pharmaceutically acceptable dosage forms by conventional methods known to those of skill in the art.

Actual dosage levels of the antibodies in the pharmaceutical compositions provided herein may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A physician or veterinarian having ordinary skill in the art can readily determine and prescribe the effective amount of the composition required. For example, the physician or veterinarian could start doses of the antibodies at levels lower than that required to achieve the desired therapeutic effect and gradually increasing the dosage until the desired effect is achieved. In general, a suitable daily dose of compositions provided herein will be that amount of the antibodies which is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of a therapeutic composition may be administered as two, three, four, five, six or more sub-doses administered separately at appropriate intervals throughout the day, optionally, in unit dosage forms. While it is possible for antibodies to be administered alone, it is preferable to administer antibodies as a formulation (composition).

Dosages and frequency of administration may vary according to factors such as the route of administration and the particular antibody used, the nature and severity of the disease to be treated, and the size and general condition of the subject. Appropriate dosages can be determined by procedures known in the pertinent art, e.g. in clinical trials that may involve dose escalation studies.

Therapeutic compositions can be administered with medical devices known in the art, such as, for example, those disclosed in U.S. Pat. Nos. 5,399,163, 5,383,851, 5,312,335, 5,064,413, 4,941,880, 4,790,824, 4,596,556, 4,487,603, 4.,486,194, 4,447,233, 4,447,224, 4,439,196, and 4,475,196.

The ability of a compound to inhibit cancer can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

Uses of the above-described anti-TNFR2 antibodies and compositions comprising the same are provided in the manufacture of a medicament for the treatment of a disease associated with TNFR2-dependent signaling. The above-described anti-TNFR2 antibodies and compositions are also provided for the treatment of cancer (or to be used in the manufacture of a medicament for the treatment of cancer). In some embodiments, the cancer is a solid tumor. Exemplary cancers include, but are not limited to, lung cancer, renal cancer, breast cancer, ovarian cancer, hepatocellular carcinoma, renal cell carcinoma, lung carcinoma, cervical cancer, prostate cancer, melanoma, head and neck cancer, lymphoma, and colorectal cancer.

In some embodiments, the anti-TNFR2 antibodies and compositions described herein are used to treat an autoimmune disease or disorder (or to be used in the manufacture of a medicament for the treatment of autoimmune disease). Exemplary autoimmune diseases and disorders include, but are not limited to, graft-versus-host disease, rheumatoid arthritis, Crohn's disease, multiple sclerosis, colitis, psoriasis, autoimmune uveitis, pemphigus, epidermolysis bullosa, and type 1 diabetes.

In some embodiments, the anti-TNFR2 antibodies and compositions described herein are used to promote graft survival or reduce graft rejection in a subject who has received or will receive a cell, tissue, or organ transplant (or to be used in the manufacture of a medicament for promoting graft survival or reduce graft rejection). In other embodiments, the anti-TNFR2 antibodies and compositions described herein are also provided to treat, prevent, or reduce graft-versus-host disease (or to be used in the manufacture of a medicament for treating, preventing, or reducing graft-versus-host disease).

Additionally, contemplated compositions may further include, or be prepared for use as a medicament in combination therapy with, an additional therapeutic agent, e.g., an additional anti-cancer agent. An “anti-cancer agent” is a drug used to treat tumors, cancers, malignancies, and the like. Drug therapy (e.g., with antibody compositions disclosed herein) may be administered without other treatment, or in combination with other treatments.

A “therapeutically effective dosage” of an anti-TNFR2 antibody or composition described herein preferably results in a decrease in severity of disease symptoms, an increase in frequency and duration of disease symptom-free periods, or a prevention of impairment or disability due to the disease affliction. In the context of cancer, a therapeutically effective dose preferably results in increased survival, and/or prevention of further deterioration of physical symptoms associated with cancer. A therapeutically effective dose may prevent or delay onset of cancer, such as may be desired when early or preliminary signs of the disease are present.

### XI. Kits

Also provided are kits comprising the anti-TNFR2 antibodies, multispecific molecules, or immunoconjugates disclosed herein, optionally contained in a single vial or container, and include, e.g., instructions for use in treating or diagnosing a disease such as cancer. The kits may include a label indicating the intended use of the contents of the kit. The term label includes any writing, marketing materials or recorded material supplied on or with the kit, or which otherwise accompanies the kit. Such kits may comprise the antibody, multispecific molecule, or immunoconjugate in unit dosage form, such as in a single dose vial or a single dose pre-loaded syringe.

### XII. Methods of Using Antibodies

The antibodies and compositions disclosed herein can be used in a broad variety of therapeutic and diagnostic applications, for example, to treat cancer (oncological applications), to treat autoimmune diseases or disorders, to promote graft survival and/or reduce graft rejection in a transplant recipient, to treat, prevent, or reduce graft-versus-host disease, or to treat infectious diseases.

Accordingly, in one embodiment, provided herein is a method of treating proliferation disorders, e.g., cancer, comprising administering to a subject an anti-TNFR2 antibody described herein in an amount effective (e.g., a therapeutically effective amount) to treat the disorder. In some embodiments, the disorder is cancer. Exemplary cancers include, but are not limited to, solid tumors, such as lung cancer, renal cancer, breast cancer, ovarian cancer, hepatocellular carcinoma, renal cell carcinoma, lung carcinoma, cervical cancer, prostate cancer, melanoma, head and neck cancer, lymphoma, and colorectal cancer. Subjects can be examined during therapy to monitor the efficacy of the anti-TNFR2 antibodies to attenuate the progression of cancer (e.g., as reflected in the reduction in volume of one or more tumors).

In some embodiments, the anti-TNFR2 antibodies described herein are capable of reducing the volume of a tumor by at least about 10%, at least about 20%, at least about 30%, at least about 40%, at least about 50%, at least about 60%, at least about 70%, at least about 80%, at least about 90%, at least about 95%, at least about 98%, or about 100%, relative to the volume of the tumor prior to initiating anti-TNFR2 antibody therapy.

In another embodiment, provided herein is a method for inhibiting the growth of a tumor or tumor cells comprising administering to a subject an anti-TNFR2 antibody described herein in an effective amount (e.g., a therapeutically effective amount) to inhibit the growth of the tumor or tumor cells.

In some embodiments, the anti-TNFR2 antibodies described herein induce a long-term anti-cancer effect. In other embodiments, the anti-TNFR2 antibodies described herein induce the development of anti-cancer memory T cells.

In another embodiment, provided is a method of enhancing the anti-tumor activity of an antibody which binds to an epitope on human TNFR2 (e.g., a human TNFR2 epitope described herein), comprising modifying the antibody to increase its effector function relative to the same antibody in unmodified form, for example, by introducing one or more amino acid substitutions in the Fc region that are known to increase effector function. In some embodiments, the increased anti-tumor activity is independent of the epitope of human TNFR2 which the antibody binds to. In other embodiments, the inhibition of tumor growth is independent of the ability of the antibody to agonize TNFR2 signaling. In other embodiments, the inhibition of tumor growth is independent of the ability of the antibody to inhibit TNF-alpha binding to TNFR2.

In another embodiment, provided herein is a method of treating cancer comprising administering to a subject in need thereof a therapeutically effective amount of an anti-TNFR2 antibody, wherein the antibody has effector function and does not significantly inhibit binding of TNF-alpha to TNFR2.

In another embodiment, provided herein is a method of treating cancer comprising administering to a subject in need thereof a therapeutically effective amount of an anti-TNFR2 antibody, wherein the antibody has effector function and agonizes TNFR2 receptor signaling.

In another embodiment, provided herein is a method of treating cancer comprising administering to a subject in need thereof a therapeutically effective amount of an anti-TNFR2 antibody, wherein the antibody has effector function.

In the methods described herein, the anti-TNFR2 antibodies can be administered alone or with one or more therapeutic agents (e.g., anti-cancer agents) or standard cancer treatment that act in conjunction with or synergistically with the antibody to treat a subject with a tumor or cancer. For example, the anti-TNFR2 antibodies described herein can be used in combination with immune checkpoint blockers. Suitable immune checkpoint blockers for use in combination with the anti-TNFR2 antibodies described herein include, for example, an anti-PD1 antibody, an anti-PD-L1 antibody, an anti-LAG-3 antibody, an anti-CTLA-4 antibody, an anti-TIGIT antibody, or an anti-TIM3 antibody.

PD-1 and PD-L1 checkpoint inhibitors offer significant promise in the treatment of cancer (Brahmer et al., NEJM 2012; 366:2455-65; Topalian et al., NEJM 2012; 366:2443-54). Unfortunately, their activity remains limited to a subset of patients in indications such as metastatic bladder cancer, non-small cell lung cancer (NSCLC), melanoma and head and neck cancers, with many progressing over time (Swaika et al., Molecular Immunology 2015; 67:4-17; Grigg et al., Journal for ImmunoTherapy of Cancer 2016; 4:48). Combinations with chemotherapy or other immunotherapies, such as the CTLA4 inhibitor, ipilimumab, have been shown to improve efficacy, but often at the expenses of significant increases in many toxicities compared to the PD-1 inhibitor alone (Weber, Oncologist 2016; 21:1230-40; Paz-Ares et al., NEJM 2018 pub ahead of print—PMID: 30280635). As shown in Example 12, a TNFR2 agonist antibody (Y9) in combination with PD-1 or PD-L1 inhibitors improves anti-tumor activity significantly, without the toxicity observed with anti-CTLA4 antibody treatment upon chronic dosing (see, Example 13). This suggests that the combination of an agonistic TNFR2 mAb with PD-1 or PD-L1 inhibitors has a significantly greater therapeutic index than that of PD-1 inhibitors with CTLA4 inhibitors, such as ipilimumab.

The anti-TNFR2 antibodies and combination antibody therapies described herein may also be used in conjunction with other well-known therapies selected for their particular usefulness against the indication being treated (e.g., cancer).

For example, the anti-TNFR2 antibodies described herein can be used in combination (e.g., simultaneously or separately) with an additional treatment, such as irradiation, surgery, chemotherapy (e.g., using camptothecin (CPT-11), 5-fluorouracil (5-FU), cisplatin, doxorubicin, irinotecan, paclitaxel, gemcitabine, cisplatin, paclitaxel, carboplatin-paclitaxel (Taxol), doxorubicin, 5-fu, or camptothecin+apo21/TRAIL (a 6× combo)), one or more proteasome inhibitors (e.g., bortezomib or MG132), one or more Bcl-2 inhibitors (e.g., BH3I-2′ (bcl-xl inhibitor), indoleamine dioxygenase-1 inhibitor (e.g., INCB24360, indoximod, NLG-919, or F001287), AT-101 (R-(−)-gossypol derivative), ABT-263 (small molecule), GX-15-070 (obatoclax), or MCL-1 (myeloid leukemia cell differentiation protein-1) antagonists), iAP (inhibitor of apoptosis protein) antagonists (e.g., smac7, smac4, small molecule smac mimetic, synthetic smac peptides (see Fulda et al., Nat Med 2002; 8:808-15), ISIS23722 (LY2181308), or AEG-35156 (GEM-640)), HDAC (histone deacetylase) inhibitors, anti-CD20 antibodies (e.g., rituximab), angiogenesis inhibitors (e.g., bevacizumab), anti-angiogenic agents targeting VEGF and VEGFR (e.g., Avastin), synthetic triterpenoids (see Hyer et al., Cancer Research 2005; 65:4799-808), c-FLIP (cellular FLICE-inhibitory protein) modulators (e.g., natural and synthetic ligands of PPARγ (peroxisome proliferator-activated receptor γ), 5809354 or 5569100), kinase inhibitors (e.g., Sorafenib), Trastuzumab, Cetuximab, Temsirolimus, mTOR inhibitors such as rapamycin and temsirolimus, Bortezomib, JAK2 inhibitors, HSP90 inhibitors, PI3K-AKT inhibitors, Lenalildomide, GSK30 inhibitors, IAP inhibitors, genotoxic drugs, targeted therapeutics, and/or cancer vaccines.

The anti-TNFR2 antibodies may also be used in combination with therapeutic antibodies useful for the treatment of cancer, such as Rituxan® (rituximab), Herceptin® (trastuzumab), Bexxar® (tositumomab), Zevalin® (ibritumomab), Campath® (alemtuzumab), Lymphocide® (eprtuzumab), Avastin® (bevacizumab), and Tarceva® (erlotinib), as well as antibodies that target a member of the TNF and TNFR family of molecules (ligands or receptors), such as CD40 and CD40L, OX-40, OX-40L, CD70, CD27L, CD30, CD30L, 4-1BBL, CD137, TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTOR, LIGHT, DcR3, HVEM, VEGI/TL1A, TRAMP/DR3, EDA1, EDA2, TNFR1, Lymphotoxin α/TNFβ, TNFα, LTOR, Lymphotoxin a 102, FAS, FASL, RELT, DR6, TROY, and NGFR.

Cytotoxic agents that are useful for treating cancer in combination with the anti-TNFR2 antibodies described herein include alkylating agents, antimetabolites, and other art-recognized anti-proliferative agents. Exemplary alkylating agents include nitrogen mustards, ethylenimine derivatives, alkyl sulfonates, nitrosoureas and triazenes, for example Uracil mustard, Chlormethine, Cyclophosphamide (CYTOXAN™) fosfamide, Melphalan, Chlorambucil, Pipobroman, Triethylenemelamine, Triethylenethiophosphoramine, Busulfan, Carmustine, Lomustine, Streptozocin, Dacarbazine, and Temozolomide. Exemplary antimetabolites include folic acid antagonists, pyrimidine analogs, purine analogs and adenosine deaminase inhibitors, for example, Methotrexate, 5-Fluorouracil, Floxuridine, Cytarabine, 6-Mercaptopurine, 6-Thioguanine, Fludarabine phosphate, Pentostatine, and Gemcitabine. Other suitable anti-proliferative agents for use in combination with the anti-TNFR2 antibodies described herein include, e.g., taxanes, paclitaxel (paclitaxel is commercially available as TAXOL™), docetaxel, discodermolide (DDM), dictyostatin (DCT), Peloruside A, epothilones, epothilone A, epothilone B, epothilone C, epothilone D, epothilone E, epothilone F, furanoepothilone D, desoxyepothilone B1, [17]-dehydrodesoxyepothilone B, [18]dehydrodesoxyepothilones B, C12,13-cyclopropyl-epothilone A, C6-C8 bridged epothilone A, trans-9,10-dehydroepothilone D, cis-9,10-dehydroepothilone D, 16-desmethylepothilone B, epothilone B10, discoderomolide, patupilone (EPO-906), KOS-862, KOS-1584, ZK-EPO, ABJ-789, XAA296A (Discodermolide), TZT-1027 (soblidotin), ILX-651 (tasidotin hydrochloride), Halichondrin B, Eribulin mesylate (E-7389), Hemiasterlin (HTI-286), E-7974, Cyrptophycins, LY-355703, Maytansinoid immunoconjugates (DM-1), MKC-1, ABT-751, T1-38067, T-900607, SB-715992 (ispinesib), SB-743921, MK-0731, STA-5312, eleutherobin, 17beta-acetoxy-2-ethoxy-6-oxo-B-homo-estra-1,3,5(10)-trien-3-ol, cyclostreptin, isolaulimalide, laulimalide, 4-epi-7-dehydroxy-14,16-didemethyl-(+)-discodermolides, and cryptothilone 1, in addition to other microtubuline stabilizing agents known in the art.

In cases where it is desirable to render aberrantly proliferative cells quiescent in conjunction with or prior to treatment with anti-TNFR2 antibodies described herein, hormones and steroids (including synthetic analogs), such as 17a-Ethinylestradiol, Diethylstilbestrol, Testosterone, Prednisone, Fluoxymesterone, Dromostanolone propionate, Testolactone, Megestrolacetate, Methylprednisolone, Methyl-testosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, ZOLADEX™, can also be administered to the patient. When employing the methods or compositions described herein, other agents used in the modulation of tumor growth or metastasis in a clinical setting, such as antimimetics, can also be administered as desired.

Anti-TNFR2 antibodies described herein may be combined with an art-recognized vaccination protocol (e.g., cancer vaccine). Many experimental strategies for vaccination against tumors have been devised (see Rosenberg, S., 2000, Development of Cancer Vaccines, ASCO Educational Book Spring: 60-62; Logothetis, C., 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita et al. (eds.), 1997, Cancer: Principles and Practice of Oncology, Fifth Edition). In some embodiments, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination (Dranoff et al. (1993) Proc. Natl. Acad. Sci U.S.A. 90: 3539-43).

The anti-TNFR2 antibodies described herein are also useful for the treatment of autoimmune disease and disorders. Accordingly, in one embodiment, provided herein is a method of treating autoimmune disease and disorders comprising administering to a subject an anti-TNFR2 antibody described herein in an amount effective (e.g., a therapeutically effective amount) to treat the autoimmune diseases and disorders. Exemplary autoimmune diseases and disorders for treatment with the anti-TNFR2 antibodies described herein include, for example, graft-versus-host disease, rheumatoid arthritis, Crohn's disease, multiple sclerosis, colitis, psoriasis, autoimmune uveitis, pemphigus, epidermolysis bullosa, and type 1 diabetes. Subjects can be examined during therapy to monitor the efficacy of the anti-TNFR2 antibodies to attenuate the symptoms or pathology of autoimmune disease. Efficacy of the treatment can be monitored by comparing the effects of the antibody and or combination treatment before and after administration.

The anti-TNFR2 antibodies described herein can be administered alone or with one or more therapeutic agents that act in conjunction with or synergistically with the antibody to treat a subject with autoimmune disease. For example, the anti-TNFR2 antibodies described herein can be used in combination with corticosteroids (e.g., prednisone, budesonide, prednisolone), calcineurin inhibitors (e.g., cyclosporine, tacrolimus); mTOR inhibitors (e.g., sirolimus, everolimus); EVIDH inhibitors (e.g., azathioprine, leflunomide, mycophenolate); biologics (e.g., abatacept, adalimumab, anakinra, certolizumab, etanercept, golimumab, infliximab, ixekizumab, natalizumab, rituximab, secukinumab, tocilizumab, ustekinumab, vedolizumab); and monoclonal antibodies (e.g., basiliximab, daclizumab, muromonab).

The anti-TNFR2 antibodies described herein are also useful in the context of transplantation (e.g., cell, tissue, or organ transplantation). Accordingly, in some embodiments, provided herein is a method of promoting graft survival and/or reducing graft rejection in a subject (e.g., a human graft recipient) who has received or will receive a cell, tissue, or organ transplant comprising administering to the subject an effective amount (e.g., a therapeutically effective amount) of an anti-TNFR2 described herein to promote graft survival and/or reduce graft rejection. In some embodiments, the graft is autologous, allogeneic, or xenogeneic to the recipient. In other embodiments, the anti-TNFR2 antibody (or combination treatment) can be administered prior to transplantation, at the time of transplantation, and/or after transplantation to promote graft survival and/or reduce graft rejection.

In some embodiments, the graft rejection is in a recipient of a cell, tissue, or organ allograft. In other embodiments, the graft recipient is a recipient of a hematopoietic cell or bone marrow transplant, an allogeneic transplant of pancreatic islet cells, or a solid organ transplant selected from the group consisting of a heart transplant, a kidney-pancreas transplant, a kidney transplant, a liver transplant, a lung transplant, and a pancreas transplant. Additional examples of grafts include but are not limited to allotransplanted cells, tissues, or organs such as vascular tissue, eye, cornea, lens, skin, bone marrow, muscle, connective tissue, gastrointestinal tissue, nervous tissue, bone, stem cells, cartilage, hepatocytes, or hematopoietic cells.

In some embodiments, the method of promoting graft survival and/or reducing graft rejection increases graft survival in the recipient by at least about 15%, by at least about 20%, by at least about 25%, by at least about 30%, by at least about 40%, or by at least about 50%, compared to the graft survival observed in a control recipient. A control recipient may be, for example, a graft recipient that does not receive a therapy post-transplant or that receives a monotherapy following transplant. In certain embodiments, a method of promoting graft survival promotes long-term graft survival (e.g., at least about 6 months, at least 1 year, at least 5 years, at least about 10 years, or longer post-transplantation.

Also provided herein is a method of treating, preventing, or reducing graft-versus-host disease (e.g., in a subject who has or will receive a cell, tissue, or organ transplant) comprising administering to a subject in need thereof an effective amount (e.g., a therapeutically effective amount) of an anti-TNFR2 described herein to treat, prevent, or reduce graft-versus-host disease. The anti-TNFR2 antibody (or combination treatment) can be administered prior to transplantation, at the time of transplantation, and/or after transplantation to treat, prevent, or reduce graft-versus-host disease.

The anti-TNFR2 antibodies described herein can be administered alone or with one or more therapeutic agents that act in conjunction with or synergistically with the antibody to promote graft survival and/or reduce graft rejection, or treat, prevent, or attenuate graft-versus-host disease. For example, the anti-TNFR2 antibodies described herein can be used in combination with an immunomodulatory or immunosuppressive agent, for example, adriamycin, azathiopurine, busulfan, bredinin, brequinar, leflunamide, cyclophosphamide, cyclosporine A, fludarabine, 5-fluorouracil, methotrexate, mycophenolate mofetil, 6-mercaptopurine, a corticosteroid, a nonsteroidal anti-inflammatory, sirolimus (rapamycin), tacrolimus (FK-506), anti-thymocyte globulin (ATG), muromonab-CD3, OKT3, alemtuzumab, basiliximab, daclizumab, rituximab, anti-thymocyte globulin and IVIg.

In the combination treatments described herein, the anti-TNFR2 antibodies described herein can be administered before, after, or concurrently with the one or more additional agents.

Also provided herein is a method of blocking TNFα binding to TNFR2 in a cell comprising contacting the cell with an effective amount of an anti-TNFR2 antibody described herein.

In another embodiment, provided herein is a method of activating TNFR2-mediated signaling in a cell comprising contacting the cell with an effective amount of an antibody described herein.

In some embodiments, provided herein is a method of activating NF-1B signaling in a cell or subject comprising contacting the cell with or administering to the subject an effective amount of an anti-TNFR2 antibody described herein to activate NF-1B signaling.

In some embodiments, provided herein is a method of promoting (e.g., increasing) T cell proliferation (e.g., CD4+ T cells, CD8+ T cells, or both CD4+ T cells and CD8+ T cells) in vitro (e.g., in culture) or in vivo (i.e., in a subject) comprising contacting cells (e.g., T cells) with or administering to the subject an effective amount of an anti-TNFR2 antibody described herein to promote T cell proliferation.

In some embodiments, provided herein is a method of co-stimulating T cells in vitro (e.g., in culture) or in vivo (i.e., in a subject) comprising contacting cells (e.g., T cells) with or administering to a subject an effective amount of an anti-TNFR2 antibody described herein to co-stimulate T cells.

In some embodiments, provided herein is a method of decreasing the abundance of regulatory T cells (e.g., in the T cell compartment) comprising contacting cells (e.g., T cells) with or administering to a subject an effective amount of an anti-TNFR2 antibody described herein to decrease the abundance of regulatory T cells. In some embodiments, the decrease in abundance of regulatory T cells involves ADCC. In some embodiments, the decrease in abundance of regulatory T cells involves inhibition or reduction of proliferation or induction of cell death.

Also provided herein are methods of detecting the presence of TNFR2 in a sample. In some embodiments, the method comprises contacting the sample with an anti-TNFR2 antibody described herein under conditions that allow for formation of a complex between the antibody and TNFR2 protein, and detecting the formation of a complex. In some embodiments, the anti-TNFR2 antibodies described herein can be used to detect the presence or expression levels of TNFR2 proteins on the surface of cells in cell culture or in a cell population. In another embodiment, the anti-TNFR2 antibodies described herein can be used to detect the amount of TNFR2 proteins in a biological sample (e.g., a biopsy). In yet another embodiment, the anti-TNFR2 antibodies described herein can be used in in vitro assays (e.g., immunoassays such as Western blot, radioimmunoassays, ELISA) to detect TNFR2 proteins. The anti-TNFR2 antibodies described herein can also be used for fluorescence activated cell sorting (FACS).

The present invention is further illustrated by the following examples which should not be construed as further limiting. The contents of Sequence Listing, figures and all references, patents and published patent applications cited throughout this application are expressly incorporated herein by reference.

### Examples

Commercially available reagents referred to in the Examples below were used according to manufacturer's instructions unless otherwise indicated. Unless otherwise noted, the present invention uses art-recognized procedures of recombinant DNA technology, such as those described hereinabove and in the following textbooks: Sambrook et al., supra; Ausubel et al., Current Protocols in Molecular Biology (Green Publishing Associates and Wiley Interscience, N.Y., 1989); Innis et al., PCR Protocols: A Guide to Methods and Applications (Academic Press, Inc.: N.Y., 1990); Harlow et al., Antibodies: A Laboratory Manual (Cold Spring Harbor Press: Cold Spring Harbor, 1988); Gait, Oligonucleotide Synthesis (IRL Press: Oxford, 1984); Freshney, Animal Cell Culture, 1987; Coligan et al., Current Protocols in Immunology, 1991.

### Example 1. Binding of Anti-Mouse TNFR2 Antibodies to Mouse TNFR2

This example describes the binding of anti-mouse TNFR2 antibodies to mouse TNFR2 expressed on cells.

Chinese hamster ovary (CHO) cells were transiently transfected with mouse TNFR2 plasmids and maintained in culture. The mean fluorescence intensity (MFI) of mouse TNFR2 expression on CHO cells was measured by flow cytometry and cells were sorted using FACS Aria to select CHO cells expressing >90% of mouse TNFR2. A 3-fold serial dilution of each antibody was prepared in 50 μL in a 96-well plate in FACS buffer starting at 2 μM. CHO cells were filtered through a 70 μm strainer and resuspended in FACS buffer at a concentration of 2×10⁶/mL. One hundred thousand cells were added to each well so that the starting concentration of mouse TNFR2 antibodies was 1 μM. Antibodies and cells were co-incubated at room temperature for 2 hours with gentle agitation. After a total of three washes in FACS buffer, 10 μg/mL of a goat anti-mouse IgG (H+L) secondary antibody conjugated to Alexa647 was added to each well and incubated for 45 minutes at 4 degrees with gentle agitation. After three more washes, cells were resuspended in propidium iodide in FACS buffer, and the binding of anti-mouse TNFR2 antibodies was determined by flow cytometry as a MFI of Alexa647.

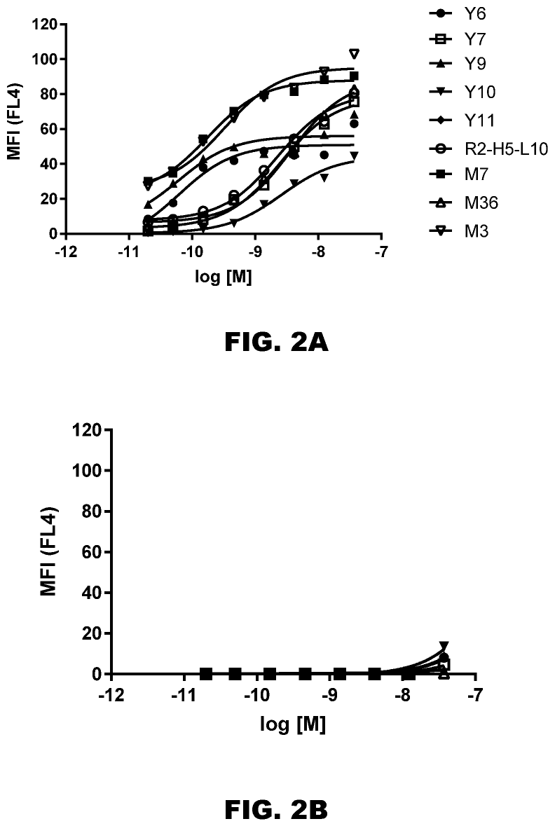

FIGS. 2A and 2B show the non-linear fit curve of on-cell binding on CHO cells overexpressing TNFR2 (FIG. 2A) and wild type CHO cells (FIG. 2B) of anti-mouse TNFR2 antibodies. All antibodies tested bound strongly to CHO cells overexpressing TNFR2, with negligible non-specific binding to wild type CHO cells.

### Example 2. Binding Affinity of Anti-TNFR2 Antibodies

In this example, binding affinities (monovalent K_{D}) of anti-mouse TNFR2 antibodies were determined using the ForteBio assay (bio-layer interferometry).

Briefly, IgG Fc capture sensor tips were hydrated for 10 minutes at room temperature in PBS. A kinetic assay was performed in 96-well plates and ran using the following times for each step: a) baseline: 30 seconds in PBS, b) loading with anti-mouse TNFR2 antibodies at 25 μg/mL in duplicate for 180 seconds, c) dissociation in PBS for 60 seconds, d) association with his-tagged mouse TNFR2 at 75 μg/mL for 180 seconds, and e) dissociation in PBS for 180 seconds.

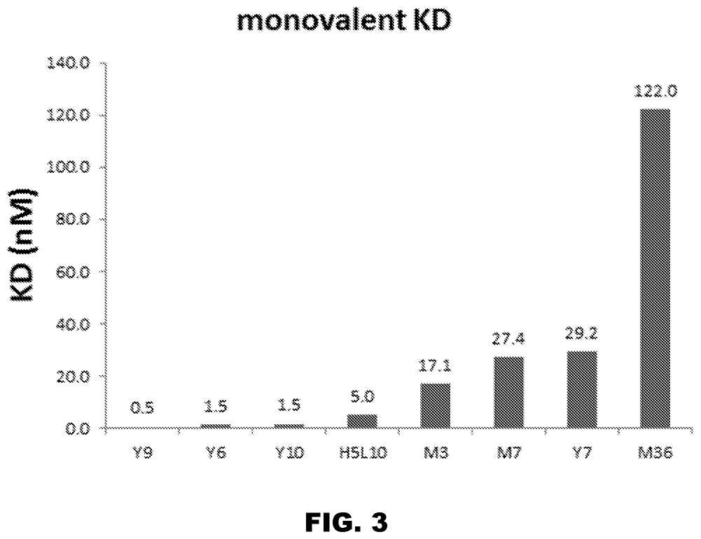

As shown in FIG. 3, all 8 antibodies tested showed specific binding to mouse TNFR2. Specifically, antibodies Y6, Y9, and Y10 showed monovalent K_{D}S of about 1 nM; R2-H5-L10 had a monovalent K_{D} of about 5 nM; Y7, M3, and M7 had monovalent K_{D}S of about 20 nM; and M36 had a monovalent K_{D} of about 122 nM.

### Example 3. Ligand Blocking of Anti-TNFR2 Antibodies

In this example, the ability of anti-mouse TNFR2 antibodies to block the binding of TNF to TNFR2 was tested by ELISA.

Ninety-six-well plates were coated overnight at 4° C. with 50 μL of 2 μg/mL His-tagged mouse TNFR2 protein. Each well was then blocked for one hour with Pierce protein free blocking buffer to prevent non-specific binding of antibodies followed by an incubation with a 2-fold serial dilution of anti-mouse TNFR2 antibodies starting from 500 nM for 2 hours. Recombinant mouse TNFα was incubated for 1 hour and detected with a biotinylated anti-mouse TNFα followed by the incubation with a streptavidin HRP antibody for 40 minutes. Luminescence, which is the result of the activity of a peroxidase conjugated to mouse TNFα, was determined and absorbance was measured at 450 nm. Three washes were performed between each incubation using PBS containing 0.05% TWEEN-20. Assays were performed at room temperature unless indicated otherwise, and proteins were diluted in PBS containing 0.05% TWEEN-20.

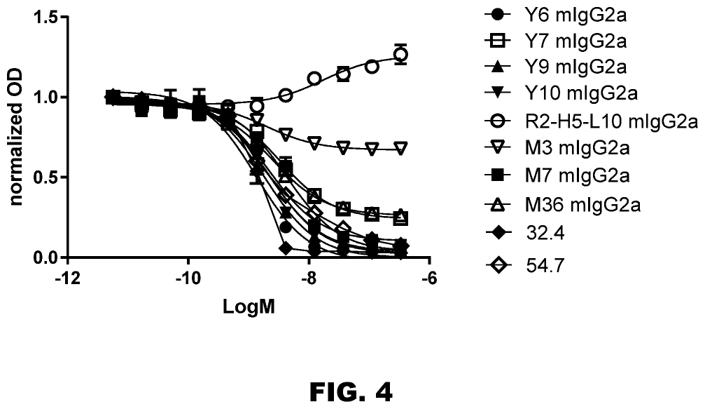

FIG. 4 shows the optical density at 450 nm (OD) normalized for all antibodies at all concentrations tested. Antibodies M3 and R2-H5-L10 did not compete for binding to TNFR2 with TNFα, whereas Y7 and M36 partially blocked and Y6, Y9, Y10, and M3 fully blocked the binding of TNFα to TNFR2.

### Example 4. Epitope Binning of Anti-TNFR2 Antibodies

This example describes the epitope binning of newly generated anti-mouse TNFR2 antibodies relative to commercially available antibodies 32.4 (BioLegend) and 54.7 (Bioxcell) using the ForteBio assay.

Briefly, streptavidin capture sensor tips were hydrated for 10 minutes at room temperature in PBS. A kinetic assay was performed in 96-well plate and run using the following time for each step: a) baseline: 360 seconds in PBS, b) loading with previously biotinylated 32.4 or 54.7 antibodies at 30 μg/mL in duplicate for 120 seconds, c) dissociation in PBS for 10 seconds, d) association with his-tagged mouse TNFR2 at 150 μg/mL for 180 seconds, e) dissociation in PBS for 5 seconds, and f) association with each anti-mouse TNFR2 antibody at 50 μg/mL for 180 seconds.

The results are summarized in Table 3. While antibodies Y6, Y7, Y10, M7, and M36 were grouped into the same bin as antibody 32.4, antibodies Y9 and M3 were grouped into the same bin as 54.7. Interestingly, antibody H5L10 does not overlap with 32.4 or 54.7 bins, suggesting that it recognizes a new bin that differs from the bins recognized by the two commercially available antibodies.

TABLE 3

| | 32.4 | 54.7 |
|---|---|---|
| Y6 | O | NO |
| Y7 | O | NO |
| Y9 | NO | O |
| Y10 | O | NO |
| H5L10 | NO | NO |
| M3 | NO | O |
| M7 | O | NO |
| 32.4 | O | NO |
| M36 | O | NO |
| 54.7 | NO | O |

O = overlapping bin;
NO = non-overlapping bin

### Example 5. Epitope Mapping Using Chimeric Receptor Constructs

This example describes the use of mouse/human chimeric TNFR2 constructs to more specifically define the epitope targeted by anti-mouse TNFR2 antibodies.

Briefly, Fc fusions of chimeric receptors of mouse and human TNFR2 were synthesized by ATUM (Newark, Calif.) (FIG. 5A), and each protein was generated using the Expi 293 transient transfection method followed by protein A purification. Anti-His biosensor tips were hydrated for 10 minutes at room temperature in PBS. A kinetic assay was performed in 96-well plates and run using the following time for each step: a) baseline: 60 seconds in PBS, b) loading with each chimeric protein (0-9), mouse TNFR2, and human TNFR2 at 50 μg/mL for 180 seconds, c) dissociation in PBS for 60 seconds, d) association with each anti-mouse TNFR2 antibody at 250 nM for 180 seconds, and e) dissociation in PBS for 180 seconds.

As shown in FIG. 5B, all antibodies bound to mouse TFNR2, but not to human TNFR2. Based on the pattern of binding to each chimeric construct, the specific epitope region targeted by each antibody was defined in more detail.

### Example 6. Epitope Mapping of Antibody M3

This Example describes the fine epitope mapping of antibody M3 using yeast surface display.

C-terminal flag-tagged mouse TNFR2 was synthesized by fusing a carboxy terminal FLAG epitope tag to the nucleic acid sequence encoding mouse TNFR2 (23-258). The yeast display vector pMYD1200 (Xu et al., MAbs 2013; 5:237-54) was digested with the restriction enzymes, XhoI and KasI, and gel purified using WIZARD SV Gel and PCR Clean-Up kit (Promega). Chemically competent EBYZ cells (Xu et al., supra) were prepared using the Frozen-EZ Yeast Transformation II kit (Zymoresearch) and transformed with 500 ng of insert and 2 μg digested vector and plated on CM Glucose plates lacking tryptophan (Teknova). Single colonies were selected and transferred to SDCAA media (dextrose-20 mg/ml, casamino acids-10 mg/ml, yeast nitrogen base—3.4 mg/ml, ammonium sulfate—10 mg/ml, Na₂HPO₄—5.4 mg/ml and NaH₂PO₄—7.4 mg/ml) for 24 hours at 30° C. with shaking (225 rpm). Cells were then pelleted and resuspended in SDGAA media (galactose-20 mg/ml, casamino acids-10 mg/ml, yeast nitrogen base—3.4 mg/ml, ammonium sulfate—10 mg/ml, Na₂HPO₄—5.4 mg/ml and NaH₂PO₄—7.4 mg/ml) and grown for an additional 48 hours at 20° C. with shaking to induce expression of TNFR2 on the yeast cell surface.

M3 IgG was labeled using an Alexa Fluor 647 Labeling Kit (ThermoFisher Scientific) according to manufacturer's instructions. Yeast displaying TNFR2 point mutants (0.5e6 cells) were incubated with a serial dilution M3/Alexa Fluor 647 (0.01-400 nM) or 1 μM of anti-FLAG M2/Alexa Fluor647 (Cell Signaling Technology) for 1 hour. Cells were then washed with wash buffer (PBS, pH 7.4 containing 0.5% BSA) and fluorescence was measured using FACS-CANTO cytometer (BD Biosciences). To normalize M3 binding to the display level for each TNFR2 point mutant, a ratio of mean fluorescence intensity (MFI) of M3/anti-FLAG was calculated and plotted as a function of M3 concentration. Non-linear regression was fitted using a one-site hyperbola fit using PRISM software (GraphPad).

Domain level mapping identified the epitope of M3 antibody to the CRD1 and CRD2 regions of mouse TNFR2 (Example 5). A fine epitope mapping strategy was used to further define the epitope with amino acid resolution (Levy et al., JMB 2007; 365:196-210). A total of 28 TNFR2 mutants, each containing a single amino acid substitution at surface exposed positions, were displayed on the surface of yeast. To assess the contribution of each position to M3 binding, substitutions at each position were made to either alanine or aspartate (Table 4).

TABLE 4
TNFR2 mutant panel

| | | Corresp. Human |
|---|---|---|
| Substitution | M3 Binding^{A} | Residue |
| G37D | + | G37 |
| E39A | +++ | T39 |
| Q41A | +++ | R41 |
| I42A | ++ | L42 |
| S43A | ++ | — |
| Q44A | ++ | R43 |
| E45A | + | E44 |
| Q52A | + | Q51 |
| M53A | + | M52 |
| A56D | − | S55 |
| P59A | + | S58 |
| P60A | + | P59 |
| Q62A | + | Q61 |
| Y63A | + | H62 |
| K65A | ++ | K64 |
| H66A | ++ | V65 |
| K70A | ++ | K69 |
| T71A | ++ | T70 |
| S72A | ++ | S71 |
| D73A | − | D72 |
| V75A | + | V74 |
| A77D | + | D76 |
| D78A | + | S78 |
| E80A | ++ | E79 |
| A81D | +++ | D80 |
| W88A | + | W87 |
| K113A | +++ | R112 |
| Q114A | +++ | E113 |

^{A}+++, no reduction in M3 binding; ++, 0-50% reduction; +, 50-90% reduction; −, >90% reduction

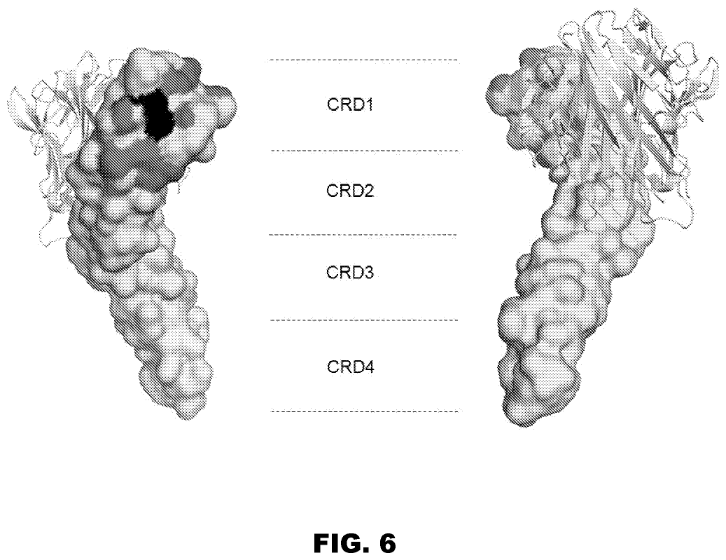

Binding isotherms to M3 (400 nM) were determined for all 28 mutants and the wild-type sequence (Table 4). The positions at which M3 was significantly disrupted (− and +) were mapped onto the homology model of mouse TNFR2 (FIG. 6). Consistent with the domain mapping results, the critical positions for M3 binding were located within the B2 module of CRD1 and A1 module of CRD2. The M3 epitope was also located on the opposite side of the TNFα binding interface and is consistent with the result that M3 does not significantly inhibit ligand binding.

### Example 7. Therapeutic Efficacy of Neutralizing Anti-Mouse TNFR2 Antibodies in a Syngeneic Tumor Model

This example shows the effects of anti-mouse TNFR2 antibodies in a syngeneic mouse tumor model.

6-8 week-old female Balb/C mice were housed in a pathogen-free environment under controlled conditions. Colorectal CT26 tumors were established by subcutaneous injection of 5×10E5 cells in 100 μL PBS into the flank (7 mice/group). All indicated antibodies were injected i.p. in mice harboring tumors with an average size of 80-90 mm³. Tumor growth was monitored using calipers, and volumes were estimated as half the product of the length multiplied by the width squared. Anti-mouse TNFR2 antibodies were injected intraperitoneally in a total volume of 200-300 μL at the indicated times. Antibodies 32.4 and 54.7 were administered at 300 μg on days 0, 2, and 4. Antibodies M3 and M36 were administered at 1000 μg on days 0, 2, 4, 6, and 8. Antibody M7 was administered at 1000 μg on days 0, 2, and 4. Re-challenge experiments were conducted on mice in which tumors regressed completely (cured mice). Mice were re-implanted with 2.5×10E5 CT 26 cells in the contralateral flank at least 60 days after tumors were last detectable.

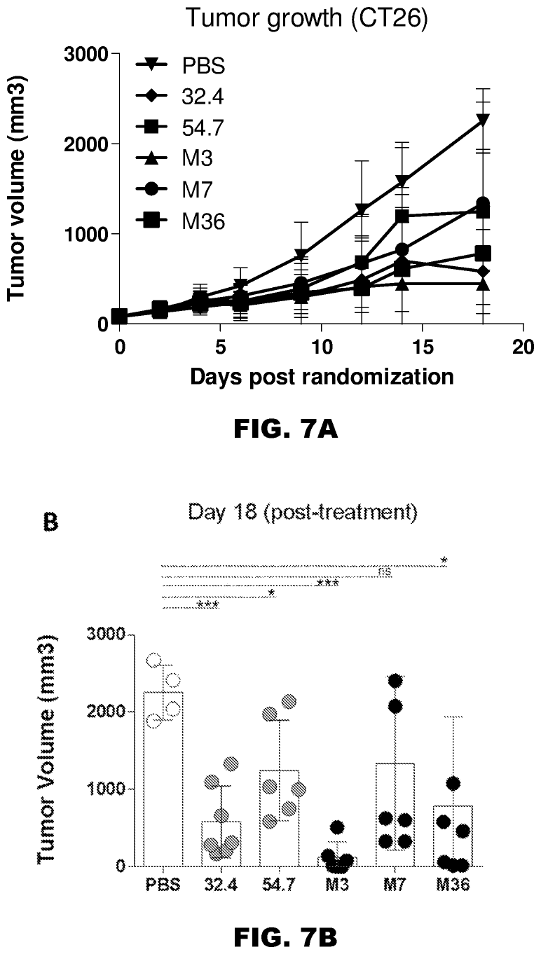

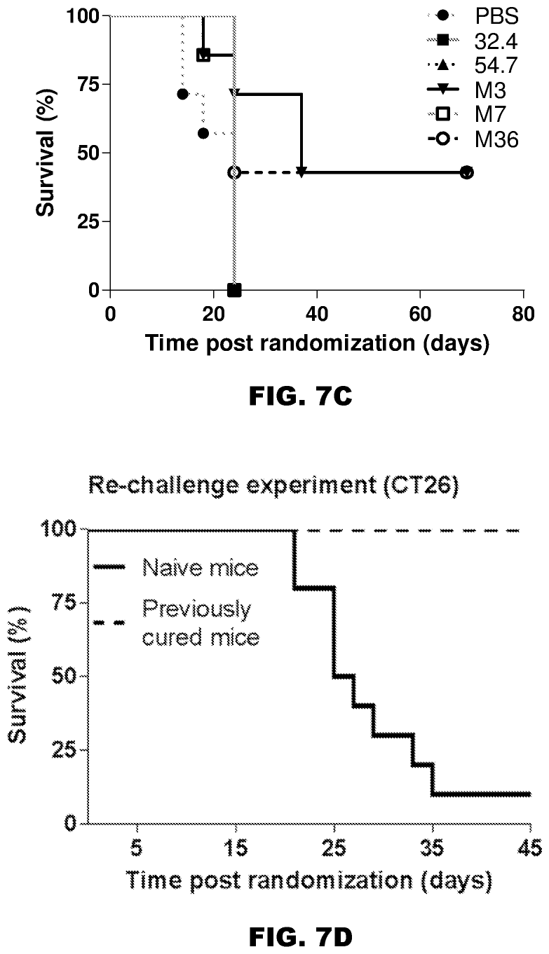

As shown in FIG. 7, commercial monoclonal antibodies to mouse TNFR2 (i.e., clones #32.4 and #54.7) significantly reduced tumor growth in the CT26 model of colorectal cancer in mice (FIGS. 7A and 7B). Newly generated anti-mouse TNFR2 antibodies M3, M36, and to a lesser extent M7, significantly reduced tumor growth in the same model and cured several animals. Mice previously cured with M3 and M36 treatment were re-challenged with CT26 cells in the opposite flank to evaluate long-term therapeutic protection. Animals cured with M3 and M36 treatment (FIGS. 7A-7C) were resistant to re-challenge with the same tumor cell line CT26 (FIG. 7D). These results collectively suggest that newly generated anti-mouse TNFR2 antibodies potently reduce tumor growth and establish long-term protection in vivo.

### Example 8. Fc-Mediated Effector Function Enhances the Therapeutic Efficacy of Anti-Mouse TNFR2 Antibodies in a Syngeneic Tumor Model

This Example shows the effects of Fc effector function on anti-mouse TNFR2 antibody efficacy in a syngeneic tumor model.

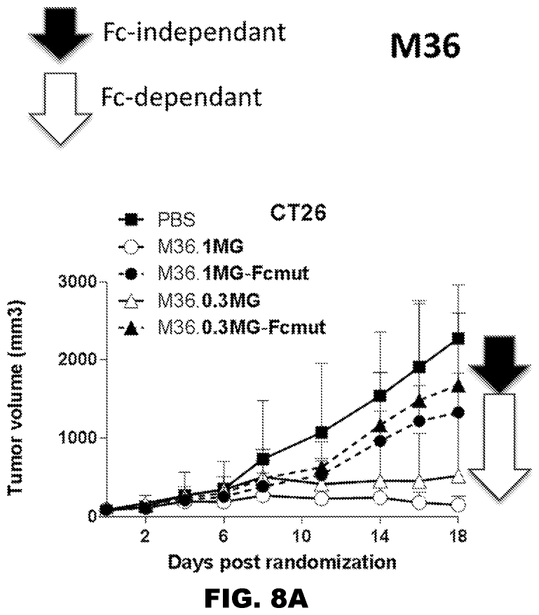

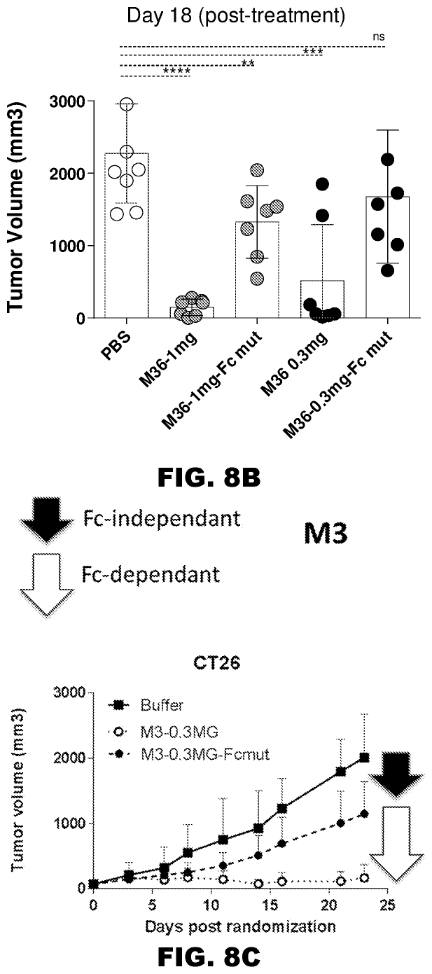

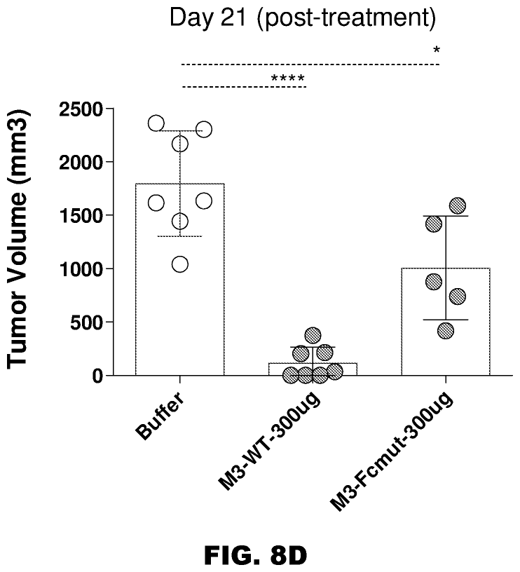

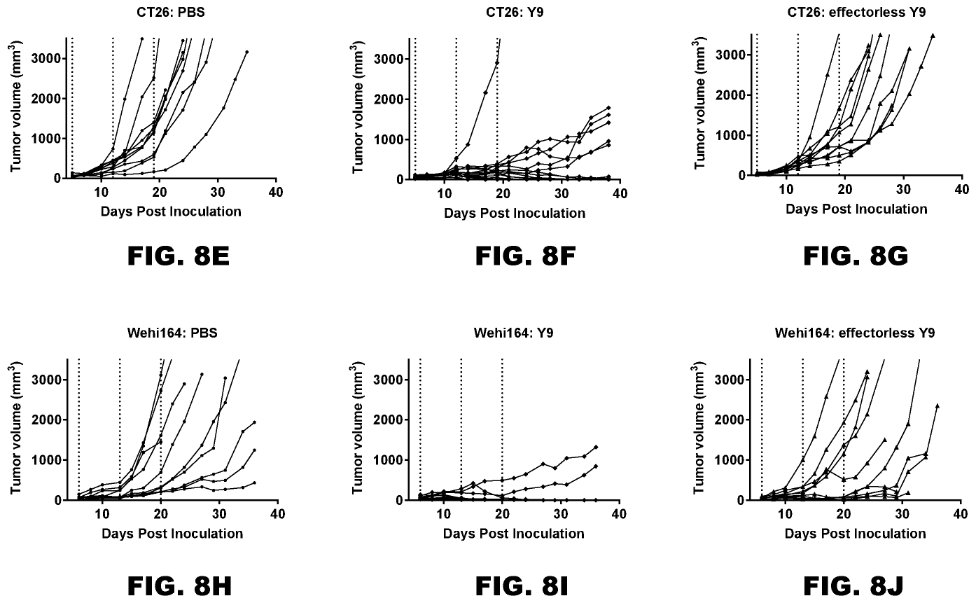

CT26 tumors were established in mice and antibodies M3 and M36 (wild type of Fc-mutated) were administered to the mice as described in Example 7. The Fc mutants harbor two single amino acid substitutions D265A and N297G, which abrogate Fc-mediated effector functions. CT26 cells (5×10E5) were inoculated subcutaneously in 6-week-old female Balb/c mice (7 mice/group). The indicated antibodies were injected i.p. in mice harboring tumors with an average size of 80-90 mm³. Antibody M36 (wild type or Fc-mutated) was tested at two different dose-regimen (i) 1000 μg on days 0, 2, 4, 6, and 8 or (ii) 300 μg on days 0, 2, 4, 6, and 8. Antibody M3 was administered at 300 μg on days 0, 2, 4, 6, and 8. As shown in FIGS. 8A-8D, Fc-mediated effector function was required to reach maximum anti-cancer therapeutic efficacy of the anti-mouse TNFR2 antibodies in the CT26 mouse model.

Additionally, similar results were observed with Y9. CT26 and Wehi164 tumors were established in mice as described in Example 7, and Y9 or Fc-mutated (D265A and N297A) Y9 were injected i.p. in mice harboring tumors with an average size of 60-90 mm³ in three doses of 0.3 mg once per week (n=15 per group). As shown in FIGS. 8E-8J, the antitumor effect of Y9 was severely abrogated by the Fc mutation.

Antibodies Y9, M3 and M36 target distinct epitopes on mouse TNFR2. Additionally, M3 is a non-ligand competitor and M36 is a partial ligand-competitor. Importantly, maximal anti-cancer therapeutic efficacy was achieved independent of the epitope targeted and ligand-competition property.

### Example 9. Therapeutic Efficacy of Anti-Mouse TNFR2 Antibodies Targeting Distinct Epitopes in Syngeneic Tumor Models

This Example demonstrates the therapeutic efficacy of several candidate anti-mouse TNFR2 antibodies that target distinct epitopes on mouse TNFR2.

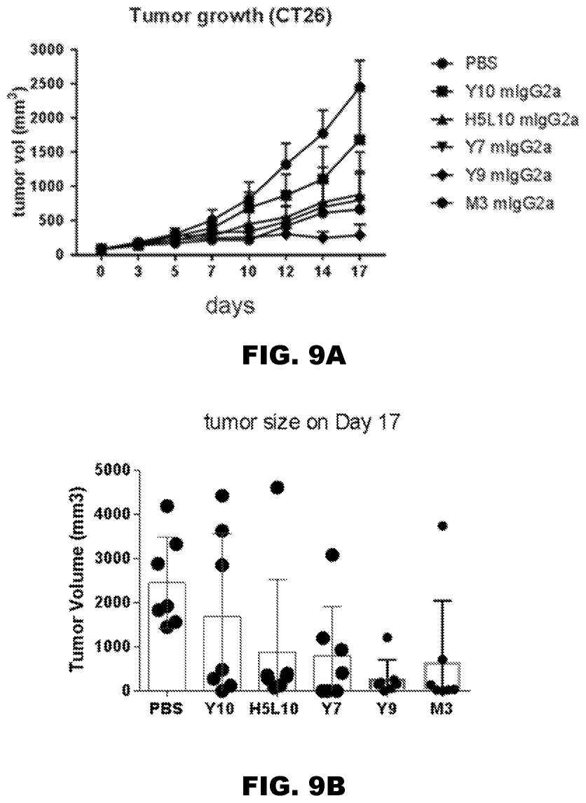

CT26 tumors were established in mice as described in Example 7, and the indicated antibodies were administered at 1 mg on day 0. All antibodies tested were equally potent at saturating doses (not shown), but at sub-optimal doses, antibodies Y9 and M3 showed the best anti-tumor effects in vivo (FIGS. 9A and 9B), with Y9 being superior.

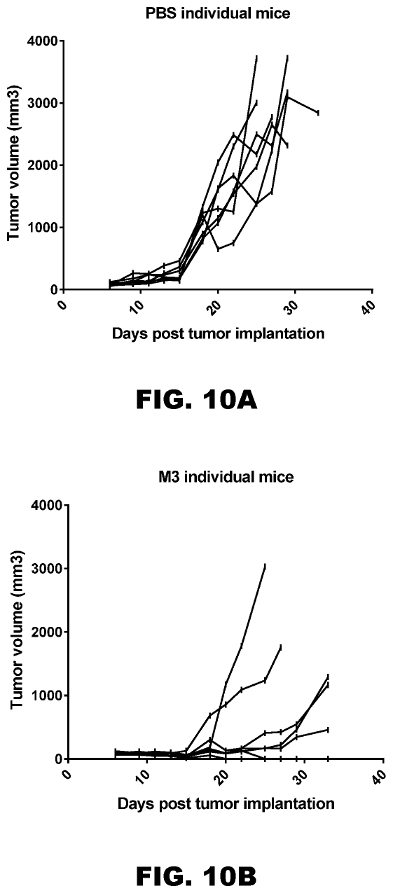

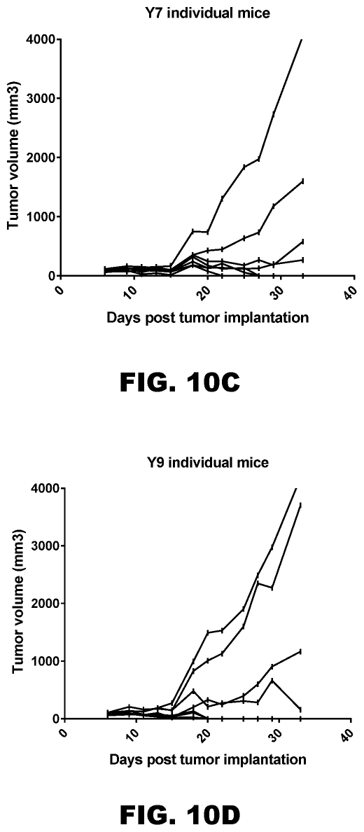

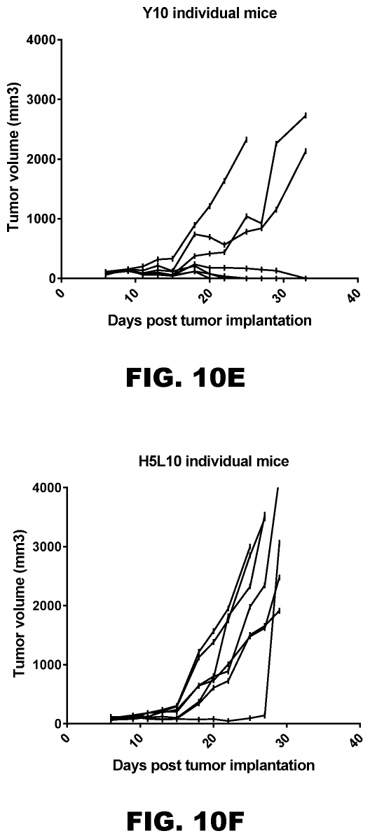

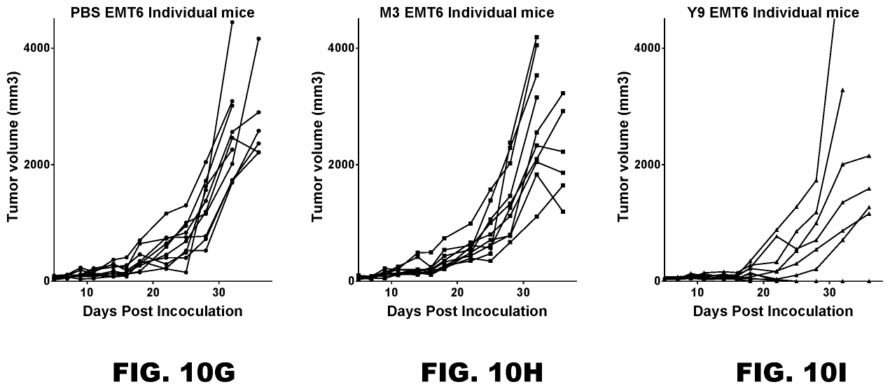

In a separate experiment, EMT6 tumors were established in mice as described in Example 7, and the indicated antibodies were administered in a single dose at 1 mg (FIGS. 10A-10F) or 0.3 mg (FIGS. 10G-10I). Antibodies Y9 and M3 showed the best anti-tumor effects in vivo, with Y9 again being superior, particularly at the lower dose level.

### Example 10. Therapeutic Efficacy of Neutralizing Anti-Mouse TNFR2 Antibodies in a Mouse Model of Breast Cancer

This example shows the effects of anti-mouse TNFR2 antibodies in a mouse model of breast cancer.

Breast EMT6 tumors are established in mice as described in Example 8. TNFR2 antibodies are injected i.p. in mice harboring tumors with an average size of 80-90 mm³. Tumor growth is monitored using calipers, and volumes are estimated as half the product of the length multiplied by the width squared. Anti-mouse TNFR2 antibodies are injected intraperitoneally in a total volume of 200-300 μL at the indicated times. Antibodies 32.4 and 54.7 are administered at 300 μg on days 0, 2, and 4. Antibodies M3 and M36 are administered at 1000 μg on days 0, 2, 4, 6, and 8. Antibody M7 is administered at 1000 μg on days 0, 2, and 4. The antibodies significantly reduce tumor growth.

### Example 11. Therapeutic Efficacy of Antibody Y9 in Anti-PD-1 Sensitive and Resistant Syngeneic Mouse Models

This example compared the efficacy of antibody Y9 and an anti-PD-1 antibody in syngeneic mouse models that are sensitive or resistant to anti-PD-1 therapy.

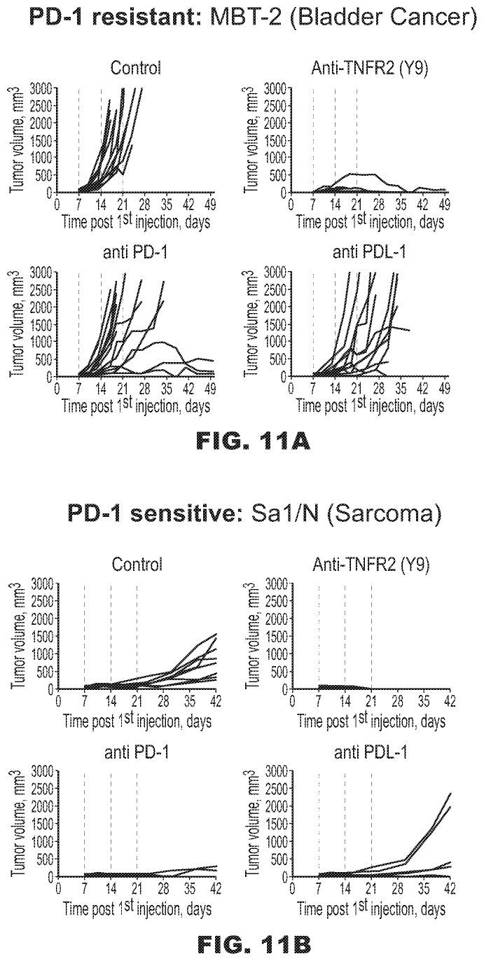

To evaluate the activity of antibody Y9 relative to an anti-PD-1 antibody, a murine version of the hamster anti-mouse PD-1 antibody (J43 clone; Agata et al. Int Immunol. 1996; 8:765-72) was generated by replacing the hamster Fc with a murine IgG2a Fc having D265A and N297A substitutions. Both antibodies were tested in anti-PD-1 sensitive (SaI/N) and resistant (MBT-2) syngeneic mouse models. 6- to 8-week-old female mice were housed in a pathogen-free environment under controlled conditions. Tumors were established by subcutaneous injection of 1×10⁶ MBT-2 (C3H bladder) or 5×10⁶ SaI/N (NCI 1/JCR fibrosarcoma) cells in 200 μL PBS into the right flank (10⁻¹⁵ mice/group). Tumor growth was monitored using calipers, and volumes were calculated according to the formula: π/6×(length×width×width). When tumors reached an average size of 50-100 mm³, 300 μg of antibody was injected i.p. as indicated once weekly for three weeks in a total volume of 200 μL. In both SaI/N (anti-PD-1 sensitive) and MBT-2 (anti-PD-1 resistant) models, anti-TNFR2 (Y9) treatment alone led to complete tumor regression in all treated animals. However, treatment of the MBT-2 bladder model with the anti-PD-1 mAb resulted in only limited activity (FIG. 11).

### Example 12. Therapeutic Efficacy of Combination Therapy with Antibody Y9 and an Anti-PD-1 or Anti-PD-L1 Antibody in Syngeneic Mouse Models

This example describes combination therapy with antibody Y9 and an anti-PD-1 or anti-PD-L1 antibody in various syngeneic mouse models.

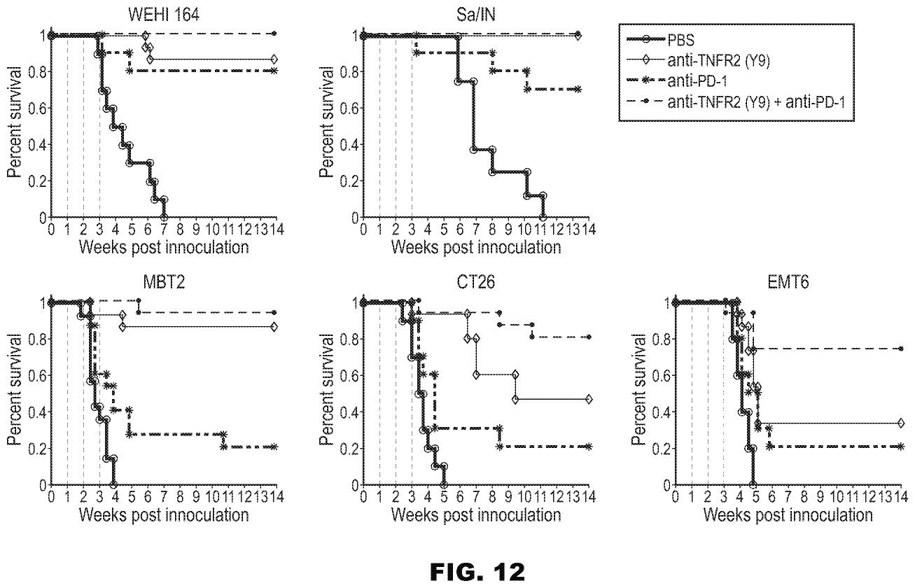

To evaluate whether treatment with murine surrogate anti-TNFR2 antibody (Y9) would synergize with anti-PD-1 or anti-PD-L1 antibody treatment, a murine version of J43 was generated as described in Example 11. A murine version of the PD-L1 antibody, MPDL3280a (Powles et al., Nature 2014; 515:558-62), was also generated by replacing the human Fc with a murine IgG2a Fc with D265A and N297A substitutions. The antibody combinations were tested for activity in syngeneic mouse models. 6- to 8-week-old female mice were housed in a pathogen-free environment under controlled conditions. Tumors were established by subcutaneous injection of 3×10⁵ CT26 (Balb/C colon), EMT6 (Balb/C breast), or Wehi164 (Balb/C fibrosarcoma) cells, 1×10⁶ MBT-2 (C3H bladder) cells, or 5×10⁶ SaI/N (NCI 1/JCR fibrosarcoma) cells in 200 μL PBS into the right flank (7-15 mice/group). Tumor growth was monitored using calipers, and volumes were calculated according to the formula: π/6×(length×width×width). When tumors reached an average size of 50-100 mm³, 300 μg of antibody was injected i.p. as indicated once weekly for three weeks in a total volume of 200 μL. In WEHI164, SaI/N, and MBT2 models, long-term survival was driven by anti-TNFR2 (Y9) treatment alone, whereas in the CT26 and EMT6 models, the combination of anti-TNFR2 (Y9) and anti-PD-1 treatment showed the greatest long-term survival (FIG. 12). Similar results were obtained for anti-PD-L1 treatment, alone and in combination with Y9 (data not shown).

### Example 13. Safety Profile of Antibody Y9 in Comparison with that of an Anti-CTLA4 Antibody

This Example describes various safety/toxicity parameters of antibody Y9 in comparison with an anti-CTLA4 antibody.

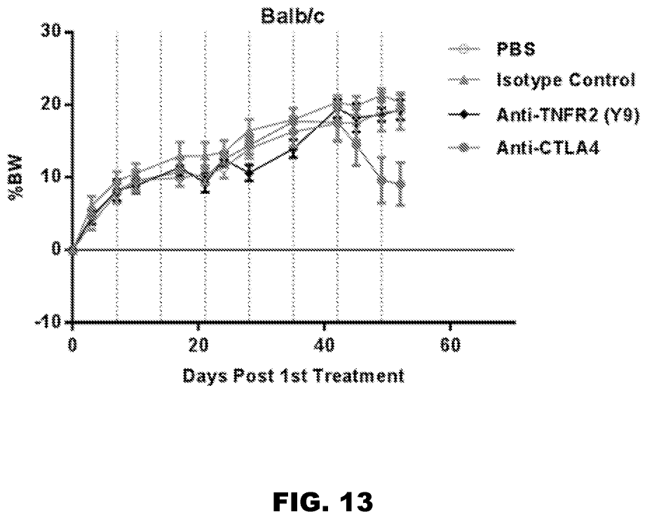

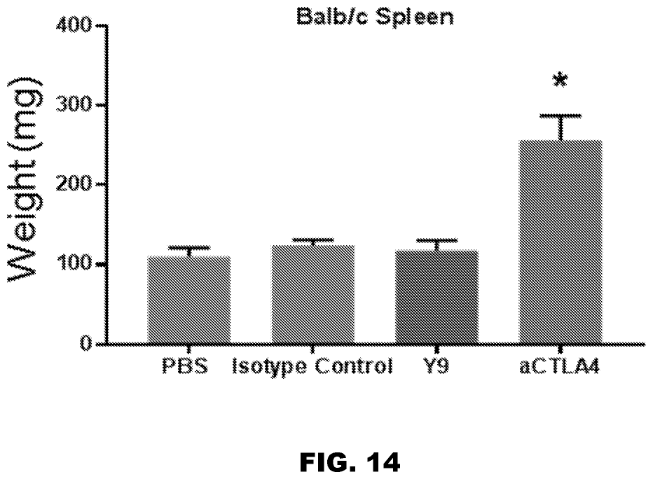

To compare the toxicity profile of antibody Y9 with an anti-CTLA4 antibody, a recombinant version of the mouse anti-mouse CTLA-4 antibody, 9D9 clone (Quezada et al. 2006), with a mouse IgG2a Fc was generated (same isotype as antibody Y9). A long-term exposure study using the antibodies was performed in twenty 6- to 8-week-old Balb/c female mice. Mice were housed in a pathogen-free environment under controlled conditions. For a total of 8 weeks, mice were injected i.p. with 1 mg of antibody (PBS, mouse IgG2a isotype control, anti-TNFR2 (Y9), or anti-CTLA4, n=5 per group) once per week in a total volume of 200 pl. Mouse weight was measured twice per week, and physical well-being of the mice were tracked throughout the study. Saphenous blood from all groups was collected once per week, following the treatment schedule, and one pre-treatment bleed was performed to serve as a baseline control. All mice were sacrificed 48 hours following the final (8^{th}) weekly treatment, whereby spleens were harvested and weighed, and blood was collected via cardiac puncture. As shown in FIG. 13, no difference in weight was detected across groups for the first 6 weeks of treatment, but after the 7^{th} dose of antibody, the anti-CTLA4 group lost weight rapidly, while all other groups had no weight change. Splenomegaly was observed in mice treated with anti-CTLA4 antibody, which was reflected in the significant increase of spleen weight in the anti-CTLA4 group, when compared to Y9 or the control groups (FIG. 14).

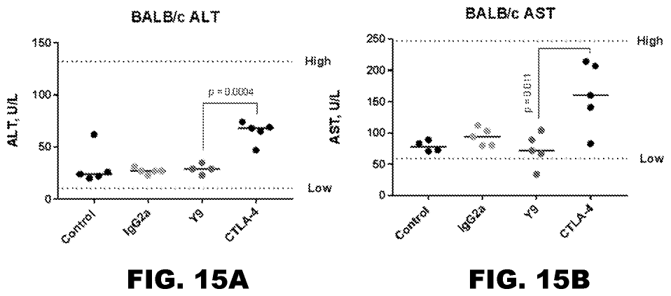

Levels of liver enzymes in the blood were evaluated using Catalyst Dx Chemistry Analyzer (IDEXX, Westbrook, Me.). Briefly, blood samples were collected by cardiac puncture and transferred into lithium heparin whole blood separators (IDEXX, #98-14323-00). Blood levels of ALT (alanine aminotransferase) and AST (aspartate aminotransferase) were analyzed using NSAID 6 CLIP (IDEXX, #98-11007-01). Significant increases in blood ALT (FIG. 15A) and AST (FIG. 15B) were observed in the anti-CTLA4 group, although all groups were within the normal range.

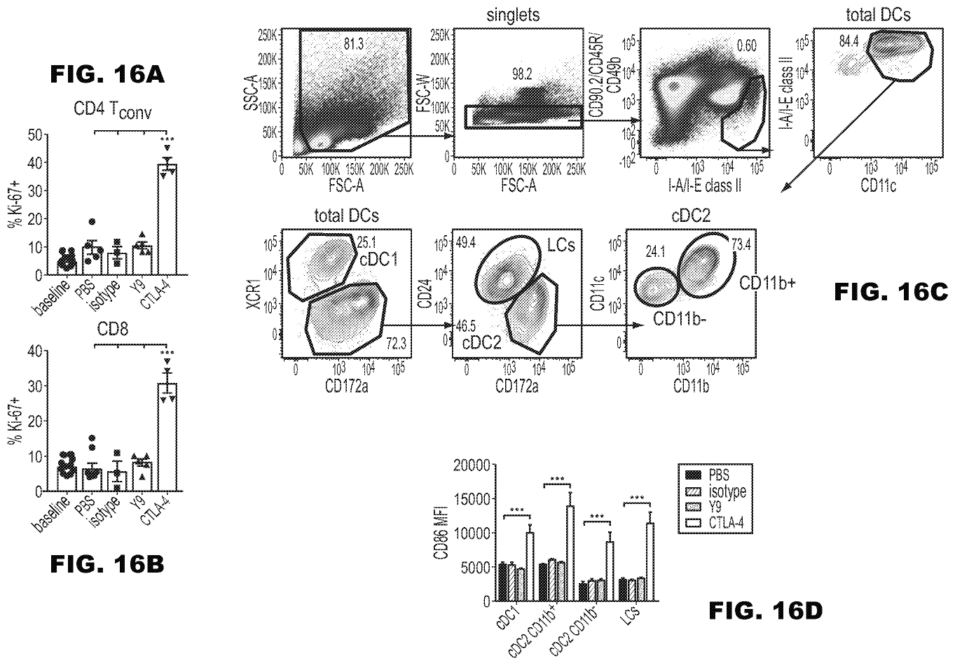

To profile the effect of treatment on immune cell phenotype, peripheral blood lymphocytes and dendritic cells from skin-draining lymph nodes 48 hrs after the final treatment were analyzed by flow cytometry (FIGS. 16A-16D). To prepare blood for flow cytometry, red blood cells were lysed using ACK lysing buffer (Lonza) and washed in flow cytometry buffer (PBS with 1% FCS and 0.02% sodium azide). For DC analysis, skin-draining lymph nodes were digested using the Spleen Dissociation Kit (Miltenyi Biotec) following the manufacturer's instructions. Single cell suspensions were first stained with Fc-Block and live/dead stain in PBS for 10 min at 4° C. Cells were then stained for extracellular markers for 30 min at 4° C. To identify CD4 Tregs, cells were fixed and permeabilized using the Foxp3 Staining Kit (BioLegend) following manufacturer's instructions and stained intracellularly for Ki-67, Foxp3, and CTLA-4. Expression of Ki-67, which is expressed at all stages of the cell cycle except GO, was used to assess T cell proliferation. In mice treated with anti-CTLA-4 antibody, the frequency of CD4 and CD8 T cells proliferating substantially increased relative to isotype controls (FIGS. 16A and 16B). In contract, mice treated with Y9 showed no increase in T cell proliferation, indicating that, unlike the anti-CTLA-4 antibody, Y9 does not cause spontaneous activation and proliferation of peripheral T cells. Consistent with this, Y9 did not upregulate CD86 (B7.2) expression, a co-stimulatory molecule important for dendritic cell activation of T cells, whereas the anti-CTLA-4 antibody did (FIG. 16D). Taken together, these data indicate that administration of anti-TNFR2 antibody Y9 does not lead to spontaneous immune cell activation in healthy mice.

### Example 14. Comparison of Therapeutic Efficacy of Antibody Y9 in Different Engineered Mouse Models and Between Different Antibody Isotype Variants

As described in Example 8, Fcγ receptor engagement of the murine surrogate anti-TNFR2 antibody Y9 is important for its activity in vivo. Fcγ receptor engagement can indicate: 1) contribution of effector functions of the antibody such as antibody dependent cellular cytotoxicity (ADCC) or antibody dependent cellular phagocytosis (ADCP) via activating Fcγ receptors mFcγRI, mFcγRIII, or mFcγRIV; or 2) enhanced agonism via clustering of the antibody on Fcγ receptor-expression cell types (Nimmerjahn et al., Trends in Immunology 2015; 36:325-36). For the latter, the inhibitory Fcγ receptor mFcγRII is considered to be the most important to facilitate agonism (see, e.g., Dahan et al., Cancer Cell 2016; 29:820-31).

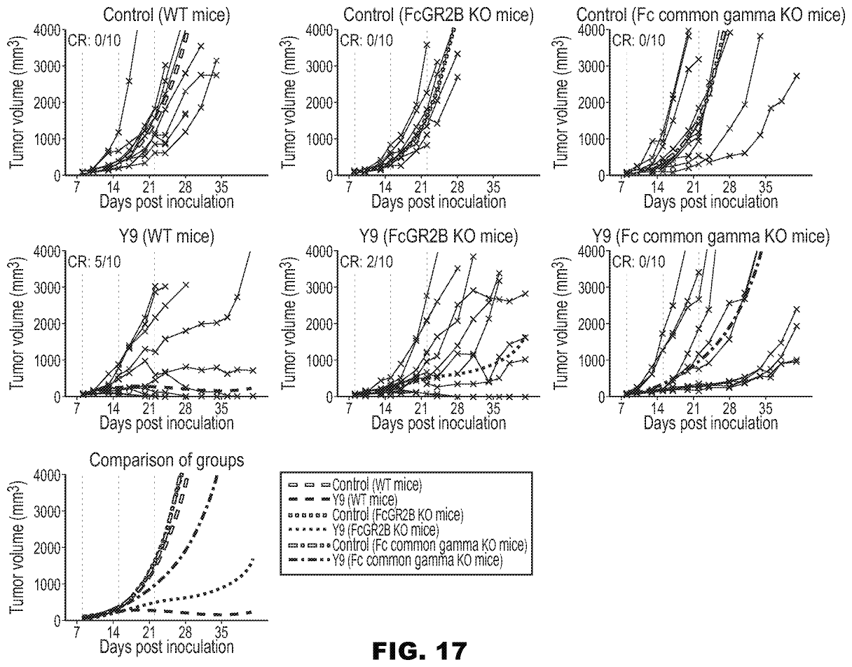

To evaluate which Fcγ receptors are most important for the efficacy of Y9, syngeneic mouse models that are wildtype for the Fcγ receptors (“WT”, Balb/C), lack mFcγRII (“FcGR2B KO”; Fcgr2b—Model 579, Taconic), or lack the common Fc-gamma chain (“Fc common gamma KO”; Fcer1g—Model 584, Taconic) were used. Fc common gamma KO mice are deficient in expression of mFcγRI, mFcγRIII, or mFcγRIV. 6- to 8-week-old female mice were housed in a pathogen-free environment under controlled conditions. Tumors were established by subcutaneous injection of 3×10⁵ CT26 (colon) cells in 200 μL PBS into the right flank (10 mice/group). Tumor growth was monitored using calipers, and volumes were calculated according to the formula: π/6×(length×width×width). When tumors reached an average size of 50-100 mm³, 300 ug of Y9 antibody or PBS as control was injected i.p. as indicated once weekly for three weeks in a total volume of 200 μL. As shown in FIG. 17, Y9 activity was reduced both in FcGR2B KO and Fc common gamma KO mice. This data suggests that both enhanced agonistic activity by clustering by Fcγ receptors as well as ADCC or ADCP potentially contribute to the activity of Y9 in vivo.

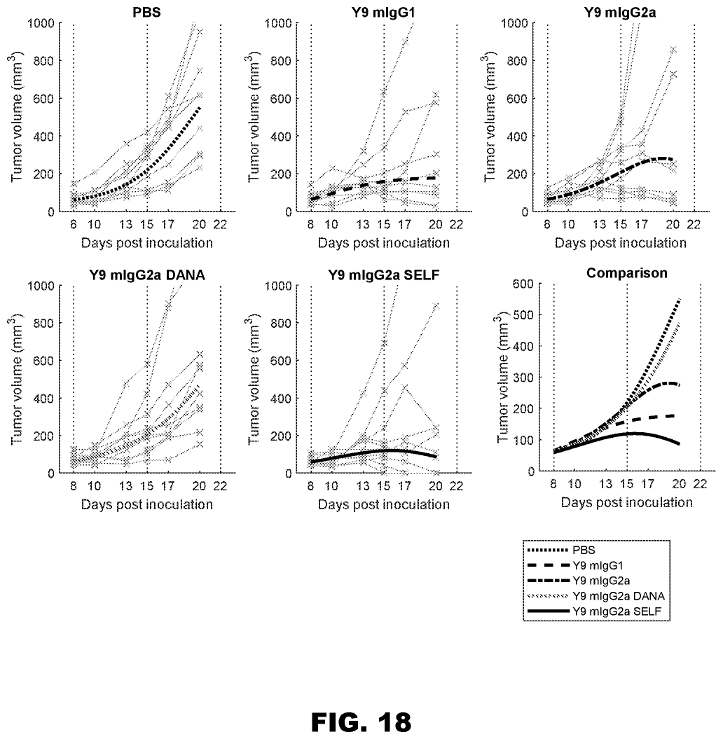

To evaluate which antibody isotype confers the highest activity via engagement of Fcγ receptors, variants of Y9 were created using differ Fc isotypes and mutated isotypes: 1) murine IgG2a which has high affinity for mFcγRI, mFcγRIII, and mFcγRIV; 2) murine IgG1 which has intermediate affinity for mFcγRII and mFcγRIII; murine IgG2a with D265A and N297A mutations (DANA) which does not bind any mFcγRs; and murine IgG2a with S267E and L328F mutations (SELF) which does has increase affinity for mFcγRII. The activity of the different variants was compared in the CT26 (colon) syngeneic mouse model. 6- to 8-week-old female mice were housed in a pathogen-free environment under controlled conditions. Generation of the CT26 model and conditions for administration of Y9 variants were as described above. As shown in FIG. 18, the SELF variant had highest activity, followed by the mIgG1 isotype, then the mIgG2a isotype. The DANA variant lacked efficacy. This data suggests that enhanced agonistic activity by clustering is the major contributor to Fcγ receptor-mediated activity.

### Example 15. Co-Stimulatory Activity of Antibody Y9 and Effects on Proliferation and Functionality of CD8+ T Cells In Vitro

This example describes the direct effects of Y9-mediated cross-linking of CD8+ T cells on co-stimulatory activity, proliferation, and functionality of CD8+ T cells.

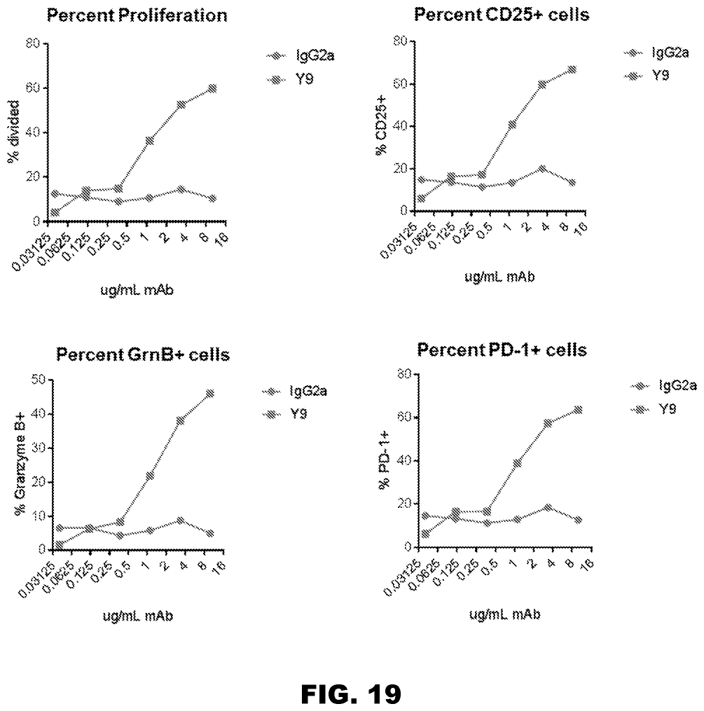

Murine CD8 T cells were stimulated in vitro with anti-CD3/CD28 in the presence of titrated concentrations of Y9. 96-well flat bottom plates were incubated overnight at 4° C. with titrated amounts of functional-grade anti-CD3 (clone 17A2; ThermoFisher Scientific) and Y9 suspended in PBS. Total CD8+ T cells were purified via negative selection (CD8+ T Cell Isolation Kit, mouse; Miltenyi Biotec) from spleens and skin-draining lymph nodes of a BALB/c mouse. CD8 T cells were then labelled with 5 μM CellTrace Violet (Invitrogen). Prior to adding cells, antibody was aspirated from the 96-well plate, wells were blocked for 10 min at room temperature with RPMI containing 10% FCS, and then aspirated again. 4×10⁴ CD8+ T cells were added per well along with 1 μg/mL soluble anti-CD28 (clone 37.51) and incubated at 37° C. for 72 h. Cells were then stained for activation markers and intracellular granzyme B and analyzed by flow cytometry. As shown in FIG. 19, Y9 exhibited co-stimulatory activity, and increased the proliferation and functionality of CD8+ T cells in vitro. Data shown used 1.67 μg/mL plate-bound anti-CD3, 1 μg/mL anti-CD28, and titrated concentrations of Y9. Proliferation was defined as cells undergoing at least 1 round of division indicated by 2-fold dilution of CellTrace Violet mean fluorescence intensity.

### Example 16. Epitope Mapping of Antibody Y9

This Example describes the fine epitope mapping of antibody Y9 using yeast surface display.

Domain level mapping identified the epitope of Y9 antibody to the CRD1 region of mouse TNFR2 (Example 5). A fine epitope mapping strategy as described in Example 6 was used to further define the epitope with amino acid resolution (Levy et al., JMB 2007; 365:196-210). A total of fifteen TNFR2 mutants, each containing a single amino acid substitution at surface exposed positions, were displayed on the surface of yeast. To assess the contribution of each position to Y9 binding, substitutions at each position were made to either alanine or aspartate (Table 5).

TABLE 5
TNFR2 mutant panel

| | | Corresp. Human |
|---|---|---|
| Substitution | Y9 Binding^{A} | Residue |
| G37D | +++ | G37 |
| E39A | +++ | T39 |
| I42A | +++ | L42 |
| R49A | − | Q48 |
| K50A | +++ | T49 |
| Q52A | +++ | Q51 |
| K57A | +++ | K56 |
| H66A | +++ | V65 |
| F67A | +++ | F66 |
| N69A | − | T68 |
| K70A | +++ | K69 |
| V87A | +++ | L86 |
| Q90A | +++ | W89 |
| F91A | ++ | V90 |
| R92A | +++ | P91 |

^{A}+++, no reduction in Y9 binding; ++, 0-50% reduction; +, 50-90% reduction; −, >90% reduction

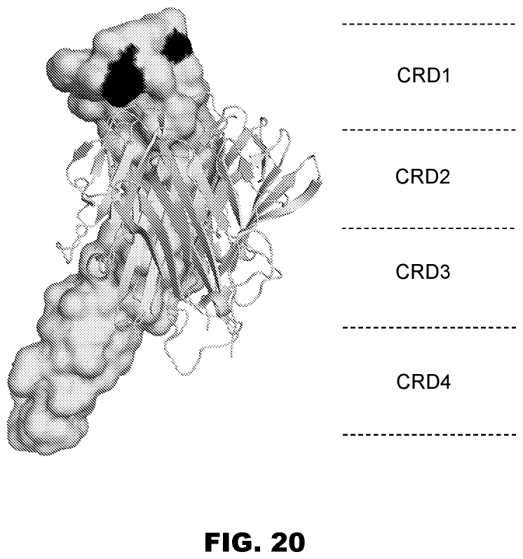

Binding isotherms to Y9 (400 nM) were determined for all fifteen mutants and the wild-type sequence (Table 5). The positions at which Y9 binding was significantly disrupted (−) were mapped onto the homology model of mouse TNFR2 (FIG. 20). The proximity of R49 to the receptor/ligand interface is consistent with the observation that Y9 can compete with ligand for binding to TNFR2.

### Example 17. Anti-Tumor Effects of a Single Dose of Anti-Mouse TNFR2 Antibody in Syngeneic Tumor Models

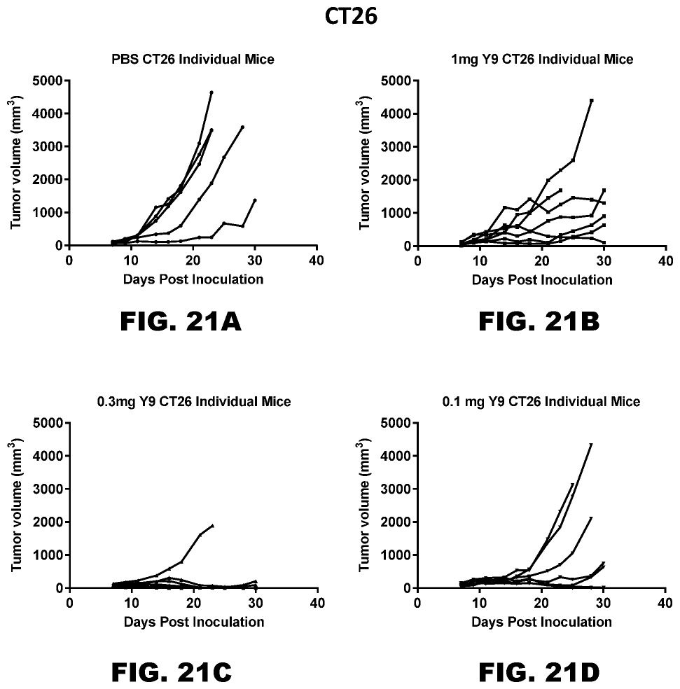

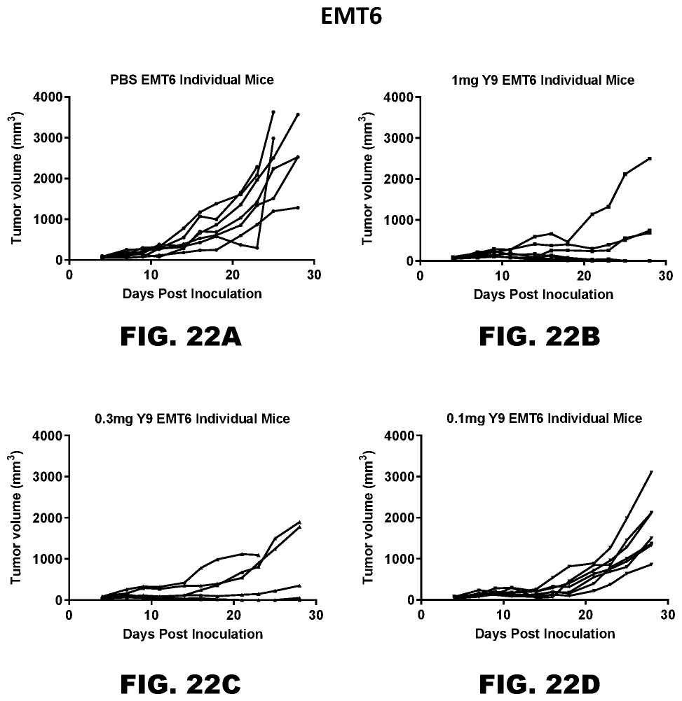

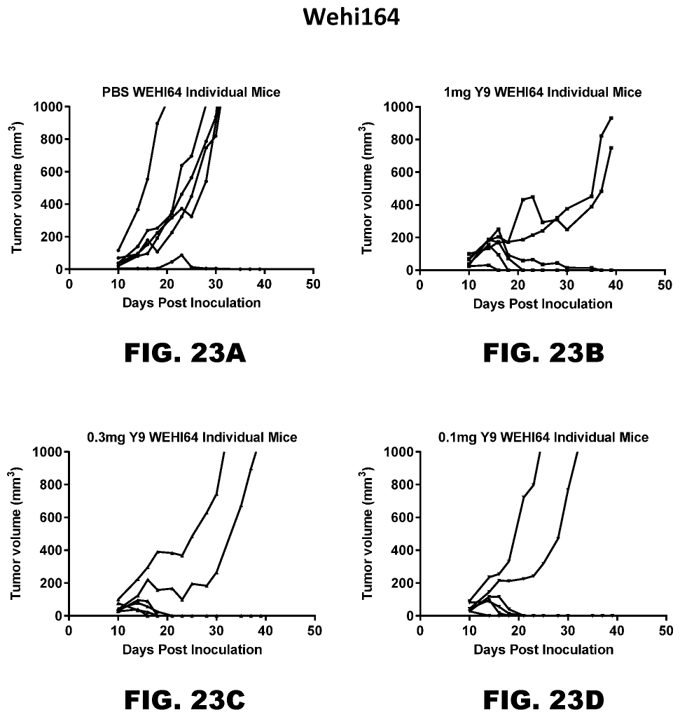

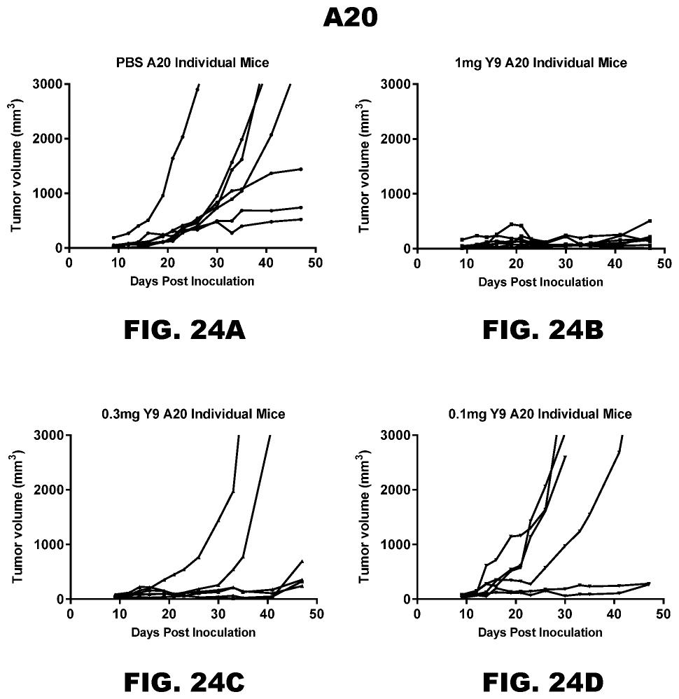

This example demonstrates the antitumor response of a single dose of anti-TNFR2 antibody in multiple syngeneic tumor models. 6-8 week-old female Balb/C mice were housed in a pathogen-free environment under controlled conditions. Tumors were established by subcutaneous injection of 3×10⁵ CT26 (colon), EMT6 (breast), Wehi64 (fibrosarcoma), or A20 (B cell lymphoma) cells in 200 μL PBS into the right flank (6-7 mice/group). Tumor growth was monitored using calipers, and volumes were calculated according to the formula: π/6×(length×width×width). When the tumors reached an average size of 50-70 mm³, Y9 antibody was injected i.p. as a single dose (0.1 mg, 0.3 mg, or 1 mg) in a total volume of 200 μL. Significant antitumor activity was seen with only one dose of antibody in all four models (Table 6, FIGS. 21A-21D, 22A-22D, and 23A-23D, and 24A-24D).

TABLE 6
Anti-tumor effects of single dose of anti-mouse TNFR2 antibody

| | PBS | | 0.1 mg Y9 | | 0.3 mg Y9 | | 1 mg Y9 | |
|---|---|---|---|---|---|---|---|---|
| Model | PR | CR | PR | CR | PR | CR | PR | CR |
| CT26 | 1/7 | 0/7 | 3/7 | 1/7 | 2/7 | 4/7 | 5/7 | 0/7 |
| EMT6 | 0/7 | 0/7 | 0/7 | 0/7 | 4/7 | 3/7 | 2/7 | 4/7 |
| Wehi64 | 0/6 | 1/6 | 0/6 | 4/6 | 1/6 | 4/6 | 2/6 | 4/6 |
| A20 | 2/7 | 0/7 | 2/6 | 0/6 | 3/6 | 0/6 | 7/7 | 0/7 |

PBS: phosphate buffered saline,
PR: partial response,
CR: complete response

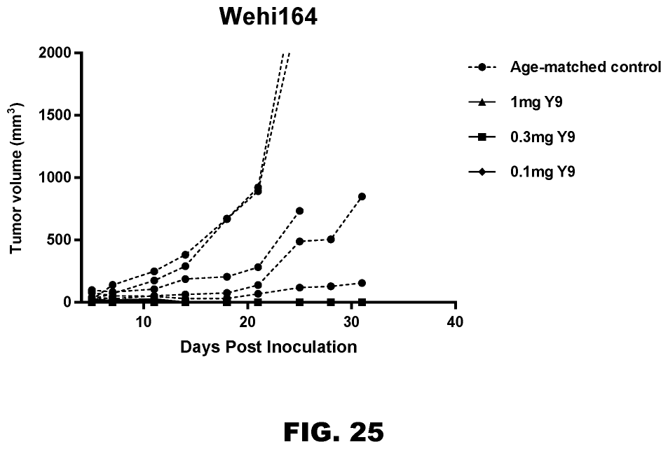

The eleven Wehi64 complete responders were subjected to rechallenge to determine whether a lasting antitumor response was elicited. At day 214 after the initial inoculation, the CR mice and age-matched control mice (5) were rechallenged by subcutaneous injection of 3×10⁵ Wehi64 cells in 200 μL PBS into the left flank, opposite the initial inoculation. Tumor size was monitored as described above. Mice originally administered any of 0.1, 0.3, or 1 mg Y9 experienced no tumor growth, whereas the age-matched controls all had tumor growth (FIG. 25).

This example shows that a single dose of anti-TNFR2 antibodies demonstrate antitumor effects in multiple syngeneic tumor models and that the effects may be retained after tumor clearance.

### Example 18. Effects of Anti-TNFR2 Antibodies on Surface CTLA4 Expression

This example describes the effects of an anti-mouse TNFR2 antibody on CTLA4 expression on T cells.

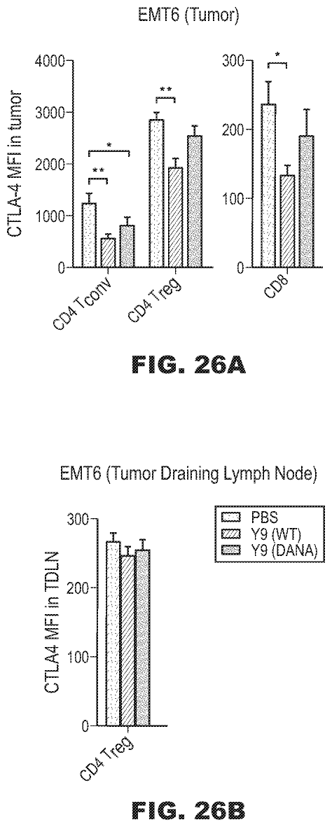

C57BL/6 mice were subcutaneously injected with 3×10⁵ EMT-6 cells. When tumors reached an average size of 200-300 mm³, mice were treated with PBS or 300 μg Y9 or Y9-DANA (i.e., Y9 with an Fc region having D265A and N297A substitutions). Tumors were harvested 36 hours later, digested using the Tumor Dissociation Kit, mouse (Miltenyi Biotec) following the manufacturer's instructions, and stained for T cell lineage markers and CTLA-4 (clone UC10⁻⁴B9, BioLegend). As shown in FIGS. 26A and 26B, Y9 treatment (and to a lesser extent, Y9 DANA treatment) significantly reduced the surface expression of CTLA4 in CD4+ conventional T cells, Tregs, and CD8+ T cells in tumors, whereas no change was observed in the tumor draining lymph node.

### Example 19. Effects of Anti-TNFR2 Antibodies on GITR, GARP, and PD-1 Expression in Tumors

This example describes the effects of anti-mouse TNFR2 antibodies on GITR, GARP, and PD-1 expression in tumors.

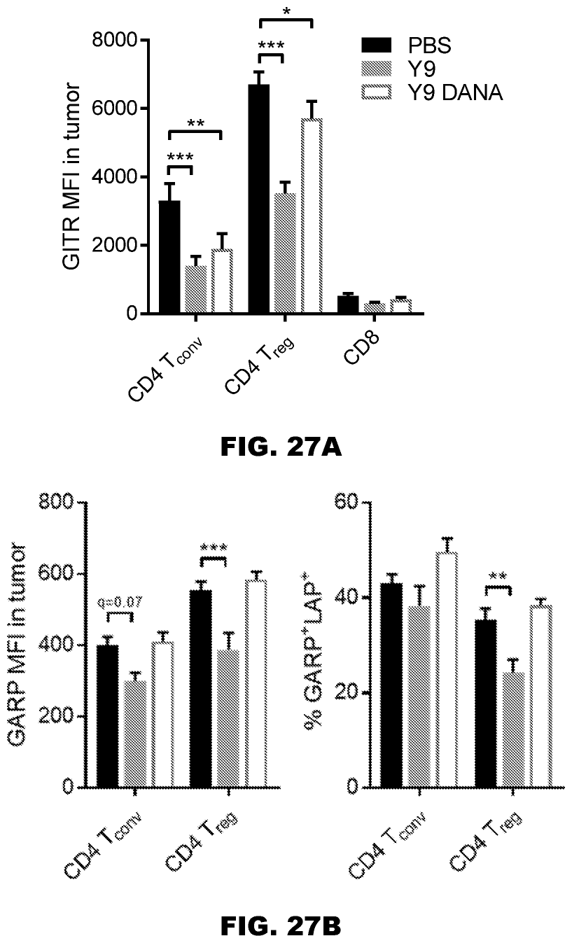

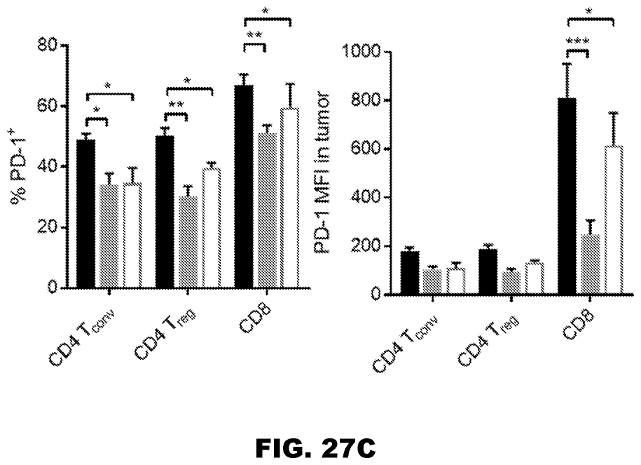

C57BL/6 mice were subcutaneously injected with 3×10⁵ EMT-6 cells. When tumors reached an average size of 200-300 mm³, mice were treated with PBS or 300 μg Y9 or Y9-DANA. Tumors were harvested 36 hours later, digested using the Tumor Dissociation Kit, mouse (Miltenyi Biotec) following the manufacturer's instructions, and stained for T cell lineage markers, GITR (clone DTA-1, BioLegend), GARP (clone F011-5, BioLegend), LAP (TW7-16B4, BioLegend), and PD-1 (RMP1-30, BioLegend). There was a significant decrease in the surface expression of GITR with Y9 treatment, and to a lesser extent, with Y9 DANA (FIG. 27A). Y9, but not Y9 DANA, caused a coordinated decrease in GARP expression, which serves as a docking station for latent TGF-b, as well as LAP (latency-associated peptide) which is associated with TGF-b (FIG. 27B). Similar to GITR, Y9 caused decreased frequencies of PD-1+ effector T cells as well as a notable decrease in the per cell expression on CD8 T cells (shown as median fluorescence intensity) (FIG. 27C).

### Example 20. Effects of Anti-TNFR2 Antibodies on TNFR2 Expression

This example describes the effects of anti-mouse TNFR2 antibodies on TNFR2 expression in tumors.

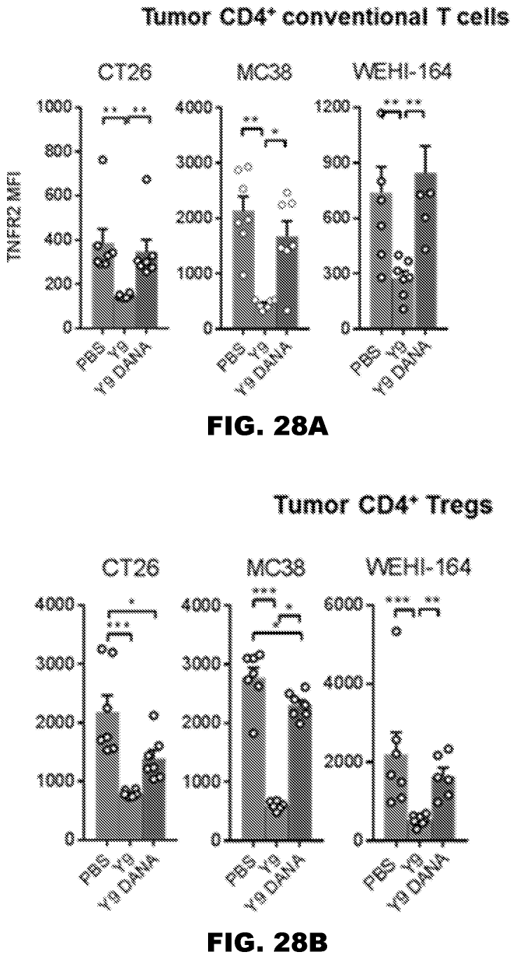

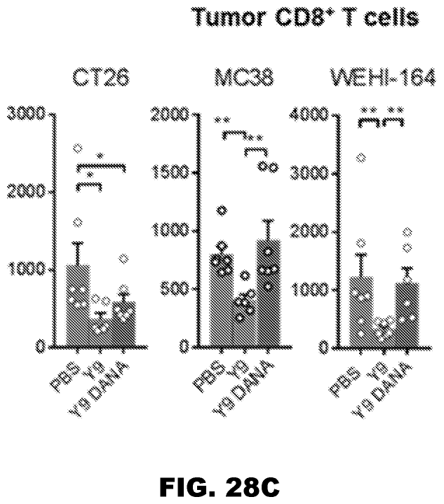

C57BL/6 mice were subcutaneously injected with 3×10⁵ cells for CT26, MC38 and WEHI-164 syngeneic tumor models. When tumors reached an average size of 200-300 mm³, mice were treated with PBS or 300 μg Y9 or Y9-DANA. Tumors were harvested 36 hours (CT26) or 24 hours (MC38 and WEHI-164) later, digested using the Tumor Dissociation Kit, mouse (Miltenyi Biotec) following the manufacturer's instructions, and stained for T cell lineage markers and TNFR2 (clone TR75-89, BioLegend). As shown in FIGS. 28A-28C, a significant decrease was observed in the surface expression of TNFR2 with Y9 treatment, and to a lesser extent, with Y9 DANA treatment.

### Example 21. Anti-Human TNFR2 Antibodies and Chimera Binding

Mouse immunizations were performed at Abveris (Canton, Mass.). Briefly, human TNFR2-His was emulsified with Freund's Complete Adjuvant and four DIVERSIMAB mice were immunized with 100 μg. Booster injections of 100 μg of TNFR2-His in Freund's Incomplete Adjuvant, were given at day 14, 28, 42 and 56. Antibody titers were tested for TNFR2 reactivity by ELISA following the last immunization. Fusions between antibody-producing B-cells and myeloma cells were performed at day 60 and plated onto 96-well plates. Fusions were cultured for 10⁻¹⁴ days and then screened against human TNFR2-His, human TNFR2-Fc, cyno TNFR2-Fc and irrelevant Fc fusion protein. Hybridomas that were positive for all TNFR2 proteins and not the irrelevant Fc protein were then subcloned using limited dilution. Subcloned hybridoma clones were again tested for TNFR2 reactivity by ELISA and positive clones were expanded for antibody production. Antibodies were purified from media using Protein G. Hybridoma sequencing was performed at Genscript (Piscataway, N.J.). Briefly, total RNA was isolated from hybridoma cells and reverse transcribed into cDNA. Antibody sequences were amplified using RACE and cloned into sequencing vector for sequencing.

TABLE 7
Anti-human TNFR2 hybridomas

| Hybridoma | Isotype |
|---|---|
| ABV3 | IgG1 |
| ABV4 | IgG1 |
| ABV7 | IgM |
| ABV12 | IgG1 |
| ABV13 | IgG1 |
| ABV14 | IgM |
| ABV15 | IgM |
| ABV18 | IgG2b |
| ABV19 | IgG1 |

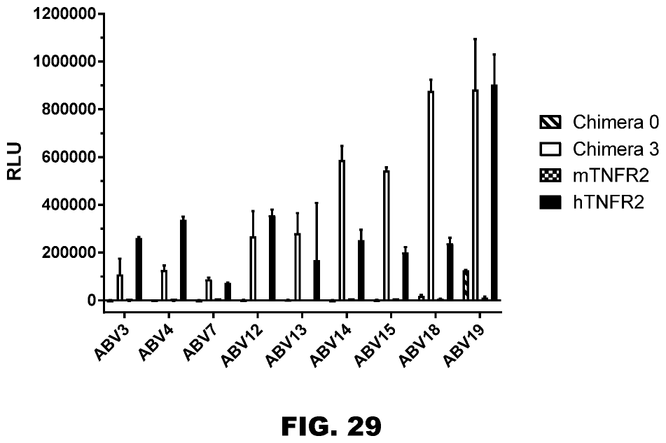

Hybridoma antibodies against human TNFR2 were characterized for binding to chimera 0, chimera 3, mouse TNFR2, and human TNFR2 by ELISA. Briefly, black 384-well microplates (Grenier Bio-one) were coated overnight at 4° C. with chimera 0, chimera 3, mouse TNFR2-His and human TNFR2-His diluted at 1 μg/ml in PBS. Plates were then blocked with PBS/1% BSA for 1 hour followed by PBS/0.05% TWEEN 20 washes. Plates were subsequently incubated with media containing hybridoma antibodies for 1 hour then washed again before addition of AFFINIPURE Goat Anti-Mouse IgG (Jackson ImmunoResearch Laboratories, Inc.) for 1 hour. Following wash, SUPERSIGNAL ELISA Pico chemiluminescent substrate (Thermo Scientific) was added to the wells and luminescence was detected using SYNERGY H1 plate reader (BioTek). As shown in FIG. 29, several mouse antibodies were identified that bound to chimera 3 and human TNFR2 and did not bind to chimera 0 and mouse TNFR2. This example demonstrates the production of anti-human TNFR2 antibodies that bind to a region of human TNFR2 corresponding to the human cognate of the Y9 epitope.

### Example 22. Humanization of Anti-Human TNFR2 Antibodies

This example describes the humanization of mouse anti-human TNFR2 antibody ABV2, which was generated using the method described in Example 21. A chimeric version of ABV2 (ABV2c) was generated by fusing the VH and VL sequence of ABV2 with human IgG1 and human kappa constant regions.

The VH and VL sequences of mouse parental ABV2 and the full-length sequences of the chimera ABV2c are provided in Tables 8 and 9.

TABLE 8

| | | | Enhanced | |
|---|---|---|---|---|
| ABV2 CDR | Kabat | Chothia | Chothia | IMGT |
| sequences | (SEQ ID) | (SEQ ID) | (SEQ ID) | (SEQ ID) |
| VHCDR1 | TFGMS (47) | GYTFTTF | GYTFTTFGMS | GYTFTTF (65) |
| | | (53) | (59) | |
| VHCDR2 | WINTYSGVPTY | NTYSGV | WINTYSGVPT | INTYSGVP (66) |
| | ADDFKG (48) | (54) | (60) | |
| VHCDR3 | RSNFAY (49) | RSNFAY | RSNFAY (61) | ARRSNFAY |
| | | (55) | | (67) |
| VLCDR1 | RASESVDSSGN | RASESVD | RASESVDSSGNS | ESVDSSGNSF |
| | SFMH (50) | SSGNSFM | FMH (62) | (68) |
| | | H (56) | | |
| VLCDR2 | RASNLES (51) | RASNLES | RASNLES (63) | RAS (69) |
| | | (57) | | |
| VLCDR3 | QQSNEDPWT | QQSNEDP | QQSNEDPWT | CQQSNEDPWT |
| | (52) | WT (58) | (64) | (70) |

| | |
|---|---|
| VH | QIQLVQSGPELKKPGETVKISCKASGYTFTTFGMSWVKQAPGKGL |
| | KWMGWINTYSGVPTYADDFKGRFAFSLETSASTAYLQINNLKNED |
| | TATYFCARRSNFAYWGQGTLVTVSA (SEQ ID NO: 71) |
| VL | DIVLTQSPASLAVSLGQRATISCRASESVDSSGNSFMHWYQQKAG |
| | QSFKLLIYRASNLESGIPARFSGSGSRTDFTLTINPVEADDVATYYC |
| | QQSNEDPWTFGGGTKLEIK (SEQ ID NO: 72) |
| Heavy chain of | QIQLVQSGPELKKPGETVKISCKASGYTFTTFGMSWVKQAPGKGL |
| ABV2 chimera | KWMGWINTYSGVPTYADDFKGRFAFSLETSASTAYLQINNLKNED |
| | TATYFCARRSNFAYWGQGTLVTVSAASTKGPSVFPLAPSSKSTSG |
| | GTAALGCLVKDYFPEPVTVSWNSGALTSGVHTFPAVLQSSGLYSL |
| | SSVVTVPSSSLGTQTYICNVNHKPSNTKVDKKVEPKSCDKTHTCPP |
| | CPAPELLGGPSVFLFPPKPKDTLMISRTPEVTCVVVDVSHEDPEVK |
| | FNWYVDGVEVHNAKTKPREEQYNSTYRVVSVLTVLHQDWLNGK |
| | EYKCKVSNKALPAPIEKTISKAKGQPREPQVYTLPPSREEMTKNQV |
| | SLTCLVKGFYPSDIAVEWESNGQPENNYKTTPPVLDSDGSFFLYSK |
| | LTVDKSRWQQGNVFSCSVMHEALHNHYTQKSLSLSPG (SEQ ID |
| | NO: 101) |
| Light chain of | DIVLTQSPASLAVSLGQRATISCRASESVDSSGNSFMHWYQQKAG |
| ABV2chimera | QSFKLLIYRASNLESGIPARFSGSGSRTDFTLTINPVEADDVATYYC |
| | QQSNEDPWTFGGGTKLEIKRTVAAPSVFIFPPSDEQLKSGTASVVC |
| | LLNNFYPREAKVQWKVDNALQSGNSQESVTEQDSKDSTYSLSSTL |
| | TLSKADYEKHKVYACEVTHQGLSSPVTKSFNRGEC (SEQ ID NO: |
| | 102) |

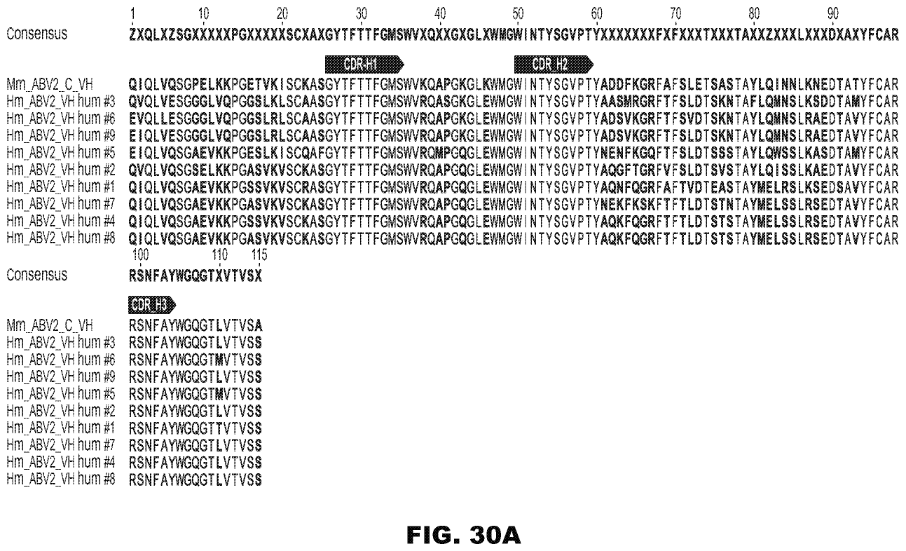

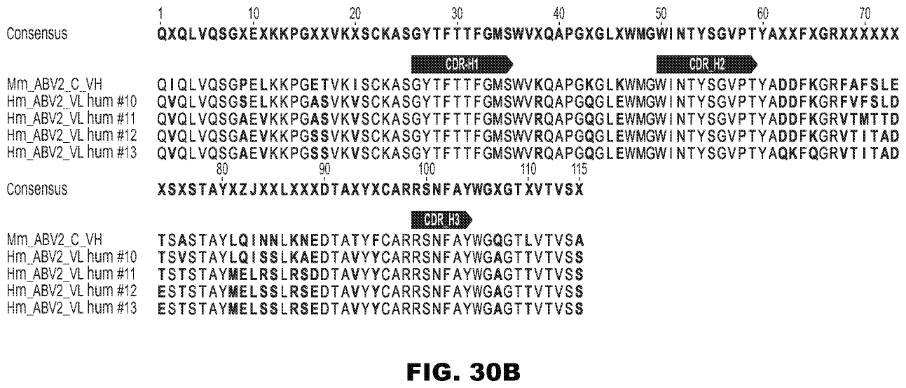

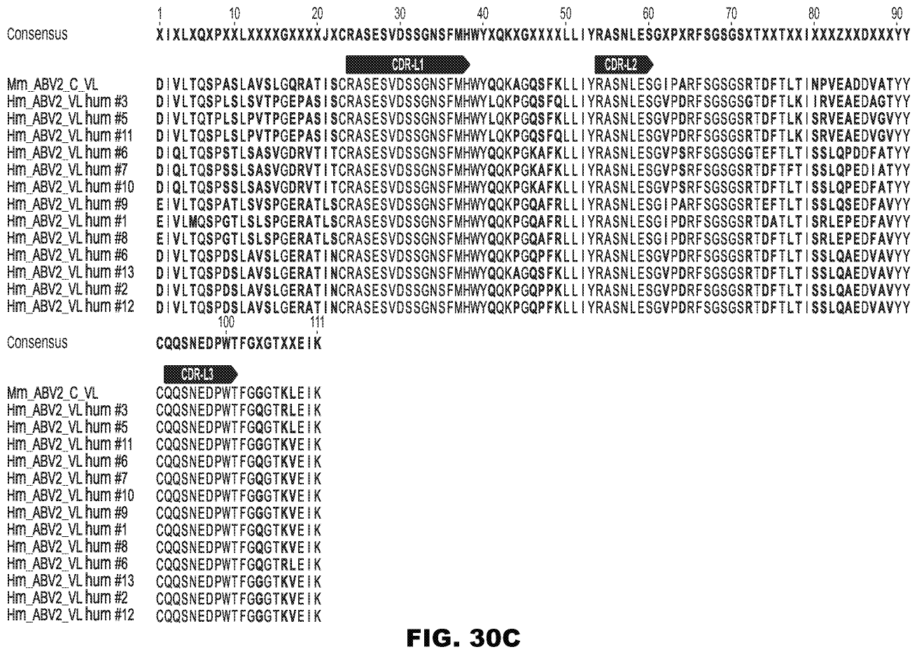

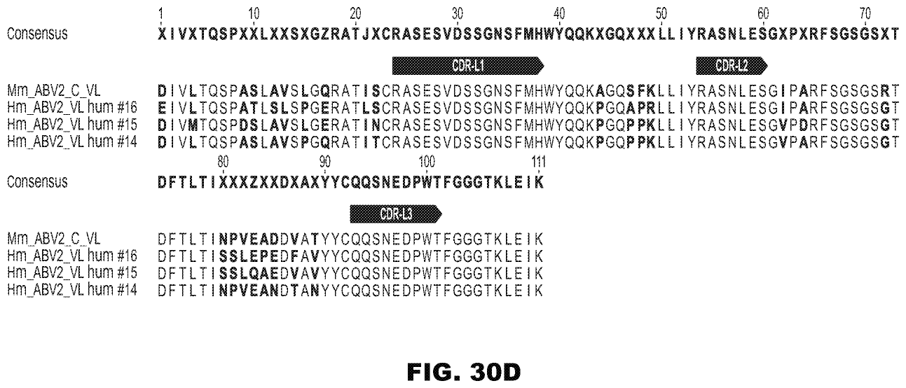

For humanization of ABV2, mouse antibody variable sequences were used as an input to generate a homology models using Schrodinger's Bioluminate software. Enhanced Chothia nomenclature were used to define CDRs and framework boundaries. Different humanization templates from the protein data bank were chosen based on various sequence and structural parameters such as but not limited to overall identity, similarity, CDR stem geometry. In addition, human germline gene sequences with highest identity to the mouse parental antibody sequences were obtained and utilized for framework selection. Human antibody templates with highest scores were chosen, and framework replacement was performed but residues that are part of vernier zone, canonical structure, and interface were unchanged (mouse parental). Following framework replacement, the humanized homology model was energy minimized using gromos force field and residues with very high total energy were further examined. Non-conserved residues that exhibited steric clashes were either mutated back to the corresponding parental mouse sequence or a substitution was made based on residue distribution statistics among human antibody sequences from antibody database. After these residue changes, the model was again subjected to energy minimization and frameworks with acceptable energy scores were chosen for testing. Humanized heavy and light chain variable region sequences of ABV2 generated using the process described above are shown below. Alignments of the humanized sequences are provided in FIGS. 30A-30D.

HUMANIZED HEAVY CHAIN VARIABLE REGIONS
ABV2 VH_hum#1 (HD1) (SEQ ID NO: 73)
QIQLVQSGAEVKKPGSSVKVSCRASGYTFTTFGMSWVRQAPGQGLEWMGWINTYSGV
PTYAQNFQGRFAFTVDTEASTAYMELRSLKSEDSAVYFCARRSNFAYWGQGTTVTVSS
ABV2 VH_hum#2 (HD3) (SEQ ID NO: 74)
QVQLVQSGSELKKPGASVKVSCKASGYTFTTFGMSWVRQAPGQGLEWMGWINTYSGV
PTYAQGFTGRFVFSLDTSVSTAYLQISSLKAEDTAVYFCARRSNFAYWGQGTLVTVSS
ABV2 VH_hum#3 (HD4) (SEQ ID NO: 75)
QVQLVESGGGLVQPGGSLKLSCAASGYTFTTFGMSWVRQASGKGLEWMGWINTYSGV
PTYAASMRGRFTFSLDTSKNTAFLQMNSLKSDDTAMYFCARRSNFAYWGQGTLVTVSS
ABV2 VH_hum#4 (HD5) (SEQ ID NO: 76)
QIQLVQSGAEVKKPGSSVKVSCKASGYTFTTFGMSWVRQAPGQGLEWMGWINTYSGV
PTYAQKFQGRFTFTLDTSTSTAYMELSSLRSEDTAVYFCARRSNFAYWGQGTLVTVSS
ABV2 VH_hum#5 (HD6) (SEQ ID NO: 77)
EIQLVQSGAEVKKPGESLKISCQAFGYTFTTFGMSWVRQMPGQGLEWMGWINTYSGVP
TYNENFKGQFTFSLDTSSSTAYLQWSSLKASDTAMYFCARRSNFAYWGQGTMVTVSS
ABV2 VH_hum#6 (HD8) (SEQ ID NO: 78)
EVQLLESGGGLVQPGGSLRLSCAASGYTFTTFGMSWVRQAPGKGLEWMGWINTYSGV
PTYNENFKGRFTFSVDTSKNTAYLQMNSLRAEDTAVYFCARRSNFAYWGQGTMVTVSS
ABV2 VH_hum#7 (HD9) (SEQ ID NO: 79)
QIQLVQSGAEVKKPGASVKVSCKASGYTFTTFGMSWVRQAPGQGLEWMGWINTYSGV
PTYNEKFKSKFTFTLDTSTNTAYMELSSLRSEDTAVYFCARRSNFAYWGQGTLVTVSS
ABV2 VH_hum#8 (HD13) (SEQ ID NO: 80)
QIQLVQSGAEVKKPGASVKVSCKASGYTFTTFGMSWVRQAPGQGLEWMGWINTYSGV
PTYAQKFQGRFTFTLDTSTSTAYMELSSLRSEDTAVYFCARRSNFAYWGQGTLVTVSS
ABV2 VH_hum#9 (HD15) (SEQ ID NO: 81)
EIQLVESGGGLVQPGGSLRLSCAASGYTFTTFGMSWVRQAPGKGLEWMGWINTYSGVP
TYADSVKGRFTFSLDTSKNTAYLQMNSLRAEDTAVYFCARRSNFAYWGQGTLVTVSS
ABV2 VH_hum#10 (GBM01) (SEQ ID NO: 82)
QVQLVQSGSELKKPGASVKVSCKASGYTFTTFGMSWVRQAPGQGLEWMGWINTYSGV
PTYADDFKGRFVFSLDTSVSTAYLQISSLKAEDTAVYYCARRSNFAYWGAGTTVTVSS
ABV2 VH_hum#11 (GBM02) (SEQ ID NO: 83)
QVQLVQSGAEVKKPGASVKVSCKASGYTFTTFGMSWVRQAPGQGLEWMGWINTYSG
VPTYADDFKGRVTMTTDTSTSTAYMELRSLRSDDTAVYYCARRSNFAYWGAGTTVTV
SS
ABV2 VH_hum#12 (GBM04) (SEQ ID NO: 84)
QVQLVQSGAEVKKPGSSVKVSCKASGYTFTTFGMSWVRQAPGQGLEWMGWINTYSGV
PTYAQKFQGRVTITADESTSTAYMELSSLRSEDTAVYYCARRSNFAYWGAGTTVTVSS
HUMANIZED LIGHT CHAIN VARIABLE REGIONS
ABV2 VL_hum#1 (HD1) (SEQ ID NO: 85)
EIVLMQSPGTLSLSPGERATLSCRASESVDSSGNSFMHWYQQKPGQAFRLLIYRASNLES
GIPDRFSGSGSRTDATLTISRLEPEDFAVYYCQQSNEDPWTFGQGTKVEIK
ABV2 VL_hum#2 (HD3) (SEQ ID NO: 86)
DIVLTQSPDSLAVSLGERATINCRASESVDSSGNSFMHWYQQKPGQPPKLLIYRASNLES
GVPDRFSGSGSRTDFTLTISSLQAEDVAVYYCQQSNEDPWTFGGGTKVEIK
ABV2 VL_hum#3 (HD4) (SEQ ID NO: 87)
DIVLTQSPLSLSVTPGEPASISCRASESVDSSGNSFMHWYLQKPGQSFQLLIYRASNLESG
VPDRFSGSGSGTDFTLKIIRVEAEDAGTYYCQQSNEDPWTFGQGTRLEIK
ABV2 VL_hum#4 (HD5) (SEQ ID NO: 88)
DIVLTQSPDSLAVSLGERATINCRASESVDSSGNSFMHWYQQKPGQPFKLLIYRASNLES
GVPDRFSGSGSRTDFTLTISSLQAEDVAVYYCQQSNEDPWTFGQGTRLEIK
ABV2 VL_hum#5 (HD6) (SEQ ID NO: 89)
DIVLTQTPLSLPVTPGEPASISCRASESVDSSGNSFMHWYLQKPGQSFKLLIYRASNLESG
VPDRFSGSGSRTDFTLKISRVEAEDVGVYYCQQSNEDPWTFGQGTKLEIK
ABV2 VL_hum#6 (HD8) (SEQ ID NO: 90)
DIQLTQSPSTLSASVGDRVTITCRASESVDSSGNSFMHWYQQKPGKAFKLLIYRASNLES
GVPSRFSGSGSGTEFTLTISSLQPDDFATYYCQQSNEDPWTFGQGTKVEIK
ABV2 VL_hum#7 (HD9) (SEQ ID NO: 91)
DIQLTQSPSSLSASVGDRVTITCRASESVDSSGNSFMHWYQQKPGKAFKLLIYRASNLES
GVPSRFSGSGSRTDFTFTISSLQPEDIATYYCQQSNEDPWTFGQGTKVEIK
ABV2 VL_hum#8 (HD10) (SEQ ID NO: 92)
EIVLTQSPGTLSLSPGERATLSCRASESVDSSGNSFMHWYQQKPGQAFRLLIYRASNLES
GIPDRFSGSGSRTDFTLTISRLEPEDFAVYYCQQSNEDPWTFGQGTKVEIK
ABV2 VL_hum#9 (HD13) (SEQ ID NO: 93)
EIVLTQSPATLSVSPGERATLSCRASESVDSSGNSFMHWYQQKPGQAFRLLIYRASNLES
GIPARFSGSGSRTEFTLTISSLQSEDFAVYYCQQSNEDPWTFGGGTKVEIK
ABV2 VL_hum#10 (HD14) (SEQ ID NO: 94)
DIQLTQSPSSLSASVGDRVTITCRASESVDSSGNSFMHWYQQKPGKAFKLLIYRASNLES
GVPSRFSGSGSRTDFTLTISSLQPEDFATYYCQQSNEDPWTFGGGTKVEIK
ABV2 VL_hum#11 (HD15) (SEQ ID NO: 95)
DIVLTQSPLSLPVTPGEPASISCRASESVDSSGNSFMHWYLQKPGQSFQLLIYRASNLESG
VPDRFSGSGSRTDFTLKISRVEAEDVGVYYCQQSNEDPWTFGGGTKVEIK
ABV2 VL_hum#12 (HD17) (SEQ ID NO: 96)
DIVLTQSPDSLAVSLGERATINCRASESVDSSGNSFMHWYQQKPGQPFKLLIYRASNLES
GVPDRFSGSGSRTDFTLTISSLQAEDVAVYYCQQSNEDPWTFGGGTKVEIK
ABV2 VL_hum#13 (HD25) (SEQ ID NO: 97)
DIVLTQSPDSLAVSLGERATINCRASESVDSSGNSFMHWYQQKAGQSFKLLIYRASNLES
GVPDRFSGSGSRTDFTLTISSLQAEDVAVYYCQQSNEDPWTFGGGTKVEIK
ABV2 VL_hum#14 (GBM01) (SEQ ID NO: 98)
DIVLTQSPASLAVSPGQRATITCRASESVDSSGNSFMHWYQQKPGQPPKLLIYRASNLES
GVPARFSGSGSGTDFTLTINPVEANDTANYYCQQSNEDPWTFGGGTKLEIK
ABV2 VL_hum#15 (GBM02) (SEQ ID NO: 99)
DIVMTQSPDSLAVSLGERATINCRASESVDSSGNSFMHWYQQKPGQPPKLLIYRASNLES
GVPDRFSGSGSGTDFTLTISSLQAEDVAVYYCQQSNEDPWTFGGGTKLEIK
ABV2 VL_hum#16 (GBM03) (SEQ ID NO: 100)
EIVLTQSPATLSLSPGERATLSCRASESVDSSGNSFMHWYQQKPGQAPRLLIYRASNLES
GIPARFSGSGSGTDFTLTISSLEPEDFAVYYCQQSNEDPWTFGGGTKLEIK

### Example 23. In Vitro Testing of Anti-Human TNFR2 Antibodies in NF-κB Reporter Cell Line Assay

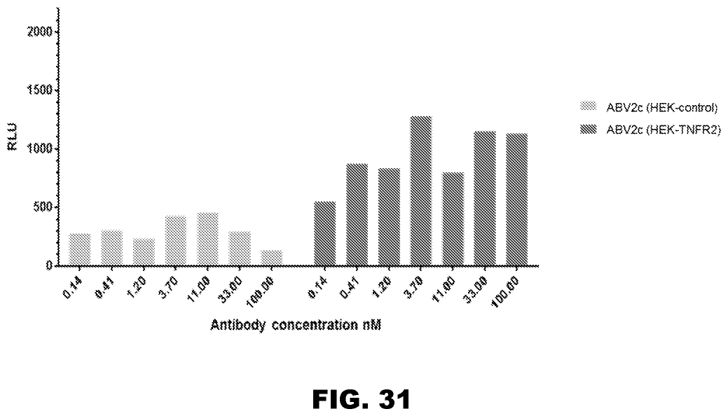

This example describes the generation of a human TNFR2 reporter cell line to assess the agonistic activity of human anti-TNFR2 antibodies. To test the agonistic activity of the human anti-TNFR2 antibodies in a signaling assay, a human TNFR2 reporter cell line was generated. Briefly, GloResponse™ NF-κB-RE-luc2p HEK293 cell lines (Promega) were transfected with full length human TNFR2 gene (Origene) using Lipofectamine 3000 (Thermofisher) and allowed to recover in DMEM/10% FBS. Two days following transfection, media was replaced with media containing Geneticin® (0.2 mg/ml). After 14 days of cultures in geneticin containing media, stable expression of human TNFR2 was confirmed by flow cytometry. To measure TNFR2 induced NF-kB signaling, human TNFR2 reporter cells and vector control cells (1×104) were incubated with ABV2c (0.14-100 nM) for 5 hours at 37° C. ONE-Glo™ luciferase reagent was then added, and luminescence was measured on a SYNERGY H1 plate reader (BioTek). A dose-dependent increase in NF-κB signaling was observed after incubation with anti-human TNFR2 antibody ABV2c (FIG. 31).

### Example 24. Effects of Anti-Human TNFR2 Antibodies on Treg Cells in Ovarian Cancer Ascites In Vitro

Treg cells from patients with ovarian cancer have been reported to have high levels of TNFR2 and to be highly immunosuppressive. Others have shown that treatment with anti-TNFR2 antibodies reduces the viability of ascites Treg cells (Torrey et al., Sci Signal 2017; 10:eaaf8608). This example describes the effects of anti-human TNFR2 antibodies on Treg cells in ovarian cancer ascites.

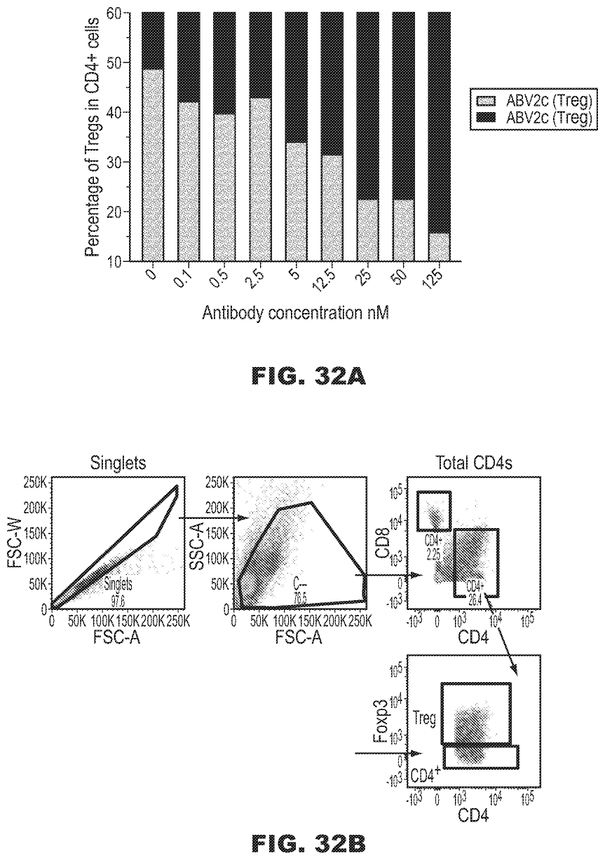

Ovarian cancer ascites were obtained and whole ascites were cultured with the indicated concentrations of anti-TNFR2 antibody ABV2c for 48 hours. Flow cytometry was used to determine the relative abundance of Treg cells in the CD4+ T cell compartment following treatment (FIGS. 32A and 32B). As shown in FIG. 32A, ABV2c decreased the percentage of cells expressing the Treg-lineage marker Foxp3 within the CD4 compartment, suggesting that ABV2c selectively inhibits Treg cells but not effector CD4 T cells.

### Example 25. Effects of Anti-Human TNFR2 Antibodies on ADCC In Vitro

This example describes the effects of anti-human TNFR2 antibody ABV2c on ADCC using an in vitro assay.

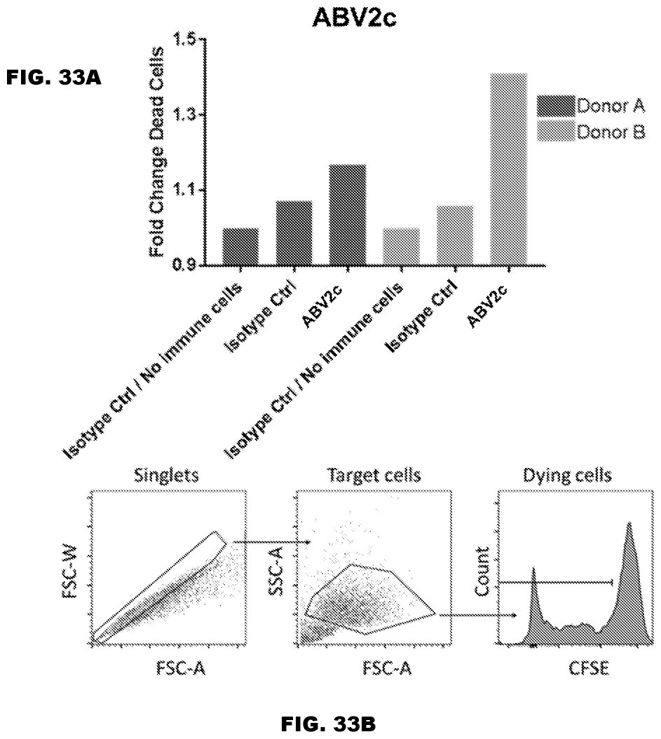

As shown in Examples 8 and 14, Fcγ-receptor binding is required for the anti-tumor activity of mouse surrogate anti-TNFR2 antibodies in syngeneic mouse tumor models. NK cells are important effectors of antibody-dependent cellular cytotoxicity (ADCC). To test whether ABV2c mediates ADCC of human cells, NK cells (RosetteSep Human NK cell Enrichment Cocktail, StemCell) were isolated from peripheral blood from healthy donors and cultured with carboxyfluorescein succinimidyl ester (CFSE)-labeled JJN3 (plasma cell myeloma) target cells, which express high levels of TNFR2, at a 5:1 effector (NK cell) to target cell ratio for four hours in the presence or absence of ABV2c at a concentration of 5 μg/mL. As target cells die, the cell membrane becomes permeable and intracellular proteins leak out, causing a drop in the per-cell fluorescence of CFSE that can be quantified by flow cytometry. As shown in FIGS. 33A and 33B, in the presence of NK cells, ABV2c increased the number of dead cells compared to target cells alone with isotype control antibody, or target cells plus NK with isotype control antibody, across multiple donors, suggesting that ABV2c mediates ADCC of human target cells.

### Example 26: Effects of Human Anti-TNFR2 Antibodies on Co-Stimulatory Activity, Proliferation, and Functionality of CD4+ and CD8+ T Cells

The effects of human anti-TNFR2 antibodies on various aspects of T cell function were tested as follows.

Briefly, 96-well flat bottom plates (Corning) were coated with titrated amounts of functional-grade anti-CD3 (clone OKT3, BioLegend) and human anti-TNFR2 antibodies. Mononuclear cells were isolated in 50 mL SepMate-50 tubes (StemCell Technologies) over a Ficoll-Paque Plus density gradient (GE Healthcare). Total CD8 T cells or naïve CD45RA+CD4 T cells were purified via negative selection (human CD8+ T cell isolation kit or Naïve CD4+ T cell isolation kit II, Miltenyi) and labelled with 5 μM CellTrace Violet (ThermoFisher Scientific). 2-5×10⁴ cells (typically >85% purity for CD8 T cells and >90% for CD4 T cells) were added per well along with 1 μg/mL soluble anti-CD28 (clone CD28.2, BioLegend) in RPMI 1640 (Gibco) supplemented with 10% FBS, 5 mM HEPES (Gibco), pen/strep (Gibco), 50 μM 3-ME (G-Biosciences), 2 mM L-glutamine (Gibco), and incubated at 37° C. for 72 or 96 hrs as indicated. The golgi inhibitor Brefeldin A (BioLegend) was added to CD8+ T cell cultures for the final 5 hrs. Cells were then stained for activation markers and intracellular cytokines and analyzed by flow cytometry. Cells were first incubated and stained with the following antibodies from BioLegend: CD4 (OKT4), CD8 (SK1 or HIT8a), CD25 (BC96), PD-1 (EH12.2H7). Single cell suspensions were first incubated with Fc Block (BD Biosciences) and live/dead Ghost Dye red710 (Tonbo Biosciences) in PBS for 10 min at 4° C. Cells were then stained for extracellular markers for 30 min at 4° C. in FACS buffer (PBS with 1% FBS and 0.02% sodium azide). When staining CD8+ T cells for intracellular cytosolic proteins, cells were permeabilized using BioLegend's Fixation and Intracellular Staining Perm Buffer. Samples were run on an LSR Fortessa flow cytometer (BD Biosciences) and data were analyzed using FlowJo analysis software (Tree Star) version 10.5.3. Data were analyzed using a two-way ANOVA with Dunnett's multiple comparisons post-test. Data were plotted as mean±S.E.M. Statistically significant difference from Isotype is indicated (*p<0.05, **p<0.01, ***p<0.001).

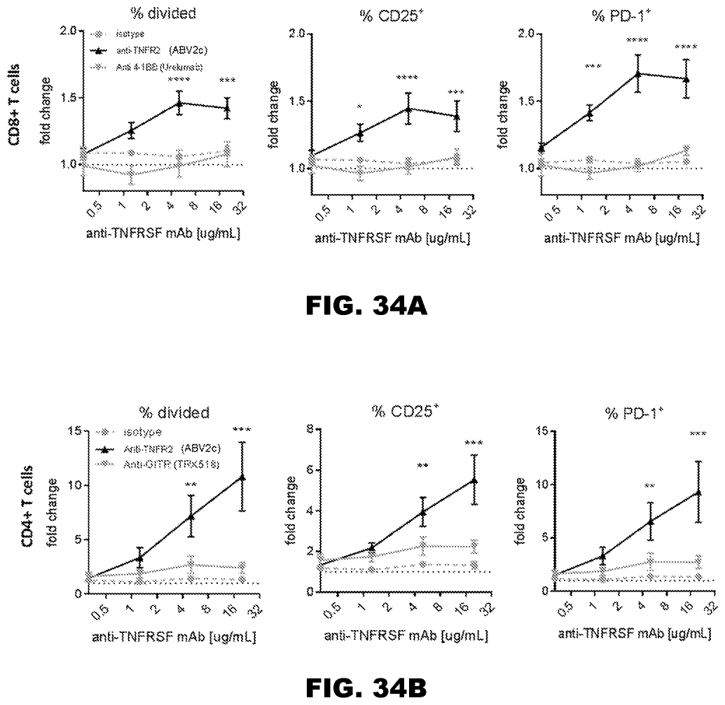

As shown in FIGS. 34A-34C, the human anti-TNFR2 antibody ABV2c expanded and induced activation markers on CD4+ and CD8+ T cells in vitro. Moreover, ABV2c led to greater expansion and induction of activation markers than an anti-GITR antibody (TRX518) or anti-4-1BB antibody (Urelumab).

### Example 27: Effects of Human Anti-TNFR2 Antibodies in a Graft-Versus-Host Disease Model

The ability of human anti-TNFR2 antibodies to protect against disease was tested using a xenogenic GvHD model as follows.

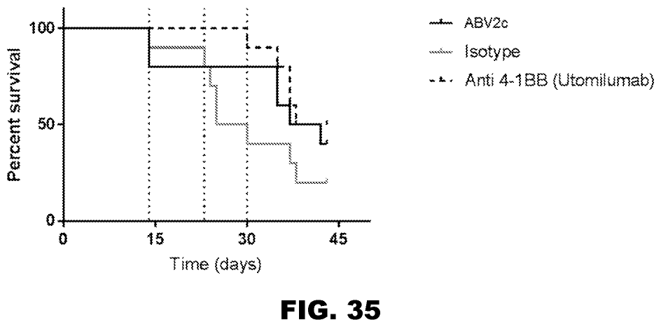

Briefly, three to six-week-old female NSG-SGM3 (NOD Cg-Prkdc^{scid} IL2rg^{tm1Wjl} Tg(CMV-IL-3, CSF2, KITLG)1Eav/MloySz) mice were administered 10⁷ PBMCs from healthy donors i.v. and monitored daily for weight loss and changes in body condition. Animals were euthanized if >20% initial weight loss or significant deterioration in body condition was observed. On days 14, 23, and 30, mice were treated i.p. with 300 μg anti-TNFR2 (ABV2c), anti-4-1BB (Utomilumab), or isotype control antibody. Comparisons were made between control and treatment groups using the log rank test. Statistically significant difference from PBS is indicated (*p<0.05, **p<0.01, ***p<0.001). As shown in FIG. 35, ABV2c increased survival in the xenogeneic GvHD model. The protective effect was greater than that of the agonistic anti-4-1BB antibody (Utomilumab).

### Example 28: High Resolution Epitope Mapping of Human and Mouse Anti-TNFR2 Antibodies

High resolution mapping of the epitopes on TNFR2 recognized by Y9 and ABV2c was performed as follows.

The surface residues of the CRD1 region of both human and mouse TNFR2 were subjected to mutational scanning. Based on the homology model of the mouse TNFR2, surface exposed positions within CRD1 were mutated to alanine or aspartate. For human TNFR2, more disruptive amino acid substitutions were introduced (Grantham, R. (1974). Amino Acid Difference Formula to Help Explain Protein Evolution. Science, 185(4154), 862-864). Wild-type human TNFR2 ECD (24-257), wild-type mouse TNFR2 ECD (23-258) and all corresponding point mutants were fused to a C-terminal FLAG epitope tag and expressed using yeast surface display. Flow cytometric analysis was performed on yeast (1e6 cells) stained with anti-mouse TNFR2 antibody, Y9, or anti-human TNFR2 antibody, ABV2c. Surface expression was normalized using anti-FLAG antibody (Sigma Aldrich). The ratio of MFI for antibody binding/FLAG detection was plotted as a function of antibody concentration in PRISM (GraphPad).

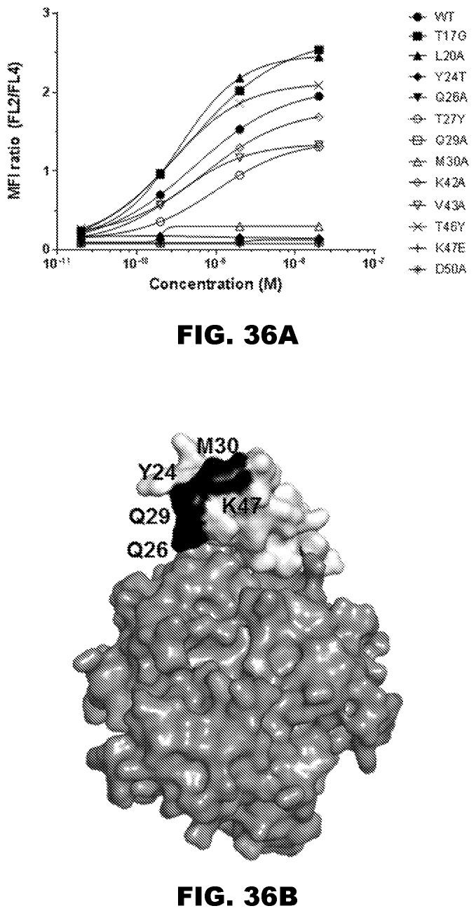

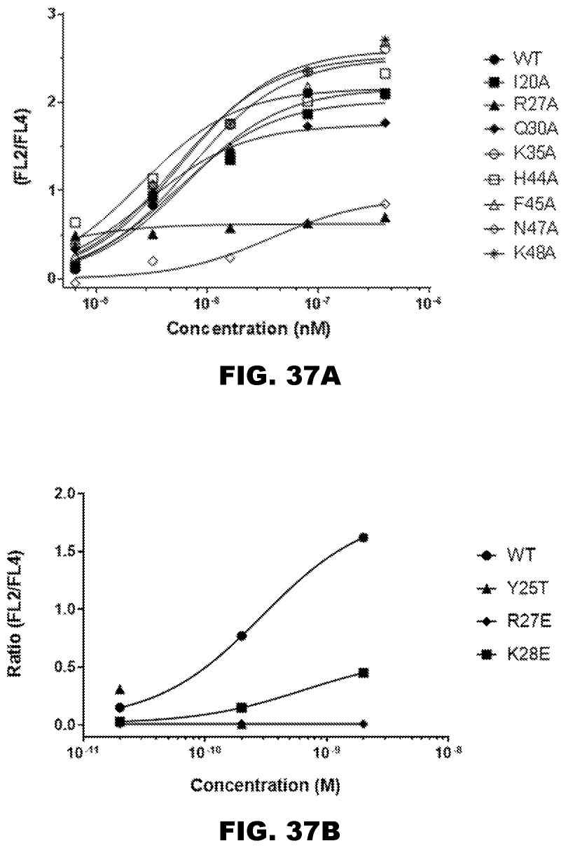

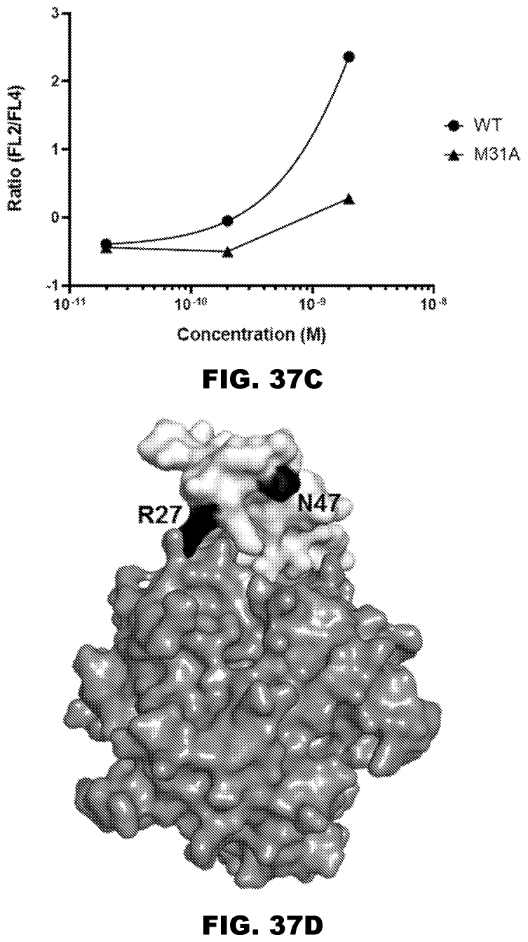

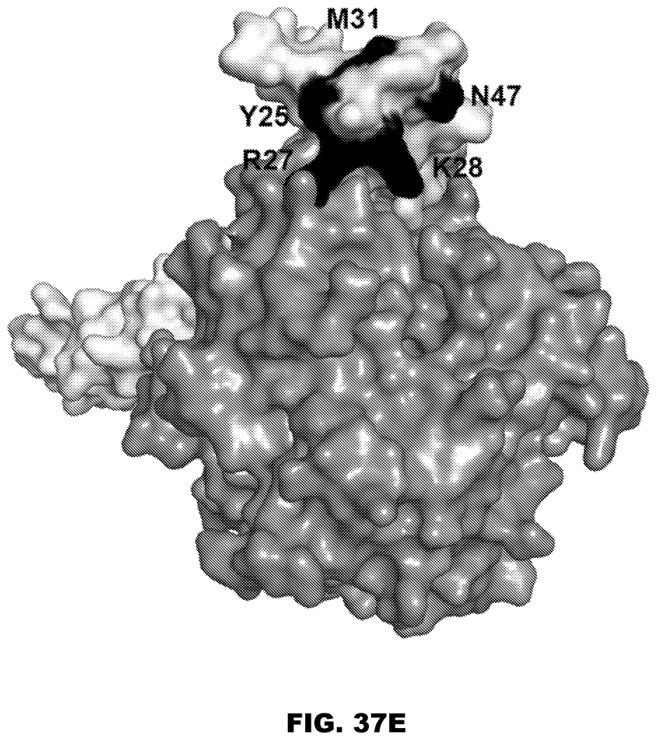

Several positions critical for ABV2c and Y9 binding were identified (FIGS. 36A and 37A, respectively). For ABV2c, these positions correspond to Y24, Q26, Q29, M30, and K47 of human TNFR2 without the leader sequence (SEQ ID NO: 104). For Y9, these positions correspond to Y25, R27, K28, M31, and N47 of mouse TNFR2 without the leader sequence (SEQ ID NO: 107). Using the crystal structure of human TNFR2/TNF complex (PDB: 3ALQ) and a homology model of the mouse TNFR2/TNF complex, both antibody epitopes were visualized in relation to TNF binding (FIG. 36B for ABV2c; 37D and 37E for Y9, respectively). Remarkably, ABV2c and Y9 both interact with the structurally equivalent positions in human (Y24, Q26, and M30) and mouse (Y25, R27 and M31). The proximity of both the ABV2c and Y9 epitope to the human/mouse TNF binding interface suggests that these antibodies potentially compete with TNF through steric occlusion. Another possibility is that these antibodies may also prevent TNF binding by inducing a conformational change in the receptor.

### Example 29: Anti-Tumor Activity of Anti-Human TNFR2 Antibody in Patient-Derived Xenograft Model in Humanized Mice

To test the activity of anti-human TNFR2 antibody in a tumor models, 3-week-old NSG-SGM3 female mice (Jackson Laboratories) were irradiated with 140cGy and then injected i.v. with 2×10⁴ human cord blood CD34⁺ stem cells from mixed donors (AllCells) the same day. After resting for 12 weeks to allow hematopoietic stem cell engraftment and reconstitution with a human immune system, peripheral blood was screened for human immune cell engraftment by staining with flow antibodies for anti-human CD45 and anti-mouse CD45. Mice were considered humanized when ≥25% of total CD45⁺ cells were of human origin. Humanized mice were injected s.c. with 5×10⁶ cells of the patient-derived xenograft cell line LG1306 (Jackson Laboratories). When the average tumor size was −75 mm³, mice were equally distributed into 3 treatment groups and injected with 0.3 mg i.p. of human isotype IgG1 (BioXCell), nivolumab (anti-PD-1 IgG1) alone, or nivolumab plus ABV2c (IgG1) in combination for a total of 5 injections every 7 days. Tumor volumes were measured every 2-3 days.

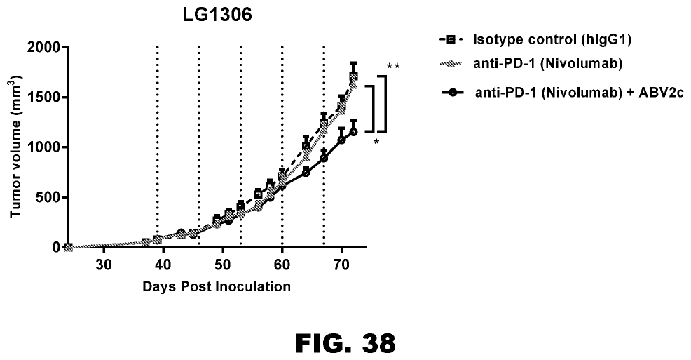

As shown in FIG. 38, statistically significant differences (ANOVA, Tukey's honestly significant difference procedure) in tumor volume were observed between isotype control and nivolumab plus ABV2c arms, as well as between nivolumab and nivolumab plus ABV2c arms.

### Example 30: Affinity Maturation of Anti-Human TNFR2 Antibodies

ABV2 VH_hum #2 (HD3) (SEQ ID NO: 74) and ABV2 VL_hum #2 (HD3) (SEQ ID NO: 86) were subjected to affinity maturation. The amino acid sequences of both heavy and light chain variable regions were optimized for E. coli, synthesized, and cloned into a Fab phagemid. Examples of commercially available Fab phagemids are pComb3, pC3C and pADL-23c. The following positions (numbered using Chothia) were randomized using mutagenic primers: heavy chain CDR1 (26, 27, 28, 29, 30, 31 and 32), heavy chain CDR2 (positions 50, 51, 52A, 53, 54, 55, 56, 57 and 58), heavy chain CDR3 (95, 96, 97, 98, 101, and 102), light chain CDR1 (24, 25, 26, 27, 28, 29, 30, 30A, 30B, 30C, 30D, 31, 32, 33, and 34), light chain CDR2 (50, 51, 52, 53, 54, 55, and 56), and light chain CDR3 (89, 90, 91, 92, 93, 94, 95, 96, and 97). Each primer contained degenerate codons NNS or VNS at 4 positions in a single CDR in either the heavy or light chain variable region. Primers were combined and PCR-based mutagenesis was used to create the 1^{st} generation mutant Fab library. Following two rounds of panning on human TNFR2-His, the enriched phage-infected bacterial clones were pooled and DNA was isolated. A second round of PCR mutagenesis was performed to create a 2^{nd} generation mutant Fab library, followed by an additional two rounds of panning on antigen. Individual E. coli clones infected from the final output phage were selected from plates and grown in 96-well cultures. Periplasmic extracts containing soluble Fab protein were screened for TNFR2-His binding by ELISA. To estimate the affinity of the Fabs, a competition ELISA was simultaneously performed. Fab extracts were incubated with or without soluble competitor TNFR2-His (5 nM) for 1 hr prior to incubation on TNFR2-coated wells. Clones with a reduced ELISA signal in the presence of competitor were considered to have a greater affinity (<5 nM) and were chosen for further analysis. Heavy and light chain variable regions were cloned and expressed in Expi293 cells as human IgG1 antibodies. Full length sequences, variable region sequences, and CDR sequences of these affinity matured antibodies (ABV2.7, ABV2.13, and ABV2.15) are provided in Table 10.

ABV2.7VH
(SEQ ID NO: 126)
QVQLVQSGSELKKPGASVKVSCKASGYTFTTFGMSWVRQAPGQGLEWMGW
INTYSGVPTYAQGFTGRFVFSLDTSVSTAYLQISSLKAEDTAVYFCARRS
NFAYWGQGTLVTVSS
ABV2.7VL
(SEQ ID NO: 127)
DIVLTQSPDSLAVSLGERATINCRASESLTASGNSFMHWYQQKPGQPPKW
YRASNLESGVPDRFSGSGSRTDFTLTISSLQAEDVAVYYCQQSRHVNWTF
GGGTKVEIK
ABV2.13VH
(SEQ ID NO: 148)
QVQLVQSGSELKKPGASVKVSCKASGYTFTTFGMSWVRQAPGQGLEWMGW
INTYSGVPHYAQGFTGRFVFSLDTSVSTAYLQISSLKAEDTAVYFCARRS
NFAYWGQGTLVTVSS
ABV2.13VL
(SEQ ID NO: 149)
DIVLTQSPDSLAVSLGERATINCRASQTVDSSGNSFMHWYQQKPGQPPKW
YLGNRLESGVPDRFSGSGSRTDFTLTISSLQAEDVAVYYCQQSNEDPWTF
GGGTKVEIK
ABV2.15VH
(SEQ ID NO: 170)
QVQLVQSGSELKKPGASVKVSCKASGYTFTTFGMSWVRQAPGQGLEWMGW
INTYSGVPHYAQGFTGRFVFSLDTSVSTAYLQISSLKAEDTAVYFCARRS
NFAYWGQGTLVTVSS
ABV2.15VL
(SEQ ID NO: 171)
DIVLTQSPDSLAVSLGERATINCRASESLTASGNSFMHWYQQKPGQPPKW
YRASNLESGVPDRFSGSGSRTDFTLTISSLQAEDVAVYYCQQSRHVNWTF
GGGTKVEIK

### Example 31: Binding Affinity of Affinity-Matured Anti-Human TNFR2 Antibodies for Human TNFR2

Binding affinities for human TNFR2 of the affinity-matured anti-human TNFR2 antibodies generated in Example 30 were measured by bio-layer interferometry (BLI). Briefly, a BLI kinetic assay was performed using anti-Human Fc Capture (AHC) biosensors (ForteBio) under the following conditions: (a) loading of antibody (4 μg/mL) for 60 sec, (b) baseline for 60 sec, (d) association with human TNFR2-His (4 mg/ml) for 300 sec, and (e) dissociation for 300 sec. As shown in Table 9, the binding affinity of the affinity-matured antibodies was substantially higher than that for the parental antibody (ABV2.1, which has heavy and light chain variable region sequences of SEQ ID NOs: 74 and 86, respectively), with ABV2.15 showing the strongest affinity for human TNFR2 (K_{D}=0.158 nM).

TABLE 9

| Antibody | K_{D} (nM) |
|---|---|
| ABV2c | 0.689 |
| ABV2.1 (ABV2 hum #2 HD3) | 4.42 |
| ABV2.7 | 0.3 |
| ABV2.13 | 1.59 |
| ABV2.15 | 0.158 |

### Example 32: T Cell Co-Stimulation by Affinity-Matured Anti-Human TNFR2 Antibodies

Various aspects of T cell co-stimulation were tested using the affinity-matured anti-human TNFR2 antibodies generated in Example 30.

### Proliferation, Expansion, and Upregulation of PD1

Human naïve CD45RA+CD4 T cells from 3 healthy donors were enriched via negative selection using the human Naïve CD4+ T cell Isolation Kit II (Miltenyi) and then labeled with 5 mM CellTrace Violet. 96 well flat-bottom plates (Costar) were coated with 5 mg/mL anti-CD3 (clone OKT3, BioLegend) and titrated amounts of anti-TNFR2 at 37° C. for 2 hrs. Plates were then washed with complete RPMI, blocked at room temperature for >10 min at room temperature, and 4×10⁴ cells were added along with 1 mg/mL soluble anti-CD28 (BioLegend). Cells were stimulated for 4 days and then analyzed by flow cytometry. Live CD4+ T cells were assessed for proliferation, expansion, and upregulation of the acute activation marker PD-1.

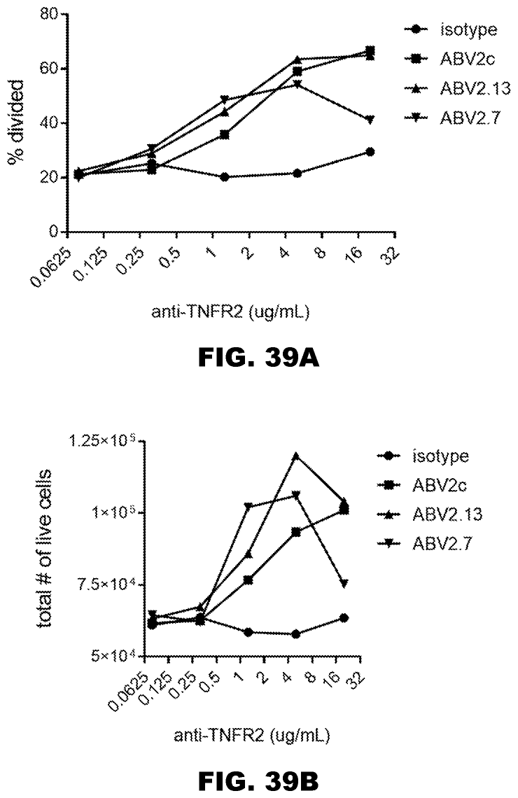

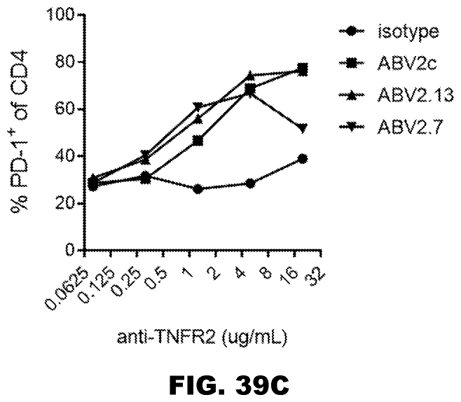

As shown in FIGS. 39A-39C, despite having lowest affinity of the three antibodies tested, ABV2.13 showed a similar ability to promote CD4+ T cell proliferation, expansion, and PD-1 upregulation compared to ABV2c.

### Cytokine Producing Cells

Naïve CD4 T cells from a healthy donor were stimulated with anti-CD3/28 as described above and titrated amounts of plate-bound IgG1 isotype, chimeric ABV2c, and humanized variants ABV2.13 and ABV2.7. During the final 5 hrs of stimulation, brefeldin A was added to the culture to assess the percentage of CD4+ cells producing IFN-γ and IL-2 by flow cytometry.

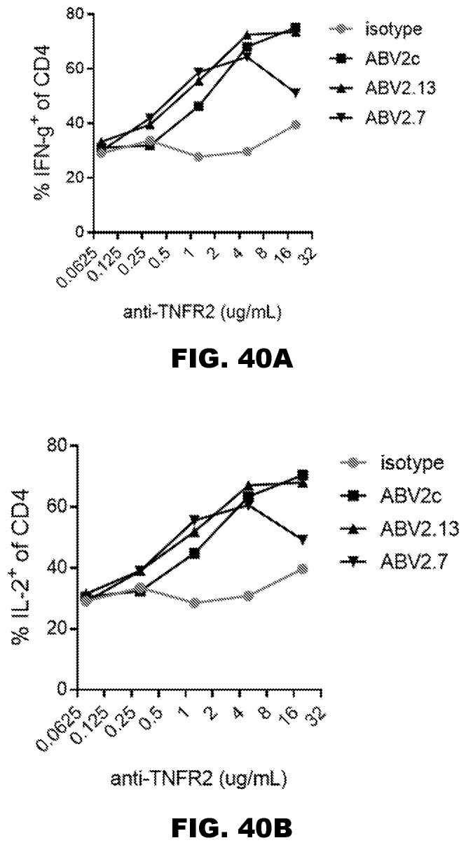

As shown in FIGS. 40A and 40B, ABV2.13 increased the number of IFN-γ and IL-2 producing cells to a similar degree as ABV2c and ABV2.7.

### Cytokine Production

Following in vitro stimulation of isolated human naïve CD4 T cells described above (in FIGS. 39A-39C), supernatants were collected and assayed for cytokines (IL-2, IFN-γ, TNF, GM-CSF, IL-4, IL-5, and IL-13) using the Luminex platform (ThermoFisher Invitrogen: Th1/Th2 Cytokine 18-Plex Human ProcartaPlex Panel 1C, 18 analytes).

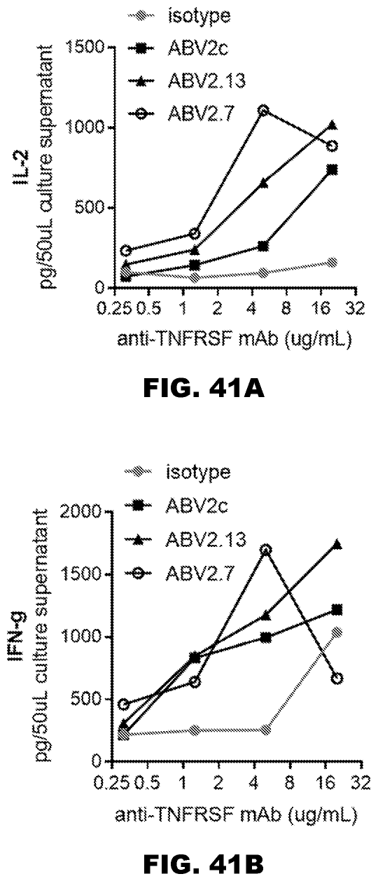

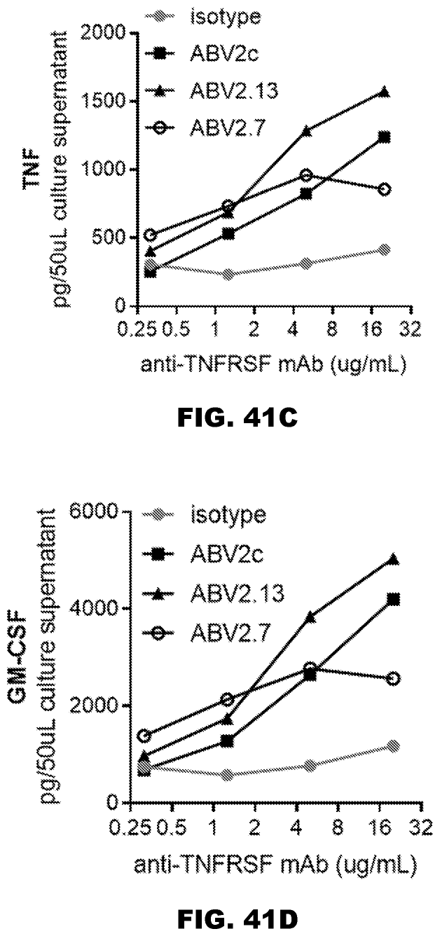

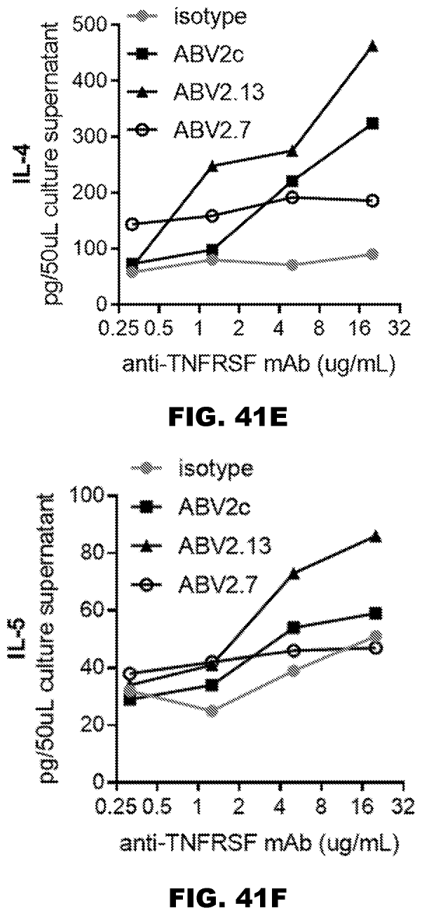

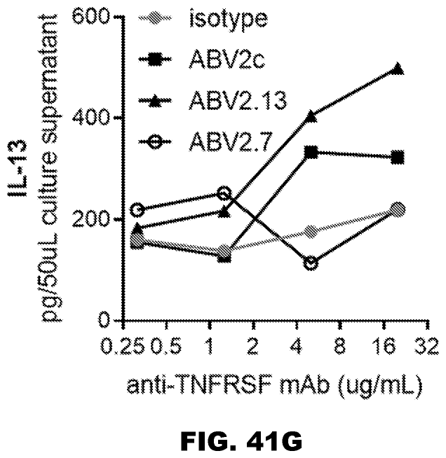

As shown in FIGS. 41A-41G, cytokine production was highest in CD4+ T cells treated with ABV2.13, compared to ABV2c. With the exception of IL-2 induction, ABV2.7, despite having the highest affinity of the antibodies tested, stimulated the least amount of cytokine production.

### Stimulation of NF-kB Activity

A human TNFR2 reporter cell line was generated using GloResponse™ NF-kB-RE-luc2p HEK293 cells (Promega) that were stably transfected with either full-length murine TNFR2 gene (Origene) using Lipofectamine 3000 (ThermoFisher) or vector control. Cells were maintained in DMEM/10% FBS containing geneticin (0.2 mg/mL). 96 well black-walled tissue culture plates were coated with titrated concentrations of anti-TNFR2 mAb for 2 hrs at 37° C. and then washed and blocked with complete culture media. 4×10⁴ TNFR2-expressing or control HEK293 cells were added per well in a volume of 50 mL, cultured at 370 for 5 hrs, and 50 mL ONE-Glo luciferase reagent was then added per well. Luminescence was measure on a SYNERGY H1 plate reader (BioTek).

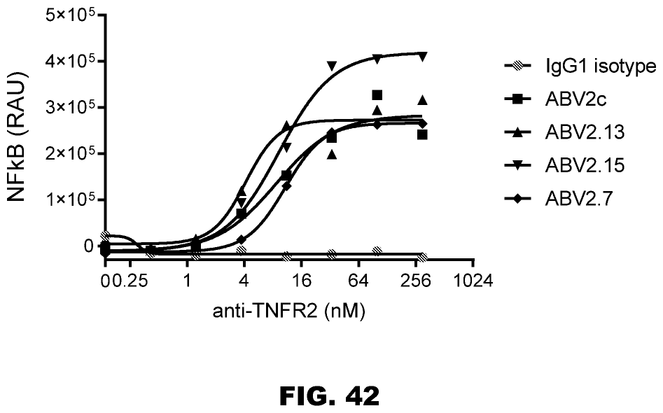

As shown in FIG. 42, all antibodies showed relatively similar EC50 (g/ml) in stimulating NF-kB activity—ABVc (EC50=8.6 μg/ml). ABV2.7 (EC50=10.8 μg/ml), ABV2.13 (EC50=4.1 μg/ml) and ABV2.15 (EC50=9.3 μg/ml). However, ABV2.15 induced the highest level of NF-kB activity.

### Example 33: Superior T Cell Co-Stimulation by ABV2 Antibodies Relative to Comparator Prior Art Antibodies

Various aspects of T cell co-stimulation were compared between a low affinity anti-human TNFR2 antibody (ABV2.1, also referred to as AVB2human_Dsn3), an affinity-matured version (ABV2.15), and comparator prior art anti-human TNFR2 antibodies A-C.

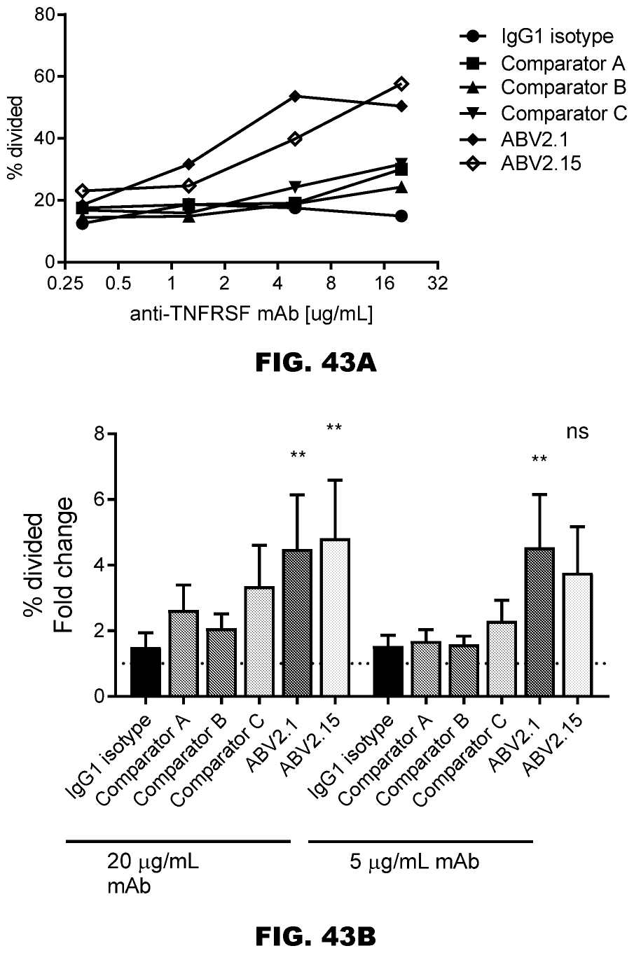

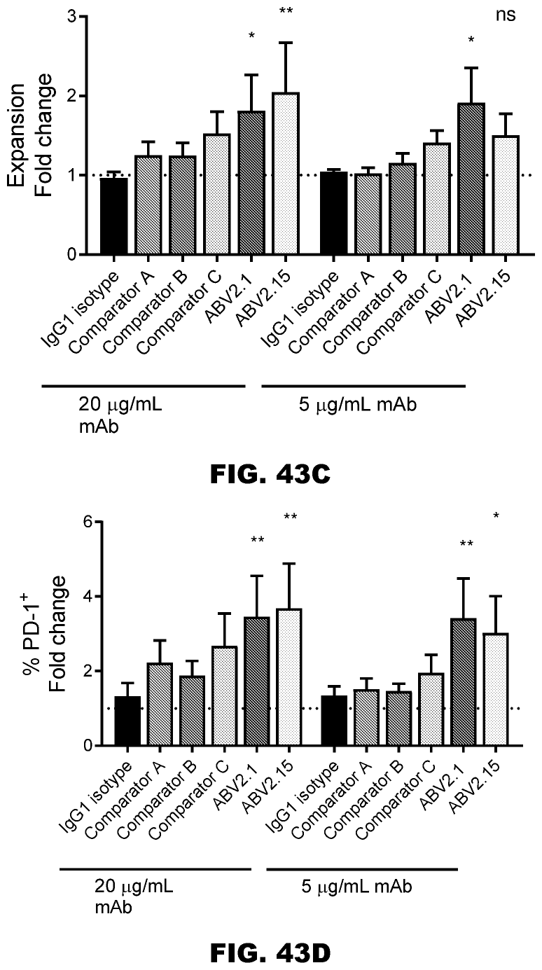

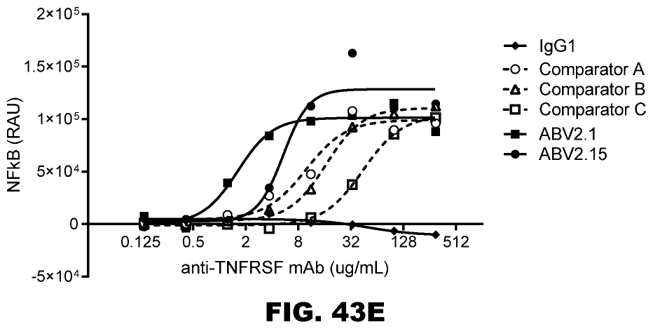

CD4+ T cells were assessed for proliferation, expansion, PD-1 upregulation, and stimulation of NF-kB activity as described in Example 32. Both ABV2.1 and ABV2.15 stimulated ≥50% of CD4+ T cells to divide compared to compared to 30% for comparator A, 24% for comparator B, 32% for comparator C, and 15% for isotype control antibody at the highest concentration tested (FIG. 43A). The mean fold-change in cell proliferation induced by 20 μg/ml of ABV2.1 (4.5-fold) and ABV2.15 (4.8-fold) compared to isotype control (1.5-fold) was determined to be significant (p<0.05) by two-way ANOVA. In contrast, the mean-fold change for comparator A (2.6-fold), comparator B (2.0-fold), and comparator C (3.3-fold) were not significant compared to isotype control (FIG. 43B).

The mean fold-change in CD4+ T cell expansion induced by 20 μg/ml of ABV2.1 (1.8-fold) and ABV2.15 (2.0-fold) compared to isotype control (0.96-fold) was determined to be significant (p<0.05) by two-way ANOVA. In contrast, the mean-fold change for comparator A (1.2-fold), comparator B (1.2-fold), and comparator C (1.5-fold) were not significant compared to isotype control (FIG. 43C).

The mean fold-change in PD-1 upregulation on CD4+ T cells induced by 20 μg/ml of ABV2.1 (3.4-fold) and ABV2.15 (3.6-fold) compared to isotype control (1.3-fold) was determined to be significant (p<0.01) by two-way ANOVA. In contrast, the mean-fold change for comparator A (2.2-fold), comparator B (1.8-fold), and comparator C (2.6-fold) were not significant compared to isotype control (FIG. 43D).

ABV2.1 and ABV2.15 induced NF-kB activity with an EC50 of 1.6 and 5.3 μg/ml, respectively, and was found to be more active than comparator A (EC50=9.7 μg/ml), comparator B (EC50=16.6 μg/ml), and comparator C (EC50=44 μg/ml) (FIG. 43E).

Overall, ABV2.1 and its affinity matured version ABV2.15 were superior to comparator prior art antibodies A-C.

TABLE 10
SUMMARY OF SEQUENCES

| SEQ | | |
|---|---|---|
| ID | Description | Sequence |
| 1 | Human TNFR2 | MAPVAVWAALAVGLELWAAAHALPAQVAFTPYAPEPGSTCRLREYY |
| | (leader sequence is | DQTAQMCCSKCSPGQHAKVFCTKTSDTVCDSCEDSTYTQLWNWVPE |
| | underlined) | CLSCGSRCSSDQVETQACTREQNRICTCRPGWYCALSKQEGCRLCA |
| | | PLRKCRPGFGVARPGTETSDVVCKPCAPGTFSNTTSSTDICRPHQI |
| | | CNVVAIPGNASMDAVCTSTSPTRSMAPGAVHLPQPVSTRSQHTQPT |
| | | PEPSTAPSTSFLLPMGPSPPAEGSTGDFALPVGLIVGVTALGLLII |
| | | GVVNCVIMTQVKKKPLCLQREAKVPHLPADKARGTQGPEQQHLLIT |
| | | APSSSSSSLESSASALDRRAPTRNQPQAPGVEASGAGEARASTGSS |
| | | DSSPGGHGTQVNVTCIVNVCSSSDHSSQCSSQASSTMGDTDSSPSE |
| | | SPKDEQVPFSKEECAFRSQLETPETLLGSTEEKPLPLGVPDAGMKP |
| | | S |
| 2 | Human TNFR2 | LPAQVAFTPYAPEPGSTCRLREYYDQTAQMCCSKCSPGQHAKVFCT |
| | (extracellular | KTSDTVCDSCEDSTYTQLWNWVPECLSCGSRCSSDQVETQACTREQ |
| | domain) | NRICTCRPGWYCALSKQEGCRLCAPLRKCRPGFGVARPGTETSDVV |
| | | CKPCAPGTFSNTTSSTDICRPHQICNVVAIPGNASMDAVCTSTSPT |
| | | RSMAPGAVHLPQPVSTRSQHTQPTPEPSTAPSTSFLLPMGPSPPAE |
| | | GSTGD |
| 3 | Mouse TNFR2 | MAPAALWVALVFELQLWATGHTVPAQVVLTPYKPEPGYECQISQEY |
| | (leader sequence is | YDRKAQMCCAKCPPGQYVKHFCNKTSDTVCADCEASMYTQVWNQFR |
| | underlined) | TCLSCSSSCTTDQVEIRACTKQQNRVCACEAGRYCALKTHSGSCRQ |
| | | CMRLSKCGPGFGVASSRAPNGNVLCKACAPGTFSDTTSSTDVCRPH |
| | | RICSILAIPGNASTDAVCAPESPTLSAIPRTLYVSQPEPTRSQPLD |
| | | QEPGPSQTPSILTSLGSTPIIEQSTKGGISLPIGLIVGVTSLGLLM |
| | | LGLVNCIILVQRKKKPSCLQRDAKVPHVPDEKSQDAVGLEQQHLLT |
| | | TAPSSSSSSLESSASAGDRRAPPGGHPQARVMAEAQGFQEARASSR |
| | | ISDSSHGSHGTHVNVTCIVNVCSSSDHSSQCSSQASATVGDPDAKP |
| | | SASPKDEQVPFSQEECPSQSPCETTETLQSHEKPLPLGVPDMGMKP |
| | | SQAGWFDQIAVKVA |
| 4 | Mouse TNFR2 | VPAQVVLTPYKPEPGYECQISQEYYDRKAQMCCAKCPPGQYVKHFC |
| | (extracellular | NKTSDTVCADCEASMYTQVWNQFRTCLSCSSSCTTDQVEIRACTKQ |
| | domain) | QNRVCACEAGRYCALKTHSGSCRQCMRLSKCGPGFGVASSRAPNGN |
| | | VLCKACAPGTFSDTTSSTDVCRPHRICSILAIPGNASTDAVCAPES |
| | | PTLSAIPRTLYVSQPEPTRSQPLDQEPGPSQTPSILTSLGSTPIIE |
| | | QSTKGG |
| 5 | TNFR2 Chimera 0 | VPAQVVLTPYKPEPGYECQISQEYYDRKAQMCCSKCSPGQHAKVFC |
| | | TKTSDTVCDSCEDSTYTQLWNWVPECLSCGSRCSSDQVETQACTRE |
| | | QNRICTCRPGWYCALSKQEGCRLCAPLRKCRPGFGVARPGTETSDV |
| | | VCKPCAPGTFSNTTSSTDICRPHQICNVVAIPGNASMDAVCTSTSP |
| | | TRSMAPGAVHLPQPVSTRSQHTQPTPEPSTAPSTSFLLPMGPSPPA |
| | | EGSTGDIEGRMDPHHHHHH |
| | | mouse 23-55 (51-54); human 55 (50-54)-257 |
| 6 | TNFR2 Chimera 0 | VPAQVVLTPYKPEPGYECQISQEYYDRKAQMCCSKCSPGQHAKVFC |
| | Fc fusion | TKTSDTVCDSCEDSTYTQLWNWVPECLSCGSRCSSDQVETQACTRE |
| | | QNRICTCRPGWYCALSKQEGCRLCAPLRKCRPGFGVARPGTETSDV |
| | | VCKPCAPGTFSNTTSSTDICRPHQICNVVAIPGNASMDAVCTSTSP |
| | | TRSMAPGAVHLPQPVSTRSQHTQPTPEPSTAPSTSFLLPMGPSPPA |
| | | EGSTGDIEGRMDPKSSDKTHTCPPCPAPELLGGPSVFLFPPKPKDT |
| | | LMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQ |
| | | PREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALHN |
| | | HYTQKSLSLSPGHHHHHH |
| 7 | TNFR2 Chimera 1 | VPAQVVLTPYKPEPGYECQISQEYYDRKAQMCCAKCPPGQYVKHFC |
| | | NKTSDTVCADCEDSTYTQLWNWVPECLSCGSRCSSDQVETQACTRE |
| | | QNRICTCRPGWYCALSKQEGCRLCAPLRKCRPGFGVARPGTETSDV |
| | | VCKPCAPGTFSNTTSSTDICRPHQICNVVAIPGNASMDAVCTSTSP |
| | | TRSMAPGAVHLPQPVSTRSQHTQPTPEPSTAPSTSFLLPMGPSPPA |
| | | EGSTGDIEGRMDPHHHHHH |
| | | mouse 23-78 (79, 80); human 78 (79, 80)-257 |
| 8 | TNFR2 Chimera 1 | VPAQVVLTPYKPEPGYECQISQEYYDRKAQMCCAKCPPGQYVKHFC |
| | Fc fusion | NKTSDTVCADCEDSTYTQLWNWVPECLSCGSRCSSDQVETQACTRE |
| | | QNRICTCRPGWYCALSKQEGCRLCAPLRKCRPGFGVARPGTETSDV |
| | | VCKPCAPGTFSNTTSSTDICRPHQICNVVAIPGNASMDAVCTSTSP |
| | | TRSMAPGAVHLPQPVSTRSQHTQPTPEPSTAPSTSFLLPMGPSPPA |
| | | EGSTGDIEGRMDPKSSDKTHTCPPCPAPELLGGPSVFLFPPKPKDT |
| | | LMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQ |
| | | PREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALHN |
| | | HYTQKSLSLSPGHHHHHH |
| 9 | TNFR2 Chimera 2 | VPAQVVLTPYKPEPGYECQISQEYYDRKAQMCCAKCPPGQYVKHFC |
| | | NKTSDTVCADCEASMYTQVWNQFRTCLSCSSSCTTDQVEIRACTKQ |
| | | QNRVCTCRPGWYCALSKQEGCRLCAPLRKCRPGFGVARPGTETSDV |
| | | VCKPCAPGTFSNTTSSTDICRPHQICNVVAIPGNASMDAVCTSTSP |
| | | TRSMAPGAVHLPQPVSTRSQHTQPTPEPSTAPSTSFLLPMGPSPPA |
| | | EGSTGDIEGRMDPHHHHHH |
| | | mouse 23-118 (119); human 118 (119)-257 |
| 10 | TNFR2 Chimera 2 | VPAQVVLTPYKPEPGYECQISQEYYDRKAQMCCAKCPPGQYVKHFC |
| | Fc fusion | NKTSDTVCADCEASMYTQVWNQFRTCLSCSSSCTTDQVEIRACTKQ |
| | | QNRVCTCRPGWYCALSKQEGCRLCAPLRKCRPGFGVARPGTETSDV |
| | | VCKPCAPGTFSNTTSSTDICRPHQICNVVAIPGNASMDAVCTSTSP |
| | | TRSMAPGAVHLPQPVSTRSQHTQPTPEPSTAPSTSFLLPMGPSPPA |
| | | EGSTGDIEGRMDPKSSDKTHTCPPCPAPELLGGPSVFLFPPKPKDT |
| | | LMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQ |
| | | PREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALHN |
| | | HYTQKSLSLSPGHHHHHH |
| 11 | TNFR2 Chimera 3 | LPAQVAFTPYAPEPGSTCRLREYYDQTAQMCCAKCPPGQYVKHFCN |
| | | KTSDTVCADCEASMYTQVWNQFRTCLSCSSSCTTDQVEIRACTKQQ |
| | | NRVCACEAGRYCALKTHSGSCRQCMRLSKCGPGFGVASSRAPNGNV |
| | | LCKACAPGTFSDTTSSTDVCRPHRICSILAIPGNASTDAVCAPESP |
| | | TLSAIPRTLYVSQPEPTRSQPLDQEPGPSQTPSILTSLGSTPIIEQ |
| | | STKGGIEGRMDPHHHHHH |
| | | human 23-54 (49-53); mouse 56 (51-55)-258 |
| 12 | TNFR2 Chimera 3 | LPAQVAFTPYAPEPGSTCRLREYYDQTAQMCCAKCPPGQYVKHFCN |
| | Fc fusion | KTSDTVCADCEASMYTQVWNQFRTCLSCSSSCTTDQVEIRACTKQQ |
| | | NRVCACEAGRYCALKTHSGSCRQCMRLSKCGPGFGVASSRAPNGNV |
| | | LCKACAPGTFSDTTSSTDVCRPHRICSILAIPGNASTDAVCAPESP |
| | | TLSAIPRTLYVSQPEPTRSQPLDQEPGPSQTPSILTSLGSTPIIEQ |
| | | STKGGIEGRMDPKSSDKTHTCPPCPAPELLGGPSVFLFPPKPKDTL |
| | | MISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQP |
| | | REPQVYTLPPSREEMTKNQVSLTCLVKGFYPSDIAVEWESNGQPEN |
| | | NYKTTPPVLDSDGSFFLYSKLTVDKSRWQQGNVFSCSVMHEALHNH |
| | | YTQKSLSLSPGHHHHHH |
| 13 | TNFR2 Chimera 4 | LPAQVAFTPYAPEPGSTCRLREYYDQTAQMCCSKCSPGQHAKVFCT |
| | | KTSDTVCADCEASMYTQVWNQFRTCLSCSSSCTTDQVEIRACTKQQ |
| | | NRVCACEAGRYCALKTHSGSCRQCMRLSKCGPGFGVASSRAPNGNV |
| | | LCKACAPGTFSDTTSSTDVCRPHRICSILAIPGNASTDAVCAPESP |
| | | TLSAIPRTLYVSQPEPTRSQPLDQEPGPSQTPSILTSLGSTPIIEQ |
| | | STKGGIEGRMDPHHHHHH |
| | | human 23-75 (69-74); mouse 77 (70-76)-258 |
| 14 | TNFR2 Chimera 4 | LPAQVAFTPYAPEPGSTCRLREYYDQTAQMCCSKCSPGQHAKVFCT |
| | Fc fusion | KTSDTVCADCEASMYTQVWNQFRTCLSCSSSCTTDQVEIRACTKQQ |
| | | NRVCACEAGRYCALKTHSGSCRQCMRLSKCGPGFGVASSRAPNGNV |
| | | LCKACAPGTFSDTTSSTDVCRPHRICSILAIPGNASTDAVCAPESP |
| | | TLSAIPRTLYVSQPEPTRSQPLDQEPGPSQTPSILTSLGSTPIIEQ |
| | | STKGGIEGRMDPKSSDKTHTCPPCPAPELLGGPSVFLFPPKPKDTL |
| | | MISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQP |
| | | REPQVYTLPPSREEMTKNQVSLTCLVKGFYPSDIAVEWESNGQPEN |
| | | NYKTTPPVLDSDGSFFLYSKLTVDKSRWQQGNVFSCSVMHEALHNH |
| | | YTQKSLSLSPGHHHHHH |
| 15 | TNFR2 Chimera 5 | LPAQVAFTPYAPEPGSTCRLREYYDQTAQMCCSKCSPGQHAKVFCT |
| | | KTSDTVCDSCEDSTYTQLWNWVPECLSCGSRCSSDQVETQACTREQ |
| | | NRICTCRPGWYCALSKQEGCRLCAPLRKCRPGFGVARPGTETSDVV |
| | | CKPCAPGTFSNTTSSTDICRPHQICNVVAIPGNASMDAVCAPESPT |
| | | LSAIPRTLYVSQPEPTRSQPLDQEPGPSQTPSILTSLGSTPIIEQS |
| | | TKGGIEGRMDPHHHHHH |
| | | human 23-200 (197-199); mouse 203 (199-202)-258 |
| 16 | TNFR2 Chimera 5 | LPAQVAFTPYAPEPGSTCRLREYYDQTAQMCCSKCSPGQHAKVFCT |
| | Fc fusion | KTSDTVCDSCEDSTYTQLWNWVPECLSCGSRCSSDQVETQACTREQ |
| | | NRICTCRPGWYCALSKQEGCRLCAPLRKCRPGFGVARPGTETSDVV |
| | | CKPCAPGTFSNTTSSTDICRPHQICNVVAIPGNASMDAVCAPESPT |
| | | LSAIPRTLYVSQPEPTRSQPLDQEPGPSQTPSILTSLGSTPIIEQS |
| | | TKGGIEGRMDPKSSDKTHTCPPCPAPELLGGPSVFLFPPKPKDTLM |
| | | ISRTPEVTCVVVDVSHEDPEVKFNWYVDGVEVHNAKTKPREEQYNS |
| | | TYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENN |
| | | YKTTPPVLDSDGSFFLYSKLTVDKSRWQQGNVFSCSVMHEALHNHY |
| | | TQKSLSLSPGHHHHHH |
| 17 | TNFR2 Chimera 6 | VPAQVVLTPYKPEPGYECQISQEYYDRKAQMCCAKCPPGQYVKHFC |
| | | NKTSDTVCADCEASMYTQVWNQFRTCLSCSSSCTTDQVEIRACTKQ |
| | | QNRVCACEAGRYCALKTHSGSCRQCMRLSKCGPGFGVASSRAPNGN |
| | | VLCKACAPGTFSDTTSSTDVCRPHRICSILAIPGNASTDAVCTSTS |
| | | PTRSMAPGAVHLPQPVSTRSQHTQPTPEPSTAPSTSFLLPMGPSPP |
| | | AEGSTGDIEGRMDPHHHHHH |
| | | mouse 23-202 (199-201); human 201 (197-200)- |
| | | 257 |
| 18 | TNFR2 Chimera 6 | VPAQVVLTPYKPEPGYECQISQEYYDRKAQMCCAKCPPGQYVKHFC |
| | Fc fusion | NKTSDTVCADCEASMYTQVWNQFRTCLSCSSSCTTDQVEIRACTKQ |
| | | QNRVCACEAGRYCALKTHSGSCRQCMRLSKCGPGFGVASSRAPNGN |
| | | VLCKACAPGTFSDTTSSTDVCRPHRICSILAIPGNASTDAVCTSTS |
| | | PTRSMAPGAVHLPQPVSTRSQHTQPTPEPSTAPSTSFLLPMGPSPP |
| | | AEGSTGDIEGRMDPKSSDKTHTCPPCPAPELLGGPSVFLFPPKPKD |
| | | TLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKG |
| | | QPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALH |
| | | NHYTQKSLSLSPGHHHHHH |
| 19 | TNFR2 Chimera 7 | LPAQVAFTPYAPEPGSTCRLREYYDQTAQMCCSKCSPGQHAKVFCT |
| | | KTSDTVCDSCEDSTYTQLWNWVPECLSCSSSCTTDQVEIRACTKQQ |
| | | NRVCACEAGRYCALKTHSGSCRQCMRLSKCGPGFGVASSRAPNGNV |
| | | LCKACAPGTFSDTTSSTDVCRPHRICSILAIPGNASTDAVCAPESP |
| | | TLSAIPRTLYVSQPEPTRSQPLDQEPGPSQTPSILTSLGSTPIIEQ |
| | | STKGGIEGRMDPHHHHHH |
| | | human 23-96 (93-95); mouse 98 (94-97)-258 |
| 20 | TNFR2 Chimera 7 | LPAQVAFTPYAPEPGSTCRLREYYDQTAQMCCSKCSPGQHAKVFCT |
| | Fc fusion | KTSDTVCDSCEDSTYTQLWNWVPECLSCSSSCTTDQVEIRACTKQQ |
| | | NRVCACEAGRYCALKTHSGSCRQCMRLSKCGPGFGVASSRAPNGNV |
| | | LCKACAPGTFSDTTSSTDVCRPHRICSILAIPGNASTDAVCAPESP |
| | | TLSAIPRTLYVSQPEPTRSQPLDQEPGPSQTPSILTSLGSTPIIEQ |
| | | STKGGIEGRMDPKSSDKTHTCPPCPAPELLGGPSVFLFPPKPKDTL |
| | | MISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQP |
| | | REPQVYTLPPSREEMTKNQVSLTCLVKGFYPSDIAVEWESNGQPEN |
| | | NYKTTPPVLDSDGSFFLYSKLTVDKSRWQQGNVFSCSVMHEALHNH |
| | | YTQKSLSLSPGHHHHHH |
| 21 | TNFR2 Chimera 8 | VPAQVVLTPYKPEPGYECQISQEYYDRKAQMCCAKCPPGQYVKHFC |
| | | NKTSDTVCADCEASMYTQVWNQFRTCLSCGSRCSSDQVETQACTRE |
| | | QNRICTCRPGWYCALSKQEGCRLCAPLRKCRPGFGVARPGTETSDV |
| | | VCKPCAPGTFSNTTSSTDICRPHQICNVVAIPGNASMDAVCTSTSP |
| | | TRSMAPGAVHLPQPVSTRSQHTQPTPEPSTAPSTSFLLPMGPSPPA |
| | | EGSTGDIEGRMDPHHHHHH |
| | | mouse 23-97 (94-96); human 97 (93-96)-257 |
| 22 | TNFR2 Chimera 8 | VPAQVVLTPYKPEPGYECQISQEYYDRKAQMCCAKCPPGQYVKHFC |
| | Fc fusion | NKTSDTVCADCEASMYTQVWNQFRTCLSCGSRCSSDQVETQACTRE |
| | | QNRICTCRPGWYCALSKQEGCRLCAPLRKCRPGFGVARPGTETSDV |
| | | VCKPCAPGTFSNTTSSTDICRPHQICNVVAIPGNASMDAVCTSTSP |
| | | TRSMAPGAVHLPQPVSTRSQHTQPTPEPSTAPSTSFLLPMGPSPPA |
| | | EGSTGDIEGRMDPKSSDKTHTCPPCPAPELLGGPSVFLFPPKPKDT |
| | | LMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQ |
| | | PREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALHN |
| | | HYTQKSLSLSPGHHHHHH |
| 23 | TNFR2 Chimera 9 | LPAQVAFTPYAPEPGSTCRLREYYDQTAQMCCSKCSPGQHAKVFCT |
| | | KTSDTVCDSCEDSTYTQLWNWVPECLSCGSRCSSDQVETQACTREQ |
| | | NRICACEAGRYCALKTHSGSCRQCMRLSKCGPGFGVASSRAPNGNV |
| | | LCKACAPGTFSDTTSSTDVCRPHRICSILAIPGNASTDAVCAPESP |
| | | TLSAIPRTLYVSQPEPTRSQPLDQEPGPSQTPSILTSLGSTPIIEQ |
| | | STKGGIEGRMDPHHHHHH |
| | | human 23-118 (117); mouse 120 (119)-258 |
| 24 | TNFR2 Chimera 9 | LPAQVAFTPYAPEPGSTCRLREYYDQTAQMCCSKCSPGQHAKVFCT |
| | Fc fusion | KTSDTVCDSCEDSTYTQLWNWVPECLSCGSRCSSDQVETQACTREQ |
| | | NRICACEAGRYCALKTHSGSCRQCMRLSKCGPGFGVASSRAPNGNV |
| | | LCKACAPGTFSDTTSSTDVCRPHRICSILAIPGNASTDAVCAPESP |
| | | TLSAIPRTLYVSQPEPTRSQPLDQEPGPSQTPSILTSLGSTPIIEQ |
| | | STKGGIEGRMDPKSSDKTHTCPPCPAPELLGGPSVFLFPPKPKDTL |
| | | MISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQP |
| | | REPQVYTLPPSREEMTKNQVSLTCLVKGFYPSDIAVEWESNGQPEN |
| | | NYKTTPPVLDSDGSFFLYSKLTVDKSRWQQGNVFSCSVMHEALHNH |
| | | YTQKSLSLSPGHHHHHH |
| 25 | Signal peptide | MGTPAQLLFLLLLWLPDTTG (Fc fusions were expressed |
| | | with the signal peptide, which was later cleaved |
| | | off) |
| 26 | TNFR2 Chimera 0 | TACCGGTGTCCCCGCCCAAGTCGTGCTTACTCCCTACAAGCCAGAA |
| | | CCTGGATATGAATGTCAGATTTCCCAAGAGTACTACGACCGGAAGG |
| | | CGCAGATGTGCTGTTCAAAGTGCAGCCCGGGCCAGCACGCCAAAGT |
| | | GTTCTGCACCAAGACCTCCGACACCGTGTGCGACAGCTGCGAGGAC |
| | | TCCACATACACTCAGCTCTGGAACTGGGTGCCAGAATGCCTGTCCT |
| | | GTGGCTCCCGCTGCTCCTCCGATCAAGTGGAGACTCAGGCCTGCAC |
| | | CAGGGAACAGAACAGAATCTGTACGTGCCGGCCGGGGTGGTACTGT |
| | | GCTCTGTCGAAGCAGGAGGGATGCAGACTGTGCGCCCCGTTGCGGA |
| | | AGTGCCGCCCTGGATTTGGTGTCGCGCGCCCGGGTACCGAAACCAG |
| | | CGATGTGGTCTGCAAGCCGTGCGCACCCGGGACCTTCTCAAACACC |
| | | ACCTCCTCGACCGACATCTGTCGGCCGCATCAGATTTGCAACGTGG |
| | | TGGCAATCCCTGGCAATGCCTCTATGGATGCTGTGTGCACTAGCAC |
| | | CTCCCCTACTCGCTCCATGGCGCCCGGAGCCGTGCACCTCCCGCAA |
| | | CCCGTGTCGACCAGGAGCCAGCACACTCAGCCTACCCCCGAACCCT |
| | | CCACCGCCCCTTCGACTTCATTCCTGCTGCCTATGGGACCATCCCC |
| | | GCCGGCCGAGGGCAGCACCGGAGACATTGAAGGCCGCATGGATCCG |
| | | CATCATCATCATCATCATTAATGAGCGGCCGC |
| 27 | TNFR2 Chimera 0 | GTCCCCGCCCAAGTCGTGCTTACTCCCTACAAGCCAGAACCTGGAT |
| | Fc fusion | ATGAATGTCAGATTTCCCAAGAGTACTACGACCGGAAGGCGCAGAT |
| | | GTGCTGTTCAAAGTGCAGCCCGGGCCAGCACGCCAAAGTGTTCTGC |
| | | ACCAAGACCTCCGACACCGTGTGCGACAGCTGCGAGGACTCCACAT |
| | | ACACTCAGCTCTGGAACTGGGTGCCAGAATGCCTGTCCTGTGGCTC |
| | | CCGCTGCTCCTCCGATCAAGTGGAGACTCAGGCCTGCACCAGGGAA |
| | | CAGAACAGAATCTGTACGTGCCGGCCGGGGTGGTACTGTGCTCTGT |
| | | CGAAGCAGGAGGGATGCAGACTGTGCGCCCCGTTGCGGAAGTGCCG |
| | | CCCTGGATTTGGTGTCGCGCGCCCGGGTACCGAAACCAGCGATGTG |
| | | GTCTGCAAGCCGTGCGCACCCGGGACCTTCTCAAACACCACCTCCT |
| | | CGACCGACATCTGTCGGCCGCATCAGATTTGCAACGTGGTGGCAAT |
| | | CCCTGGCAATGCCTCTATGGATGCTGTGTGCACTAGCACCTCCCCT |
| | | ACTCGCTCCATGGCGCCCGGAGCCGTGCACCTCCCGCAACCCGTGT |
| | | CGACCAGGAGCCAGCACACTCAGCCTACCCCCGAACCCTCCACCGC |
| | | CCCTTCGACTTCATTCCTGCTGCCTATGGGACCATCCCCGCCGGCC |
| | | GAGGGCAGCACCGGAGACATTGAAGGCCGCATGGATCCGAAATCGT |
| | | CTGATAAGACACATACATGCCCTCCATGTCCGGCGCCCGAGTTGCT |
| | | TGGAGGACCTTCGGTGTTTCTTTTTCCCCCGAAGCCAAAAGATACA |
| | | CTGATGATTTCACGGACGCCCGAGGTGACTTGTGTCGTCGTGGACG |
| | | TCAGCCACGAGGACCCAGAAGTCAAGTTTAACTGGTATGTAGATGG |
| | | GGTGGAGGTACACAATGCGAAAACGAAACCGAGAGAGGAGCAGTAC |
| | | AATTCGACGTATAGGGTGGTCAGCGTGCTGACGGTGTTGCACCAGG |
| | | ACTGGCTGAACGGGAAAGAGTATAAGTGCAAAGTGTCGAACAAGGC |
| | | CCTCCCCGCACCCATCGAAAAGACGATATCCAAAGCCAAGGGCCAA |
| | | CCGCGCGAGCCGCAAGTGTACACGCTGCCTCCCTCGCGAGAAGAGA |
| | | TGACCAAGAACCAGGTGTCCCTTACGTGCTTGGTGAAAGGATTCTA |
| | | CCCTTCGGACATCGCCGTAGAATGGGAAAGCAATGGGCAGCCAGAG |
| | | AACAATTACAAAACCACACCGCCTGTGCTCGACTCGGACGGTTCCT |
| | | TTTTCTTGTATTCCAAGTTGACAGTGGACAAGTCACGGTGGCAACA |
| | | GGGGAACGTATTCTCGTGTTCCGTCATGCACGAAGCGCTGCATAAC |
| | | CACTACACTCAGAAGTCGCTAAGCTTGTCGCCGGGTCATCATCATC |
| | | ATCATCATTAATGAGCGGCCGCT |
| 28 | TNFR2 Chimera 1 | TACCGGTGTCCCCGCCCAAGTCGTGCTCACCCCCTACAAGCCAGAA |
| | | CCCGGATACGAGTGTCAGATCAGCCAGGAGTATTACGACCGGAAGG |
| | | CCCAGATGTGCTGCGCCAAGTGTCCTCCGGGCCAATACGTGAAACA |
| | | CTTCTGCAACAAGACTTCCGATACCGTGTGCGCCGACTGCGAGGAT |
| | | TCAACGTACACCCAGCTGTGGAACTGGGTGCCTGAGTGCCTGTCTT |
| | | GCGGTAGCAGATGTAGCTCCGACCAGGTCGAAACCCAAGCCTGCAC |
| | | CCGCGAACAGAACAGGATTTGCACCTGTCGCCCGGGATGGTACTGC |
| | | GCTCTGTCGAAGCAGGAAGGTTGCCGCCTGTGCGCGCCTCTCCGGA |
| | | AGTGTAGACCGGGATTCGGCGTGGCCCGCCCCGGGACTGAAACTTC |
| | | CGATGTCGTGTGCAAGCCCTGCGCCCCCGGGACCTTTAGCAACACC |
| | | ACTTCCTCCACGGACATCTGTAGGCCCCATCAGATTTGCAACGTGG |
| | | TGGCGATCCCGGGCAATGCCAGCATGGACGCCGTGTGCACTTCCAC |
| | | CTCACCGACCCGGTCAATGGCACCTGGAGCTGTGCACTTGCCACAA |
| | | CCAGTGTCCACCCGGTCGCAGCACACCCAGCCCACCCCGGAGCCGT |
| | | CGACTGCACCTTCCACATCCTTCCTTCTGCCTATGGGACCGTCGCC |
| | | GCCTGCGGAAGGCTCCACTGGAGACATTGAAGGCCGCATGGATCCG |
| | | CATCATCATCATCATCATTAATGAGCGGCCGC |
| 29 | TNFR2 Chimera 1 | GTCCCCGCCCAAGTCGTGCTCACCCCCTACAAGCCAGAACCCGGAT |
| | Fc fusion | ACGAGTGTCAGATCAGCCAGGAGTATTACGACCGGAAGGCCCAGAT |
| | | GTGCTGCGCCAAGTGTCCTCCGGGCCAATACGTGAAACACTTCTGC |
| | | AACAAGACTTCCGATACCGTGTGCGCCGACTGCGAGGATTCAACGT |
| | | ACACCCAGCTGTGGAACTGGGTGCCTGAGTGCCTGTCTTGCGGTAG |
| | | CAGATGTAGCTCCGACCAGGTCGAAACCCAAGCCTGCACCCGCGAA |
| | | CAGAACAGGATTTGCACCTGTCGCCCGGGATGGTACTGCGCTCTGT |
| | | CGAAGCAGGAAGGTTGCCGCCTGTGCGCGCCTCTCCGGAAGTGTAG |
| | | ACCGGGATTCGGCGTGGCCCGCCCCGGGACTGAAACTTCCGATGTC |
| | | GTGTGCAAGCCCTGCGCCCCCGGGACCTTTAGCAACACCACTTCCT |
| | | CCACGGACATCTGTAGGCCCCATCAGATTTGCAACGTGGTGGCGAT |
| | | CCCGGGCAATGCCAGCATGGACGCCGTGTGCACTTCCACCTCACCG |
| | | ACCCGGTCAATGGCACCTGGAGCTGTGCACTTGCCACAACCAGTGT |
| | | CCACCCGGTCGCAGCACACCCAGCCCACCCCGGAGCCGTCGACTGC |
| | | ACCTTCCACATCCTTCCTTCTGCCTATGGGACCGTCGCCGCCTGCG |
| | | GAAGGCTCCACTGGAGACATTGAAGGCCGCATGGATCCGAAATCGT |
| | | CTGATAAGACACATACATGCCCTCCATGTCCGGCGCCCGAGTTGCT |
| | | TGGAGGACCTTCGGTGTTTCTTTTTCCCCCGAAGCCAAAAGATACA |
| | | CTGATGATTTCACGGACGCCCGAGGTGACTTGTGTCGTCGTGGACG |
| | | TCAGCCACGAGGACCCAGAAGTCAAGTTTAACTGGTATGTAGATGG |
| | | GGTGGAGGTACACAATGCGAAAACGAAACCGAGAGAGGAGCAGTAC |
| | | AATTCGACGTATAGGGTGGTCAGCGTGCTGACGGTGTTGCACCAGG |
| | | ACTGGCTGAACGGGAAAGAGTATAAGTGCAAAGTGTCGAACAAGGC |
| | | CCTCCCCGCACCCATCGAAAAGACGATATCCAAAGCCAAGGGCCAA |
| | | CCGCGCGAGCCGCAAGTGTACACGCTGCCTCCCTCGCGAGAAGAGA |
| | | TGACCAAGAACCAGGTGTCCCTTACGTGCTTGGTGAAAGGATTCTA |
| | | CCCTTCGGACATCGCCGTAGAATGGGAAAGCAATGGGCAGCCAGAG |
| | | AACAATTACAAAACCACACCGCCTGTGCTCGACTCGGACGGTTCCT |
| | | TTTTCTTGTATTCCAAGTTGACAGTGGACAAGTCACGGTGGCAACA |
| | | GGGGAACGTATTCTCGTGTTCCGTCATGCACGAAGCGCTGCATAAC |
| | | CACTACACTCAGAAGTCGCTAAGCTTGTCGCCGGGTCATCATCATC |
| | | ATCATCATTAA |
| 30 | TNFR2 Chimera 2 | TACCGGTGTCCCCGCCCAAGTCGTGCTTACCCCATACAAACCCGAA |
| | | CCCGGTTACGAATGTCAGATTAGCCAGGAGTACTATGATAGGAAGG |
| | | CCCAGATGTGTTGCGCGAAGTGCCCGCCCGGGCAGTACGTGAAGCA |
| | | CTTTTGCAACAAGACCTCCGACACTGTGTGCGCCGACTGCGAGGCT |
| | | TCGATGTACACTCAAGTCTGGAACCAGTTCAGAACATGCCTGTCCT |
| | | GCTCGTCCTCATGTACCACTGACCAAGTGGAAATCCGGGCTTGCAC |
| | | TAAGCAGCAGAACCGCGTGTGCACTTGCCGCCCTGGATGGTACTGT |
| | | GCCTTGAGCAAGCAGGAGGGATGCCGGCTCTGTGCCCCGCTGAGAA |
| | | AGTGCAGGCCTGGCTTCGGCGTGGCGCGCCCGGGAACCGAAACCTC |
| | | CGATGTCGTGTGCAAGCCGTGTGCCCCCGGGACTTTCAGCAACACC |
| | | ACCTCCTCCACCGACATCTGCCGGCCGCACCAGATTTGCAATGTGG |
| | | TGGCCATCCCTGGCAACGCCAGCATGGACGCCGTGTGCACCTCCAC |
| | | GTCACCGACCCGGTCGATGGCACCCGGAGCAGTGCATCTGCCACAA |
| | | CCTGTGTCTACCCGGAGCCAGCACACCCAGCCTACCCCTGAACCTT |
| | | CGACCGCGCCATCCACCTCCTTCCTCCTGCCCATGGGCCCGTCCCC |
| | | GCCCGCCGAGGGTAGCACTGGAGATATTGAAGGCCGCATGGATCCG |
| | | CATCATCATCATCATCATTAATGAGCGGCCGC |
| 31 | TNFR2 Chimera 2 | GTCCCCGCCCAAGTCGTGCTTACCCCATACAAACCCGAACCCGGTT |
| | Fc fusion | ACGAATGTCAGATTAGCCAGGAGTACTATGATAGGAAGGCCCAGAT |
| | | GTGTTGCGCGAAGTGCCCGCCCGGGCAGTACGTGAAGCACTTTTGC |
| | | AACAAGACCTCCGACACTGTGTGCGCCGACTGCGAGGCTTCGATGT |
| | | ACACTCAAGTCTGGAACCAGTTCAGAACATGCCTGTCCTGCTCGTC |
| | | CTCATGTACCACTGACCAAGTGGAAATCCGGGCTTGCACTAAGCAG |
| | | CAGAACCGCGTGTGCACTTGCCGCCCTGGATGGTACTGTGCCTTGA |
| | | GCAAGCAGGAGGGATGCCGGCTCTGTGCCCCGCTGAGAAAGTGCAG |
| | | GCCTGGCTTCGGCGTGGCGCGCCCGGGAACCGAAACCTCCGATGTC |
| | | GTGTGCAAGCCGTGTGCCCCCGGGACTTTCAGCAACACCACCTCCT |
| | | CCACCGACATCTGCCGGCCGCACCAGATTTGCAATGTGGTGGCCAT |
| | | CCCTGGCAACGCCAGCATGGACGCCGTGTGCACCTCCACGTCACCG |
| | | ACCCGGTCGATGGCACCCGGAGCAGTGCATCTGCCACAACCTGTGT |
| | | CTACCCGGAGCCAGCACACCCAGCCTACCCCTGAACCTTCGACCGC |
| | | GCCATCCACCTCCTTCCTCCTGCCCATGGGCCCGTCCCCGCCCGCC |
| | | GAGGGTAGCACTGGAGATATTGAAGGCCGCATGGATCCGAAATCGT |
| | | CTGATAAGACACATACATGCCCTCCATGTCCGGCGCCCGAGTTGCT |
| | | TGGAGGACCTTCGGTGTTTCTTTTTCCCCCGAAGCCAAAAGATACA |
| | | CTGATGATTTCACGGACGCCCGAGGTGACTTGTGTCGTCGTGGACG |
| | | TCAGCCACGAGGACCCAGAAGTCAAGTTTAACTGGTATGTAGATGG |
| | | GGTGGAGGTACACAATGCGAAAACGAAACCGAGAGAGGAGCAGTAC |
| | | AATTCGACGTATAGGGTGGTCAGCGTGCTGACGGTGTTGCACCAGG |
| | | ACTGGCTGAACGGGAAAGAGTATAAGTGCAAAGTGTCGAACAAGGC |
| | | CCTCCCCGCACCCATCGAAAAGACGATATCCAAAGCCAAGGGCCAA |
| | | CCGCGCGAGCCGCAAGTGTACACGCTGCCTCCCTCGCGAGAAGAGA |
| | | TGACCAAGAACCAGGTGTCCCTTACGTGCTTGGTGAAAGGATTCTA |
| | | CCCTTCGGACATCGCCGTAGAATGGGAAAGCAATGGGCAGCCAGAG |
| | | AACAATTACAAAACCACACCGCCTGTGCTCGACTCGGACGGTTCCT |
| | | TTTTCTTGTATTCCAAGTTGACAGTGGACAAGTCACGGTGGCAACA |
| | | GGGGAACGTATTCTCGTGTTCCGTCATGCACGAAGCGCTGCATAAC |
| | | CACTACACTCAGAAGTCGCTAAGCTTGTCGCCGGGTCATCATCATC |
| | | ATCATCATTAA |
| 32 | TNFR2 Chimera 3 | TACCGGTCTGCCTGCCCAAGTCGCCTTCACCCCCTACGCACCCGAA |
| | | CCTGGTTCCACTTGTCGGTTGAGAGAGTACTACGACCAGACTGCGC |
| | | AGATGTGCTGCGCCAAGTGCCCGCCCGGTCAATACGTGAAGCACTT |
| | | CTGCAACAAGACCAGCGATACAGTGTGCGCGGATTGTGAAGCCTCC |
| | | ATGTATACTCAAGTCTGGAACCAGTTTCGCACCTGTCTGTCATGCT |
| | | CCTCGTCCTGCACCACCGACCAAGTGGAAATCCGGGCTTGCACCAA |
| | | GCAGCAGAATCGCGTGTGCGCCTGCGAGGCCGGACGGTACTGCGCG |
| | | CTTAAGACTCACTCCGGGTCGTGTCGGCAGTGCATGAGGCTCTCAA |
| | | AATGCGGCCCCGGATTCGGAGTGGCTTCCTCCCGCGCCCCAAACGG |
| | | CAACGTGCTGTGCAAGGCTTGTGCCCCTGGAACCTTCAGCGACACC |
| | | ACTTCCTCGACCGACGTCTGTCGCCCGCATCGGATCTGCTCCATTC |
| | | TCGCCATTCCCGGAAACGCCAGCACCGACGCCGTGTGCGCACCGGA |
| | | ATCGCCGACCCTGTCTGCGATCCCAAGGACTCTCTACGTGTCACAG |
| | | CCTGAGCCTACTAGATCCCAGCCACTGGATCAGGAGCCGGGCCCCA |
| | | GCCAGACCCCGAGCATTCTGACGTCGCTGGGCAGCACCCCGATCAT |
| | | CGAACAGTCCACCAAGGGGGGAATTGAAGGCCGCATGGATCCGCAT |
| | | CATCATCATCATCATTAATGAGCGGCCGC |
| 33 | TNFR2 Chimera 3 | CTGCCTGCCCAAGTCGCCTTCACCCCCTACGCACCCGAACCTGGTT |
| | Fc fusion | CCACTTGTCGGTTGAGAGAGTACTACGACCAGACTGCGCAGATGTG |
| | | CTGCGCCAAGTGCCCGCCCGGTCAATACGTGAAGCACTTCTGCAAC |
| | | AAGACCAGCGATACAGTGTGCGCGGATTGTGAAGCCTCCATGTATA |
| | | CTCAAGTCTGGAACCAGTTTCGCACCTGTCTGTCATGCTCCTCGTC |
| | | CTGCACCACCGACCAAGTGGAAATCCGGGCTTGCACCAAGCAGCAG |
| | | AATCGCGTGTGCGCCTGCGAGGCCGGACGGTACTGCGCGCTTAAGA |
| | | CTCACTCCGGGTCGTGTCGGCAGTGCATGAGGCTCTCAAAATGCGG |
| | | CCCCGGATTCGGAGTGGCTTCCTCCCGCGCCCCAAACGGCAACGTG |
| | | CTGTGCAAGGCTTGTGCCCCTGGAACCTTCAGCGACACCACTTCCT |
| | | CGACCGACGTCTGTCGCCCGCATCGGATCTGCTCCATTCTCGCCAT |
| | | TCCCGGAAACGCCAGCACCGACGCCGTGTGCGCACCGGAATCGCCG |
| | | ACCCTGTCTGCGATCCCAAGGACTCTCTACGTGTCACAGCCTGAGC |
| | | CTACTAGATCCCAGCCACTGGATCAGGAGCCGGGCCCCAGCCAGAC |
| | | CCCGAGCATTCTGACGTCGCTGGGCAGCACCCCGATCATCGAACAG |
| | | TCCACCAAGGGGGGAATTGAAGGCCGCATGGATCCGAAATCGTCTG |
| | | ATAAGACACATACATGCCCTCCATGTCCGGCGCCCGAGTTGCTTGG |
| | | AGGACCTTCGGTGTTTCTTTTTCCCCCGAAGCCAAAAGATACACTG |
| | | ATGATTTCACGGACGCCCGAGGTGACTTGTGTCGTCGTGGACGTCA |
| | | GCCACGAGGACCCAGAAGTCAAGTTTAACTGGTATGTAGATGGGGT |
| | | GGAGGTACACAATGCGAAAACGAAACCGAGAGAGGAGCAGTACAAT |
| | | TCGACGTATAGGGTGGTCAGCGTGCTGACGGTGTTGCACCAGGACT |
| | | GGCTGAACGGGAAAGAGTATAAGTGCAAAGTGTCGAACAAGGCCCT |
| | | CCCCGCACCCATCGAAAAGACGATATCCAAAGCCAAGGGCCAACCG |
| | | CGCGAGCCGCAAGTGTACACGCTGCCTCCCTCGCGAGAAGAGATGA |
| | | CCAAGAACCAGGTGTCCCTTACGTGCTTGGTGAAAGGATTCTACCC |
| | | TTCGGACATCGCCGTAGAATGGGAAAGCAATGGGCAGCCAGAGAAC |
| | | AATTACAAAACCACACCGCCTGTGCTCGACTCGGACGGTTCCTTTT |
| | | TCTTGTATTCCAAGTTGACAGTGGACAAGTCACGGTGGCAACAGGG |
| | | GAACGTATTCTCGTGTTCCGTCATGCACGAAGCGCTGCATAACCAC |
| | | TACACTCAGAAGTCGCTAAGCTTGTCGCCGGGTCATCATCATCATC |
| | | ATCATTAA |
| 34 | TNFR2 Chimera 4 | TACCGGTCTGCCCGCCCAAGTCGCCTTTACCCCCTACGCCCCCGAG |
| | | CCTGGTTCCACCTGTCGCCTGCGCGAATACTACGACCAGACAGCGC |
| | | AGATGTGCTGCTCAAAGTGCTCGCCCGGACAGCATGCAAAGGTGTT |
| | | CTGCACCAAGACTTCCGATACCGTGTGCGCCGACTGTGAAGCTAGC |
| | | ATGTACACCCAAGTCTGGAACCAGTTCCGGACTTGCTTGTCCTGTT |
| | | CGTCATCCTGTACGACCGACCAGGTCGAGATCAGGGCGTGCACCAA |
| | | GCAGCAAAACCGCGTGTGCGCTTGCGAGGCTGGAAGATATTGTGCG |
| | | CTCAAGACCCACTCCGGGAGCTGCAGGCAGTGCATGCGGCTCTCTA |
| | | AGTGCGGACCTGGATTCGGAGTGGCCTCCTCGCGGGCCCCTAACGG |
| | | CAACGTGCTTTGTAAAGCCTGCGCCCCGGGCACTTTCAGCGACACC |
| | | ACTAGCTCGACTGACGTGTGCCGCCCGCACCGGATCTGCAGCATCC |
| | | TCGCGATTCCCGGCAATGCCAGCACGGATGCAGTGTGCGCCCCGGA |
| | | GTCCCCTACCCTGTCCGCCATTCCGCGGACTCTGTACGTGTCGCAA |
| | | CCTGAACCGACCAGATCCCAGCCGCTGGATCAGGAGCCCGGGCCGT |
| | | CCCAGACTCCATCCATCCTGACCTCACTGGGTTCCACCCCAATCAT |
| | | TGAACAGTCCACCAAGGGCGGAATTGAAGGCCGCATGGATCCGCAT |
| | | CATCATCATCATCATTAATGAGCGGCCGC |
| 35 | TNFR2 Chimera 4 | CTGCCCGCCCAAGTCGCCTTTACCCCCTACGCCCCCGAGCCTGGTT |
| | Fc fusion | CCACCTGTCGCCTGCGCGAATACTACGACCAGACAGCGCAGATGTG |
| | | CTGCTCAAAGTGCTCGCCCGGACAGCATGCAAAGGTGTTCTGCACC |
| | | AAGACTTCCGATACCGTGTGCGCCGACTGTGAAGCTAGCATGTACA |
| | | CCCAAGTCTGGAACCAGTTCCGGACTTGCTTGTCCTGTTCGTCATC |
| | | CTGTACGACCGACCAGGTCGAGATCAGGGCGTGCACCAAGCAGCAA |
| | | AACCGCGTGTGCGCTTGCGAGGCTGGAAGATATTGTGCGCTCAAGA |
| | | CCCACTCCGGGAGCTGCAGGCAGTGCATGCGGCTCTCTAAGTGCGG |
| | | ACCTGGATTCGGAGTGGCCTCCTCGCGGGCCCCTAACGGCAACGTG |
| | | CTTTGTAAAGCCTGCGCCCCGGGCACTTTCAGCGACACCACTAGCT |
| | | CGACTGACGTGTGCCGCCCGCACCGGATCTGCAGCATCCTCGCGAT |
| | | TCCCGGCAATGCCAGCACGGATGCAGTGTGCGCCCCGGAGTCCCCT |
| | | ACCCTGTCCGCCATTCCGCGGACTCTGTACGTGTCGCAACCTGAAC |
| | | CGACCAGATCCCAGCCGCTGGATCAGGAGCCCGGGCCGTCCCAGAC |
| | | TCCATCCATCCTGACCTCACTGGGTTCCACCCCAATCATTGAACAG |
| | | TCCACCAAGGGCGGAATTGAAGGCCGCATGGATCCGAAATCGTCTG |
| | | ATAAGACACATACATGCCCTCCATGTCCGGCGCCCGAGTTGCTTGG |
| | | AGGACCTTCGGTGTTTCTTTTTCCCCCGAAGCCAAAAGATACACTG |
| | | ATGATTTCACGGACGCCCGAGGTGACTTGTGTCGTCGTGGACGTCA |
| | | GCCACGAGGACCCAGAAGTCAAGTTTAACTGGTATGTAGATGGGGT |
| | | GGAGGTACACAATGCGAAAACGAAACCGAGAGAGGAGCAGTACAAT |
| | | TCGACGTATAGGGTGGTCAGCGTGCTGACGGTGTTGCACCAGGACT |
| | | GGCTGAACGGGAAAGAGTATAAGTGCAAAGTGTCGAACAAGGCCCT |
| | | CCCCGCACCCATCGAAAAGACGATATCCAAAGCCAAGGGCCAACCG |
| | | CGCGAGCCGCAAGTGTACACGCTGCCTCCCTCGCGAGAAGAGATGA |
| | | CCAAGAACCAGGTGTCCCTTACGTGCTTGGTGAAAGGATTCTACCC |
| | | TTCGGACATCGCCGTAGAATGGGAAAGCAATGGGCAGCCAGAGAAC |
| | | AATTACAAAACCACACCGCCTGTGCTCGACTCGGACGGTTCCTTTT |
| | | TCTTGTATTCCAAGTTGACAGTGGACAAGTCACGGTGGCAACAGGG |
| | | GAACGTATTCTCGTGTTCCGTCATGCACGAAGCGCTGCATAACCAC |
| | | TACACTCAGAAGTCGCTAAGCTTGTCGCCGGGTCATCATCATCATC |
| | | ATCATTAA |
| 36 | TNFR2 Chimera 5 | TACCGGTCTGCCTGCCCAAGTCGCCTTCACTCCCTACGCCCCCGAA |
| | | CCCGGCTCCACCTGTCGCCTGAGAGAGTACTACGATCAGACCGCGC |
| | | AGATGTGCTGTTCCAAGTGTTCCCCGGGACAGCACGCGAAAGTGTT |
| | | CTGCACCAAGACCAGCGACACCGTGTGCGATTCCTGCGAGGACTCC |
| | | ACATACACTCAGCTCTGGAACTGGGTCCCAGAATGTCTGTCCTGCG |
| | | GTAGCCGGTGTTCCTCGGACCAAGTGGAAACCCAGGCCTGCACTCG |
| | | CGAGCAGAATCGGATTTGCACTTGCCGGCCTGGGTGGTATTGCGCC |
| | | CTGTCAAAGCAGGAGGGCTGCCGGCTCTGCGCACCTCTGAGGAAGT |
| | | GCAGACCCGGATTTGGAGTGGCCCGCCCGGGAACCGAAACCAGCGA |
| | | CGTCGTGTGCAAGCCGTGTGCCCCGGGGACCTTCAGCAACACCACG |
| | | TCCTCGACCGATATTTGCCGGCCGCATCAGATCTGCAACGTGGTGG |
| | | CAATTCCGGGAAACGCTTCAATGGACGCTGTGTGCGCCCCCGAGTC |
| | | TCCAACTTTGAGCGCGATCCCTCGCACTCTCTACGTGTCCCAACCG |
| | | GAGCCCACCAGGTCACAGCCACTGGACCAAGAACCTGGCCCGAGCC |
| | | AGACTCCTTCGATCCTTACTTCCCTGGGTTCGACCCCCATCATCGA |
| | | ACAGTCCACCAAGGGAGGCATTGAAGGCCGCATGGATCCGCATCAT |
| | | CATCATCATCATTAATGAGCGGCCGC |
| 37 | TNFR2 Chimera 5 | CTGCCTGCCCAAGTCGCCTTCACTCCCTACGCCCCCGAACCCGGCT |
| | Fc fusion | CCACCTGTCGCCTGAGAGAGTACTACGATCAGACCGCGCAGATGTG |
| | | CTGTTCCAAGTGTTCCCCGGGACAGCACGCGAAAGTGTTCTGCACC |
| | | AAGACCAGCGACACCGTGTGCGATTCCTGCGAGGACTCCACATACA |
| | | CTCAGCTCTGGAACTGGGTCCCAGAATGTCTGTCCTGCGGTAGCCG |
| | | GTGTTCCTCGGACCAAGTGGAAACCCAGGCCTGCACTCGCGAGCAG |
| | | AATCGGATTTGCACTTGCCGGCCTGGGTGGTATTGCGCCCTGTCAA |
| | | AGCAGGAGGGCTGCCGGCTCTGCGCACCTCTGAGGAAGTGCAGACC |
| | | CGGATTTGGAGTGGCCCGCCCGGGAACCGAAACCAGCGACGTCGTG |
| | | TGCAAGCCGTGTGCCCCGGGGACCTTCAGCAACACCACGTCCTCGA |
| | | CCGATATTTGCCGGCCGCATCAGATCTGCAACGTGGTGGCAATTCC |
| | | GGGAAACGCTTCAATGGACGCTGTGTGCGCCCCCGAGTCTCCAACT |
| | | TTGAGCGCGATCCCTCGCACTCTCTACGTGTCCCAACCGGAGCCCA |
| | | CCAGGTCACAGCCACTGGACCAAGAACCTGGCCCGAGCCAGACTCC |
| | | TTCGATCCTTACTTCCCTGGGTTCGACCCCCATCATCGAACAGTCC |
| | | ACCAAGGGAGGCATTGAAGGCCGCATGGATCCGAAATCGTCTGATA |
| | | AGACACATACATGCCCTCCATGTCCGGCGCCCGAGTTGCTTGGAGG |
| | | ACCTTCGGTGTTTCTTTTTCCCCCGAAGCCAAAAGATACACTGATG |
| | | ATTTCACGGACGCCCGAGGTGACTTGTGTCGTCGTGGACGTCAGCC |
| | | ACGAGGACCCAGAAGTCAAGTTTAACTGGTATGTAGATGGGGTGGA |
| | | GGTACACAATGCGAAAACGAAACCGAGAGAGGAGCAGTACAATTCG |
| | | ACGTATAGGGTGGTCAGCGTGCTGACGGTGTTGCACCAGGACTGGC |
| | | TGAACGGGAAAGAGTATAAGTGCAAAGTGTCGAACAAGGCCCTCCC |
| | | CGCACCCATCGAAAAGACGATATCCAAAGCCAAGGGCCAACCGCGC |
| | | GAGCCGCAAGTGTACACGCTGCCTCCCTCGCGAGAAGAGATGACCA |
| | | AGAACCAGGTGTCCCTTACGTGCTTGGTGAAAGGATTCTACCCTTC |
| | | GGACATCGCCGTAGAATGGGAAAGCAATGGGCAGCCAGAGAACAAT |
| | | TACAAAACCACACCGCCTGTGCTCGACTCGGACGGTTCCTTTTTCT |
| | | TGTATTCCAAGTTGACAGTGGACAAGTCACGGTGGCAACAGGGGAA |
| | | CGTATTCTCGTGTTCCGTCATGCACGAAGCGCTGCATAACCACTAC |
| | | ACTCAGAAGTCGCTAAGCTTGTCGCCGGGTCATCATCATCATCATC |
| | | ATTAA |
| 38 | TNFR2 Chimera 6 | TACCGGTGTCCCCGCCCAAGTCGTCCTCACCCCATACAAGCCTGAA |
| | | CCCGGATACGAGTGCCAGATTAGCCAAGAGTACTACGACCGCAAGG |
| | | CTCAGATGTGCTGTGCGAAGTGCCCACCGGGACAATACGTGAAGCA |
| | | CTTCTGCAACAAGACCAGCGACACCGTGTGTGCCGATTGCGAAGCG |
| | | TCCATGTATACCCAGGTCTGGAATCAGTTCAGAACCTGTCTTTCAT |
| | | GTTCCTCCTCCTGCACTACCGACCAAGTGGAGATCCGGGCCTGCAC |
| | | TAAGCAGCAGAACCGCGTGTGCGCTTGCGAGGCCGGCCGGTACTGC |
| | | GCGCTCAAGACCCACTCAGGGTCGTGCCGGCAGTGCATGCGGCTGT |
| | | CCAAATGTGGCCCGGGATTTGGCGTGGCATCGAGCAGGGCGCCTAA |
| | | CGGGAACGTGCTGTGCAAGGCCTGCGCCCCCGGAACATTCTCCGAT |
| | | ACTACTTCCTCCACGGACGTGTGCAGGCCACACCGCATCTGTTCTA |
| | | TCTTGGCCATTCCGGGAAACGCCAGCACCGATGCTGTGTGCACCTC |
| | | CACTTCGCCTACTCGGTCCATGGCCCCGGGTGCAGTGCATCTGCCG |
| | | CAGCCCGTGTCAACCAGATCGCAGCACACTCAGCCTACCCCCGAAC |
| | | CCAGCACCGCCCCTAGCACCTCGTTCCTGCTGCCTATGGGACCGTC |
| | | CCCGCCCGCCGAAGGTTCCACCGGCGACATTGAAGGCCGCATGGAT |
| | | CCGCATCATCATCATCATCATTAATGAGCGGCCGC |
| 39 | TNFR2 Chimera 6 | GTCCCCGCCCAAGTCGTCCTCACCCCATACAAGCCTGAACCCGGAT |
| | Fc fusion | ACGAGTGCCAGATTAGCCAAGAGTACTACGACCGCAAGGCTCAGAT |
| | | GTGCTGTGCGAAGTGCCCACCGGGACAATACGTGAAGCACTTCTGC |
| | | AACAAGACCAGCGACACCGTGTGTGCCGATTGCGAAGCGTCCATGT |
| | | ATACCCAGGTCTGGAATCAGTTCAGAACCTGTCTTTCATGTTCCTC |
| | | CTCCTGCACTACCGACCAAGTGGAGATCCGGGCCTGCACTAAGCAG |
| | | CAGAACCGCGTGTGCGCTTGCGAGGCCGGCCGGTACTGCGCGCTCA |
| | | AGACCCACTCAGGGTCGTGCCGGCAGTGCATGCGGCTGTCCAAATG |
| | | TGGCCCGGGATTTGGCGTGGCATCGAGCAGGGCGCCTAACGGGAAC |
| | | GTGCTGTGCAAGGCCTGCGCCCCCGGAACATTCTCCGATACTACTT |
| | | CCTCCACGGACGTGTGCAGGCCACACCGCATCTGTTCTATCTTGGC |
| | | CATTCCGGGAAACGCCAGCACCGATGCTGTGTGCACCTCCACTTCG |
| | | CCTACTCGGTCCATGGCCCCGGGTGCAGTGCATCTGCCGCAGCCCG |
| | | TGTCAACCAGATCGCAGCACACTCAGCCTACCCCCGAACCCAGCAC |
| | | CGCCCCTAGCACCTCGTTCCTGCTGCCTATGGGACCGTCCCCGCCC |
| | | GCCGAAGGTTCCACCGGCGACATTGAAGGCCGCATGGATCCGAAAT |
| | | CGTCTGATAAGACACATACATGCCCTCCATGTCCGGCGCCCGAGTT |
| | | GCTTGGAGGACCTTCGGTGTTTCTTTTTCCCCCGAAGCCAAAAGAT |
| | | ACACTGATGATTTCACGGACGCCCGAGGTGACTTGTGTCGTCGTGG |
| | | ACGTCAGCCACGAGGACCCAGAAGTCAAGTTTAACTGGTATGTAGA |
| | | TGGGGTGGAGGTACACAATGCGAAAACGAAACCGAGAGAGGAGCAG |
| | | TACAATTCGACGTATAGGGTGGTCAGCGTGCTGACGGTGTTGCACC |
| | | AGGACTGGCTGAACGGGAAAGAGTATAAGTGCAAAGTGTCGAACAA |
| | | GGCCCTCCCCGCACCCATCGAAAAGACGATATCCAAAGCCAAGGGC |
| | | CAACCGCGCGAGCCGCAAGTGTACACGCTGCCTCCCTCGCGAGAAG |
| | | AGATGACCAAGAACCAGGTGTCCCTTACGTGCTTGGTGAAAGGATT |
| | | CTACCCTTCGGACATCGCCGTAGAATGGGAAAGCAATGGGCAGCCA |
| | | GAGAACAATTACAAAACCACACCGCCTGTGCTCGACTCGGACGGTT |
| | | CCTTTTTCTTGTATTCCAAGTTGACAGTGGACAAGTCACGGTGGCA |
| | | ACAGGGGAACGTATTCTCGTGTTCCGTCATGCACGAAGCGCTGCAT |
| | | AACCACTACACTCAGAAGTCGCTAAGCTTGTCGCCGGGTCATCATC |
| | | ATCATCATCATTAA |
| 40 | TNFR2 Chimera 7 | TACCGGTCTGCCTGCCCAAGTCGCCTTCACCCCGTACGCCCCCGAA |
| | | CCCGGTTCAACCTGTCGCCTGAGAGAGTATTACGACCAGACCGCGC |
| | | AGATGTGCTGCTCCAAGTGTTCCCCGGGACAGCATGCTAAGGTCTT |
| | | TTGCACCAAAACAAGCGACACTGTGTGCGACTCCTGCGAGGATTCC |
| | | ACCTACACCCAACTGTGGAACTGGGTGCCCGAGTGTCTGAGCTGCT |
| | | CCTCCTCCTGTACTACGGATCAAGTGGAGATTCGGGCCTGCACCAA |
| | | GCAGCAAAACCGGGTCTGCGCCTGTGAAGCCGGCCGCTACTGCGCA |
| | | CTCAAGACTCACTCGGGCTCATGCAGGCAGTGTATGCGGCTGTCTA |
| | | AGTGCGGACCCGGCTTCGGAGTGGCCAGCTCCAGAGCCCCTAATGG |
| | | CAACGTGTTGTGCAAGGCCTGCGCGCCGGGGACCTTCTCGGATACT |
| | | ACTAGCTCCACCGACGTGTGCCGCCCCCACCGGATCTGCAGCATCC |
| | | TGGCTATCCCTGGAAACGCGTCGACCGACGCCGTGTGCGCGCCGGA |
| | | ATCACCGACCCTCTCGGCAATTCCGCGCACTCTCTACGTGTCGCAG |
| | | CCAGAACCCACCAGGTCCCAGCCACTGGACCAGGAACCAGGACCTA |
| | | GCCAGACTCCGTCCATCCTTACCTCCCTGGGAAGCACCCCTATCAT |
| | | TGAGCAGTCCACCAAGGGGGGTATTGAAGGCCGCATGGATCCGCAT |
| | | CATCATCATCATCATTAATGAGCGGCCGC |
| 41 | TNFR2 Chimera 7 | CTGCCTGCCCAAGTCGCCTTCACCCCGTACGCCCCCGAACCCGGTT |
| | Fc fusion | CAACCTGTCGCCTGAGAGAGTATTACGACCAGACCGCGCAGATGTG |
| | | CTGCTCCAAGTGTTCCCCGGGACAGCATGCTAAGGTCTTTTGCACC |
| | | AAAACAAGCGACACTGTGTGCGACTCCTGCGAGGATTCCACCTACA |
| | | CCCAACTGTGGAACTGGGTGCCCGAGTGTCTGAGCTGCTCCTCCTC |
| | | CTGTACTACGGATCAAGTGGAGATTCGGGCCTGCACCAAGCAGCAA |
| | | AACCGGGTCTGCGCCTGTGAAGCCGGCCGCTACTGCGCACTCAAGA |
| | | CTCACTCGGGCTCATGCAGGCAGTGTATGCGGCTGTCTAAGTGCGG |
| | | ACCCGGCTTCGGAGTGGCCAGCTCCAGAGCCCCTAATGGCAACGTG |
| | | TTGTGCAAGGCCTGCGCGCCGGGGACCTTCTCGGATACTACTAGCT |
| | | CCACCGACGTGTGCCGCCCCCACCGGATCTGCAGCATCCTGGCTAT |
| | | CCCTGGAAACGCGTCGACCGACGCCGTGTGCGCGCCGGAATCACCG |
| | | ACCCTCTCGGCAATTCCGCGCACTCTCTACGTGTCGCAGCCAGAAC |
| | | CCACCAGGTCCCAGCCACTGGACCAGGAACCAGGACCTAGCCAGAC |
| | | TCCGTCCATCCTTACCTCCCTGGGAAGCACCCCTATCATTGAGCAG |
| | | TCCACCAAGGGGGGTATTGAAGGCCGCATGGATCCGAAATCGTCTG |
| | | ATAAGACACATACATGCCCTCCATGTCCGGCGCCCGAGTTGCTTGG |
| | | AGGACCTTCGGTGTTTCTTTTTCCCCCGAAGCCAAAAGATACACTG |
| | | ATGATTTCACGGACGCCCGAGGTGACTTGTGTCGTCGTGGACGTCA |
| | | GCCACGAGGACCCAGAAGTCAAGTTTAACTGGTATGTAGATGGGGT |
| | | GGAGGTACACAATGCGAAAACGAAACCGAGAGAGGAGCAGTACAAT |
| | | TCGACGTATAGGGTGGTCAGCGTGCTGACGGTGTTGCACCAGGACT |
| | | GGCTGAACGGGAAAGAGTATAAGTGCAAAGTGTCGAACAAGGCCCT |
| | | CCCCGCACCCATCGAAAAGACGATATCCAAAGCCAAGGGCCAACCG |
| | | CGCGAGCCGCAAGTGTACACGCTGCCTCCCTCGCGAGAAGAGATGA |
| | | CCAAGAACCAGGTGTCCCTTACGTGCTTGGTGAAAGGATTCTACCC |
| | | TTCGGACATCGCCGTAGAATGGGAAAGCAATGGGCAGCCAGAGAAC |
| | | AATTACAAAACCACACCGCCTGTGCTCGACTCGGACGGTTCCTTTT |
| | | TCTTGTATTCCAAGTTGACAGTGGACAAGTCACGGTGGCAACAGGG |
| | | GAACGTATTCTCGTGTTCCGTCATGCACGAAGCGCTGCATAACCAC |
| | | TACACTCAGAAGTCGCTAAGCTTGTCGCCGGGTCATCATCATCATC |
| | | ATCATTAA |
| 42 | TNFR2 Chimera 8 | TACCGGTGTCCCCGCCCAAGTCGTGCTGACCCCCTACAAACCCGAG |
| | | CCAGGATATGAATGCCAGATCTCCCAAGAGTACTACGACCGCAAGG |
| | | CCCAGATGTGTTGCGCGAAGTGTCCGCCGGGGCAGTACGTGAAGCA |
| | | CTTCTGCAACAAGACCTCCGATACCGTGTGCGCCGATTGCGAAGCG |
| | | TCCATGTACACTCAAGTCTGGAACCAGTTCAGAACTTGCCTGTCTT |
| | | GTGGCTCGAGGTGCTCAAGCGACCAGGTGGAAACTCAGGCTTGCAC |
| | | GCGGGAGCAGAATCGCATTTGCACTTGCCGGCCGGGCTGGTACTGC |
| | | GCCTTGTCAAAGCAGGAAGGTTGCAGGCTGTGTGCCCCACTGCGGA |
| | | AGTGTCGGCCTGGTTTCGGAGTGGCTCGCCCGGGCACCGAGACTTC |
| | | AGACGTGGTCTGCAAGCCCTGCGCGCCCGGAACCTTTAGCAACACC |
| | | ACCTCCTCGACCGACATTTGTAGACCGCACCAGATCTGCAACGTGG |
| | | TGGCCATCCCCGGGAACGCCTCGATGGATGCAGTGTGCACCAGCAC |
| | | TAGCCCGACCCGCTCCATGGCCCCTGGAGCCGTGCACCTCCCCCAA |
| | | CCTGTGTCCACCCGGTCCCAGCATACACAGCCTACCCCTGAACCAT |
| | | CCACCGCACCGTCCACTTCCTTCCTTCTCCCTATGGGCCCGAGCCC |
| | | GCCCGCCGAGGGATCGACCGGAGACATTGAAGGCCGCATGGATCCG |
| | | CATCATCATCATCATCATTAATGAGCGGCCGC |
| 43 | TNFR2 Chimera 8 | GTCCCCGCCCAAGTCGTGCTGACCCCCTACAAACCCGAGCCAGGAT |
| | Fc fusion | ATGAATGCCAGATCTCCCAAGAGTACTACGACCGCAAGGCCCAGAT |
| | | GTGTTGCGCGAAGTGTCCGCCGGGGCAGTACGTGAAGCACTTCTGC |
| | | AACAAGACCTCCGATACCGTGTGCGCCGATTGCGAAGCGTCCATGT |
| | | ACACTCAAGTCTGGAACCAGTTCAGAACTTGCCTGTCTTGTGGCTC |
| | | GAGGTGCTCAAGCGACCAGGTGGAAACTCAGGCTTGCACGCGGGAG |
| | | CAGAATCGCATTTGCACTTGCCGGCCGGGCTGGTACTGCGCCTTGT |
| | | CAAAGCAGGAAGGTTGCAGGCTGTGTGCCCCACTGCGGAAGTGTCG |
| | | GCCTGGTTTCGGAGTGGCTCGCCCGGGCACCGAGACTTCAGACGTG |
| | | GTCTGCAAGCCCTGCGCGCCCGGAACCTTTAGCAACACCACCTCCT |
| | | CGACCGACATTTGTAGACCGCACCAGATCTGCAACGTGGTGGCCAT |
| | | CCCCGGGAACGCCTCGATGGATGCAGTGTGCACCAGCACTAGCCCG |
| | | ACCCGCTCCATGGCCCCTGGAGCCGTGCACCTCCCCCAACCTGTGT |
| | | CCACCCGGTCCCAGCATACACAGCCTACCCCTGAACCATCCACCGC |
| | | ACCGTCCACTTCCTTCCTTCTCCCTATGGGCCCGAGCCCGCCCGCC |
| | | GAGGGATCGACCGGAGACATTGAAGGCCGCATGGATCCGAAATCGT |
| | | CTGATAAGACACATACATGCCCTCCATGTCCGGCGCCCGAGTTGCT |
| | | TGGAGGACCTTCGGTGTTTCTTTTTCCCCCGAAGCCAAAAGATACA |
| | | CTGATGATTTCACGGACGCCCGAGGTGACTTGTGTCGTCGTGGACG |
| | | TCAGCCACGAGGACCCAGAAGTCAAGTTTAACTGGTATGTAGATGG |
| | | GGTGGAGGTACACAATGCGAAAACGAAACCGAGAGAGGAGCAGTAC |
| | | AATTCGACGTATAGGGTGGTCAGCGTGCTGACGGTGTTGCACCAGG |
| | | ACTGGCTGAACGGGAAAGAGTATAAGTGCAAAGTGTCGAACAAGGC |
| | | CCTCCCCGCACCCATCGAAAAGACGATATCCAAAGCCAAGGGCCAA |
| | | CCGCGCGAGCCGCAAGTGTACACGCTGCCTCCCTCGCGAGAAGAGA |
| | | TGACCAAGAACCAGGTGTCCCTTACGTGCTTGGTGAAAGGATTCTA |
| | | CCCTTCGGACATCGCCGTAGAATGGGAAAGCAATGGGCAGCCAGAG |
| | | AACAATTACAAAACCACACCGCCTGTGCTCGACTCGGACGGTTCCT |
| | | TTTTCTTGTATTCCAAGTTGACAGTGGACAAGTCACGGTGGCAACA |
| | | GGGGAACGTATTCTCGTGTTCCGTCATGCACGAAGCGCTGCATAAC |
| | | CACTACACTCAGAAGTCGCTAAGCTTGTCGCCGGGTCATCATCATC |
| | | ATCATCAT |
| 44 | TNFR2 Chimera 9 | TACCGGTCTGCCCGCACAAGTCGCCTTCACCCCATACGCCCCTGAA |
| | | CCCGGATCAACTTGCCGCCTGAGAGAGTACTACGATCAGACCGCCC |
| | | AGATGTGTTGCTCCAAGTGCAGCCCTGGCCAACACGCGAAGGTGTT |
| | | CTGTACCAAGACGTCCGACACCGTGTGCGACAGCTGCGAGGACTCC |
| | | ACCTATACTCAGCTCTGGAACTGGGTGCCCGAATGCTTGTCCTGCG |
| | | GTAGCCGCTGTAGCTCGGATCAGGTCGAAACCCAGGCCTGTACTCG |
| | | GGAGCAGAACAGAATTTGCGCGTGCGAAGCGGGACGGTACTGCGCT |
| | | CTGAAAACACATTCCGGCTCGTGTCGGCAGTGCATGAGGCTGTCGA |
| | | AGTGCGGCCCGGGATTCGGCGTGGCCTCGTCCCGGGCTCCGAACGG |
| | | GAATGTGCTGTGCAAGGCCTGCGCCCCTGGCACCTTTTCCGACACT |
| | | ACTTCCTCCACCGACGTGTGCCGGCCCCACCGCATTTGCTCCATCC |
| | | TGGCAATCCCGGGGAACGCCAGCACCGATGCCGTGTGTGCCCCGGA |
| | | ATCCCCGACCCTGTCCGCCATCCCTCGCACTCTCTACGTGTCTCAG |
| | | CCGGAGCCTACTAGGTCACAGCCCCTTGACCAAGAACCAGGACCCA |
| | | GCCAAACCCCATCAATCCTGACCTCCCTCGGATCGACCCCGATTAT |
| | | CGAGCAGAGCACCAAGGGTGGAATTGAAGGCCGCATGGATCCGCAT |
| | | CATCATCATCATCATTAATGAGCGGCCGC |
| 45 | TNFR2 Chimera 9 | CTGCCCGCACAAGTCGCCTTCACCCCATACGCCCCTGAACCCGGAT |
| | Fc fusion | CAACTTGCCGCCTGAGAGAGTACTACGATCAGACCGCCCAGATGTG |
| | | TTGCTCCAAGTGCAGCCCTGGCCAACACGCGAAGGTGTTCTGTACC |
| | | AAGACGTCCGACACCGTGTGCGACAGCTGCGAGGACTCCACCTATA |
| | | CTCAGCTCTGGAACTGGGTGCCCGAATGCTTGTCCTGCGGTAGCCG |
| | | CTGTAGCTCGGATCAGGTCGAAACCCAGGCCTGTACTCGGGAGCAG |
| | | AACAGAATTTGCGCGTGCGAAGCGGGACGGTACTGCGCTCTGAAAA |
| | | CACATTCCGGCTCGTGTCGGCAGTGCATGAGGCTGTCGAAGTGCGG |
| | | CCCGGGATTCGGCGTGGCCTCGTCCCGGGCTCCGAACGGGAATGTG |
| | | CTGTGCAAGGCCTGCGCCCCTGGCACCTTTTCCGACACTACTTCCT |
| | | CCACCGACGTGTGCCGGCCCCACCGCATTTGCTCCATCCTGGCAAT |
| | | CCCGGGGAACGCCAGCACCGATGCCGTGTGTGCCCCGGAATCCCCG |
| | | ACCCTGTCCGCCATCCCTCGCACTCTCTACGTGTCTCAGCCGGAGC |
| | | CTACTAGGTCACAGCCCCTTGACCAAGAACCAGGACCCAGCCAAAC |
| | | CCCATCAATCCTGACCTCCCTCGGATCGACCCCGATTATCGAGCAG |
| | | AGCACCAAGGGTGGAATTGAAGGCCGCATGGATCCGAAATCGTCTG |
| | | ATAAGACACATACATGCCCTCCATGTCCGGCGCCCGAGTTGCTTGG |
| | | AGGACCTTCGGTGTTTCTTTTTCCCCCGAAGCCAAAAGATACACTG |
| | | ATGATTTCACGGACGCCCGAGGTGACTTGTGTCGTCGTGGACGTCA |
| | | GCCACGAGGACCCAGAAGTCAAGTTTAACTGGTATGTAGATGGGGT |
| | | GGAGGTACACAATGCGAAAACGAAACCGAGAGAGGAGCAGTACAAT |
| | | TCGACGTATAGGGTGGTCAGCGTGCTGACGGTGTTGCACCAGGACT |
| | | GGCTGAACGGGAAAGAGTATAAGTGCAAAGTGTCGAACAAGGCCCT |
| | | CCCCGCACCCATCGAAAAGACGATATCCAAAGCCAAGGGCCAACCG |
| | | CGCGAGCCGCAAGTGTACACGCTGCCTCCCTCGCGAGAAGAGATGA |
| | | CCAAGAACCAGGTGTCCCTTACGTGCTTGGTGAAAGGATTCTACCC |
| | | TTCGGACATCGCCGTAGAATGGGAAAGCAATGGGCAGCCAGAGAAC |
| | | AATTACAAAACCACACCGCCTGTGCTCGACTCGGACGGTTCCTTTT |
| | | TCTTGTATTCCAAGTTGACAGTGGACAAGTCACGGTGGCAACAGGG |
| | | GAACGTATTCTCGTGTTCCGTCATGCACGAAGCGCTGCATAACCAC |
| | | TACACTCAGAAGTCGCTAAGCTTGTCGCCGGGTCATCATCATCATC |
| | | ATCATTAA |
| 46 | Signal peptide | ATGGGCACTCCAGCTCAGTTGCTGTTCCTCCTTCTTCTTTGGCTCC |
| | | CAGACACTACCGGT |
| 47 | ABV2 VHCDR1 | TFGMS |
| | (Kabat) | |
| 48 | ABV2 VHCDR2 | WINTYSGVPTYADDFKG |
| | (Kabat) | |
| 49 | ABV2 VHCDR3 | RSNFAY |
| | (Kabat) | |
| 50 | ABV2 VLCDR1 | RASESVDSSGNSFMH |
| | (Kabat) | |
| 51 | ABV2 VLCDR2 | RASNLES |
| | (Kabat) | |
| 52 | ABV2 VLCDR3 | QQSNEDPWT |
| | (Kabat) | |
| 53 | ABV2 VHCDR1 | GYTFTTF |
| | (Chothia) | |
| 54 | ABV2 VHCDR2 | NTYSGV |
| | (Chothia) | |
| 55 | ABV2 VHCDR3 | RSNFAY |
| | (Chothia) | |
| 56 | ABV2 VLCDR1 | RASESVDSSGNSFMH |
| | (Chothia) | |
| 57 | ABV2 VLCDR2 | RASNLES |
| | (Chothia) | |
| 58 | ABV2 VLCDR3 | QQSNEDPWT |
| | (Chothia) | |
| 59 | ABV2 VHCDR1 | GYTFTTFGMS |
| | (enhanced Chothia) | |
| 60 | ABV2 VHCDR2 | WINTYSGVPT |
| | (enhanced Chothia) | |
| 61 | ABV2 VHCDR3 | RSNFAY |
| | (enhanced Chothia) | |
| 62 | ABV2 VLCDR1 | RASESVDSSGNSFMH |
| | (enhanced Chothia) | |
| 63 | ABV2 VLCDR2 | RASNLES |
| | (enhanced Chothia) | |
| 64 | ABV2 VLCDR3 | QQSNEDPWT |
| | (enhanced Chothia) | |
| 65 | ABV2 VHCDR1 | GYTFTTF |
| | (IMGT) | |
| 66 | ABV2 VHCDR2 | INTYSGVP |
| | (IMGT) | |
| 67 | ABV2 VHCDR3 | ARRSNFAY |
| | (IMGT) | |
| 68 | ABV2 VLCDR1 | ESVDSSGNSF |
| | (IMGT) | |
| 69 | ABV2 VLCDR2 | RAS |
| | (IMGT) | |
| 70 | ABV2 VLCDR3 | CQQSNEDPWT |
| | (IMGT) | |
| 71 | ABV2 VH | QTQLVQSGPELKKPGETVKISCKASGYTFTTFGMSWVKQAPGKGLK |
| | | WMGWINTYSGVPTYADDFKGRFAFSLETSASTAYLQINNLKNEDTA |
| | | TYFCARRSNFAYWGQGTLVTVSA |
| 72 | ABV2 VL | DIVLTQSPASLAVSLGQRATISCRASESVDSSGNSFMHWYQQKAGQ |
| | | SFKLLIYRASNLESGIPARFSGSGSRTDFTLTINPVEADDVATYYC |
| | | QQSNEDPWTFGGGTKLEIK |
| 73 | ABV2 VH_hum#1 | QTQLVQSGAEVKKPGSSVKVSCRASGYTFTTFGMSWVRQAPGQGLE |
| | | WMGWINTYSGVPTYAQNFQGRFAFTVDTEASTAYMELRSLKSEDSA |
| | | VYFCARRSNFAYWGQGTTVTVSS |
| 74 | ABV2 VH_hum#2 | QVQLVQSGSELKKPGASVKVSCKASGYTFTTFGMSWVRQAPGQGLE |
| | | WMGWINTYSGVPTYAQGFTGRFVFSLDTSVSTAYLQISSLKAEDTA |
| | | VYFCARRSNFAYWGQGTLVTVSS |
| 75 | ABV2 VH_hum#3 | QVQLVESGGGLVQPGGSLKLSCAASGYTFTTFGMSWVRQASGKGLE |
| | | WMGWINTYSGVPTYAASMRGRFTFSLDTSKNTAFLQMNSLKSDDTA |
| | | MYFCARRSNFAYWGQGTLVTVSS |
| 76 | ABV2 VH_hum#4 | QTQLVQSGAEVKKPGSSVKVSCKASGYTFTTFGMSWVRQAPGQGLE |
| | | WMGWINTYSGVPTYAQKFQGRFTFTLDTSTSTAYMELSSLRSEDTA |
| | | VYFCARRSNFAYWGQGTLVTVSS |
| 77 | ABV2 VH_hum#5 | ETQLVQSGAEVKKPGESLKISCQAFGYTFTTFGMSWVRQMPGQGLE |
| | | WMGWINTYSGVPTYNENFKGQFTFSLDTSSSTAYLQWSSLKASDTA |
| | | MYFCARRSNFAYWGQGTMVTVSS |
| 78 | ABV2 VH_hum#6 | EVQLLESGGGLVQPGGSLRLSCAASGYTFTTFGMSWVRQAPGKGLE |
| | | WMGWINTYSGVPTYNENFKGRFTFSVDTSKNTAYLQMNSLRAEDTA |
| | | VYFCARRSNFAYWGQGTMVTVSS |
| 79 | ABV2 VH_hum#7 | QTQLVQSGAEVKKPGASVKVSCKASGYTFTTFGMSWVRQAPGQGLE |
| | | WMGWINTYSGVPTYNEKFKSKFTFTLDTSTNTAYMELSSLRSEDTA |
| | | VYFCARRSNFAYWGQGTLVTVSS |
| 80 | ABV2 VH_hum#8 | QTQLVQSGAEVKKPGASVKVSCKASGYTFTTFGMSWVRQAPGQGLE |
| | | WMGWINTYSGVPTYAQKFQGRFTFTLDTSTSTAYMELSSLRSEDTA |
| | | VYFCARRSNFAYWGQGTLVTVSS |
| 81 | ABV2 VH_hum#9 | EIQLVESGGGLVQPGGSLRLSCAASGYTFTTFGMSWVRQAPGKGLE |
| | | WMGWINTYSGVPTYADSVKGRFTFSLDTSKNTAYLQMNSLRAEDTA |
| | | VYFCARRSNFAYWGQGTLVTVSS |
| 82 | ABV2 VH_hum#10 | QVQLVQSGSELKKPGASVKVSCKASGYTFTTFGMSWVRQAPGQGLE |
| | | WMGWINTYSGVPTYADDFKGRFVFSLDTSVSTAYLQISSLKAEDTA |
| | | VYYCARRSNFAYWGAGTTVTVSS |
| 83 | ABV2 VH_hum#11 | QVQLVQSGAEVKKPGASVKVSCKASGYTFTTFGMSWVRQAPGQGLE |
| | | WMGWINTYSGVPTYADDFKGRVTMTTDTSTSTAYMELRSLRSDDTA |
| | | VYYCARRSNFAYWGAGTTVTVSS |
| 84 | ABV2 VL_hum#12 | QVQLVQSGAEVKKPGSSVKVSCKASGYTFTTFGMSWVRQAPGQGLE |
| | | WMGWINTYSGVPTYAQKFQGRVTITADESTSTAYMELSSLRSEDTA |
| | | VYYCARRSNFAYWGAGTTVTVSS |
| 85 | ABV2 VL_hum#1 | EIVLMQSPGTLSLSPGERATLSCRASESVDSSGNSFMHWYQQKPGQ |
| | | AFRLLIYRASNLESGIPDRFSGSGSRTDATLTISRLEPEDFAVYYC |
| | | QQSNEDPWTFGQGTKVEIK |
| 86 | ABV2 VL_hum#2 | DIVLTQSPDSLAVSLGERATINCRASESVDSSGNSFMHWYQQKPGQ |
| | | PPKLLIYRASNLESGVPDRFSGSGSRTDFTLTISSLQAEDVAVYYC |
| | | QQSNEDPWTFGGGTKVEIK |
| 87 | ABV2 VL_hum#3 | DIVLTQSPLSLSVTPGEPASISCRASESVDSSGNSFMHWYLQKPGQ |
| | | SFQLLIYRASNLESGVPDRFSGSGSGTDFTLKIIRVEAEDAGTYYC |
| | | QQSNEDPWTFGQGTRLEIK |
| 88 | ABV2 VL_hum#4 | DIVLTQSPDSLAVSLGERATINCRASESVDSSGNSFMHWYQQKPGQ |
| | | PFKLLIYRASNLESGVPDRFSGSGSRTDFTLTISSLQAEDVAVYYC |
| | | QQSNEDPWTFGQGTRLEIK |
| 89 | ABV2 VL_hum#5 | DIVLTQTPLSLPVTPGEPASISCRASESVDSSGNSFMHWYLQKPGQ |
| | | SFKLLIYRASNLESGVPDRFSGSGSRTDFTLKISRVEAEDVGVYYC |
| | | QQSNEDPWTFGQGTKLEIK |
| 90 | ABV2 VL_hum#6 | DIQLTQSPSTLSASVGDRVTITCRASESVDSSGNSFMHWYQQKPGK |
| | | AFKLLIYRASNLESGVPSRFSGSGSGTEFTLTISSLQPDDFATYYC |
| | | QQSNEDPWTFGQGTKVEIK |
| 91 | ABV2 VL_hum#7 | DIQLTQSPSSLSASVGDRVTITCRASESVDSSGNSFMHWYQQKPGK |
| | | AFKLLIYRASNLESGVPSRFSGSGSRTDFTFTISSLQPEDIATYYC |
| | | QQSNEDPWTFGQGTKVEIK |
| 92 | ABV2 VL_hum#8 | EIVLTQSPGTLSLSPGERATLSCRASESVDSSGNSFMHWYQQKPGQ |
| | | AFRLLIYRASNLESGIPDRFSGSGSRTDFTLTISRLEPEDFAVYYC |
| | | QQSNEDPWTFGQGTKVEIK |
| 93 | ABV2 VL_hum#9 | EIVLTQSPATLSVSPGERATLSCRASESVDSSGNSFMHWYQQKPGQ |
| | | AFRLLIYRASNLESGIPARFSGSGSRTEFTLTISSLQSEDFAVYYC |
| | | QQSNEDPWTFGGGTKVEIK |
| 94 | ABV2 VL_hum#10 | DIQLTQSPSSLSASVGDRVTITCRASESVDSSGNSFMHWYQQKPGK |
| | | AFKLLIYRASNLESGVPSRFSGSGSRTDFTLTISSLQPEDFATYYC |
| | | QQSNEDPWTFGGGTKVEIK |
| 95 | ABV2 VL_hum#11 | DIVLTQSPLSLPVTPGEPASISCRASESVDSSGNSFMHWYLQKPGQ |
| | | SFQLLIYRASNLESGVPDRFSGSGSRTDFTLKISRVEAEDVGVYYC |
| | | QQSNEDPWTFGGGTKVEIK |
| 96 | ABV2 VL_hum#12 | DIVLTQSPDSLAVSLGERATINCRASESVDSSGNSFMHWYQQKPGQ |
| | | PFKLLIYRASNLESGVPDRFSGSGSRTDFTLTISSLQAEDVAVYYC |
| | | QQSNEDPWTFGGGTKVEIK |
| 97 | ABV2 VL_hum#13 | DIVLTQSPDSLAVSLGERATINCRASESVDSSGNSFMHWYQQKAGQ |
| | | SFKLLIYRASNLESGVPDRFSGSGSRTDFTLTISSLQAEDVAVYYC |
| | | QQSNEDPWTFGGGTKVEIK |
| 98 | ABV2 VL_hum#14 | DIVLTQSPASLAVSPGQRATITCRASESVDSSGNSFMHWYQQKPGQ |
| | | PPKLLIYRASNLESGVPARFSGSGSGTDFTLTINPVEANDTANYYC |
| | | QQSNEDPWTFGGGTKLEIK |
| 99 | ABV2 VL_hum#15 | DIVMTQSPDSLAVSLGERATINCRASESVDSSGNSFMHWYQQKPGQ |
| | | PPKLLIYRASNLESGVPDRFSGSGSGTDFTLTISSLQAEDVAVYYC |
| | | QQSNEDPWTFGGGTKLEIK |
| 100 | ABV2 VL_hum#16 | EIVLTQSPATLSLSPGERATLSCRASESVDSSGNSFMHWYQQKPGQ |
| | | APRLLIYRASNLESGIPARFSGSGSGTDFTLTISSLEPEDFAVYYC |
| | | QQSNEDPWTFGGGTKLEIK |
| 101 | Heavy chain of ABV2 | QIQLVQSGPELKKPGETVKISCKASGYTFTTFGMSWVKQAPGKGLK |
| | chimera | WMGWINTYSGVPTYADDFKGRFAFSLETSASTAYLQINNLKNEDTA |
| | | TYFCARRSNFAYWGQGTLVTVSAASTKGPSVFPLAPSSKSTSGGTA |
| | | ALGCLVKDYFPEPVTVSWNSGALTSGVHTFPAVLQSSGLYSLSSVV |
| | | TVPSSSLGTQTYICNVNHKPSNTKVDKKVEPKSCDKTHTCPPCPAP |
| | | ELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVK |
| | | GFYPSDIAVEWESNGQPENNYKTTPPVLDSDGSFFLYSKLTVDKSR |
| | | WQQGNVFSCSVMHEALHNHYTQKSLSLSPG |
| 102 | Light chain of ABV2 | DIVLTQSPASLAVSLGQRATISCRASESVDSSGNSFMHWYQQKAGQ |
| | chimera | SFKLLIYRASNLESGIPARFSGSGSRTDFTLTINPVEADDVATYYC |
| | | QQSNEDPWTFGGGTKLEIKRTVAAPSVFIFPPSDEQLKSGTASVVC |
| | | LLNNFYPREAKVQWKVDNALQSGNSQESVTEQDSKDSTYSLSSTLT |
| | | LSKADYEKHKVYACEVTHQGLSSPVTKSFNRGEC |
| 103 | Human IgG1 heavy | ASTKGPSVFPLAPSSKSTSGGTAALGCLVKDYFPEPVTVSWNSGALTSGVH |
| | chain | TFPAVLQSSGLYSLSSVVTVPSSSLGTQTYICNVNHKPSNTKVDKKVEPKS |
| | | CDKTHTCPPCPAPELLGGPSVFLFPPKPKDTLMISRTPEVTCVVVDVSHED |
| | | PEVKFNWYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNVF |
| | | SCSVMHEALHNHYTQKSLSLSPG |
| 104 | Human TNFR2 | LPAQVAFTPYAPEPGSTCRLREYYDQTAQMCCSKCSPGQHAKVFCT |
| | without leader | KTSDTVCDSCEDSTYTQLWNWVPECLSCGSRCSSDQVETQACTREQ |
| | sequence | NRICTCRPGWYCALSKQEGCRLCAPLRKCRPGFGVARPGTETSDVV |
| | | CKPCAPGTFSNTTSSTDICRPHQICNVVAIPGNASMDAVCTSTSPT |
| | | RSMAPGAVHLPQPVSTRSQHTQPTPEPSTAPSTSFLLPMGPSPPAE |
| | | GSTGDFALPVGLIVGVTALGLLIIGVVNCVIMTQVKKKPLCLQREA |
| | | KVPHLPADKARGTQGPEQQHLLITAPSSSSSSLESSASALDRRAPT |
| | | RNQPQAPGVEASGAGEARASTGSSDSSPGGHGTQVNVTCIVNVCSS |
| | | SDHSSQCSSQASSTMGDTDSSPSESPKDEQVPFSKEECAFRSQLET |
| | | PETLLGSTEEKPLPLGVPDAGMKPS |
| 105 | Mouse TNFR2 | VPAQVVLTPYKPEPGYECQISQEYYDRKAQMCCAKCPPGQYVKHFC |
| | without leader | NKTSDTVCADCEASMYTQVWNQFRTCLSCSSSCTTDQVEIRACTKQ |
| | sequence | QNRVCACEAGRYCALKTHSGSCRQCMRLSKCGPGFGVASSRAPNGN |
| | | VLCKACAPGTFSDTTSSTDVCRPHRICSILAIPGNASTDAVCAPES |
| | | PTLSAIPRTLYVSQPEPTRSQPLDQEPGPSQTPSILTSLGSTPIIE |
| | | QSTKGGISLPIGLIVGVTSLGLLMLGLVNCIILVQRKKKPSCLQRD |
| | | AKVPHVPDEKSQDAVGLEQQHLLTTAPSSSSSSLESSASAGDRRAP |
| | | PGGHPQARVMAEAQGFQEARASSRISDSSHGSHGTHVNVTCIVNVC |
| | | SSSDHSSQCSSQASATVGDPDAKPSASPKDEQVPFSQEECPSQSPC |
| | | ETTETLQSHEKPLPLGVPDMGMKPSQAGWFDQIAVKVA |
| 106 | ABV2.7 HC | QVQLVQSGSELKKPGASVKVSCKASGYTFTTFGMSWVRQAPGQGLEWMGWI |
| | | NTYSGVPTYAQGFTGRFVFSLDTSVSTAYLQISSLKAEDTAVYFCARRSNF |
| | | AYWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHKP |
| | | SNTKVDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYSKL |
| | | TVDKSRWQQGNVFSCSVMHEALHNHYTQKSLSLSPG |
| 107 | ABV2.7 LC | DIVLTQSPDSLAVSLGERATINCRASESLTASGNSFMHWYQQKPGQPPKLL |
| | | IYRASNLESGVPDRFSGSGSRTDFTLTISSLQAEDVAVYYCQQSRHVNWTF |
| | | GGGTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTHQG |
| | | LSSPVTKSFNRGEC |
| 108 | ABV2.7 VHCDR1 | GYTFTTF |
| | (Chothia) | |
| 109 | ABV2.7 VHCDR2 | NTYSGV |
| | (Chothia) | |
| 110 | ABV2.7 VHCDR3 | RSNFAY |
| | (Chothia) | |
| 111 | ABV2.7 VLCDR1 | RASESLTASGNSFMH |
| | (Chothia) | |
| 112 | ABV2.7 VLCDR2 | RASNLES |
| | (Chothia) | |
| 113 | ABV2.7 VLCDR3 | QQSRHVNWT |
| | (Chothia) | |
| 114 | ABV2.7 VHCDR1 | TFGMS |
| | (Kabat) | |
| 115 | ABV2.7 VHCDR2 | WINTYSGVPTYAQGFTG |
| | (Kabat) | |
| 116 | ABV2.7 VHCDR3 | RSNFAY |
| | (Kabat) | |
| 117 | ABV2.7 VLCDR1 | RASESLTASGNSFMH |
| | (Kabat) | |
| 118 | ABV2.7 VLCDR2 | RASNLES |
| | (Kabat) | |
| 119 | ABV2.7 VLCDR3 | QQSRHVNWT |
| | (Kabat) | |
| 120 | ABV2.7 VHCDR1 | GYTFTTFG |
| | (IMGT) | |
| 121 | ABV2.7 VHCDR2 | INTYSGVP |
| | (IMGT) | |
| 122 | ABV2.7 VHCDR3 | ARRSNFAY |
| | (IMGT) | |
| 123 | ABV2.7 VLCDR1 | ESLTASGNSF |
| | (IMGT) | |
| 124 | ABV2.7 VLCDR2 | PAS |
| | (IMGT) | |
| 125 | ABV2.7 VLCDR3 | QQSRHVNWT |
| | (IMGT) | |
| 126 | ABV2.7 VH | QVQLVQSGSELKKPGASVKVSCKASGYTFTTFGMSWVRQAPGQGLEWMGWI |
| | | NTYSGVPTYAQGFTGRFVFSLDTSVSTAYLQISSLKAEDTAVYFCARRSNF |
| | | AYWGQGTLVTVSS |
| 127 | ABV2.7 VL | DIVLTQSPDSLAVSLGERATINCRASESLTASGNSFMHWYQQKPGQPPKLL |
| | | IYRASNLESGVPDRFSGSGSRTDFTLTISSLQAEDVAVYYCQQSRHVNWTF |
| | | GGGTKVEIK |
| 128 | ABV2.13 HC | QVQLVQSGSELKKPGASVKVSCKASGYTFTTFGMSWVRQAPGQGLEWMGWI |
| | | NTYSGVPHYAQGFTGRFVFSLDTSVSTAYLQISSLKAEDTAVYFCARRSNF |
| | | AYWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHKP |
| | | SNTKVDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYSKL |
| | | TVDKSRWQQGNVFSCSVMHEALHNHYTQKSLSLSPG |
| 129 | ABV2.13 LC | DIVLTQSPDSLAVSLGERATINCRASQTVDSSGNSFMHWYQQKPGQPPKLL |
| | | IYLGNRLESGVPDRFSGSGSRTDFTLTISSLQAEDVAVYYCQQSNEDPWTF |
| | | GGGTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTHQG |
| | | LSSPVTKSFNRGEC |
| 130 | ABV2.13 VHCDR1 | GYTFTTF |
| | (Chothia) | |
| 131 | ABV2.13 VHCDR2 | NTYSGV |
| | (Chothia) | |
| 132 | ABV2.13 VHCDR3 | RSNFAY |
| | (Chothia) | |
| 133 | ABV2.13 VLCDR1 | RASQTVDSSGNSFMH |
| | (Chothia) | |
| 134 | ABV2.13 VLCDR2 | LGNRLES |
| | (Chothia) | |
| 135 | ABV2.13 VLCDR3 | QQSNEDPWT |
| | (Chothia) | |
| 136 | ABV2.13 VHCDR1 | TFGMS |
| | (Kabat) | |
| 137 | ABV2.13 VHCDR2 | WINTYSGVPHYAQGFTG |
| | (Kabat) | |
| 138 | ABV2.13 VHCDR3 | RSNFAY |
| | (Kabat) | |
| 139 | ABV2.13 VLCDR1 | RASQTVDSSGNSFMH |
| | (Kabat) | |
| 140 | ABV2.13 VLCDR2 | LGNRLES |
| | (Kabat) | |
| 141 | ABV2.13 VLCDR3 | QQSNEDPWT |
| | (Kabat) | |
| 142 | ABV2.13 VHCDR1 | GYTFTTFG |
| | (IMGT) | |
| 143 | ABV2.13 VHCDR2 | INTYSGVP |
| | (IMGT) | |
| 144 | ABV2.13 VHCDR3 | ARRSNFAY |
| | (IMGT) | |
| 145 | ABV2.13 VLCDR1 | QTVDSSGNSF |
| | (IMGT) | |
| 146 | ABV2.13 VLCDR2 | LGN |
| | (IMGT) | |
| 147 | ABV2.13 VLCDR3 | QQSNEDPWT |
| | (IMGT) | |
| 148 | ABV2.13 VH | QVQLVQSGSELKKPGASVKVSCKASGYTFTTFGMSWVRQAPGQGLEWMGWI |
| | | NTYSGVPHYAQGFTGRFVFSLDTSVSTAYLQISSLKAEDTAVYFCARRSNF |
| | | AYWGQGTLVTVSS |
| 149 | ABV2.13 VL | DIVLTQSPDSLAVSLGERATINCRASQTVDSSGNSFMHWYQQKPGQPPKLL |
| | | IYLGNRLESGVPDRFSGSGSRTDFTLTISSLQAEDVAVYYCQQSNEDPWTF |
| | | GGGTKVEIK |
| 150 | ABV2.15 HC | QVQLVQSGSELKKPGASVKVSCKASGYTFTTFGMSWVRQAPGQGLEWMGWI |
| | | NTYSGVPHYAQGFTGRFVFSLDTSVSTAYLQISSLKAEDTAVYFCARRSNF |
| | | AYWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHKP |
| | | SNTKVDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYSKL |
| | | TVDKSRWQQGNVFSCSVMHEALHNHYTQKSLSLSPG |
| 151 | ABV2.15 LC | DIVLTQSPDSLAVSLGERATINCRASESLTASGNSFMHWYQQKPGQPPKLL |
| | | IYRASNLESGVPDRFSGSGSRTDFTLTISSLQAEDVAVYYCQQSRHVNWTF |
| | | GGGTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTHQG |
| | | LSSPVTKSFNRGEC |
| 152 | ABV2.15 VHCDR1 | GYTFTTF |
| | (Chothia) | |
| 153 | ABV2.15 VHCDR2 | NTYSGV |
| | (Chothia) | |
| 154 | ABV2.15 VHCDR3 | RSNFAY |
| | (Chothia) | |
| 155 | ABV2.15 VLCDR1 | RASESLTASGNSFMH |
| | (Chothia) | |
| 156 | ABV2.15 VLCDR2 | RASNLES |
| | (Chothia) | |
| 157 | ABV2.15 VLCDR3 | QQSRHVNWT |
| | (Chothia) | |
| 158 | ABV2.15 VHCDR1 | TFGMS |
| | (Kabat) | |
| 159 | ABV2.15 VHCDR2 | WINTYSGVPHYAQGFTG |
| | (Kabat) | |
| 160 | ABV2.15 VHCDR3 | RSNFAY |
| | (Kabat) | |
| 161 | ABV2.15 VLCDR1 | RASESLTASGNSFMH |
| | (Kabat) | |
| 162 | ABV2.15 VLCDR2 | RASNLES |
| | (Kabat) | |
| 163 | ABV2.15 VLCDR3 | QQSRHVNWT |
| | (Kabat) | |
| 164 | ABV2.15 VHCDR1 | GYTFTTFG |
| | (IMGT) | |
| 165 | ABV2.15 VHCDR2 | INTYSGVP |
| | (IMGT) | |
| 166 | ABV2.15 VHCDR3 | ARRSNFAY |
| | (IMGT) | |
| 167 | ABV2.15 VLCDR1 | ESLTASGNSF |
| | (IMGT) | |
| 168 | ABV2.15 VLCDR2 | PAS |
| | (IMGT) | |
| 169 | ABV2.15 VLCDR3 | QQSRHVNWT |
| | (IMGT) | |
| 170 | ABV2.15 VH | QVQLVQSGSELKKPGASVKVSCKASGYTFTTFGMSWVRQAPGQGLEWMGWI |
| | | NTYSGVPHYAQGFTGRFVFSLDTSVSTAYLQISSLKAEDTAVYFCARRSNF |
| | | AYWGQGTLVTVSS |
| 171 | ABV2.15 VL | DIVLTQSPDSLAVSLGERATINCRASESLTASGNSFMHWYQQKPGQPPKLL |
| | | IYRASNLESGVPDRFSGSGSRTDFTLTISSLQAEDVAVYYCQQSRHVNWTF |
| | | GGGTKVEIK |

### EQUIVALENTS

Those skilled in the art will recognize, or be able to ascertain using no more than routine experimentation, many equivalents of the specific embodiments disclosed herein. Such equivalents are intended to be encompassed by the following claims.

1. An isolated antibody that:
(a) binds all or a portion of amino acid residues 23-54 of human TNFR2 (SEQ ID NO: 1), and does not bind one or more amino acid residues within 55-77 of human TNFR2 (SEQ ID NO: 1);
(b) binds all or a portion of amino acid residues 23-54 of human TNFR2 (SEQ ID NO: 1), and does not bind one or more amino acid residues within 60-77, 65-77, 70-77, 75-77, 55-75, 55-70, 55-65, or 55-60 of human TNFR2 (SEQ ID NO: 1);
(c) binds all or a portion of amino acid residues 23-54 of human TNFR2 (SEQ ID NO: 1), and does not bind one or more amino acid residues within 70-77 of human TNFR2 (SEQ ID NO: 1);
(d) binds to TNFR2 chimera 3 (SEQ ID NO: 11 or 12), and does not bind TNFR2 chimera 0 (SEQ ID NO: 5 or 6);
(e) exhibits reduced binding to a mutant human TNFR2 comprising a substitution at one or more amino acid residues selected from the group consisting of residues 48 and 68 of human TNFR2 (SEQ ID NO: 1), as compared to wild-type human TNFR2 (SEQ ID NO: 1);
(f) binds all or a portion of amino acid residues 55-96 of human TNFR2 (SEQ ID NO: 1), and does not significantly inhibit binding of TNF-alpha to human TNFR2;
(g) binds all or a portion of amino acid residues 55-96 of human TNFR2 (SEQ ID NO: 1), and does not bind one or more amino acid residues within 23-54 of human TNFR2 (SEQ ID NO: 1);
(h) binds all or a portion of amino acid residues 55-96 of human TNFR2 (SEQ ID NO: 1), and does not bind one or more amino acid residues within 23-44, 23-36, 23-30, 23-25, 25-44, 30-44, 35-44, or 40-44 of human TNFR2 (SEQ ID NO: 1);
(i) exhibits reduced binding to a mutant human TNFR2 comprising a substitution at one or more amino acid residues selected from the group consisting of residues 37, 44, 51, 52, 55, 58, 59, 61, 62, 72, 74, 76, 78, and 87 of human TNFR2 (SEQ ID NO: 1), as compared to wild-type human TNFR2 (SEQ ID NO: 1);
(j) binds TNFR2 chimera 7 (SEQ ID NO: 19 or 20), and does not bind TNFR2 chimera 4 (SEQ ID NO: 13 or 14) (e.g., does not bind TNFR2 chimera 4 with a K_{D} of less than 1×10⁻⁷ M);
(k) binds all or a portion of amino acid residues 78-118 of human TNFR2 (SEQ ID NO: 1), and does not bind one or more amino acid residues within 23-77 or 119-200 of human TNFR2 (SEQ ID NO: 1);
(l) binds TNFR2 chimera 1 (SEQ ID NO: 7 or 8) and does not bind TNFR2 chimera 2 (SEQ ID NO: 9 or 10) (e.g., does not bind TNFR2 chimera 2 (SEQ ID NO: 9 or 10) with a K_{D} of less than 1×10⁻⁷ M);
(m) binds all or a portion of amino acid residues 120-257 of human TNFR2 (SEQ ID NO: 1), and does not significantly inhibit binding of TNF-alpha to human TNFR2;
(n) binds all or a portion of amino acid residues 120-257 of human TNFR2 (SEQ ID NO: 1), does not bind one or more amino acid residues within 78-118 of human TNFR2 (SEQ ID NO: 1), and does not inhibit the binding of TNF-alpha to human TNFR2; or
(o) binds to one or more of the following positions on human TNFR2: Y24, Q26, Q29, M30, and K47, wherein the numbering is according to SEQ ID NO: 104;
optionally wherein the antibody is modified to enhance its effector function relative to the same antibody in unmodified form, and optionally wherein the antibody agonizes TNFR2 activity.

2-35. (canceled)

36. An isolated antibody which binds to human TNFR2 comprising:
(a) a heavy chain CDR1, CDR2, and CDR3 sequences comprising SEQ ID NOs: 152, 153, and 154, respectively, and light chain CDR1, CDR2, and CDR3 sequences comprising SEQ ID NOs: 155, 156, and 157, respectively;
(b) a heavy chain CDR1, CDR2, and CDR3 sequences comprising SEQ ID NOs: 158, 159, and 160, respectively, and light chain CDR1, CDR2, and CDR3 sequences comprising SEQ ID NOs: 161, 162, and 163, respectively;
(c) a heavy chain CDR1, CDR2, and CDR3 sequences comprising SEQ ID NOs: 164, 165, and 166, respectively, and light chain CDR1, CDR2, and CDR3 sequences comprising SEQ ID NOs: 167, 168, and 169, respectively;
(d) a heavy chain CDR1, CDR2, and CDR3 sequences comprising SEQ ID NOs: 47, 48, and 49, respectively, and light chain CDR1, CDR2, and CDR3 sequences comprising SEQ ID NOs: 50, 51, and 52, respectively;
(e) a heavy chain CDR1, CDR2, and CDR3 sequences comprising SEQ ID NOs: 53, 54, and 55, respectively, and light chain CDR1, CDR2, and CDR3 sequences comprising SEQ ID NOs: 56, 57, and 58, respectively;
(f) a heavy chain CDR1, CDR2, and CDR3 sequences comprising SEQ ID NOs: 59, 60, and 61, respectively, and light chain CDR1, CDR2, and CDR3 sequences comprising SEQ ID NOs: 62, 63, and 64, respectively;
(g) a heavy chain CDR1, CDR2, and CDR3 sequences comprising SEQ ID NOs: 65, 66, and 67, respectively, and light chain CDR1, CDR2, and CDR3 sequences comprising SEQ ID NOs: 68, 69, and 70, respectively;
(h) a heavy chain CDR1, CDR2, and CDR3 sequences comprising SEQ ID NOs: 130, 131, and 132, respectively, and light chain CDR1, CDR2, and CDR3 sequences comprising SEQ ID NOs: 133, 134, and 135, respectively; or
(i) a heavy chain CDR1, CDR2, and CDR3 sequences comprising SEQ ID NOs: 108, 109, and 110, respectively, and light chain CDR1, CDR2, and CDR3 sequences comprising SEQ ID NOs: 111, 112, and 113, respectively.

37-39. (canceled)

40. The isolated antibody of claim 1, which comprises heavy and light chain variable region sequences which are at least 95% identical to the amino acid sequences set forth in (a) SEQ ID NOs: 170 and 171, respectively; (b) SEQ ID NOs: 71 and 72, respectively, (c) SEQ ID NOs: 74 and 86, respectively; (d) SEQ ID NOs: 148 and 149, respectively; or (e) SEQ ID NOs: 126 and 127, respectively.

41. (canceled)

42. The isolated antibody of claim 1, which comprises heavy and light chain sequences which are at least 95% identical to the amino acid sequences set forth in (a) SEQ ID NOs: 150 and 151, respectively, (b) SEQ ID NOs: 128 and 129, respectively, (c) SEQ ID NOs: 106 and 107, respectively, or (d) SEQ ID NOs: 101 and 102, respectively.

43-44. (canceled)

45. The isolated antibody of claim 36, wherein the antibody is selected from the group consisting of an IgG1, an IgG2, an IgG3, and an IgG4, or variant thereof.

46. The isolated antibody of claim 45, wherein the antibody comprises a variant Fc region, wherein the variant Fc region optionally increases binding to Fcγ receptors relative to binding observed with the corresponding non-variant Fc region.

47-51. (canceled)

52. The isolated antibody of claim 46, wherein the variant Fc region is a variant IgG1 Fc region, wherein the variant IgG1 Fc region optionally comprises a substitution or substitutions selected from the group consisting of:
(a) S267E,
(b) S267E/L328F,
(c) G237D/P238D/P271G/A330R,
(d) E233D/P238D/H268D/P271G/A330R,
(e) G237D/P238D/H268D/P271G/A330R, and
(f) E233D/G237D/P238D/H268D/P271G/A330R.

53. (canceled)

54. The antibody of claim 36, wherein the antibody:
(a) activates NF-κB signaling,
(b) promotes T cell proliferation,
(c) co-stimulates T cells,
(d) promotes CD4+ and CD8+ T cell proliferation,
(e) decreases the abundance of regulatory T cells,
(f) induces a long-term anti-cancer effect, and/or
(g) induces the development of anti-cancer memory T cells.

55-60. (canceled)

61. The isolated antibody of claim 36, wherein the antibody is a single-chain antibody, Fab, Fab′, F(ab′)2, Fd, Fv, or a domain antibody.

62. The antibody of claim 36, wherein the antibody is a human, humanized, or chimeric antibody.

63-65. (canceled)

66. A bispecific antibody comprising the antigen binding region of the antibody of claim 36, and a second different antigen binding region.

67. An immunoconjugate comprising the antibody of claim 36, linked to an agent.

68. A nucleic acid encoding the heavy and/or light chain variable region of the antibody of claim 36.

69. An expression vector comprising the nucleic acid molecule of claim 68.

70. A cell transformed with the expression vector of claim 69.

71. A composition comprising the antibody of claim 36, and a carrier.

72. (canceled)

73. A method of preparing an anti-TNFR2 antibody comprising expressing the antibody in the cell of claim 70 and isolating the antibody, or antigen binding portion thereof, from the cell.

74. A method of increasing T cell proliferation, co-stimulating an effector T cell, and/or reducing or depleting the number of regulatory T cells in a subject comprising administering an effective amount of the antibody of claim 36.

75-76. (canceled)

77. A method of treating cancer comprising administering to a subject in need thereof a therapeutically effective amount of
(a) the antibody of claim 36,
(b) an anti-TNFR2 antibody, wherein the antibody has effector function and does not significantly inhibit binding of TNF-alpha to TNFR2,
(c) an anti-TNFR2 antibody, wherein the antibody has effector function and agonizes TNFR2 receptor signaling, or
(d) an anti-TNFR2 antibody, wherein the antibody has effector function.

78-79. (canceled)

80. The method of claim 77, wherein the cancer is selected from the group consisting of: non-small cell lung cancer, breast cancer, ovarian cancer, and colorectal cancer.

81. The method of claim 77, further comprising administering one or more additional therapeutic agents.

82-100. (canceled)

101. A method of treating an autoimmune disease, promoting graft survival or reducing graft rejection, or treating, preventing, or reducing graft-versus-host disease comprising administering to a subject in need thereof a therapeutically effective amount of the antibody of claim 36.

102-115. (canceled)

116. A method of detecting the presence of TNFR2 in a sample comprising contacting the sample with the antibody of claim 36, under conditions that allow for formation of a complex between the antibody and TNFR2, and detecting the complex.


SEQUENCE LISTING (171 sequences)

SEQ ID NO: 1 - protein, 461 aa, Homo sapiens
  misc_feature: Human TNFR2
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 2 - protein, 235 aa, Homo sapiens
  misc_feature: Human TNFR2
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 3 - protein, 474 aa, Mus musculus
  misc_feature: Mouse TNFR2
MAPAALWVALVFELQLWATGHTVPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGISLPIGLIVGVTSLGLLMLGLVNCIILVQRKKKPSCLQRDAKVPHVPDEKSQDAVGLEQQHLLTTAPSSSSSSLESSASAGDRRAPPGGHPQARVMAEAQGFQEARASSRISDSSHGSHGTHVNVTCIVNVCSSSDHSSQCSSQASATVGDPDAKPSASPKDEQVPFSQEECPSQSPCETTETLQSHEKPLPLGVPDMGMKPSQAGWFDQIAVKVA

SEQ ID NO: 4 - protein, 236 aa, Mus musculus
  misc_feature: Mouse TNFR2 (extracellular domain)
VPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGG

SEQ ID NO: 5 - protein, 249 aa, Artificial Sequence
  Synthetic TNFR2 Chimera 0
VPAQVVLTPYKPEPGYECQISQEYYDRKAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDIEGRMDPHHHHHH

SEQ ID NO: 6 - protein, 478 aa, Artificial Sequence
  Synthetic TNFR2 Chimera 0  Fc fusion
VPAQVVLTPYKPEPGYECQISQEYYDRKAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDIEGRMDPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGHHHHHH

SEQ ID NO: 7 - protein, 249 aa, Artificial Sequence
  Synthetic TNFR2 Chimera 1
VPAQVVLTPYKPEPGYECQISQEYYDRKAQMCCAKCPPGQYVKHFCNKTSDTVCADCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDIEGRMDPHHHHHH

SEQ ID NO: 8 - protein, 478 aa, Artificial Sequence
  Synthetic TNFR2 Chimera 1  Fc fusion
VPAQVVLTPYKPEPGYECQISQEYYDRKAQMCCAKCPPGQYVKHFCNKTSDTVCADCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDIEGRMDPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGHHHHHH

SEQ ID NO: 9 - protein, 249 aa, Artificial Sequence
  Synthetic TNFR2 Chimera 2
VPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDIEGRMDPHHHHHH

SEQ ID NO: 10 - protein, 478 aa, Artificial Sequence
  Synthetic TNFR2 Chimera 2  Fc fusion
VPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDIEGRMDPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGHHHHHH

SEQ ID NO: 11 - protein, 248 aa, Artificial Sequence
  Synthetic TNFR2 Chimera 3
LPAQVAFTPYAPEPGSTCRLREYYDQTAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGIEGRMDPHHHHHH

SEQ ID NO: 12 - protein, 477 aa, Artificial Sequence
  Synthetic TNFR2 Chimera 3  Fc fusion
LPAQVAFTPYAPEPGSTCRLREYYDQTAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGIEGRMDPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGHHHHHH

SEQ ID NO: 13 - protein, 248 aa, Artificial Sequence
  Synthetic TNFR2 Chimera 4
LPAQVAFTPYAPEPGSTCRLREYYDQTAQMCCSKCSPGQHAKVFCTKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGIEGRMDPHHHHHH

SEQ ID NO: 14 - protein, 477 aa, Artificial Sequence
  Synthetic TNFR2 Chimera 4  Fc fusion
LPAQVAFTPYAPEPGSTCRLREYYDQTAQMCCSKCSPGQHAKVFCTKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGIEGRMDPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGHHHHHH

SEQ ID NO: 15 - protein, 247 aa, Artificial Sequence
  Synthetic TNFR2 Chimera 5
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCAPESPTLSAIPRTLYVSQPEPTRSQPLDQEPGPSQTPSILTSLGSTPIIEQSTKGGIEGRMDPHHHHHH

SEQ ID NO: 16 - protein, 476 aa, Artificial Sequence
  Synthetic TNFR2 Chimera 5  Fc fusion
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCAPESPTLSAIPRTLYVSQPEPTRSQPLDQEPGPSQTPSILTSLGSTPIIEQSTKGGIEGRMDPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGHHHHHH

SEQ ID NO: 17 - protein, 250 aa, Artificial Sequence
  Synthetic TNFR2 Chimera 6
VPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCTSTSPTRSMAPGAVHLPQPVSTRSQHTQPTPEPSTAPSTSFLLPMGPSPPAEGSTGDIEGRMDPHHHHHH

SEQ ID NO: 18 - protein, 479 aa, Artificial Sequence
  Synthetic TNFR2 Chimera 6  Fc fusion
VPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCTSTSPTRSMAPGAVHLPQPVSTRSQHTQPTPEPSTAPSTSFLLPMGPSPPAEGSTGDIEGRMDPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGHHHHHH

SEQ ID NO: 19 - protein, 248 aa, Artificial Sequence
  Synthetic TNFR2 Chimera 7
LPAQVAFTPYAPEPGSTCRLREYYDQTAQMCCSKCSPGQHAKVFCTKTSDTVCDSCEDSTYTQLWNWVPECLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGIEGRMDPHHHHHH

SEQ ID NO: 20 - protein, 477 aa, Artificial Sequence
  Synthetic TNFR2 Chimera 7  Fc fusion
LPAQVAFTPYAPEPGSTCRLREYYDQTAQMCCSKCSPGQHAKVFCTKTSDTVCDSCEDSTYTQLWNWVPECLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGIEGRMDPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGHHHHHH

SEQ ID NO: 21 - protein, 249 aa, Artificial Sequence
  Synthetic TNFR2 Chimera 8
VPAQVVLTPYKPEPGYECQISQEYYDRKAQMCCAKCPPGQYVKHFCNKTSDTVCADCEASMYTQVWNQFRTCLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDIEGRMDPHHHHHH

SEQ ID NO: 22 - protein, 478 aa, Artificial Sequence
  Synthetic TNFR2 Chimera 8  Fc fusion
VPAQVVLTPYKPEPGYECQISQEYYDRKAQMCCAKCPPGQYVKHFCNKTSDTVCADCEASMYTQVWNQFRTCLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDIEGRMDPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGHHHHHH

SEQ ID NO: 23 - protein, 248 aa, Artificial Sequence
  Synthetic TNFR2 Chimera 9
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGIEGRMDPHHHHHH

SEQ ID NO: 24 - protein, 477 aa, Artificial Sequence
  Synthetic TNFR2 Chimera 9  Fc fusion
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGIEGRMDPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGHHHHHH

SEQ ID NO: 25 - protein, 20 aa, Artificial Sequence
  Synthetic Signal peptide
MGTPAQLLFLLLLWLPDTTG

SEQ ID NO: 26 - DNA, 768 bp, Artificial Sequence
  Synthetic TNFR2 Chimera 0
taccggtgtccccgcccaagtcgtgcttactccctacaagccagaacctggatatgaatgtcagatttcccaagagtactacgaccggaaggcgcagatgtgctgttcaaagtgcagcccgggccagcacgccaaagtgttctgcaccaagacctccgacaccgtgtgcgacagctgcgaggactccacatacactcagctctggaactgggtgccagaatgcctgtcctgtggctcccgctgctcctccgatcaagtggagactcaggcctgcaccagggaacagaacagaatctgtacgtgccggccggggtggtactgtgctctgtcgaagcaggagggatgcagactgtgcgccccgttgcggaagtgccgccctggatttggtgtcgcgcgcccgggtaccgaaaccagcgatgtggtctgcaagccgtgcgcacccgggaccttctcaaacaccacctcctcgaccgacatctgtcggccgcatcagatttgcaacgtggtggcaatccctggcaatgcctctatggatgctgtgtgcactagcacctcccctactcgctccatggcgcccggagccgtgcacctcccgcaacccgtgtcgaccaggagccagcacactcagcctacccccgaaccctccaccgccccttcgacttcattcctgctgcctatgggaccatccccgccggccgagggcagcaccggagacattgaaggccgcatggatccgcatcatcatcatcatcattaatgagcggccgc

SEQ ID NO: 27 - DNA, 1449 bp, Artificial Sequence
  Synthetic TNFR2 Chimera 0  Fc fusion
gtccccgcccaagtcgtgcttactccctacaagccagaacctggatatgaatgtcagatttcccaagagtactacgaccggaaggcgcagatgtgctgttcaaagtgcagcccgggccagcacgccaaagtgttctgcaccaagacctccgacaccgtgtgcgacagctgcgaggactccacatacactcagctctggaactgggtgccagaatgcctgtcctgtggctcccgctgctcctccgatcaagtggagactcaggcctgcaccagggaacagaacagaatctgtacgtgccggccggggtggtactgtgctctgtcgaagcaggagggatgcagactgtgcgccccgttgcggaagtgccgccctggatttggtgtcgcgcgcccgggtaccgaaaccagcgatgtggtctgcaagccgtgcgcacccgggaccttctcaaacaccacctcctcgaccgacatctgtcggccgcatcagatttgcaacgtggtggcaatccctggcaatgcctctatggatgctgtgtgcactagcacctcccctactcgctccatggcgcccggagccgtgcacctcccgcaacccgtgtcgaccaggagccagcacactcagcctacccccgaaccctccaccgccccttcgacttcattcctgctgcctatgggaccatccccgccggccgagggcagcaccggagacattgaaggccgcatggatccgaaatcgtctgataagacacatacatgccctccatgtccggcgcccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtcatcatcatcatcatcattaatgagcggccgct

SEQ ID NO: 28 - DNA, 768 bp, Artificial Sequence
  Synthetic TNFR2 Chimera 1
taccggtgtccccgcccaagtcgtgctcaccccctacaagccagaacccggatacgagtgtcagatcagccaggagtattacgaccggaaggcccagatgtgctgcgccaagtgtcctccgggccaatacgtgaaacacttctgcaacaagacttccgataccgtgtgcgccgactgcgaggattcaacgtacacccagctgtggaactgggtgcctgagtgcctgtcttgcggtagcagatgtagctccgaccaggtcgaaacccaagcctgcacccgcgaacagaacaggatttgcacctgtcgcccgggatggtactgcgctctgtcgaagcaggaaggttgccgcctgtgcgcgcctctccggaagtgtagaccgggattcggcgtggcccgccccgggactgaaacttccgatgtcgtgtgcaagccctgcgcccccgggacctttagcaacaccacttcctccacggacatctgtaggccccatcagatttgcaacgtggtggcgatcccgggcaatgccagcatggacgccgtgtgcacttccacctcaccgacccggtcaatggcacctggagctgtgcacttgccacaaccagtgtccacccggtcgcagcacacccagcccaccccggagccgtcgactgcaccttccacatccttccttctgcctatgggaccgtcgccgcctgcggaaggctccactggagacattgaaggccgcatggatccgcatcatcatcatcatcattaatgagcggccgc

SEQ ID NO: 29 - DNA, 1437 bp, Artificial Sequence
  Synthetic TNFR2 Chimera 1  Fc fusion
gtccccgcccaagtcgtgctcaccccctacaagccagaacccggatacgagtgtcagatcagccaggagtattacgaccggaaggcccagatgtgctgcgccaagtgtcctccgggccaatacgtgaaacacttctgcaacaagacttccgataccgtgtgcgccgactgcgaggattcaacgtacacccagctgtggaactgggtgcctgagtgcctgtcttgcggtagcagatgtagctccgaccaggtcgaaacccaagcctgcacccgcgaacagaacaggatttgcacctgtcgcccgggatggtactgcgctctgtcgaagcaggaaggttgccgcctgtgcgcgcctctccggaagtgtagaccgggattcggcgtggcccgccccgggactgaaacttccgatgtcgtgtgcaagccctgcgcccccgggacctttagcaacaccacttcctccacggacatctgtaggccccatcagatttgcaacgtggtggcgatcccgggcaatgccagcatggacgccgtgtgcacttccacctcaccgacccggtcaatggcacctggagctgtgcacttgccacaaccagtgtccacccggtcgcagcacacccagcccaccccggagccgtcgactgcaccttccacatccttccttctgcctatgggaccgtcgccgcctgcggaaggctccactggagacattgaaggccgcatggatccgaaatcgtctgataagacacatacatgccctccatgtccggcgcccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtcatcatcatcatcatcattaa

SEQ ID NO: 30 - DNA, 768 bp, Artificial Sequence
  Synthetic TNFR2 Chimera 2
taccggtgtccccgcccaagtcgtgcttaccccatacaaacccgaacccggttacgaatgtcagattagccaggagtactatgataggaaggcccagatgtgttgcgcgaagtgcccgcccgggcagtacgtgaagcacttttgcaacaagacctccgacactgtgtgcgccgactgcgaggcttcgatgtacactcaagtctggaaccagttcagaacatgcctgtcctgctcgtcctcatgtaccactgaccaagtggaaatccgggcttgcactaagcagcagaaccgcgtgtgcacttgccgccctggatggtactgtgccttgagcaagcaggagggatgccggctctgtgccccgctgagaaagtgcaggcctggcttcggcgtggcgcgcccgggaaccgaaacctccgatgtcgtgtgcaagccgtgtgcccccgggactttcagcaacaccacctcctccaccgacatctgccggccgcaccagatttgcaatgtggtggccatccctggcaacgccagcatggacgccgtgtgcacctccacgtcaccgacccggtcgatggcacccggagcagtgcatctgccacaacctgtgtctacccggagccagcacacccagcctacccctgaaccttcgaccgcgccatccacctccttcctcctgcccatgggcccgtccccgcccgccgagggtagcactggagatattgaaggccgcatggatccgcatcatcatcatcatcattaatgagcggccgc

SEQ ID NO: 31 - DNA, 1437 bp, Artificial Sequence
  Synthetic TNFR2 Chimera 2  Fc fusion
gtccccgcccaagtcgtgcttaccccatacaaacccgaacccggttacgaatgtcagattagccaggagtactatgataggaaggcccagatgtgttgcgcgaagtgcccgcccgggcagtacgtgaagcacttttgcaacaagacctccgacactgtgtgcgccgactgcgaggcttcgatgtacactcaagtctggaaccagttcagaacatgcctgtcctgctcgtcctcatgtaccactgaccaagtggaaatccgggcttgcactaagcagcagaaccgcgtgtgcacttgccgccctggatggtactgtgccttgagcaagcaggagggatgccggctctgtgccccgctgagaaagtgcaggcctggcttcggcgtggcgcgcccgggaaccgaaacctccgatgtcgtgtgcaagccgtgtgcccccgggactttcagcaacaccacctcctccaccgacatctgccggccgcaccagatttgcaatgtggtggccatccctggcaacgccagcatggacgccgtgtgcacctccacgtcaccgacccggtcgatggcacccggagcagtgcatctgccacaacctgtgtctacccggagccagcacacccagcctacccctgaaccttcgaccgcgccatccacctccttcctcctgcccatgggcccgtccccgcccgccgagggtagcactggagatattgaaggccgcatggatccgaaatcgtctgataagacacatacatgccctccatgtccggcgcccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtcatcatcatcatcatcattaa

SEQ ID NO: 32 - DNA, 765 bp, Artificial Sequence
  Synthetic TNFR2 Chimera 3
taccggtctgcctgcccaagtcgccttcaccccctacgcacccgaacctggttccacttgtcggttgagagagtactacgaccagactgcgcagatgtgctgcgccaagtgcccgcccggtcaatacgtgaagcacttctgcaacaagaccagcgatacagtgtgcgcggattgtgaagcctccatgtatactcaagtctggaaccagtttcgcacctgtctgtcatgctcctcgtcctgcaccaccgaccaagtggaaatccgggcttgcaccaagcagcagaatcgcgtgtgcgcctgcgaggccggacggtactgcgcgcttaagactcactccgggtcgtgtcggcagtgcatgaggctctcaaaatgcggccccggattcggagtggcttcctcccgcgccccaaacggcaacgtgctgtgcaaggcttgtgcccctggaaccttcagcgacaccacttcctcgaccgacgtctgtcgcccgcatcggatctgctccattctcgccattcccggaaacgccagcaccgacgccgtgtgcgcaccggaatcgccgaccctgtctgcgatcccaaggactctctacgtgtcacagcctgagcctactagatcccagccactggatcaggagccgggccccagccagaccccgagcattctgacgtcgctgggcagcaccccgatcatcgaacagtccaccaaggggggaattgaaggccgcatggatccgcatcatcatcatcatcattaatgagcggccgc

SEQ ID NO: 33 - DNA, 1434 bp, Artificial Sequence
  Synthetic TNFR2 Chimera 3  Fc fusion
ctgcctgcccaagtcgccttcaccccctacgcacccgaacctggttccacttgtcggttgagagagtactacgaccagactgcgcagatgtgctgcgccaagtgcccgcccggtcaatacgtgaagcacttctgcaacaagaccagcgatacagtgtgcgcggattgtgaagcctccatgtatactcaagtctggaaccagtttcgcacctgtctgtcatgctcctcgtcctgcaccaccgaccaagtggaaatccgggcttgcaccaagcagcagaatcgcgtgtgcgcctgcgaggccggacggtactgcgcgcttaagactcactccgggtcgtgtcggcagtgcatgaggctctcaaaatgcggccccggattcggagtggcttcctcccgcgccccaaacggcaacgtgctgtgcaaggcttgtgcccctggaaccttcagcgacaccacttcctcgaccgacgtctgtcgcccgcatcggatctgctccattctcgccattcccggaaacgccagcaccgacgccgtgtgcgcaccggaatcgccgaccctgtctgcgatcccaaggactctctacgtgtcacagcctgagcctactagatcccagccactggatcaggagccgggccccagccagaccccgagcattctgacgtcgctgggcagcaccccgatcatcgaacagtccaccaaggggggaattgaaggccgcatggatccgaaatcgtctgataagacacatacatgccctccatgtccggcgcccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtcatcatcatcatcatcattaa

SEQ ID NO: 34 - DNA, 765 bp, Artificial Sequence
  Synthetic TNFR2 Chimera 4
taccggtctgcccgcccaagtcgcctttaccccctacgcccccgagcctggttccacctgtcgcctgcgcgaatactacgaccagacagcgcagatgtgctgctcaaagtgctcgcccggacagcatgcaaaggtgttctgcaccaagacttccgataccgtgtgcgccgactgtgaagctagcatgtacacccaagtctggaaccagttccggacttgcttgtcctgttcgtcatcctgtacgaccgaccaggtcgagatcagggcgtgcaccaagcagcaaaaccgcgtgtgcgcttgcgaggctggaagatattgtgcgctcaagacccactccgggagctgcaggcagtgcatgcggctctctaagtgcggacctggattcggagtggcctcctcgcgggcccctaacggcaacgtgctttgtaaagcctgcgccccgggcactttcagcgacaccactagctcgactgacgtgtgccgcccgcaccggatctgcagcatcctcgcgattcccggcaatgccagcacggatgcagtgtgcgccccggagtcccctaccctgtccgccattccgcggactctgtacgtgtcgcaacctgaaccgaccagatcccagccgctggatcaggagcccgggccgtcccagactccatccatcctgacctcactgggttccaccccaatcattgaacagtccaccaagggcggaattgaaggccgcatggatccgcatcatcatcatcatcattaatgagcggccgc

SEQ ID NO: 35 - DNA, 1434 bp, Artificial Sequence
  Synthetic TNFR2 Chimera 4  Fc fusion
ctgcccgcccaagtcgcctttaccccctacgcccccgagcctggttccacctgtcgcctgcgcgaatactacgaccagacagcgcagatgtgctgctcaaagtgctcgcccggacagcatgcaaaggtgttctgcaccaagacttccgataccgtgtgcgccgactgtgaagctagcatgtacacccaagtctggaaccagttccggacttgcttgtcctgttcgtcatcctgtacgaccgaccaggtcgagatcagggcgtgcaccaagcagcaaaaccgcgtgtgcgcttgcgaggctggaagatattgtgcgctcaagacccactccgggagctgcaggcagtgcatgcggctctctaagtgcggacctggattcggagtggcctcctcgcgggcccctaacggcaacgtgctttgtaaagcctgcgccccgggcactttcagcgacaccactagctcgactgacgtgtgccgcccgcaccggatctgcagcatcctcgcgattcccggcaatgccagcacggatgcagtgtgcgccccggagtcccctaccctgtccgccattccgcggactctgtacgtgtcgcaacctgaaccgaccagatcccagccgctggatcaggagcccgggccgtcccagactccatccatcctgacctcactgggttccaccccaatcattgaacagtccaccaagggcggaattgaaggccgcatggatccgaaatcgtctgataagacacatacatgccctccatgtccggcgcccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtcatcatcatcatcatcattaa

SEQ ID NO: 36 - DNA, 762 bp, Artificial Sequence
  Synthetic TNFR2 Chimera 5
taccggtctgcctgcccaagtcgccttcactccctacgcccccgaacccggctccacctgtcgcctgagagagtactacgatcagaccgcgcagatgtgctgttccaagtgttccccgggacagcacgcgaaagtgttctgcaccaagaccagcgacaccgtgtgcgattcctgcgaggactccacatacactcagctctggaactgggtcccagaatgtctgtcctgcggtagccggtgttcctcggaccaagtggaaacccaggcctgcactcgcgagcagaatcggatttgcacttgccggcctgggtggtattgcgccctgtcaaagcaggagggctgccggctctgcgcacctctgaggaagtgcagacccggatttggagtggcccgcccgggaaccgaaaccagcgacgtcgtgtgcaagccgtgtgccccggggaccttcagcaacaccacgtcctcgaccgatatttgccggccgcatcagatctgcaacgtggtggcaattccgggaaacgcttcaatggacgctgtgtgcgcccccgagtctccaactttgagcgcgatccctcgcactctctacgtgtcccaaccggagcccaccaggtcacagccactggaccaagaacctggcccgagccagactccttcgatccttacttccctgggttcgacccccatcatcgaacagtccaccaagggaggcattgaaggccgcatggatccgcatcatcatcatcatcattaatgagcggccgc

SEQ ID NO: 37 - DNA, 1431 bp, Artificial Sequence
  Synthetic TNFR2 Chimera 5  Fc fusion
ctgcctgcccaagtcgccttcactccctacgcccccgaacccggctccacctgtcgcctgagagagtactacgatcagaccgcgcagatgtgctgttccaagtgttccccgggacagcacgcgaaagtgttctgcaccaagaccagcgacaccgtgtgcgattcctgcgaggactccacatacactcagctctggaactgggtcccagaatgtctgtcctgcggtagccggtgttcctcggaccaagtggaaacccaggcctgcactcgcgagcagaatcggatttgcacttgccggcctgggtggtattgcgccctgtcaaagcaggagggctgccggctctgcgcacctctgaggaagtgcagacccggatttggagtggcccgcccgggaaccgaaaccagcgacgtcgtgtgcaagccgtgtgccccggggaccttcagcaacaccacgtcctcgaccgatatttgccggccgcatcagatctgcaacgtggtggcaattccgggaaacgcttcaatggacgctgtgtgcgcccccgagtctccaactttgagcgcgatccctcgcactctctacgtgtcccaaccggagcccaccaggtcacagccactggaccaagaacctggcccgagccagactccttcgatccttacttccctgggttcgacccccatcatcgaacagtccaccaagggaggcattgaaggccgcatggatccgaaatcgtctgataagacacatacatgccctccatgtccggcgcccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtcatcatcatcatcatcattaa

SEQ ID NO: 38 - DNA, 771 bp, Artificial Sequence
  Synthetic TNFR2 Chimera 6
taccggtgtccccgcccaagtcgtcctcaccccatacaagcctgaacccggatacgagtgccagattagccaagagtactacgaccgcaaggctcagatgtgctgtgcgaagtgcccaccgggacaatacgtgaagcacttctgcaacaagaccagcgacaccgtgtgtgccgattgcgaagcgtccatgtatacccaggtctggaatcagttcagaacctgtctttcatgttcctcctcctgcactaccgaccaagtggagatccgggcctgcactaagcagcagaaccgcgtgtgcgcttgcgaggccggccggtactgcgcgctcaagacccactcagggtcgtgccggcagtgcatgcggctgtccaaatgtggcccgggatttggcgtggcatcgagcagggcgcctaacgggaacgtgctgtgcaaggcctgcgcccccggaacattctccgatactacttcctccacggacgtgtgcaggccacaccgcatctgttctatcttggccattccgggaaacgccagcaccgatgctgtgtgcacctccacttcgcctactcggtccatggccccgggtgcagtgcatctgccgcagcccgtgtcaaccagatcgcagcacactcagcctacccccgaacccagcaccgcccctagcacctcgttcctgctgcctatgggaccgtccccgcccgccgaaggttccaccggcgacattgaaggccgcatggatccgcatcatcatcatcatcattaatgagcggccgc

SEQ ID NO: 39 - DNA, 1440 bp, Artificial Sequence
  Synthetic TNFR2 Chimera 6  Fc fusion
gtccccgcccaagtcgtcctcaccccatacaagcctgaacccggatacgagtgccagattagccaagagtactacgaccgcaaggctcagatgtgctgtgcgaagtgcccaccgggacaatacgtgaagcacttctgcaacaagaccagcgacaccgtgtgtgccgattgcgaagcgtccatgtatacccaggtctggaatcagttcagaacctgtctttcatgttcctcctcctgcactaccgaccaagtggagatccgggcctgcactaagcagcagaaccgcgtgtgcgcttgcgaggccggccggtactgcgcgctcaagacccactcagggtcgtgccggcagtgcatgcggctgtccaaatgtggcccgggatttggcgtggcatcgagcagggcgcctaacgggaacgtgctgtgcaaggcctgcgcccccggaacattctccgatactacttcctccacggacgtgtgcaggccacaccgcatctgttctatcttggccattccgggaaacgccagcaccgatgctgtgtgcacctccacttcgcctactcggtccatggccccgggtgcagtgcatctgccgcagcccgtgtcaaccagatcgcagcacactcagcctacccccgaacccagcaccgcccctagcacctcgttcctgctgcctatgggaccgtccccgcccgccgaaggttccaccggcgacattgaaggccgcatggatccgaaatcgtctgataagacacatacatgccctccatgtccggcgcccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtcatcatcatcatcatcattaa

SEQ ID NO: 40 - DNA, 765 bp, Artificial Sequence
  Synthetic TNFR2 Chimera 7
taccggtctgcctgcccaagtcgccttcaccccgtacgcccccgaacccggttcaacctgtcgcctgagagagtattacgaccagaccgcgcagatgtgctgctccaagtgttccccgggacagcatgctaaggtcttttgcaccaaaacaagcgacactgtgtgcgactcctgcgaggattccacctacacccaactgtggaactgggtgcccgagtgtctgagctgctcctcctcctgtactacggatcaagtggagattcgggcctgcaccaagcagcaaaaccgggtctgcgcctgtgaagccggccgctactgcgcactcaagactcactcgggctcatgcaggcagtgtatgcggctgtctaagtgcggacccggcttcggagtggccagctccagagcccctaatggcaacgtgttgtgcaaggcctgcgcgccggggaccttctcggatactactagctccaccgacgtgtgccgcccccaccggatctgcagcatcctggctatccctggaaacgcgtcgaccgacgccgtgtgcgcgccggaatcaccgaccctctcggcaattccgcgcactctctacgtgtcgcagccagaacccaccaggtcccagccactggaccaggaaccaggacctagccagactccgtccatccttacctccctgggaagcacccctatcattgagcagtccaccaaggggggtattgaaggccgcatggatccgcatcatcatcatcatcattaatgagcggccgc

SEQ ID NO: 41 - DNA, 1434 bp, Artificial Sequence
  Synthetic TNFR2 Chimera 7  Fc fusion
ctgcctgcccaagtcgccttcaccccgtacgcccccgaacccggttcaacctgtcgcctgagagagtattacgaccagaccgcgcagatgtgctgctccaagtgttccccgggacagcatgctaaggtcttttgcaccaaaacaagcgacactgtgtgcgactcctgcgaggattccacctacacccaactgtggaactgggtgcccgagtgtctgagctgctcctcctcctgtactacggatcaagtggagattcgggcctgcaccaagcagcaaaaccgggtctgcgcctgtgaagccggccgctactgcgcactcaagactcactcgggctcatgcaggcagtgtatgcggctgtctaagtgcggacccggcttcggagtggccagctccagagcccctaatggcaacgtgttgtgcaaggcctgcgcgccggggaccttctcggatactactagctccaccgacgtgtgccgcccccaccggatctgcagcatcctggctatccctggaaacgcgtcgaccgacgccgtgtgcgcgccggaatcaccgaccctctcggcaattccgcgcactctctacgtgtcgcagccagaacccaccaggtcccagccactggaccaggaaccaggacctagccagactccgtccatccttacctccctgggaagcacccctatcattgagcagtccaccaaggggggtattgaaggccgcatggatccgaaatcgtctgataagacacatacatgccctccatgtccggcgcccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtcatcatcatcatcatcattaa

SEQ ID NO: 42 - DNA, 768 bp, Artificial Sequence
  Synthetic TNFR2 Chimera 8
taccggtgtccccgcccaagtcgtgctgaccccctacaaacccgagccaggatatgaatgccagatctcccaagagtactacgaccgcaaggcccagatgtgttgcgcgaagtgtccgccggggcagtacgtgaagcacttctgcaacaagacctccgataccgtgtgcgccgattgcgaagcgtccatgtacactcaagtctggaaccagttcagaacttgcctgtcttgtggctcgaggtgctcaagcgaccaggtggaaactcaggcttgcacgcgggagcagaatcgcatttgcacttgccggccgggctggtactgcgccttgtcaaagcaggaaggttgcaggctgtgtgccccactgcggaagtgtcggcctggtttcggagtggctcgcccgggcaccgagacttcagacgtggtctgcaagccctgcgcgcccggaacctttagcaacaccacctcctcgaccgacatttgtagaccgcaccagatctgcaacgtggtggccatccccgggaacgcctcgatggatgcagtgtgcaccagcactagcccgacccgctccatggcccctggagccgtgcacctcccccaacctgtgtccacccggtcccagcatacacagcctacccctgaaccatccaccgcaccgtccacttccttccttctccctatgggcccgagcccgcccgccgagggatcgaccggagacattgaaggccgcatggatccgcatcatcatcatcatcattaatgagcggccgc

SEQ ID NO: 43 - DNA, 1434 bp, Artificial Sequence
  Synthetic TNFR2 Chimera 8  Fc fusion
gtccccgcccaagtcgtgctgaccccctacaaacccgagccaggatatgaatgccagatctcccaagagtactacgaccgcaaggcccagatgtgttgcgcgaagtgtccgccggggcagtacgtgaagcacttctgcaacaagacctccgataccgtgtgcgccgattgcgaagcgtccatgtacactcaagtctggaaccagttcagaacttgcctgtcttgtggctcgaggtgctcaagcgaccaggtggaaactcaggcttgcacgcgggagcagaatcgcatttgcacttgccggccgggctggtactgcgccttgtcaaagcaggaaggttgcaggctgtgtgccccactgcggaagtgtcggcctggtttcggagtggctcgcccgggcaccgagacttcagacgtggtctgcaagccctgcgcgcccggaacctttagcaacaccacctcctcgaccgacatttgtagaccgcaccagatctgcaacgtggtggccatccccgggaacgcctcgatggatgcagtgtgcaccagcactagcccgacccgctccatggcccctggagccgtgcacctcccccaacctgtgtccacccggtcccagcatacacagcctacccctgaaccatccaccgcaccgtccacttccttccttctccctatgggcccgagcccgcccgccgagggatcgaccggagacattgaaggccgcatggatccgaaatcgtctgataagacacatacatgccctccatgtccggcgcccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtcatcatcatcatcatcat

SEQ ID NO: 44 - DNA, 765 bp, Artificial Sequence
  Synthetic TNFR2 Chimera 9
taccggtctgcccgcacaagtcgccttcaccccatacgcccctgaacccggatcaacttgccgcctgagagagtactacgatcagaccgcccagatgtgttgctccaagtgcagccctggccaacacgcgaaggtgttctgtaccaagacgtccgacaccgtgtgcgacagctgcgaggactccacctatactcagctctggaactgggtgcccgaatgcttgtcctgcggtagccgctgtagctcggatcaggtcgaaacccaggcctgtactcgggagcagaacagaatttgcgcgtgcgaagcgggacggtactgcgctctgaaaacacattccggctcgtgtcggcagtgcatgaggctgtcgaagtgcggcccgggattcggcgtggcctcgtcccgggctccgaacgggaatgtgctgtgcaaggcctgcgcccctggcaccttttccgacactacttcctccaccgacgtgtgccggccccaccgcatttgctccatcctggcaatcccggggaacgccagcaccgatgccgtgtgtgccccggaatccccgaccctgtccgccatccctcgcactctctacgtgtctcagccggagcctactaggtcacagccccttgaccaagaaccaggacccagccaaaccccatcaatcctgacctccctcggatcgaccccgattatcgagcagagcaccaagggtggaattgaaggccgcatggatccgcatcatcatcatcatcattaatgagcggccgc

SEQ ID NO: 45 - DNA, 1434 bp, Artificial Sequence
  Synthetic TNFR2 Chimera 9  Fc fusion
ctgcccgcacaagtcgccttcaccccatacgcccctgaacccggatcaacttgccgcctgagagagtactacgatcagaccgcccagatgtgttgctccaagtgcagccctggccaacacgcgaaggtgttctgtaccaagacgtccgacaccgtgtgcgacagctgcgaggactccacctatactcagctctggaactgggtgcccgaatgcttgtcctgcggtagccgctgtagctcggatcaggtcgaaacccaggcctgtactcgggagcagaacagaatttgcgcgtgcgaagcgggacggtactgcgctctgaaaacacattccggctcgtgtcggcagtgcatgaggctgtcgaagtgcggcccgggattcggcgtggcctcgtcccgggctccgaacgggaatgtgctgtgcaaggcctgcgcccctggcaccttttccgacactacttcctccaccgacgtgtgccggccccaccgcatttgctccatcctggcaatcccggggaacgccagcaccgatgccgtgtgtgccccggaatccccgaccctgtccgccatccctcgcactctctacgtgtctcagccggagcctactaggtcacagccccttgaccaagaaccaggacccagccaaaccccatcaatcctgacctccctcggatcgaccccgattatcgagcagagcaccaagggtggaattgaaggccgcatggatccgaaatcgtctgataagacacatacatgccctccatgtccggcgcccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtcatcatcatcatcatcattaa

SEQ ID NO: 46 - DNA, 60 bp, Artificial Sequence
  Synthetic Signal peptide
atgggcactccagctcagttgctgttcctccttcttctttggctcccagacactaccggt

SEQ ID NO: 47 - protein, 5 aa, Artificial Sequence
  Synthetic ABV2 VHCDR1 (Kabat)
TFGMS

SEQ ID NO: 48 - protein, 17 aa, Artificial Sequence
  Synthetic ABV2  VHCDR2 (Kabat)
WINTYSGVPTYADDFKG

SEQ ID NO: 49 - protein, 6 aa, Artificial Sequence
  Synthetic ABV2  VHCDR3 (Kabat)
RSNFAY

SEQ ID NO: 50 - protein, 15 aa, Artificial Sequence
  Synthetic ABV2  VLCDR1 (Kabat)
RASESVDSSGNSFMH

SEQ ID NO: 51 - protein, 7 aa, Artificial Sequence
  Synthetic ABV2  VLCDR2 (Kabat)
RASNLES

SEQ ID NO: 52 - protein, 9 aa, Artificial Sequence
  Synthetic ABV2  VLCDR3 (Kabat)
QQSNEDPWT

SEQ ID NO: 53 - protein, 7 aa, Artificial Sequence
  Synthetic ABV2  VHCDR1 (Chothia)
GYTFTTF

SEQ ID NO: 54 - protein, 6 aa, Artificial Sequence
  Synthetic ABV2  VHCDR2 (Chothia)
NTYSGV

SEQ ID NO: 55 - protein, 6 aa, Artificial Sequence
  Synthetic ABV2  VHCDR3 (Chothia)
RSNFAY

SEQ ID NO: 56 - protein, 15 aa, Artificial Sequence
  Synthetic ABV2  VLCDR1 (Chothia)
RASESVDSSGNSFMH

SEQ ID NO: 57 - protein, 7 aa, Artificial Sequence
  Synthetic ABV2  VLCDR2 (Chothia)
RASNLES

SEQ ID NO: 58 - protein, 9 aa, Artificial Sequence
  Synthetic ABV2  VLCDR3 (Chothia)
QQSNEDPWT

SEQ ID NO: 59 - protein, 10 aa, Artificial Sequence
  Synthetic ABV2  VHCDR1 (enhanced Chothia)
GYTFTTFGMS

SEQ ID NO: 60 - protein, 10 aa, Artificial Sequence
  Synthetic ABV2  VHCDR2 (enhanced Chothia)
WINTYSGVPT

SEQ ID NO: 61 - protein, 6 aa, Artificial Sequence
  Synthetic ABV2  VHCDR3 (enhanced Chothia)
RSNFAY

SEQ ID NO: 62 - protein, 15 aa, Artificial Sequence
  Synthetic ABV2  VLCDR1 (enhanced Chothia)
RASESVDSSGNSFMH

SEQ ID NO: 63 - protein, 7 aa, Artificial Sequence
  Synthetic ABV2  VLCDR2 (enhanced Chothia)
RASNLES

SEQ ID NO: 64 - protein, 9 aa, Artificial Sequence
  Synthetic ABV2  VLCDR3 (enhanced Chothia)
QQSNEDPWT

SEQ ID NO: 65 - protein, 7 aa, Artificial Sequence
  Synthetic ABV2  VHCDR1 (IMGT)
GYTFTTF

SEQ ID NO: 66 - protein, 8 aa, Artificial Sequence
  Synthetic ABV2  VHCDR2 (IMGT)
INTYSGVP

SEQ ID NO: 67 - protein, 8 aa, Artificial Sequence
  Synthetic ABV2  VHCDR3 (IMGT)
ARRSNFAY

SEQ ID NO: 68 - protein, 10 aa, Artificial Sequence
  Synthetic ABV2  VLCDR1 (IMGT)
ESVDSSGNSF

SEQ ID NO: 69 - protein, 3 aa, Artificial Sequence
  Synthetic ABV2  VLCDR2 (IMGT)
RAS

SEQ ID NO: 70 - protein, 10 aa, Artificial Sequence
  Synthetic ABV2  VLCDR3 (IMGT)
CQQSNEDPWT

SEQ ID NO: 71 - protein, 115 aa, Artificial Sequence
  Synthetic ABV2  VH
QIQLVQSGPELKKPGETVKISCKASGYTFTTFGMSWVKQAPGKGLKWMGWINTYSGVPTYADDFKGRFAFSLETSASTAYLQINNLKNEDTATYFCARRSNFAYWGQGTLVTVSA

SEQ ID NO: 72 - protein, 111 aa, Artificial Sequence
  Synthetic ABV2  VL
DIVLTQSPASLAVSLGQRATISCRASESVDSSGNSFMHWYQQKAGQSFKLLIYRASNLESGIPARFSGSGSRTDFTLTINPVEADDVATYYCQQSNEDPWTFGGGTKLEIK

SEQ ID NO: 73 - protein, 115 aa, Artificial Sequence
  Synthetic ABV2 VH_hum#1
QIQLVQSGAEVKKPGSSVKVSCRASGYTFTTFGMSWVRQAPGQGLEWMGWINTYSGVPTYAQNFQGRFAFTVDTEASTAYMELRSLKSEDSAVYFCARRSNFAYWGQGTTVTVSS

SEQ ID NO: 74 - protein, 115 aa, Artificial Sequence
  Synthetic ABV2 VH_hum#2
QVQLVQSGSELKKPGASVKVSCKASGYTFTTFGMSWVRQAPGQGLEWMGWINTYSGVPTYAQGFTGRFVFSLDTSVSTAYLQISSLKAEDTAVYFCARRSNFAYWGQGTLVTVSS

SEQ ID NO: 75 - protein, 115 aa, Artificial Sequence
  Synthetic ABV2 VH_hum#3
QVQLVESGGGLVQPGGSLKLSCAASGYTFTTFGMSWVRQASGKGLEWMGWINTYSGVPTYAASMRGRFTFSLDTSKNTAFLQMNSLKSDDTAMYFCARRSNFAYWGQGTLVTVSS

SEQ ID NO: 76 - protein, 115 aa, Artificial Sequence
  Synthetic ABV2 VH_hum#4
QIQLVQSGAEVKKPGSSVKVSCKASGYTFTTFGMSWVRQAPGQGLEWMGWINTYSGVPTYAQKFQGRFTFTLDTSTSTAYMELSSLRSEDTAVYFCARRSNFAYWGQGTLVTVSS

SEQ ID NO: 77 - protein, 115 aa, Artificial Sequence
  Synthetic ABV2 VH_hum#5
EIQLVQSGAEVKKPGESLKISCQAFGYTFTTFGMSWVRQMPGQGLEWMGWINTYSGVPTYNENFKGQFTFSLDTSSSTAYLQWSSLKASDTAMYFCARRSNFAYWGQGTMVTVSS

SEQ ID NO: 78 - protein, 115 aa, Artificial Sequence
  Synthetic ABV2 VH_hum#6
EVQLLESGGGLVQPGGSLRLSCAASGYTFTTFGMSWVRQAPGKGLEWMGWINTYSGVPTYNENFKGRFTFSVDTSKNTAYLQMNSLRAEDTAVYFCARRSNFAYWGQGTMVTVSS

SEQ ID NO: 79 - protein, 115 aa, Artificial Sequence
  Synthetic ABV2 VH_hum#7
QIQLVQSGAEVKKPGASVKVSCKASGYTFTTFGMSWVRQAPGQGLEWMGWINTYSGVPTYNEKFKSKFTFTLDTSTNTAYMELSSLRSEDTAVYFCARRSNFAYWGQGTLVTVSS

SEQ ID NO: 80 - protein, 115 aa, Artificial Sequence
  Synthetic ABV2 VH_hum#8
QIQLVQSGAEVKKPGASVKVSCKASGYTFTTFGMSWVRQAPGQGLEWMGWINTYSGVPTYAQKFQGRFTFTLDTSTSTAYMELSSLRSEDTAVYFCARRSNFAYWGQGTLVTVSS

SEQ ID NO: 81 - protein, 115 aa, Artificial Sequence
  Synthetic ABV2 VH_hum#9
EIQLVESGGGLVQPGGSLRLSCAASGYTFTTFGMSWVRQAPGKGLEWMGWINTYSGVPTYADSVKGRFTFSLDTSKNTAYLQMNSLRAEDTAVYFCARRSNFAYWGQGTLVTVSS

SEQ ID NO: 82 - protein, 115 aa, Artificial Sequence
  Synthetic ABV2 VH_hum#10
QVQLVQSGSELKKPGASVKVSCKASGYTFTTFGMSWVRQAPGQGLEWMGWINTYSGVPTYADDFKGRFVFSLDTSVSTAYLQISSLKAEDTAVYYCARRSNFAYWGAGTTVTVSS

SEQ ID NO: 83 - protein, 115 aa, Artificial Sequence
  Synthetic ABV2 VH_hum#11
QVQLVQSGAEVKKPGASVKVSCKASGYTFTTFGMSWVRQAPGQGLEWMGWINTYSGVPTYADDFKGRVTMTTDTSTSTAYMELRSLRSDDTAVYYCARRSNFAYWGAGTTVTVSS

SEQ ID NO: 84 - protein, 115 aa, Artificial Sequence
  Synthetic ABV2 VL_hum#12
QVQLVQSGAEVKKPGSSVKVSCKASGYTFTTFGMSWVRQAPGQGLEWMGWINTYSGVPTYAQKFQGRVTITADESTSTAYMELSSLRSEDTAVYYCARRSNFAYWGAGTTVTVSS

SEQ ID NO: 85 - protein, 111 aa, Artificial Sequence
  Synthetic ABV2 VL_hum#1
EIVLMQSPGTLSLSPGERATLSCRASESVDSSGNSFMHWYQQKPGQAFRLLIYRASNLESGIPDRFSGSGSRTDATLTISRLEPEDFAVYYCQQSNEDPWTFGQGTKVEIK

SEQ ID NO: 86 - protein, 111 aa, Artificial Sequence
  Synthetic ABV2 VL_hum#2
DIVLTQSPDSLAVSLGERATINCRASESVDSSGNSFMHWYQQKPGQPPKLLIYRASNLESGVPDRFSGSGSRTDFTLTISSLQAEDVAVYYCQQSNEDPWTFGGGTKVEIK

SEQ ID NO: 87 - protein, 111 aa, Artificial Sequence
  Synthetic ABV2 VL_hum#3
DIVLTQSPLSLSVTPGEPASISCRASESVDSSGNSFMHWYLQKPGQSFQLLIYRASNLESGVPDRFSGSGSGTDFTLKIIRVEAEDAGTYYCQQSNEDPWTFGQGTRLEIK

SEQ ID NO: 88 - protein, 111 aa, Artificial Sequence
  Synthetic ABV2 VL_hum#4
DIVLTQSPDSLAVSLGERATINCRASESVDSSGNSFMHWYQQKPGQPFKLLIYRASNLESGVPDRFSGSGSRTDFTLTISSLQAEDVAVYYCQQSNEDPWTFGQGTRLEIK

SEQ ID NO: 89 - protein, 111 aa, Artificial Sequence
  Synthetic ABV2 VL_hum#5
DIVLTQTPLSLPVTPGEPASISCRASESVDSSGNSFMHWYLQKPGQSFKLLIYRASNLESGVPDRFSGSGSRTDFTLKISRVEAEDVGVYYCQQSNEDPWTFGQGTKLEIK

SEQ ID NO: 90 - protein, 111 aa, Artificial Sequence
  Synthetic ABV2 VL_hum#6
DIQLTQSPSTLSASVGDRVTITCRASESVDSSGNSFMHWYQQKPGKAFKLLIYRASNLESGVPSRFSGSGSGTEFTLTISSLQPDDFATYYCQQSNEDPWTFGQGTKVEIK

SEQ ID NO: 91 - protein, 111 aa, Artificial Sequence
  Synthetic ABV2 VL_hum#7
DIQLTQSPSSLSASVGDRVTITCRASESVDSSGNSFMHWYQQKPGKAFKLLIYRASNLESGVPSRFSGSGSRTDFTFTISSLQPEDIATYYCQQSNEDPWTFGQGTKVEIK

SEQ ID NO: 92 - protein, 111 aa, Artificial Sequence
  Synthetic ABV2 VL_hum#8
EIVLTQSPGTLSLSPGERATLSCRASESVDSSGNSFMHWYQQKPGQAFRLLIYRASNLESGIPDRFSGSGSRTDFTLTISRLEPEDFAVYYCQQSNEDPWTFGQGTKVEIK

SEQ ID NO: 93 - protein, 111 aa, Artificial Sequence
  Synthetic ABV2 VL_hum#9
EIVLTQSPATLSVSPGERATLSCRASESVDSSGNSFMHWYQQKPGQAFRLLIYRASNLESGIPARFSGSGSRTEFTLTISSLQSEDFAVYYCQQSNEDPWTFGGGTKVEIK

SEQ ID NO: 94 - protein, 111 aa, Artificial Sequence
  Synthetic ABV2 VL_hum#10
DIQLTQSPSSLSASVGDRVTITCRASESVDSSGNSFMHWYQQKPGKAFKLLIYRASNLESGVPSRFSGSGSRTDFTLTISSLQPEDFATYYCQQSNEDPWTFGGGTKVEIK

SEQ ID NO: 95 - protein, 111 aa, Artificial Sequence
  Synthetic ABV2 VL_hum#11
DIVLTQSPLSLPVTPGEPASISCRASESVDSSGNSFMHWYLQKPGQSFQLLIYRASNLESGVPDRFSGSGSRTDFTLKISRVEAEDVGVYYCQQSNEDPWTFGGGTKVEIK

SEQ ID NO: 96 - protein, 111 aa, Artificial Sequence
  Synthetic ABV2 VL_hum#12
DIVLTQSPDSLAVSLGERATINCRASESVDSSGNSFMHWYQQKPGQPFKLLIYRASNLESGVPDRFSGSGSRTDFTLTISSLQAEDVAVYYCQQSNEDPWTFGGGTKVEIK

SEQ ID NO: 97 - protein, 111 aa, Artificial Sequence
  Synthetic ABV2 VL_hum#13
DIVLTQSPDSLAVSLGERATINCRASESVDSSGNSFMHWYQQKAGQSFKLLIYRASNLESGVPDRFSGSGSRTDFTLTISSLQAEDVAVYYCQQSNEDPWTFGGGTKVEIK

SEQ ID NO: 98 - protein, 111 aa, Artificial Sequence
  Synthetic ABV2 VL_hum#14
DIVLTQSPASLAVSPGQRATITCRASESVDSSGNSFMHWYQQKPGQPPKLLIYRASNLESGVPARFSGSGSGTDFTLTINPVEANDTANYYCQQSNEDPWTFGGGTKLEIK

SEQ ID NO: 99 - protein, 111 aa, Artificial Sequence
  Synthetic ABV2 VL_hum#15
DIVMTQSPDSLAVSLGERATINCRASESVDSSGNSFMHWYQQKPGQPPKLLIYRASNLESGVPDRFSGSGSGTDFTLTISSLQAEDVAVYYCQQSNEDPWTFGGGTKLEIK

SEQ ID NO: 100 - protein, 111 aa, Artificial Sequence
  Synthetic ABV2 VL_hum#16
EIVLTQSPATLSLSPGERATLSCRASESVDSSGNSFMHWYQQKPGQAPRLLIYRASNLESGIPARFSGSGSGTDFTLTISSLEPEDFAVYYCQQSNEDPWTFGGGTKLEIK

SEQ ID NO: 101 - protein, 444 aa, Artificial Sequence
  Synthetic Heavy chain of ABV2 chimera
QIQLVQSGPELKKPGETVKISCKASGYTFTTFGMSWVKQAPGKGLKWMGWINTYSGVPTYADDFKGRFAFSLETSASTAYLQINNLKNEDTATYFCARRSNFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - protein, 218 aa, Artificial Sequence
  Synthetic Light chain of ABV2  chimera
DIVLTQSPASLAVSLGQRATISCRASESVDSSGNSFMHWYQQKAGQSFKLLIYRASNLESGIPARFSGSGSRTDFTLTINPVEADDVATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 329 aa, Artificial Sequence
  Synthetic Human IgG1 heavy chain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 104 - protein, 439 aa, Artificial Sequence
  Synthetic Human TNFR2 without leader sequence
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 105 - protein, 452 aa, Artificial Sequence
  Synthetic Mouse TNFR2 without leader sequence
VPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGISLPIGLIVGVTSLGLLMLGLVNCIILVQRKKKPSCLQRDAKVPHVPDEKSQDAVGLEQQHLLTTAPSSSSSSLESSASAGDRRAPPGGHPQARVMAEAQGFQEARASSRISDSSHGSHGTHVNVTCIVNVCSSSDHSSQCSSQASATVGDPDAKPSASPKDEQVPFSQEECPSQSPCETTETLQSHEKPLPLGVPDMGMKPSQAGWFDQIAVKVA

SEQ ID NO: 106 - protein, 444 aa, Artificial Sequence
  Synthetic ABV2.7 HC
QVQLVQSGSELKKPGASVKVSCKASGYTFTTFGMSWVRQAPGQGLEWMGWINTYSGVPTYAQGFTGRFVFSLDTSVSTAYLQISSLKAEDTAVYFCARRSNFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 107 - protein, 218 aa, Artificial Sequence
  Synthetic ABV2.7 LC
DIVLTQSPDSLAVSLGERATINCRASESLTASGNSFMHWYQQKPGQPPKLLIYRASNLESGVPDRFSGSGSRTDFTLTISSLQAEDVAVYYCQQSRHVNWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 7 aa, Artificial Sequence
  Synthetic ABV2.7 VHCDR1 (Chothia)
GYTFTTF

SEQ ID NO: 109 - protein, 6 aa, Artificial Sequence
  Synthetic ABV2.7 VHCDR2 (Chothia)
NTYSGV

SEQ ID NO: 110 - protein, 6 aa, Artificial Sequence
  Synthetic ABV2.7 VHCDR3 (Chothia)
RSNFAY

SEQ ID NO: 111 - protein, 15 aa, Artificial Sequence
  Synthetic ABV2.7 VLCDR1 (Chothia)
RASESLTASGNSFMH

SEQ ID NO: 112 - protein, 7 aa, Artificial Sequence
  Synthetic ABV2.7 VLCDR2 (Chothia)
RASNLES

SEQ ID NO: 113 - protein, 9 aa, Artificial Sequence
  Synthetic ABV2.7 VLCDR3 (Chothia)
QQSRHVNWT

SEQ ID NO: 114 - protein, 5 aa, Artificial Sequence
  Synthetic ABV2.7 VHCDR1 (Kabat)
TFGMS

SEQ ID NO: 115 - protein, 17 aa, Artificial Sequence
  Synthetic ABV2.7 VHCDR2 (Kabat)
WINTYSGVPTYAQGFTG

SEQ ID NO: 116 - protein, 6 aa, Artificial Sequence
  Synthetic ABV2.7 VHCDR3 (Kabat)
RSNFAY

SEQ ID NO: 117 - protein, 15 aa, Artificial Sequence
  Synthetic ABV2.7 VLCDR1 (Kabat)
RASESLTASGNSFMH

SEQ ID NO: 118 - protein, 7 aa, Artificial Sequence
  Synthetic ABV2.7 VLCDR2 (Kabat)
RASNLES

SEQ ID NO: 119 - protein, 9 aa, Artificial Sequence
  Synthetic ABV2.7 VLCDR3 (Kabat)
QQSRHVNWT

SEQ ID NO: 120 - protein, 8 aa, Artificial Sequence
  Synthetic ABV2.7 VHCDR1 (IMGT)
GYTFTTFG

SEQ ID NO: 121 - protein, 8 aa, Artificial Sequence
  Synthetic ABV2.7 VHCDR2 (IMGT)
INTYSGVP

SEQ ID NO: 122 - protein, 8 aa, Artificial Sequence
  Synthetic ABV2.7 VHCDR3 (IMGT)
ARRSNFAY

SEQ ID NO: 123 - protein, 10 aa, Artificial Sequence
  Synthetic ABV2.7 VLCDR1 (IMGT)
ESLTASGNSF

SEQ ID NO: 124 - protein, 3 aa, Artificial Sequence
  Synthetic ABV2.7 VLCDR2 (IMGT)
RAS

SEQ ID NO: 125 - protein, 9 aa, Artificial Sequence
  Synthetic ABV2.7 VLCDR3 (IMGT)
QQSRHVNWT

SEQ ID NO: 126 - protein, 115 aa, Artificial Sequence; identical to SEQ ID NO: 74
  Synthetic ABV2.7 VH

SEQ ID NO: 127 - protein, 111 aa, Artificial Sequence
  Synthetic ABV2.7 VL
DIVLTQSPDSLAVSLGERATINCRASESLTASGNSFMHWYQQKPGQPPKLLIYRASNLESGVPDRFSGSGSRTDFTLTISSLQAEDVAVYYCQQSRHVNWTFGGGTKVEIK

SEQ ID NO: 128 - protein, 444 aa, Artificial Sequence
  Synthetic ABV2.13 HC
QVQLVQSGSELKKPGASVKVSCKASGYTFTTFGMSWVRQAPGQGLEWMGWINTYSGVPHYAQGFTGRFVFSLDTSVSTAYLQISSLKAEDTAVYFCARRSNFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 129 - protein, 218 aa, Artificial Sequence
  Synthetic ABV2.13 LC
DIVLTQSPDSLAVSLGERATINCRASQTVDSSGNSFMHWYQQKPGQPPKLLIYLGNRLESGVPDRFSGSGSRTDFTLTISSLQAEDVAVYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 7 aa, Artificial Sequence
  Synthetic ABV2.13 VHCDR1 (Chothia)
GYTFTTF

SEQ ID NO: 131 - protein, 6 aa, Artificial Sequence
  Synthetic ABV2.13 VHCDR2 (Chothia)
NTYSGV

SEQ ID NO: 132 - protein, 6 aa, Artificial Sequence
  Synthetic ABV2.13 VHCDR3 (Chothia)
RSNFAY

SEQ ID NO: 133 - protein, 15 aa, Artificial Sequence
  Synthetic ABV2.13 VLCDR1 (Chothia)
RASQTVDSSGNSFMH

SEQ ID NO: 134 - protein, 7 aa, Artificial Sequence
  Synthetic ABV2.13 VLCDR2 (Chothia)
LGNRLES

SEQ ID NO: 135 - protein, 9 aa, Artificial Sequence
  Synthetic ABV2.13 VLCDR3 (Chothia)
QQSNEDPWT

SEQ ID NO: 136 - protein, 5 aa, Artificial Sequence
  Synthetic ABV2.13 VHCDR1 (Kabat)
TFGMS

SEQ ID NO: 137 - protein, 17 aa, Artificial Sequence
  Synthetic ABV2.13 VHCDR2 (Kabat)
WINTYSGVPHYAQGFTG

SEQ ID NO: 138 - protein, 6 aa, Artificial Sequence
  Synthetic ABV2.13 VHCDR3 (Kabat)
RSNFAY

SEQ ID NO: 139 - protein, 15 aa, Artificial Sequence
  Synthetic ABV2.13 VLCDR1 (Kabat)
RASQTVDSSGNSFMH

SEQ ID NO: 140 - protein, 7 aa, Artificial Sequence
  Synthetic ABV2.13 VLCDR2 (Kabat)
LGNRLES

SEQ ID NO: 141 - protein, 9 aa, Artificial Sequence
  Synthetic ABV2.13 VLCDR3 (Kabat)
QQSNEDPWT

SEQ ID NO: 142 - protein, 8 aa, Artificial Sequence
  Synthetic ABV2.13 VHCDR1 (IMGT)
GYTFTTFG

SEQ ID NO: 143 - protein, 8 aa, Artificial Sequence
  Synthetic ABV2.13 VHCDR2 (IMGT)
INTYSGVP

SEQ ID NO: 144 - protein, 8 aa, Artificial Sequence
  Synthetic ABV2.13 VHCDR3 (IMGT)
ARRSNFAY

SEQ ID NO: 145 - protein, 10 aa, Artificial Sequence
  Synthetic ABV2.13 VLCDR1 (IMGT)
QTVDSSGNSF

SEQ ID NO: 146 - protein, 3 aa, Artificial Sequence
  Synthetic ABV2.13 VLCDR2 (IMGT)
LGN

SEQ ID NO: 147 - protein, 9 aa, Artificial Sequence
  Synthetic ABV2.13 VLCDR3 (IMGT)
QQSNEDPWT

SEQ ID NO: 148 - protein, 115 aa, Artificial Sequence
  Synthetic ABV2.13 VH
QVQLVQSGSELKKPGASVKVSCKASGYTFTTFGMSWVRQAPGQGLEWMGWINTYSGVPHYAQGFTGRFVFSLDTSVSTAYLQISSLKAEDTAVYFCARRSNFAYWGQGTLVTVSS

SEQ ID NO: 149 - protein, 111 aa, Artificial Sequence
  Synthetic ABV2.13 VL
DIVLTQSPDSLAVSLGERATINCRASQTVDSSGNSFMHWYQQKPGQPPKLLIYLGNRLESGVPDRFSGSGSRTDFTLTISSLQAEDVAVYYCQQSNEDPWTFGGGTKVEIK

SEQ ID NO: 150 - protein, 444 aa, Artificial Sequence; identical to SEQ ID NO: 128
  Synthetic ABV2.15 HC

SEQ ID NO: 151 - protein, 218 aa, Artificial Sequence; identical to SEQ ID NO: 107
  Synthetic ABV2.15 LC

SEQ ID NO: 152 - protein, 7 aa, Artificial Sequence
  Synthetic ABV2.15 VHCDR1 (Chothia)
GYTFTTF

SEQ ID NO: 153 - protein, 6 aa, Artificial Sequence
  Synthetic ABV2.15 VHCDR2 (Chothia)
NTYSGV

SEQ ID NO: 154 - protein, 6 aa, Artificial Sequence
  Synthetic ABV2.15 VHCDR3 (Chothia)
RSNFAY

SEQ ID NO: 155 - protein, 15 aa, Artificial Sequence
  Synthetic ABV2.15 VLCDR1 (Chothia)
RASESLTASGNSFMH

SEQ ID NO: 156 - protein, 7 aa, Artificial Sequence
  Synthetic ABV2.15 VLCDR2 (Chothia)
RASNLES

SEQ ID NO: 157 - protein, 9 aa, Artificial Sequence
  Synthetic ABV2.15 VLCDR3 (Chothia)
QQSRHVNWT

SEQ ID NO: 158 - protein, 5 aa, Artificial Sequence
  Synthetic ABV2.15 VHCDR1 (Kabat)
TFGMS

SEQ ID NO: 159 - protein, 17 aa, Artificial Sequence
  Synthetic ABV2.15 VHCDR2 (Kabat)
WINTYSGVPHYAQGFTG

SEQ ID NO: 160 - protein, 6 aa, Artificial Sequence
  Synthetic ABV2.15 VHCDR3 (Kabat)
RSNFAY

SEQ ID NO: 161 - protein, 15 aa, Artificial Sequence
  Synthetic ABV2.15 VLCDR1 (Kabat)
RASESLTASGNSFMH

SEQ ID NO: 162 - protein, 7 aa, Artificial Sequence
  Synthetic ABV2.15 VLCDR2 (Kabat)
RASNLES

SEQ ID NO: 163 - protein, 9 aa, Artificial Sequence
  Synthetic ABV2.15 VLCDR3 (Kabat)
QQSRHVNWT

SEQ ID NO: 164 - protein, 8 aa, Artificial Sequence
  Synthetic ABV2.15 VHCDR1 (IMGT)
GYTFTTFG

SEQ ID NO: 165 - protein, 8 aa, Artificial Sequence
  Synthetic ABV2.15 VHCDR2 (IMGT)
INTYSGVP

SEQ ID NO: 166 - protein, 8 aa, Artificial Sequence
  Synthetic ABV2.15 VHCDR3 (IMGT)
ARRSNFAY

SEQ ID NO: 167 - protein, 10 aa, Artificial Sequence
  Synthetic ABV2.15 VLCDR1 (IMGT)
ESLTASGNSF

SEQ ID NO: 168 - protein, 3 aa, Artificial Sequence
  Synthetic ABV2.15 VLCDR2 (IMGT)
RAS

SEQ ID NO: 169 - protein, 9 aa, Artificial Sequence
  Synthetic ABV2.15 VLCDR3 (IMGT)
QQSRHVNWT

SEQ ID NO: 170 - protein, 115 aa, Artificial Sequence; identical to SEQ ID NO: 148
  Synthetic ABV2.15 VH

SEQ ID NO: 171 - protein, 111 aa, Artificial Sequence; identical to SEQ ID NO: 127
  Synthetic ABV2.15 VL